(19) United States
(12) Patent Application Publication
(10) Pub. No.: US 2025/0017106 A1
(43) Pub. Date: Jan. 9, 2025

(54) PLURALITY OF HOST MATERIALS AND ORGANIC ELECTROLUMINESCENT DEVICE COMPRISING THE SAME

(71) Applicant: DuPont Specialty Materials Korea Ltd., Chungcheongnam-do (KR)

(72) Inventors: Hyo-Jung LEE, Gyeonggi-do (KR); So-Young JUNG, Gyeonggi-do (KR); Hyun-Ju KANG, Gyeonggi-do (KR); Hee-Ryong KANG, Gyeonggi-do (KR); So-Mi PARK, Gyeonggi-do (KR); Ji-Song JUN, Gyeonggi-do (KR); Sang-Hee CHO, Gyeonggi-do (KR)

(21) Appl. No.: 18/677,049

(22) Filed: May 29, 2024

(30) Foreign Application Priority Data
Jun. 13, 2023 (KR) 10-2023-0075517
Apr. 17, 2024 (KR) 10-2024-0051457

(51) Int. Cl. H10K 85/60

(52) U.S. Cl. CPC H10K 85/6572; H10K 85/622; H10K 85/654; H10K 85/6574; H10K 85/6576

(57) ABSTRACT

The present disclosure relates to a plurality of host materials, and an organic electroluminescent device comprising the same. By comprising a specific combination of compounds according to the present disclosure as a plurality of host materials, it is possible to provide an organic electroluminescent device having improved luminous efficiency, and/or lifetime properties compared to conventional organic electroluminescent devices.

### TECHNICAL FIELD

The present disclosure relates to a plurality of host materials, and an organic electroluminescent device comprising the same.

### BACKGROUND ART

In 1987, Tang et al. of Eastman Kodak first developed a small molecular green organic electroluminescent device (OLED) by using a TPD/Alq3 bi-layer consisting of a light-emitting layer and a charge transport layer. Thereafter, the development of OLEDs proceeded rapidly, and OLEDs have since been commercialized. Currently, OLEDs mainly use phosphorescent materials having excellent luminous efficiency in panel implementation. However, in many applications such as TVs and lightings, the lifetime of OLEDs is insufficient and higher efficiency of OLEDs is still required. Typically, as the luminance of an OLED increases, the lifetime of the OLED becomes shorter. Therefore, an OLED having high luminous efficiency and/or a long lifetime is required for long-term use and high display resolution.

In order to enhance luminous efficiency, driving voltage and/or lifetime, various materials or concepts have been proposed for an organic layer of an OLED. However, these have not been satisfactory in practical use. In addition, there has been a need to develop an organic electroluminescent material having more improved performance, for example, improved driving voltage, luminous efficiency, power efficiency, and/or lifetime properties compared to a combination of specific compounds which were previously disclosed.

Meanwhile, Korean Patent Application Laid-open No. 2020-0014189 discloses a compound with a fused structure comprising benzocarbazole and azulene, and a compound with a fused structure comprising indolocarbazole and azepine as a host material. However, the aforementioned reference does not specifically disclose a plurality of host materials comprising a specific combination of compounds according to the present disclosure.

### DISCLOSURE OF INVENTION

### Technical Problem

The objective of the present disclosure is firstly, to provide a plurality of host materials capable of providing an organic electroluminescent device having low driving voltage, and/or high luminous efficiency and/or excellent lifetime properties, and secondly, to provide an organic electroluminescent device comprising the host materials.

### Solution to Problem

As a result of intensive studies to solve the technical problems, the present inventors found that the above objective can be achieved by a plurality of host materials comprising a first host material comprising a compound represented by the following Formula 1, and a second host material comprising a compound represented by the following Formula 2.

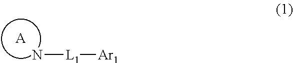

In Formula 1,
L₁ represents a single bond, a substituted or unsubstituted (C6-C30)arylene, or a substituted or unsubstituted (3- to 30-membered)heteroarylene;
Ar₁ represents hydrogen, deuterium, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, —N-(L₂-Ar₂)(L₃-Ar₃), a substituted or unsubstituted (C6-C30)aryl, or a substituted or unsubstituted (3- to 30-membered)heteroaryl;
L₂ and L₃ each independently represent a single bond, a substituted or unsubstituted (C6-C30)arylene, or a substituted or unsubstituted (3- to 30-membered)heteroarylene;
Ar₂ and Ar₃ each independently represent a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl;

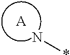

is represented by the following Formula 1-1 or 1-2;

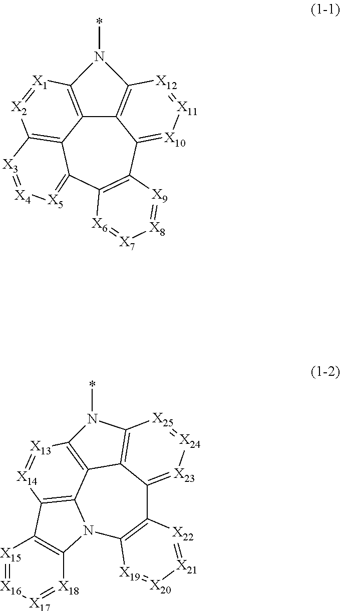

In Formulas 1-1 and 1-2,
X₁ to X₂₅ each independently represent N or CRₐ;
Rₐ each independently represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a ring(s);
* represents a site linked with L₁;

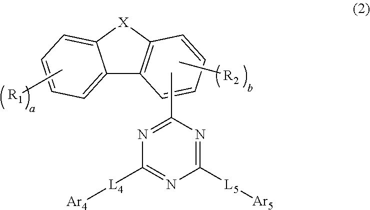

In Formula 2,
X represents O or S; L₄ and L₅ each independently represent a single bond, a substituted or unsubstituted (C6-C30)arylene, or a substituted or unsubstituted (3- to 30-membered)heteroarylene;
Ar₄ and Ar₅ each independently represent a substituted or unsubstituted (C6-C30)aryl, or a substituted or unsubstituted (3- to 30-membered)heteroaryl;
R₁ and R₂ each independently represent hydrogen, deuterium, a substituted or unsubstituted (C1-C30)alkyl, a substituted or unsubstituted (C6-C30)aryl, a substituted or unsubstituted (3- to 30-membered)heteroaryl, or —SiR₁₁R₁₂R₁₃;
a represents an integer of 1 to 4, b represents an integer of 1 to 3, and if a and b each represent an integer of 2 or more, each of R₁ and each of R₂ may be the same as or different from each other.

### Advantageous Effects of Invention

An organic electroluminescent device having low driving voltage, high luminous efficiency, and/or excellent lifetime properties can be produced by using the plurality of host materials of the present disclosure.

### MODE FOR THE INVENTION

Hereinafter, the present disclosure will be described in detail. However, the following description is intended to explain the present disclosure, and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure is a combination of at least two host materials, and may selectively further comprise conventional materials comprised in an organic electroluminescent material. At least two compounds comprised in the plurality of host materials of the present disclosure may be comprised together in one light-emitting layer or may each be comprised in different light-emitting layers. For example, the at least two host materials may be mixture-evaporated or co-evaporated, or may be individually evaporated.

Herein, the term “(C1-C30)alkyl” is meant to be a linear or branched alkyl having 1 to 30 carbon atoms constituting a chain, in which the number of carbon atoms is preferably 1 to 10, and more preferably 1 to 6. The above alkyl may include methyl, ethyl, n-propyl, isopropyl, n-butyl, isobutyl, tert-butyl, sec-butyl, etc.

Herein, the term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc.

The term “(3- to 7-membered)heterocycloalkyl” is meant to be a cycloalkyl having 3 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc.

The term “(C6-C30)aryl”, and “(C6-C30)arylene” are meant to be a monocyclic or fused ring radical derived from an aromatic hydrocarbon having 6 to 30 ring backbone carbon atoms. The above aryl, and arylene may be partially saturated. The above aryl may comprise a spiro structure. The above aryl may include phenyl, biphenyl, terphenyl, quinquephenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, spiro[cyclopentene-fluoren]yl, spiro[dihydroindene-fluoren]yl, azulenyl, tetramethyldihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl”, or “(3- to 30-membered)heteroarylene” are meant to be an aryl group having 3 to 30 ring backbone atoms, and including at least one heteroatom selected from the group consisting of B, N, O, S, Si, and P. The number of heteroatoms is preferably 1 to 4. The above heteroaryl may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, naphthooxazolyl, benzofuroquinolinyl, benzofuroquinazolinyl, benzofuronaphthyridinyl, benzofuropyrimidinyl, naphthofuropyrimidinyl, benzothienoquinolyl, benzothienoquinazolinyl, naphthyridinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, benzoquinazolinyl, quinoxalinyl, benzoquinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which each represent the relative positions of substituents. Ortho indicates that two substituents are adjacent to each other, and for example, when two substituents in a benzene derivative occupy positions 1 and 2 or positions 2 and 3, this is called an ortho position. Meta indicates that two substituents are at positions 1 and 3, and for example, when two substituents in a benzene derivative occupy positions 1 and 3, this is called a meta position. Para indicates that two substituents are at positions 1 and 4, and for example, when two substituents in a benzene derivative occupy positions 1 and 4, this is called a para position.

Herein, “substituted” in the expression “substituted or unsubstituted” means that a hydrogen atom in a certain functional group is replaced with another atom or another functional group, i.e., a substituent, and also includes cases where the hydrogen atom is replaced with a group formed by a linkage of two or more substituents of the above substituents. For example, the “group formed by a linkage of two or more substituents” may be pyridine-triazine. That is, pyridine-triazine may be interpreted as one heteroaryl substituent, or as substituents in which two heteroaryl substituents are linked. Herein, the substituent(s) of the substituted (C1-C30)alky, the substituted (C6-C30)aryl(ene), the substituted (3- to 7-membered) heteroaryl(ene), the substituted (C3-C30)cycloalkyl, the substituted (C3-C30)cycloalkenyl, the substituted (3- to 7-membered)heterocycloalkyl, the substituted (C1-C30)alkoxy, the substituted tri(C1-C30)alkylsilyl, the substituted di(C1-C30)alkyl(C6-C30)arylsilyl, the substituted (C1-C30)alkyldi(C6-C30)arylsilyl, the substituted tri(C6-C30)arylsilyl, the substituted mono- or di-(C1-C30)alkylamino, the substituted mono- or di-(C6-C30)arylamino, and the substituted (C1-C30)alkyl(C6-C30)arylamino each independently are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (5- to 30-membered)heteroaryl unsubstituted or substituted with a (C6-C30)aryl; a (C6-C30)aryl unsubstituted or substituted with a (5- to 30-membered)heteroaryl; a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino unsubstituted or substituted with a (C1-C30)alkyl; a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and (C1-C30)alkyl(C6-C30)aryl. For example, the substituent(s) each independently may be at least one selected from the group consisting of a carbazolyl, a phenyl unsubstituted or substituted with naphthyl, a biphenyl, a naphthyl, a dibenzofurany, a phenanthrenyl, a benzocarbazolyl, dimethylfluorenyl, or a naphthyl unsubstituted or substituted with phenyl.

In the formulas of the present disclosure, when a ring is formed by a linkage of adjacent substituents, the ring may be a substituted or unsubstituted, mono- or polycyclic, (3- to 30-membered) alicyclic or aromatic ring, or the combination thereof, which is formed by linkage of at least two adjacent substituents. In addition, the formed ring may contain at least one heteroatom selected from B, N, O, S, Si, and P, preferably at least one heteroatom selected from N, O, and S. According to one embodiment of the present disclosure, the number of the ring backbone atoms is 5 to 20, and according to another embodiment of the present disclosure, the number of the ring backbone atoms is 5 to 15.

In the formulas of the present disclosure, the heteroaryl or heteroarylene each independently may comprise at least one heteroatom selected from B, N, O, S, Si, and P. In addition, the heteroatom may be a combination of at least one selected form the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

Hereinafter, a plurality of host materials will be described in more detail.

A plurality of host materials according to the present disclosure comprises a first host material comprising a compound represented by the Formula 1, and a second host material comprising a compound represented by the Formula 2. The plurality of host materials may be comprised in a light-emitting layer of an organic electroluminescent device according to one example.

The first host material according to the one embodiment of the present disclosure may be represented by the following Formula 1.

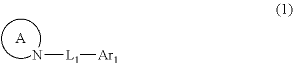

In Formula 1,

L₁ represents a single bond, a substituted or unsubstituted (C6-C30)arylene, or a substituted or unsubstituted (3- to 30-membered)heteroarylene. According to one embodiment of the present disclosure, L₁ may represent a single bond, a substituted or unsubstituted (C6-C25)arylene, or a substituted or unsubstituted (5- to 25-membered)heteroarylene, preferably a single bond, a substituted or unsubstituted (C6-C18)arylene, or a substituted or unsubstituted (5- to 18-membered)heteroarylene, more preferably a single bond, a substituted or unsubstituted phenylene, a substituted or unsubstituted biphenylene, a substituted or unsubstituted naphthylene, a substituted or unsubstituted phenanthrenylene, a substituted or unsubstituted dibenzofuranylene, a substituted or unsubstituted carbazolylene, or a substituted or unsubstituted benzocarbazolylene. For example, L₁ may represent a single bond, a carbazolylene unsubstituted or substituted with a phenyl or deuterium, a dibenzofuranylene, a naphthylene, a phenylene unsubstituted or substituted with a carbazolyl or deuterium, a biphenylene, a phenanthrenylene, or a benzocarbazolylene.

Ar₁ represents hydrogen, deuterium, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, —N-(L₂-Ar₂)(L₃-Ar₃), a substituted or unsubstituted (C6-C30)aryl, or a substituted or unsubstituted (3- to 30-membered)heteroaryl. According to one embodiment of the present disclosure, Ar₁ may represent hydrogen, deuterium, a substituted or unsubstituted (C1-C25)alkyl, a substituted or unsubstituted (C3-C30)cycloalkyl, a substituted or unsubstituted (C3-C25)cycloalkenyl, a substituted or unsubstituted (3- to 7-membered)heterocycloalkyl, —N-(L₂-Ar₂)(L₃-Ar₃), a substituted or unsubstituted (C6-C25)aryl, or a substituted or unsubstituted (3- to 30-membered)heteroaryl, preferably hydrogen, a substituted or unsubstituted carbazolyl, a substituted or unsubstituted phenyl, a substituted or unsubstituted terphenyl, a substituted or unsubstituted biphenyl, a substituted or unsubstituted phenanthrenyl, a substituted or unsubstituted naphthyl, a substituted or unsubstituted dibenzofuranyl, a substituted or unsubstituted dibenzothiophenyl, a substituted or unsubstituted benzocarbazolyl, a substituted or unsubstituted dimethylfluorenyl, a substituted or unsubstituted diphenylamino, a substituted or unsubstituted phenyldibenzofuranylamino, or —N-(L₂-Ar₂)(L₃-Ar₃). For example, Ar₁ may represent hydrogen; deuterium; a carbazolyl unsubstituted or substituted with a phenyl, a naphthyl, or biphenyl; a phenyl unsubstituted or substituted with deuterium, a dibenzofuranyl, a phenanthrenyl, a naphthyl, a biphenyl, a benzocarbazolyl, or a dimethylfluorenyl; a terphenyl; a biphenyl unsubstituted or substituted with a phenyl or deuterium; a phenanthrenyl unsubstituted or substituted with a phenyl; a naphthyl unsubstituted or substituted with a phenyl; a dibenzofuranyl unsubstituted or substituted with a phenyl, a biphenyl, or deuterium; a dibenzothiophenyl; a benzocarbazolyl substituted with a phenyl; a dimethylfluorenyl; a dimethylbenzofluorenyl; a spirobifluorenyl; a dibenzoselenophenyl; a phenylcarbazolylamino; a diphenylamino; or —N-(L₂-Ar₂)(L₃-Ar₃).

L₂ and L₃ each independently represent a single bond, a substituted or unsubstituted (C6-C30)arylene, or a substituted or unsubstituted (3- to 30-membered)heteroarylene. According to one embodiment of the present disclosure, L₂ and L₃ each independently may represent a single bond, a substituted or unsubstituted (C6-C25)arylene, or a substituted or unsubstituted (6- to 25-membered)heteroarylene, preferably a single bond, a substituted or unsubstituted (C6-C18)arylene, or a substituted or unsubstituted (6- to 18-membered)heteroarylene. For example, L₂ and L₃ each independently may represent a single bond, a phenylene, or a carbazolylene unsubstituted or substituted with deuterium.

Ar₂ and Ar₃ each independently represent a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl. According to one embodiment of the present disclosure, Ar₂ and Ar₃ each independently may represent a substituted or unsubstituted (C1-C25)alkyl, a substituted or unsubstituted (C3-C25)cycloalkyl, a substituted or unsubstituted (C3-C25)cycloalkenyl, a substituted or unsubstituted (3- to 7-membered)heterocycloalkyl, a substituted or unsubstituted (C6-C25)aryl, or a substituted or unsubstituted (3- to 25-membered)heteroaryl, preferably a substituted or unsubstituted (C3-C18)alkyl, a substituted or unsubstituted (C6-C18)cycloalkyl, a substituted or unsubstituted (C6-C18)cycloalkenyl, a substituted or unsubstituted (3- to 7-membered)heterocycloalkyl, a substituted or unsubstituted (C6-C18)aryl, or a substituted or unsubstituted (3- to 18-membered)heteroaryl, more preferably a substituted or unsubstituted phenyl, a substituted or unsubstituted carbazolyl, dibenzofuranyl, or dibenzothiophenyl. For example, Ar₂ and Ar₃ each independently may represent a phenyl unsubstituted or substituted with deuterium, a dibenzofuranyl, or a carbazolyl; a carbazolyl unsubstituted or substituted with deuterium, a phenyl, a biphenyl, or a naphthyl; a dibenzofuranyl unsubstituted or substituted with deuterium; or a dibenzothiophenyl.

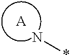

may be represented by the following formula 1-1 or formula 1-2.

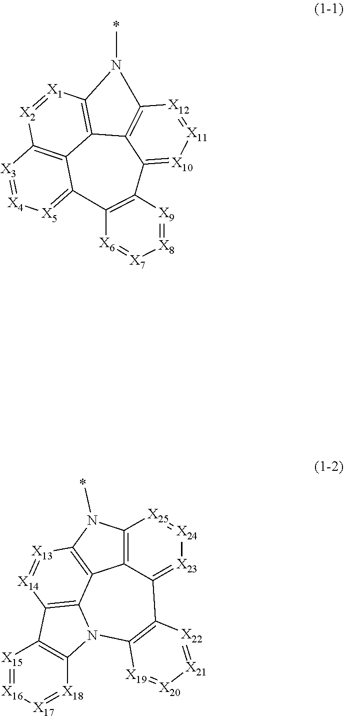

In formulas 1-1 and 1-2,
X₁ to X₂₅ each independently represent N or CRₐ.
Rₐ each independently represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a ring(s). For example, Rₐ may represent hydrogen, deuterium, a substituted or unsubstituted phenyl, or may be linked to an adjacent substituent(s) to form a substituted or unsubstituted aromatic a ring(s).

According to one embodiment of the present disclosure, X₁, X₂, X₁₀, X₁₃, and X₁₅ to X₁₈ each independently may represent CRₐ, and here Rₐ may be hydrogen or deuterium, or may be linked to an adjacent substituent(s) to form a benzene ring(s).
represents a site linked with L₁.

According to one embodiment of the present disclosure, Formula 1 may be represented by the following Formula 1-1-1.

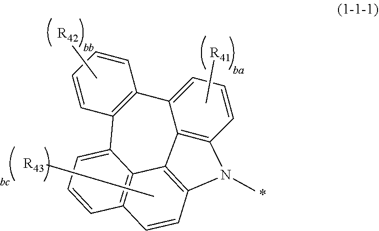

In Formula 1-1-1,
R₄₁ to R₄₃ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a ring(s). For example, R₄₁ to R₄₃ each independently may represent hydrogen, deuterium, or a substituted or unsubstituted phenyl, or may be fused with each other to form a substituted or unsubstituted aromatic a ring(s).
ba may represent an integer of 1 to 3, bb may represent an integer of 1 to 4, bc may represent an integer of 1 to 5, and if ba, bb, and bc each represent an integer of 2 or more, each of R₄₁, each of R₄₂, and each of R₄₃, may be the same as or different from one another.

According to one embodiment of the present disclosure, the Formula 1-2 may be represented by the following Formula 1-2-1.

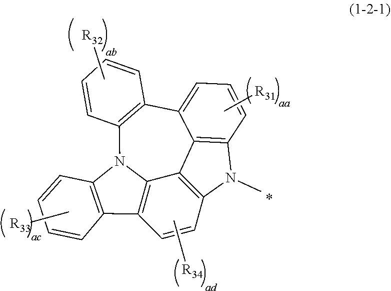

In Formula 1-2-1,
R₃₁ to R₃₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a ring(s). For example, R₃₁ to R₃₄ each independently may represent hydrogen, deuterium, or a substituted or unsubstituted phenyl, or may be fused with each other to form a substituted or unsubstituted aromatic a ring(s).
aa may represent an integer of 1 to 3, ab and ac each independently may represent an integer of 1 to 4, ad may represent an integer of 1 or 2, and if aa, ab, ac, and ad each represent an integer of 2 or more, each of R₃₁, each of R₃₂, each of R₃₃, and each of R₃₄ may be the same as or different from one another.

According to one embodiment of the present disclosure, at least one of Ar₂ and Ar₃ in the Formula 1 may be represented by any one of the following Formulas 1-3-1 to 1-3-4.

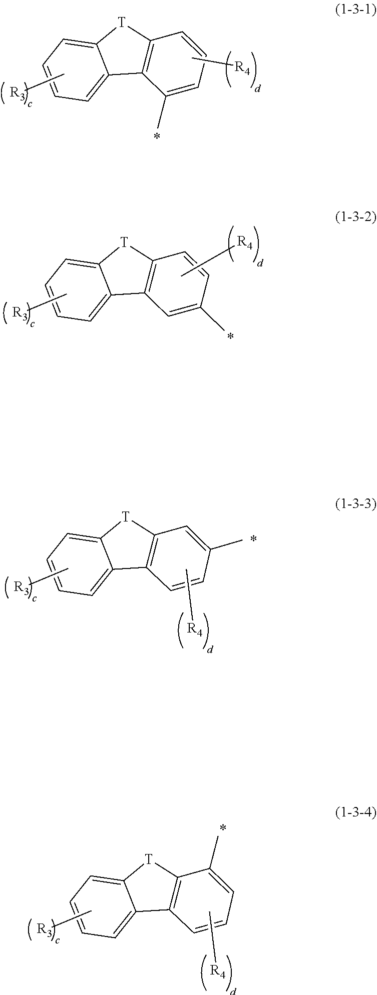

In formulas 1-3-1 to 1-3-4,
T represents O, S, CR₅R₆, NR₇, or Se.
R₃ to R₇ have the same definition as Rₐ, c represents an integer of 1 to 4, d represents an integer of 1 to 3, and if c and d each represent an integer of 2 or more, each of R₃ and each of R₄ may be the same as or different from each other.

For example, T may represent O, S, CR₅R₆, NR₇, or Se, in this case, R₅ to R₇ each independently may represent a methyl, a phenyl, a biphenyl, or a naphthyl.

As another host material according to one embodiment of the present disclosure, the second host compound is represented by the following Formula 2.

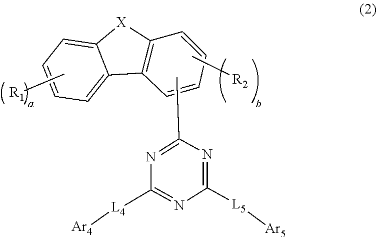

In Formula 2,
X represents O or S.
L₄ and L₅ each independently represent a single bond, a substituted or unsubstituted (C6-C30)arylene, or a substituted or unsubstituted (3- to 30-membered)heteroarylene. According to one embodiment of the present disclosure, L₄ and L₅ each independently represent a single bond, a substituted or unsubstituted (C6-C25)arylene, or a substituted or unsubstituted (3- to 25-membered)heteroarylene, preferably a single bond, a substituted or unsubstituted phenylene, a substituted or unsubstituted biphenylene, a substituted or unsubstituted terphenylene, a substituted or unsubstituted phenanthrenylene, a substituted or unsubstituted naphthylene, or a substituted or unsubstituted dibenzofuranylene. For example, L₄ and L₅ each independently may represent a single bond, a phenylene unsubstituted or substituted with a naphthyl(s), a biphenylene, a terphenylene, a phenanthrenylene, a dibenzofuranylene unsubstituted or substituted with a phenyl(s) or a naphthyl(s), a naphthylene unsubstituted or substituted with a phenyl(s), a phenanthro[3,4-d]oxazolyl, a phenanthro[4,3-d]oxazolyl, or a phenanthro[3,4-d]thiazolyl.
Ar₄ and Ar₅ each independently represent a substituted or unsubstituted (C6-C30)aryl, or a substituted or unsubstituted (3- to 30-membered)heteroaryl. According to one embodiment of the present disclosure, Ar₄ and Ar₅ each independently may represent a substituted or unsubstituted (C6-C25)aryl, preferably a substituted or unsubstituted phenyl, a substituted or unsubstituted biphenyl, a substituted or unsubstituted terphenyl, a substituted or unsubstituted quaterphenyl, a substituted or unsubstituted benzophenanthrenyl, a substituted or unsubstituted dibenzofuranyl, a substituted or unsubstituted naphthyl, a substituted or unsubstituted phenanthrenyl, a substituted or unsubstituted fluoranthrenyl, or a substituted or unsubstituted triphenylenyl. For example, Ar₄ and Ar₅ each independently may represent a phenyl unsubstituted or substituted with a naphthyl(s), a phenanthrenyl(s), biphenyl(s), terphenyl(s), or a naphthyl(s) substituted with a phenyl(s); a biphenyl unsubstituted or substituted with a phenyl(s), or a biphenyl(s); a terphenyl unsubstituted or substituted with a phenyl(s); a quaterphenyl; a benzophenanthrenyl; a dibenzofuranyl unsubstituted or substituted with a phenyl(s), a biphenyl(s), a naphthyl(s) substituted with a phenyl(s), a naphthyl(s), a phenyl(s) substituted with a naphthyl(s), or a phenanthrenyl(s); a naphthyl unsubstituted or substituted with a phenyl(s), a biphenyl(s), a naphthyl(s); a phenanthrenyl unsubstituted or substituted with a phenyl(s), a biphenyl(s), a naphthyl(s); a fluoranthrenyl; a triphenylenyl; a phenanthro[3,4-d]oxazolyl substituted with a phenyl(s); a phenanthro[4,3-d]oxazolyl substituted with a phenyl(s); or a phenanthro[3,4-d]thiazolyl substituted with a phenyl(s).
R₁ and R₂ each independently represent hydrogen, deuterium, a substituted or unsubstituted (C1-C30)alkyl, a substituted or unsubstituted (C6-C30)aryl, a substituted or unsubstituted (3- to 30-membered)heteroaryl, or —SiR₁₁R₁₂R₁₃. According to one embodiment of the present disclosure, R₁ and R₂ each independently may represent hydrogen, deuterium, a substituted or unsubstituted (C1-C25)alkyl, a substituted or unsubstituted (C6-C25)aryl, a substituted or unsubstituted (3- to 25-membered)heteroaryl, preferably hydrogen, a substituted or unsubstituted phenyl, a substituted or unsubstituted biphenyl, a substituted or unsubstituted naphthyl, or a substituted or unsubstituted phenanthrenyl. For example, R₁ and R₂ each independently ay represent hydrogen, a phenyl unsubstituted or substituted with a naphthyl(s), a biphenyl, a naphthyl unsubstituted or substituted with a phenyl(s), or a phenanthrenyl(s).
a represents an integer of 1 to 4, b represents an integer of 1 to 3, and if a and b each represent an integer of 2 or more, each of R₁ and each of R₂ may be the same as or different from each other.

According to one embodiment of the present disclosure, Formula 2 may be represented by any one of the following Formulas 2-1 to 2-4.

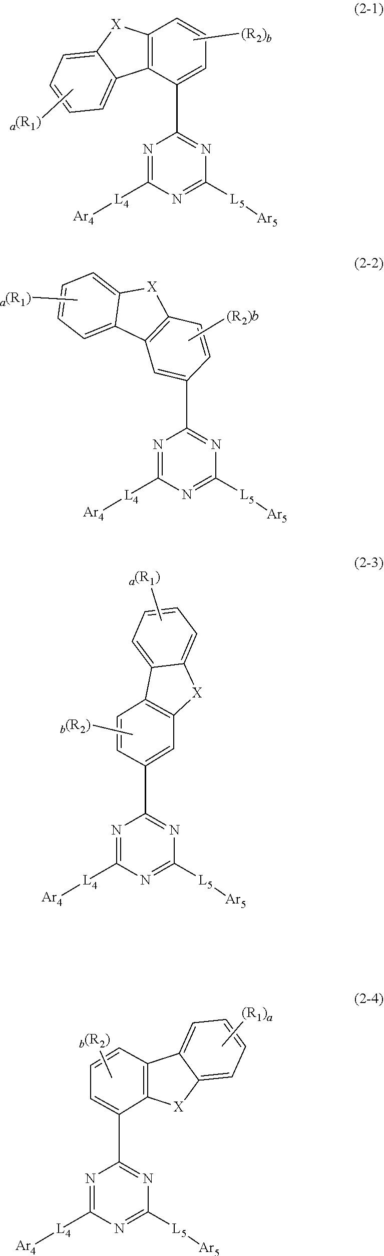

In Formulas 2-1 to 2-4,
X, R₁, R₂, L₄, L₅, Ar₄, Ar₅, a, and b have the same definitions as defined in Formula 2.

According to one embodiment of the present disclosure, the compound represented by Formula 1 may be selected from the group consisting of the following compounds, but is not limited thereto.

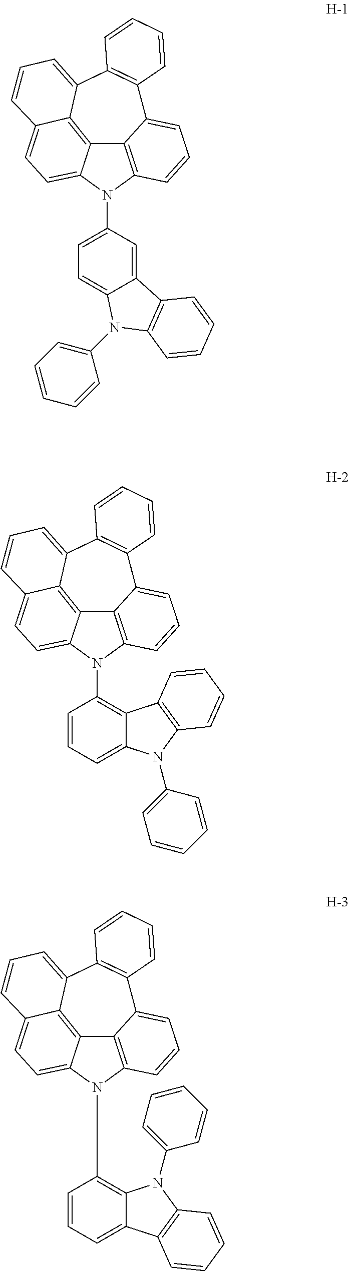 [SMILES: c1ccc(-n2c3ccccc3c3cc(-n4c5cccc6c5c5c7c(cccc7ccc54)-c4ccccc4-6)ccc32)cc1 ; c1ccc(-n2c3ccccc3c3c(-n4c5cccc6c5c5c7c(cccc7ccc54)-c4ccccc4-6)cccc32)cc1 ; c1ccc(-n2c3ccccc3c3cccc(-n4c5cccc6c5c5c7c(cccc7ccc54)-c4ccccc4-6)c32)cc1]

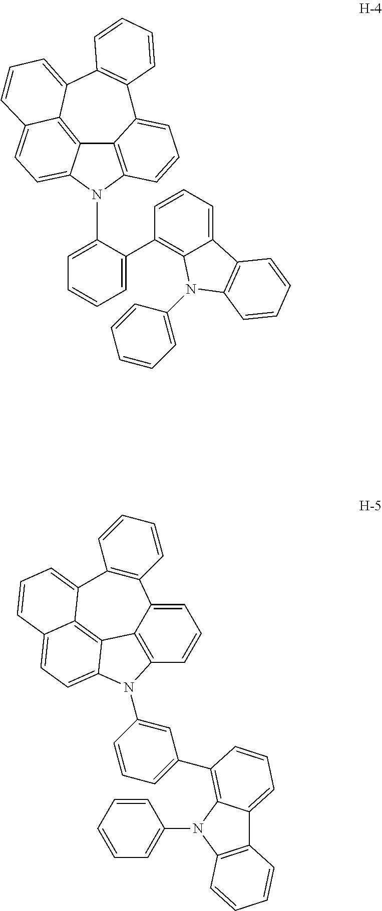 [SMILES: c1ccc(-n2c3ccccc3c3cccc(-c4ccccc4-n4c5cccc6c5c5c7c(cccc7ccc54)-c4ccccc4-6)c32)cc1 ; c1ccc(-n2c3ccccc3c3cccc(-c4cccc(-n5c6cccc7c6c6c8c(cccc8ccc65)-c5ccccc5-7)c4)c32)cc1]

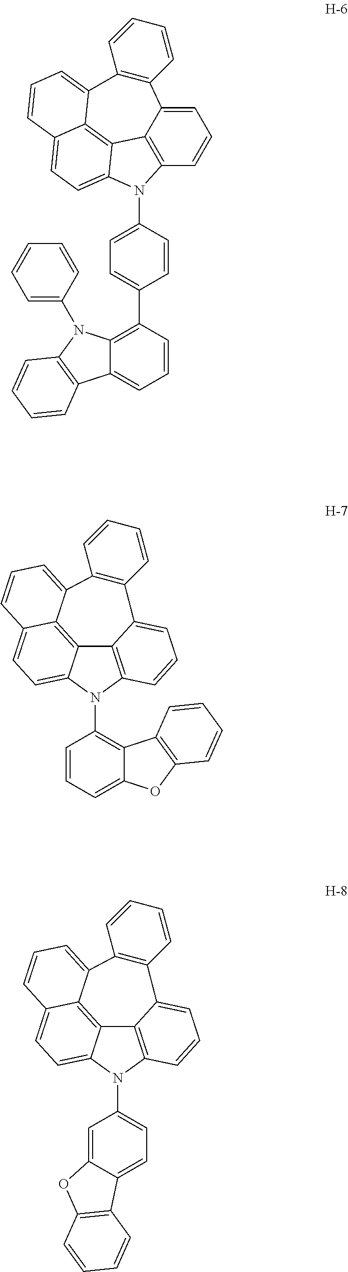 [SMILES: c1ccc(-n2c3ccccc3c3cccc(-c4ccc(-n5c6cccc7c6c6c8c(cccc8ccc65)-c5ccccc5-7)cc4)c32)cc1 ; c1ccc2c(c1)-c1cccc3ccc4c(c13)c1c-2cccc1n4-c1cccc2oc3ccccc3c12 ; c1ccc2c(c1)-c1cccc3ccc4c(c13)c1c-2cccc1n4-c1ccc2c(c1)oc1ccccc12]

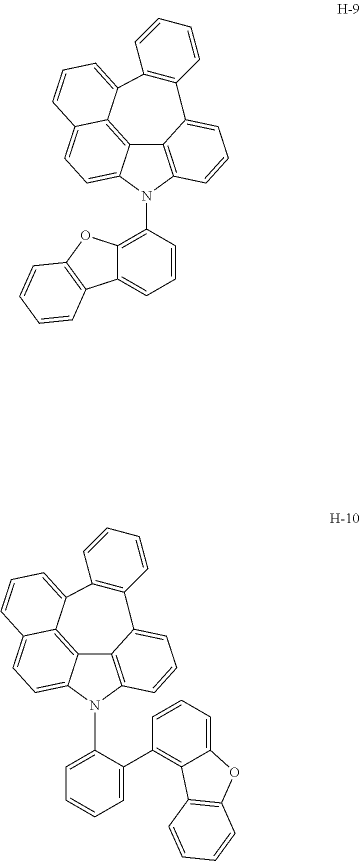 [SMILES: c1ccc2c(c1)-c1cccc3ccc4c(c13)c1c-2cccc1n4-c1cccc2c1oc1ccccc12 ; c1ccc2c(c1)-c1cccc3ccc4c(c13)c1c-2cccc1n4-c1ccccc1-c1cccc2oc3ccccc3c12]

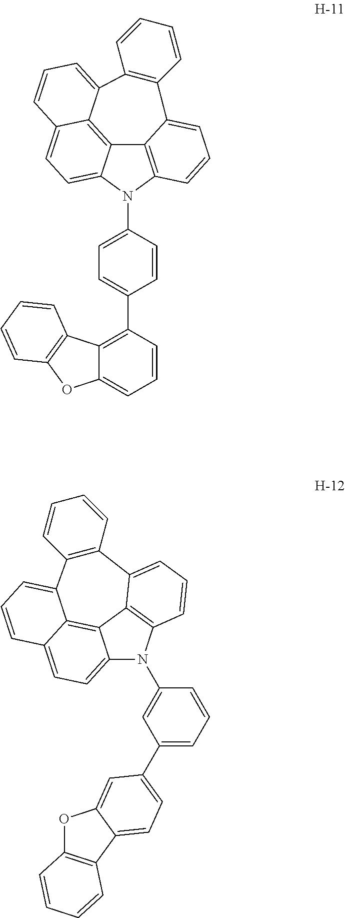 [SMILES: c1ccc2c(c1)-c1cccc3ccc4c(c13)c1c-2cccc1n4-c1ccc(-c2cccc3oc4ccccc4c23)cc1 ; c1cc(-c2ccc3c(c2)oc2ccccc23)cc(-n2c3cccc4c3c3c5c(cccc5ccc32)-c2ccccc2-4)c1]

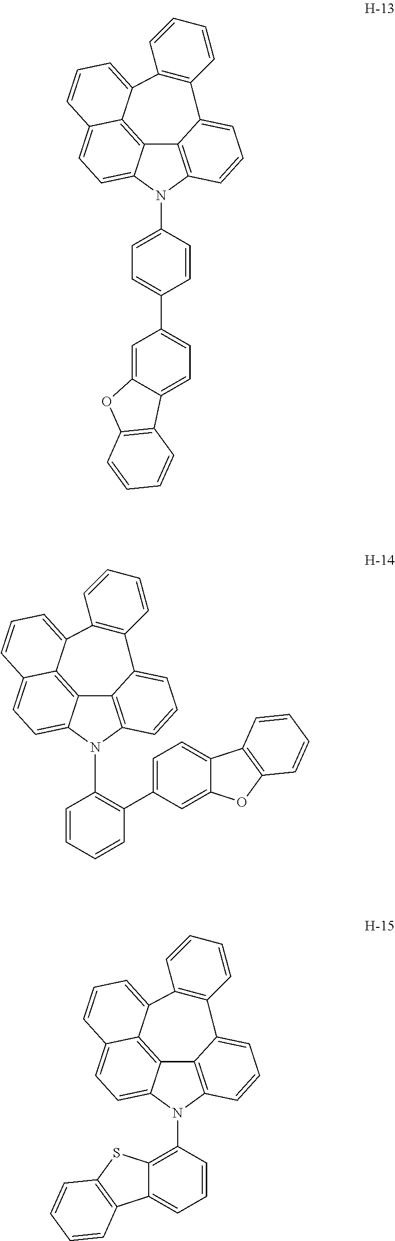 [SMILES: c1ccc2c(c1)-c1cccc3ccc4c(c13)c1c-2cccc1n4-c1ccc(-c2ccc3c(c2)oc2ccccc23)cc1 ; c1ccc2c(c1)-c1cccc3ccc4c(c13)c1c-2cccc1n4-c1ccccc1-c1ccc2c(c1)oc1ccccc12 ; c1ccc2c(c1)-c1cccc3ccc4c(c13)c1c-2cccc1n4-c1cccc2c1sc1ccccc12]

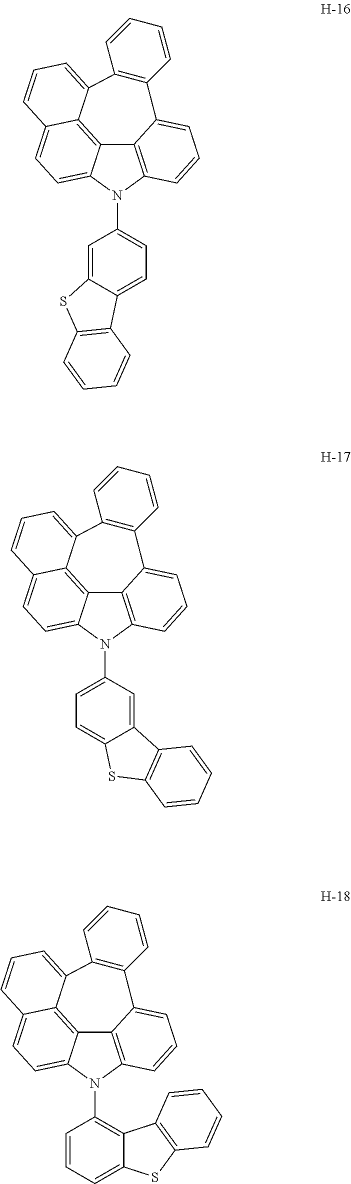 [SMILES: c1ccc2c(c1)-c1cccc3ccc4c(c13)c1c-2cccc1n4-c1ccc2c(c1)sc1ccccc12 ; c1ccc2c(c1)-c1cccc3ccc4c(c13)c1c-2cccc1n4-c1ccc2sc3ccccc3c2c1 ; c1ccc2c(c1)-c1cccc3ccc4c(c13)c1c-2cccc1n4-c1cccc2sc3ccccc3c12]

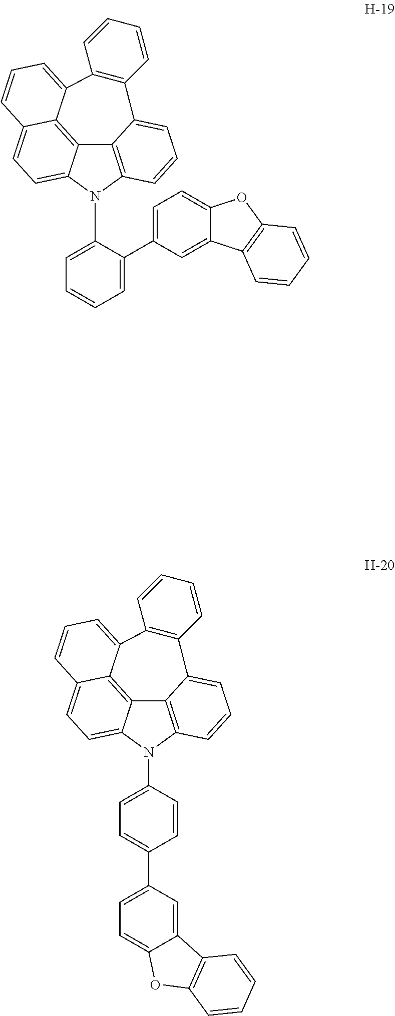 [SMILES: c1ccc2c(c1)-c1cccc3ccc4c(c13)c1c-2cccc1n4-c1ccccc1-c1ccc2oc3ccccc3c2c1 ; c1ccc2c(c1)-c1cccc3ccc4c(c13)c1c-2cccc1n4-c1ccc(-c2ccc3oc4ccccc4c3c2)cc1]

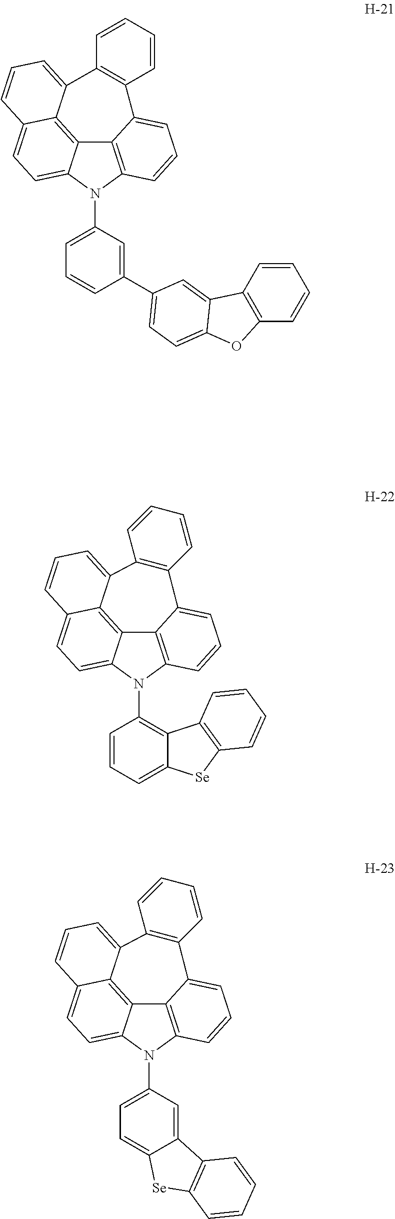 [SMILES: c1cc(-c2ccc3oc4ccccc4c3c2)cc(-n2c3cccc4c3c3c5c(cccc5ccc32)-c2ccccc2-4)c1 ; c1ccc2c(c1)-c1cccc3ccc4c(c13)c1c-2cccc1n4-c1cccc2[se]c3ccccc3c12 ; c1ccc2c(c1)-c1cccc3ccc4c(c13)c1c-2cccc1n4-c1ccc2[se]c3ccccc3c2c1]

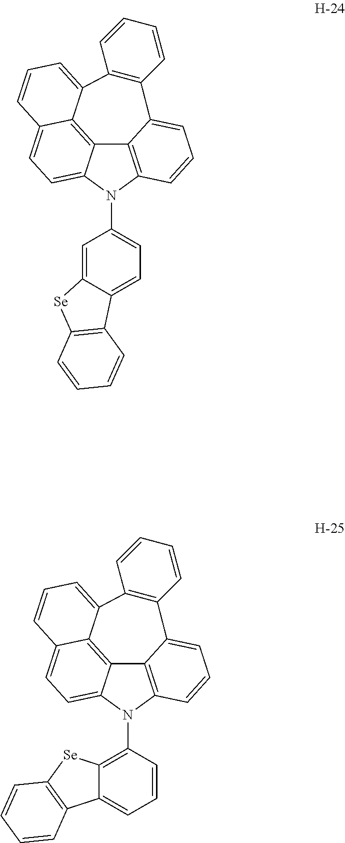 [SMILES: c1ccc2c(c1)-c1cccc3ccc4c(c13)c1c-2cccc1n4-c1ccc2c(c1)[se]c1ccccc12 ; c1ccc2c(c1)-c1cccc3ccc4c(c13)c1c-2cccc1n4-c1cccc2c1[se]c1ccccc12]

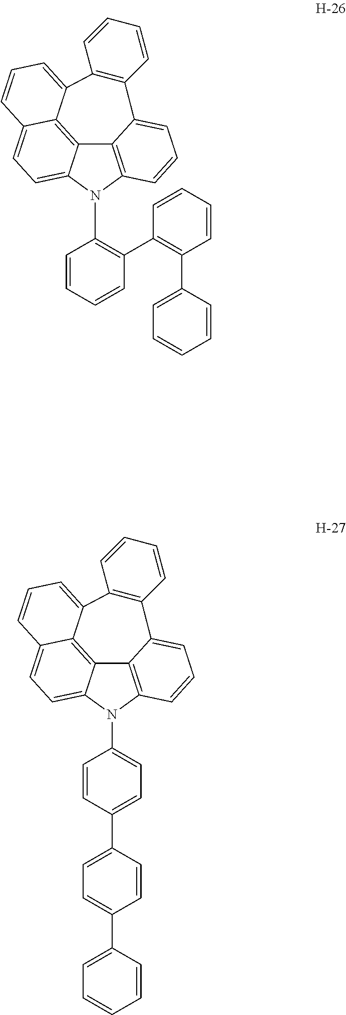 [SMILES: c1ccc(-c2ccccc2-c2ccccc2-n2c3cccc4c3c3c5c(cccc5ccc32)-c2ccccc2-4)cc1 ; c1ccc(-c2ccc(-c3ccc(-n4c5cccc6c5c5c7c(cccc7ccc54)-c4ccccc4-6)cc3)cc2)cc1]

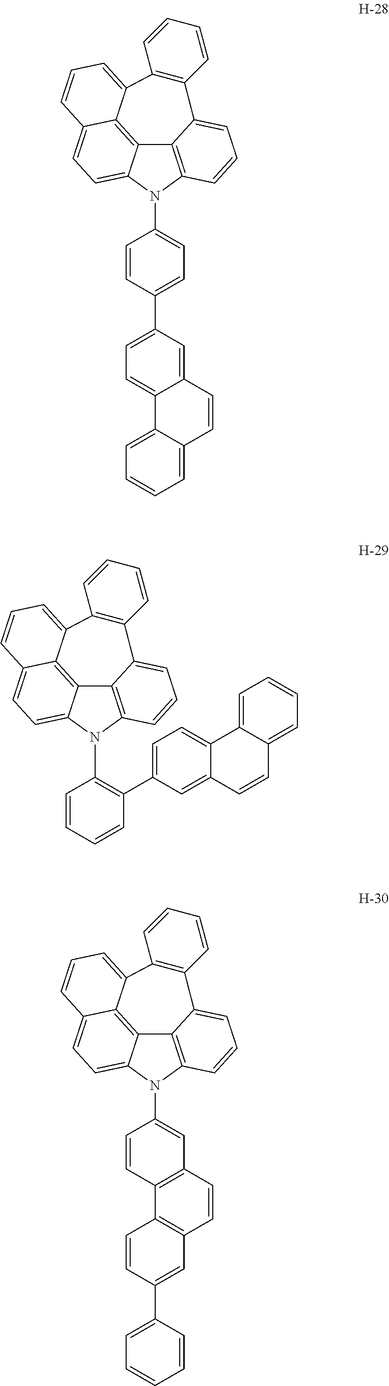 [SMILES: c1ccc2c(c1)-c1cccc3ccc4c(c13)c1c-2cccc1n4-c1ccc(-c2ccc3c(ccc4ccccc43)c2)cc1 ; c1ccc2c(c1)-c1cccc3ccc4c(c13)c1c-2cccc1n4-c1ccccc1-c1ccc2c(ccc3ccccc32)c1 ; c1ccc(-c2ccc3c(ccc4cc(-n5c6cccc7c6c6c8c(cccc8ccc65)-c5ccccc5-7)ccc43)c2)cc1]

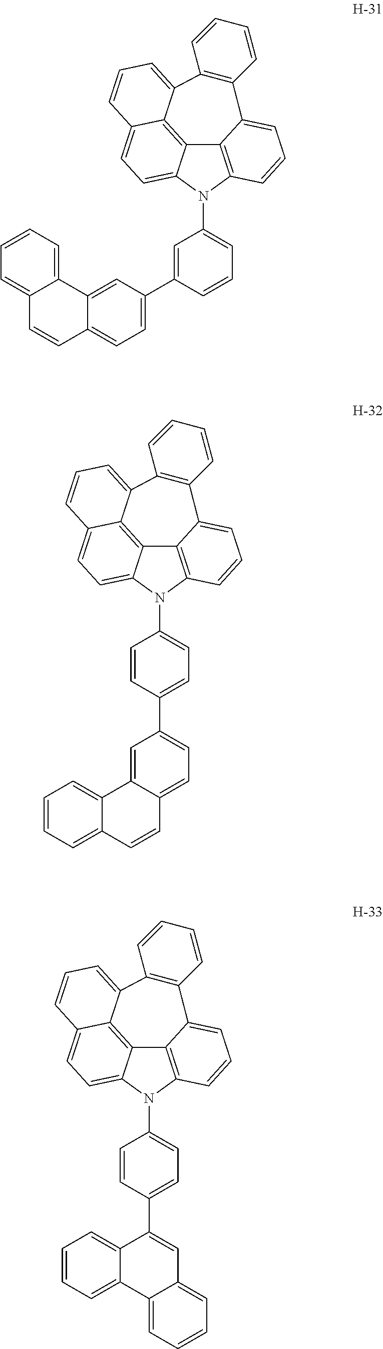 [SMILES: c1cc(-c2ccc3ccc4ccccc4c3c2)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc(-c2ccc3ccc4ccccc4c3c2)cc1 ; c1ccc2c(c1)-c1cccc3ccc4c(c13)c1c-2cccc1n4-c1ccc(-c2cc3ccccc3c3ccccc23)cc1]

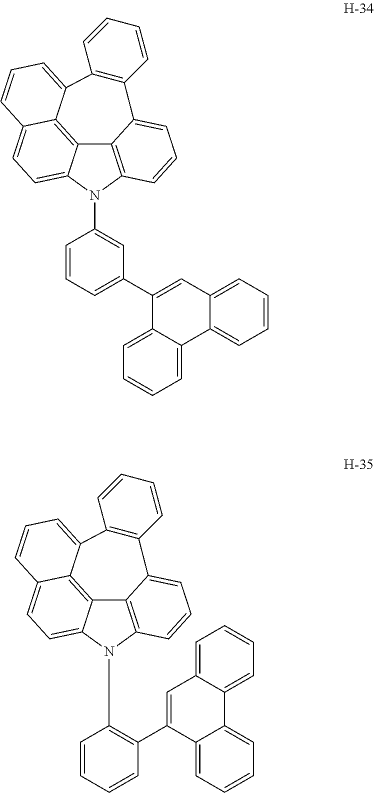 [SMILES: c1cc(-c2cc3ccccc3c3ccccc23)cc(-n2c3cccc4c3c3c5c(cccc5ccc32)-c2ccccc2-4)c1 ; c1ccc2c(c1)-c1cccc3ccc4c(c13)c1c-2cccc1n4-c1ccccc1-c1cc2ccccc2c2ccccc12]

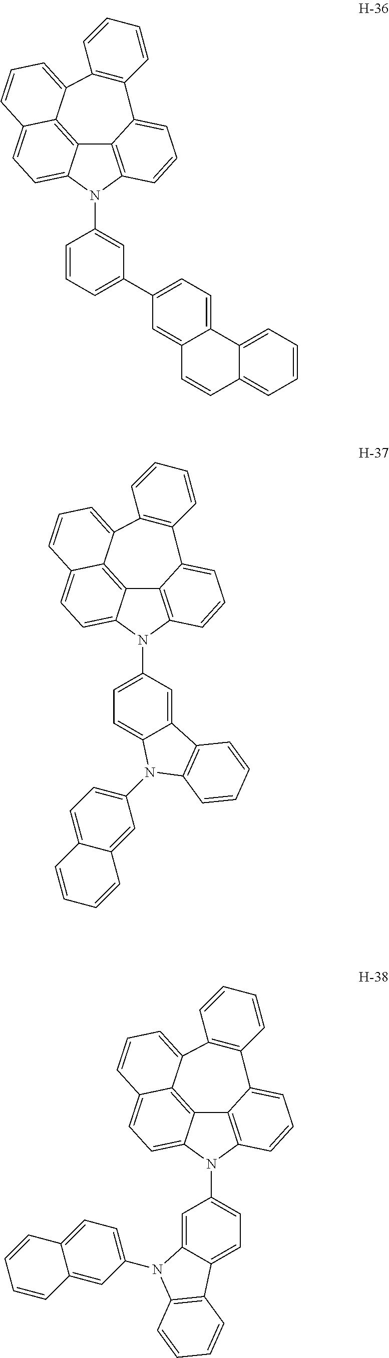 [SMILES: c1cc(-c2ccc3c(ccc4ccccc43)c2)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc2c(c1)c1ccccc1n2-c1ccc2ccccc2c1 ; c1ccc2c(c1)-c1cccc3ccc4c(c13)c1c-2cccc1n4-c1ccc2c3ccccc3n(-c3ccc4ccccc4c3)c2c1]

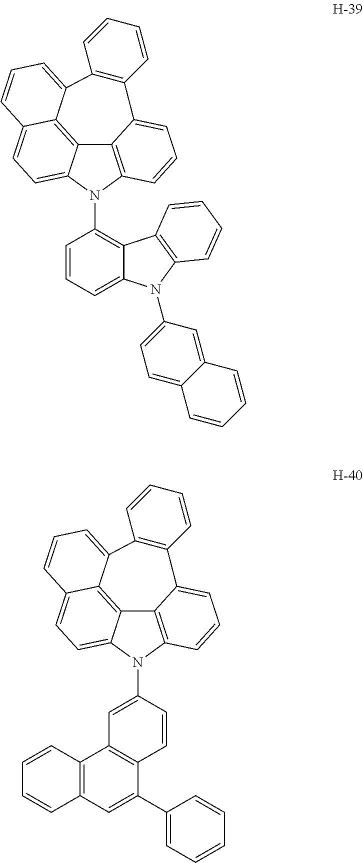 [SMILES: c1ccc2c(c1)-c1cccc3ccc4c(c13)c1c-2cccc1n4-c1cccc2c1c1ccccc1n2-c1ccc2ccccc2c1 ; c1ccc(-c2cc3ccccc3c3cc(-n4c5cccc6c5c5c7c(cccc7ccc54)-c4ccccc4-6)ccc23)cc1]

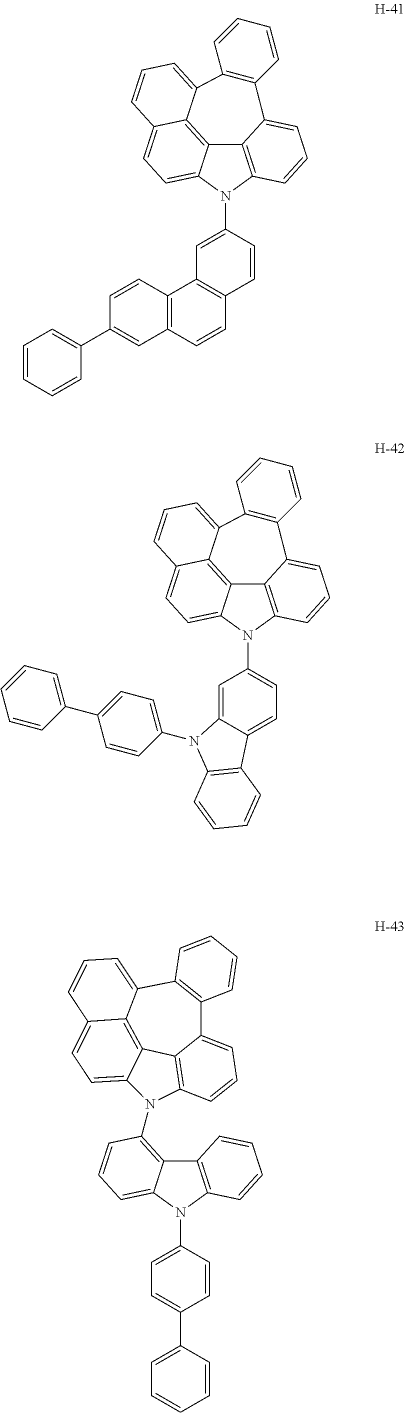 [SMILES: c1ccc(-c2ccc3c(ccc4ccc(-n5c6cccc7c6c6c8c(cccc8ccc65)-c5ccccc5-7)cc43)c2)cc1 ; c1ccc(-c2ccc(-n3c4ccccc4c4ccc(-n5c6cccc7c6c6c8c(cccc8ccc65)-c5ccccc5-7)cc43)cc2)cc1 ; c1ccc(-c2ccc(-n3c4ccccc4c4c(-n5c6cccc7c6c6c8c(cccc8ccc65)-c5ccccc5-7)cccc43)cc2)cc1]

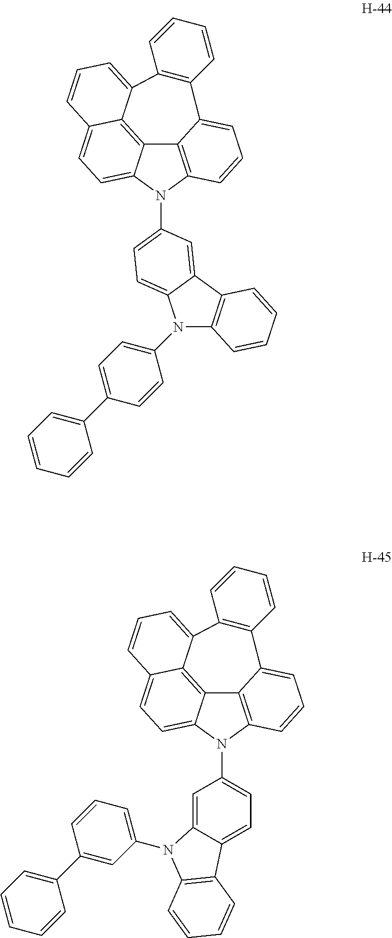 [SMILES: c1ccc(-c2ccc(-n3c4ccccc4c4cc(-n5c6cccc7c6c6c8c(cccc8ccc65)-c5ccccc5-7)ccc43)cc2)cc1 ; c1ccc(-c2cccc(-n3c4ccccc4c4ccc(-n5c6cccc7c6c6c8c(cccc8ccc65)-c5ccccc5-7)cc43)c2)cc1]

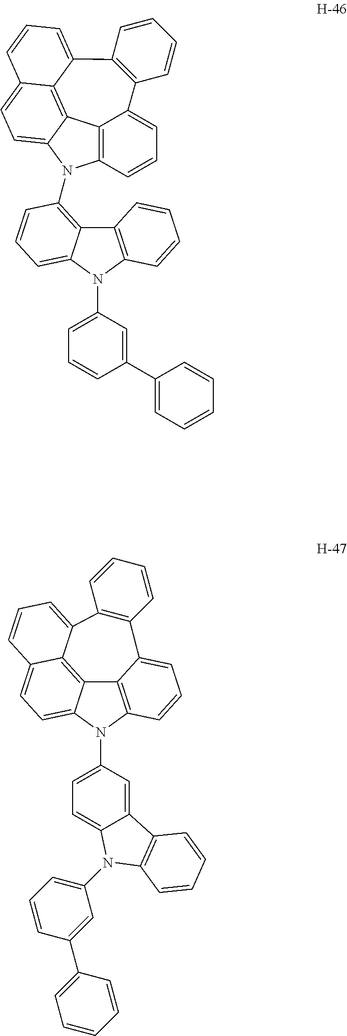 [SMILES: c1ccc(-c2cccc(-n3c4ccccc4c4c(-n5c6cccc7c6c6c8c(cccc8ccc65)-c5ccccc5-7)cccc43)c2)cc1 ; c1ccc(-c2cccc(-n3c4ccccc4c4cc(-n5c6cccc7c6c6c8c(cccc8ccc65)-c5ccccc5-7)ccc43)c2)cc1]

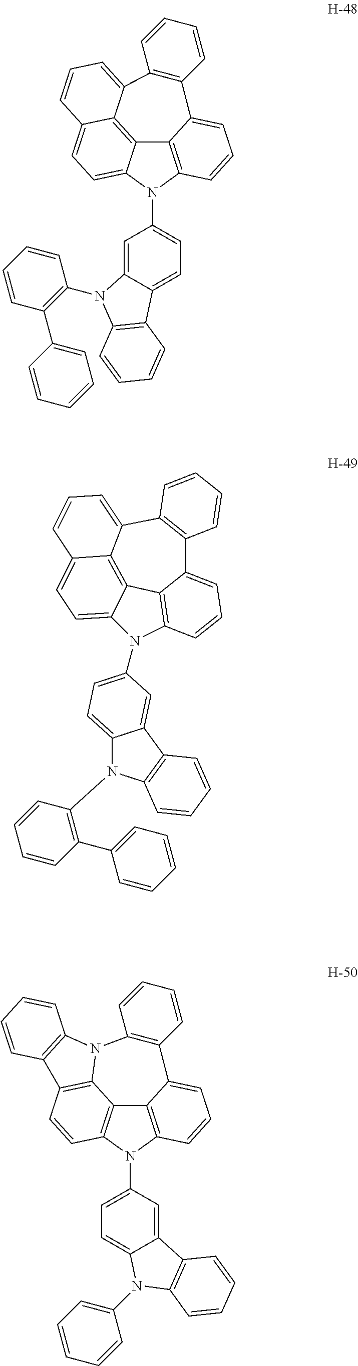 [SMILES: c1ccc(-c2ccccc2-n2c3ccccc3c3ccc(-n4c5cccc6c5c5c7c(cccc7ccc54)-c4ccccc4-6)cc32)cc1 ; c1ccc(-c2ccccc2-n2c3ccccc3c3cc(-n4c5cccc6c5c5c7c(cccc7ccc54)-c4ccccc4-6)ccc32)cc1 ; c1ccc(-n2c3ccccc3c3cc(-n4c5cccc6c7ccccc7n7c8ccccc8c8ccc4c(c65)c87)ccc32)cc1]

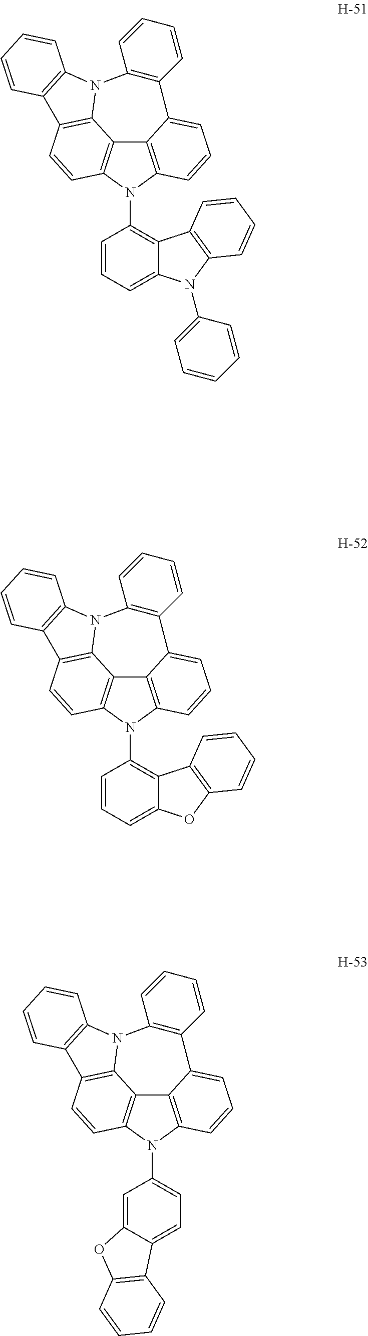 [SMILES: c1ccc(-n2c3ccccc3c3c(-n4c5cccc6c7ccccc7n7c8ccccc8c8ccc4c(c65)c87)cccc32)cc1 ; c1ccc2c(c1)oc1cccc(-n3c4cccc5c6ccccc6n6c7ccccc7c7ccc3c(c54)c76)c12 ; c1ccc2c(c1)oc1cc(-n3c4cccc5c6ccccc6n6c7ccccc7c7ccc3c(c54)c76)ccc12]

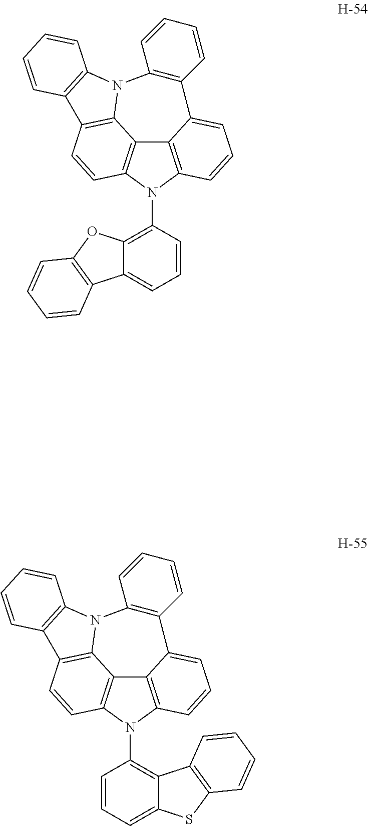 [SMILES: c1ccc2c(c1)oc1c(-n3c4cccc5c6ccccc6n6c7ccccc7c7ccc3c(c54)c76)cccc12 ; c1ccc2c(c1)sc1cccc(-n3c4cccc5c6ccccc6n6c7ccccc7c7ccc3c(c54)c76)c12]

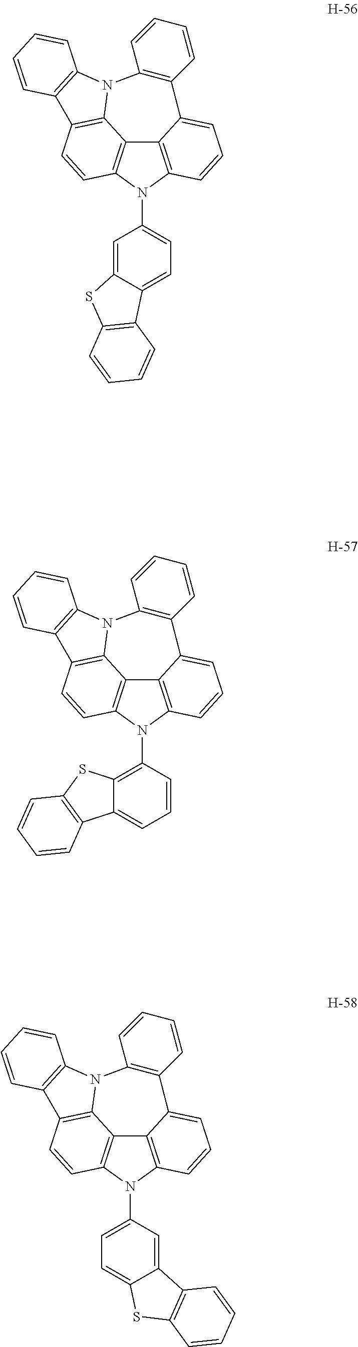 [SMILES: c1ccc2c(c1)sc1cc(-n3c4cccc5c6ccccc6n6c7ccccc7c7ccc3c(c54)c76)ccc12 ; c1ccc2c(c1)sc1c(-n3c4cccc5c6ccccc6n6c7ccccc7c7ccc3c(c54)c76)cccc12 ; c1ccc2c(c1)sc1ccc(-n3c4cccc5c6ccccc6n6c7ccccc7c7ccc3c(c54)c76)cc12]

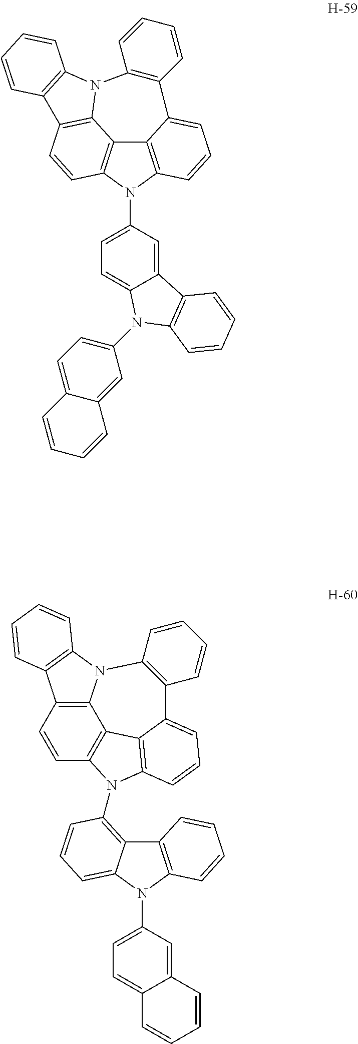 [SMILES: c1ccc2cc(-n3c4ccccc4c4cc(-n5c6cccc7c8ccccc8n8c9ccccc9c9ccc5c(c76)c98)ccc43)ccc2c1 ; c1ccc2cc(-n3c4ccccc4c4c(-n5c6cccc7c8ccccc8n8c9ccccc9c9ccc5c(c76)c98)cccc43)ccc2c1]

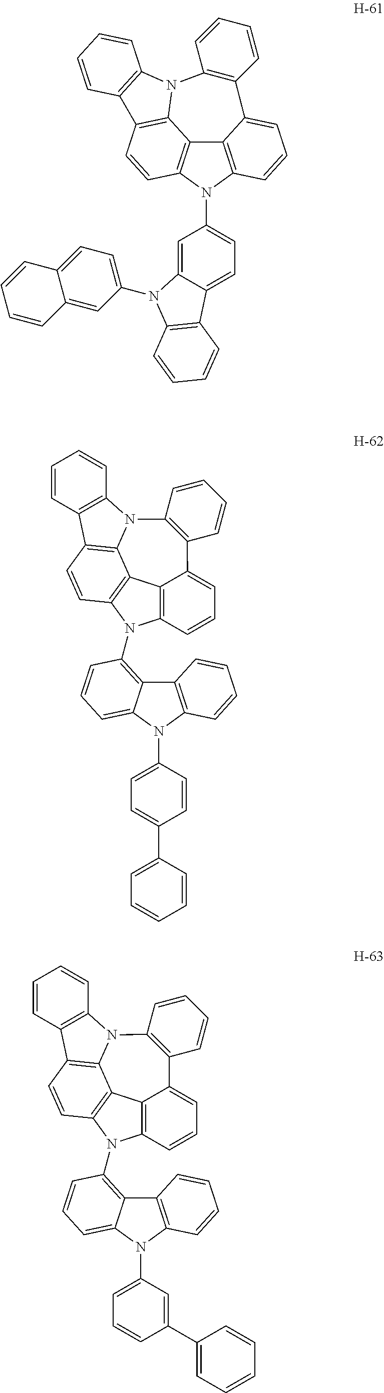 [SMILES: c1ccc2cc(-n3c4ccccc4c4ccc(-n5c6cccc7c8ccccc8n8c9ccccc9c9ccc5c(c76)c98)cc43)ccc2c1 ; c1ccc(-c2ccc(-n3c4ccccc4c4c(-n5c6cccc7c8ccccc8n8c9ccccc9c9ccc5c(c76)c98)cccc43)cc2)cc1 ; c1ccc(-c2cccc(-n3c4ccccc4c4c(-n5c6cccc7c8ccccc8n8c9ccccc9c9ccc5c(c76)c98)cccc43)c2)cc1]

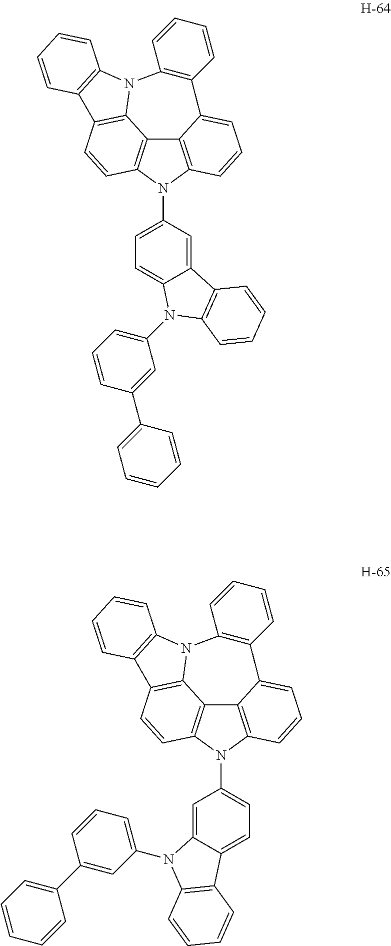 [SMILES: c1ccc(-c2cccc(-n3c4ccccc4c4cc(-n5c6cccc7c8ccccc8n8c9ccccc9c9ccc5c(c76)c98)ccc43)c2)cc1 ; c1ccc(-c2cccc(-n3c4ccccc4c4ccc(-n5c6cccc7c8ccccc8n8c9ccccc9c9ccc5c(c76)c98)cc43)c2)cc1]

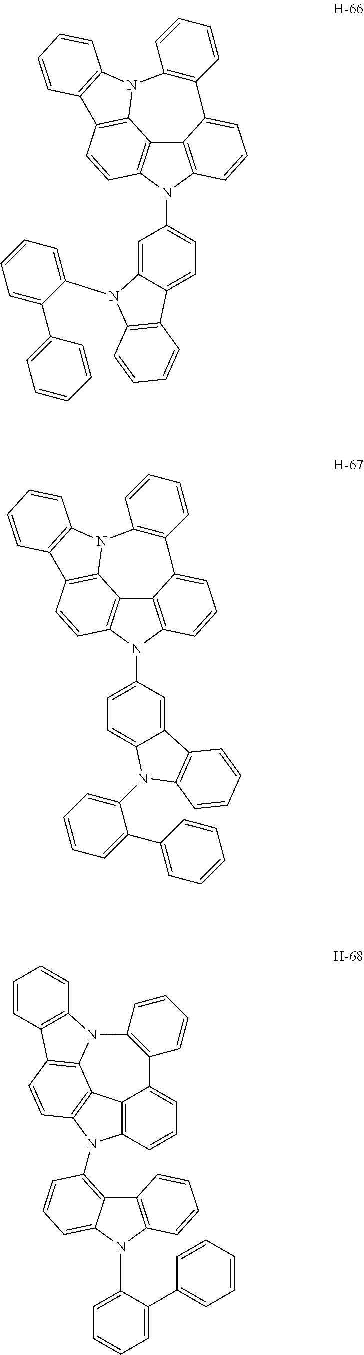 [SMILES: c1ccc(-c2ccccc2-n2c3ccccc3c3ccc(-n4c5cccc6c7ccccc7n7c8ccccc8c8ccc4c(c65)c87)cc32)cc1 ; c1ccc(-c2ccccc2-n2c3ccccc3c3cc(-n4c5cccc6c7ccccc7n7c8ccccc8c8ccc4c(c65)c87)ccc32)cc1 ; c1ccc(-c2ccccc2-n2c3ccccc3c3c(-n4c5cccc6c7ccccc7n7c8ccccc8c8ccc4c(c65)c87)cccc32)cc1]

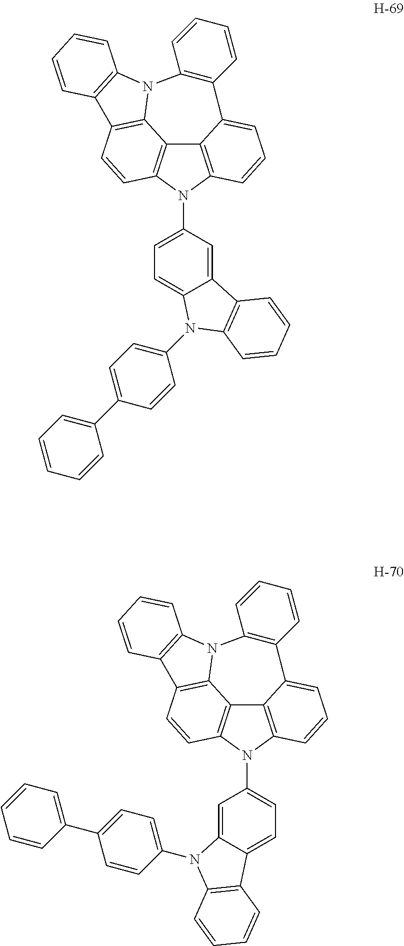 [SMILES: c1ccc(-c2ccc(-n3c4ccccc4c4cc(-n5c6cccc7c8ccccc8n8c9ccccc9c9ccc5c(c76)c98)ccc43)cc2)cc1 ; c1ccc(-c2ccc(-n3c4ccccc4c4ccc(-n5c6cccc7c8ccccc8n8c9ccccc9c9ccc5c(c76)c98)cc43)cc2)cc1]

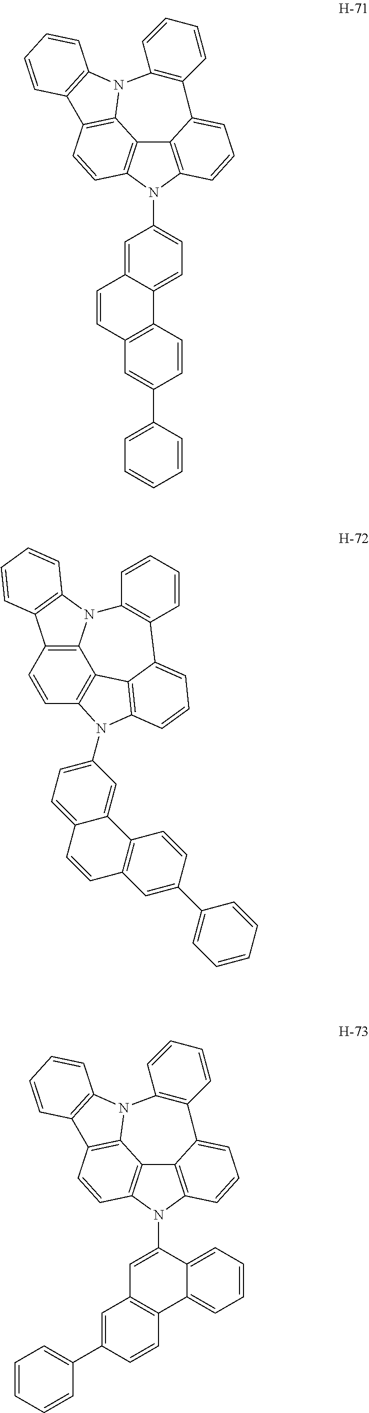 [SMILES: c1ccc(-c2ccc3c(ccc4cc(-n5c6cccc7c8ccccc8n8c9ccccc9c9ccc5c(c76)c98)ccc43)c2)cc1 ; c1ccc(-c2ccc3c(ccc4ccc(-n5c6cccc7c8ccccc8n8c9ccccc9c9ccc5c(c76)c98)cc43)c2)cc1 ; c1ccc(-c2ccc3c(c2)cc(-n2c4cccc5c6ccccc6n6c7ccccc7c7ccc2c(c54)c76)c2ccccc23)cc1]

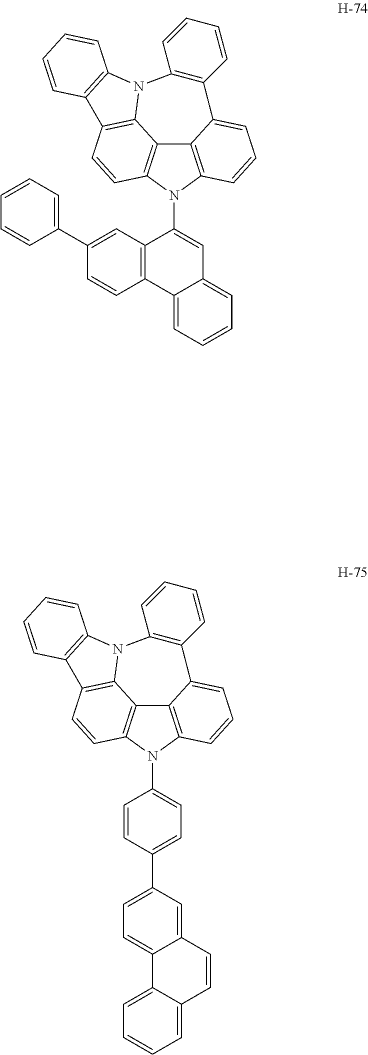 [SMILES: c1ccc(-c2ccc3c(c2)c(-n2c4cccc5c6ccccc6n6c7ccccc7c7ccc2c(c54)c76)cc2ccccc23)cc1 ; c1ccc2c(c1)ccc1cc(-c3ccc(-n4c5cccc6c7ccccc7n7c8ccccc8c8ccc4c(c65)c87)cc3)ccc12]

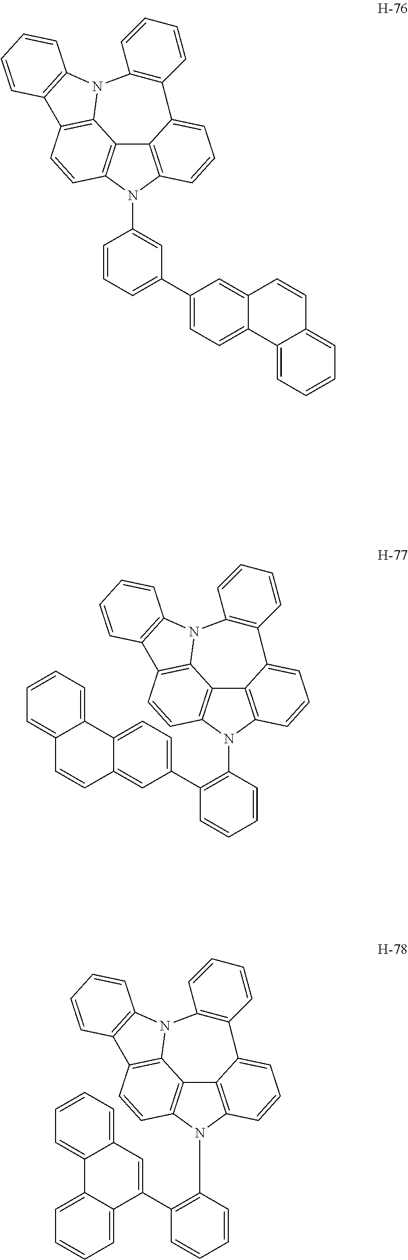 [SMILES: c1cc(-c2ccc3c(ccc4ccccc43)c2)cc(-n2c3cccc4c5ccccc5n5c6ccccc6c6ccc2c(c43)c65)c1 ; c1ccc(-n2c3cccc4c5ccccc5n5c6ccccc6c6ccc2c(c43)c65)c(-c2ccc3c(ccc4ccccc43)c2)c1 ; c1ccc(-n2c3cccc4c5ccccc5n5c6ccccc6c6ccc2c(c43)c65)c(-c2cc3ccccc3c3ccccc23)c1]

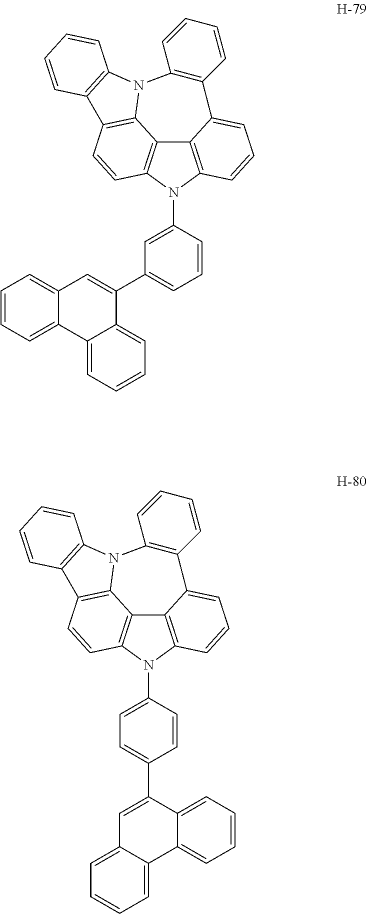 [SMILES: c1cc(-c2cc3ccccc3c3ccccc23)cc(-n2c3cccc4c5ccccc5n5c6ccccc6c6ccc2c(c43)c65)c1 ; c1ccc2c(c1)cc(-c1ccc(-n3c4cccc5c6ccccc6n6c7ccccc7c7ccc3c(c54)c76)cc1)c1ccccc12]

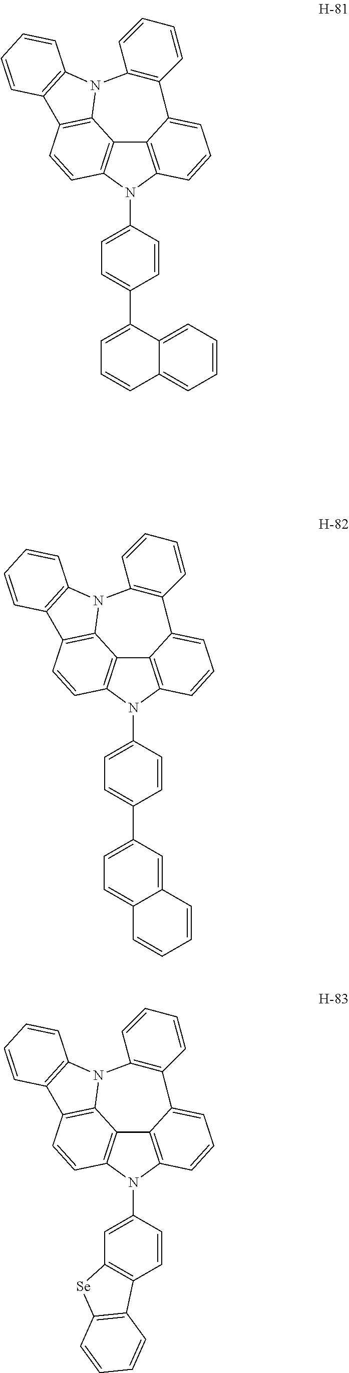 [SMILES: c1ccc2c(-c3ccc(-n4c5cccc6c7ccccc7n7c8ccccc8c8ccc4c(c65)c87)cc3)cccc2c1 ; c1ccc2cc(-c3ccc(-n4c5cccc6c7ccccc7n7c8ccccc8c8ccc4c(c65)c87)cc3)ccc2c1 ; c1ccc2c(c1)[se]c1cc(-n3c4cccc5c6ccccc6n6c7ccccc7c7ccc3c(c54)c76)ccc12]

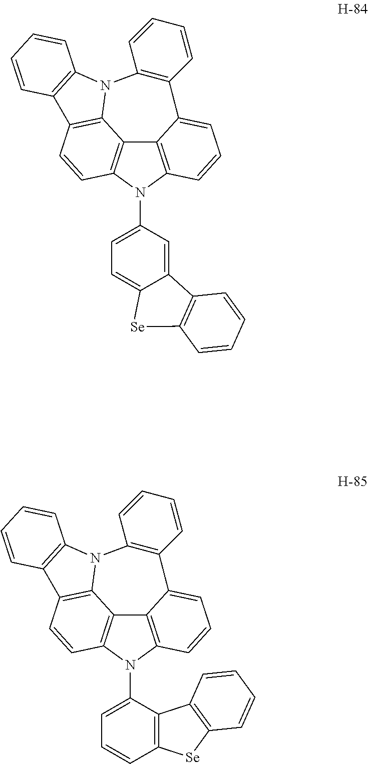 [SMILES: c1ccc2c(c1)[se]c1ccc(-n3c4cccc5c6ccccc6n6c7ccccc7c7ccc3c(c54)c76)cc12 ; c1ccc2c(c1)[se]c1cccc(-n3c4cccc5c6ccccc6n6c7ccccc7c7ccc3c(c54)c76)c12]

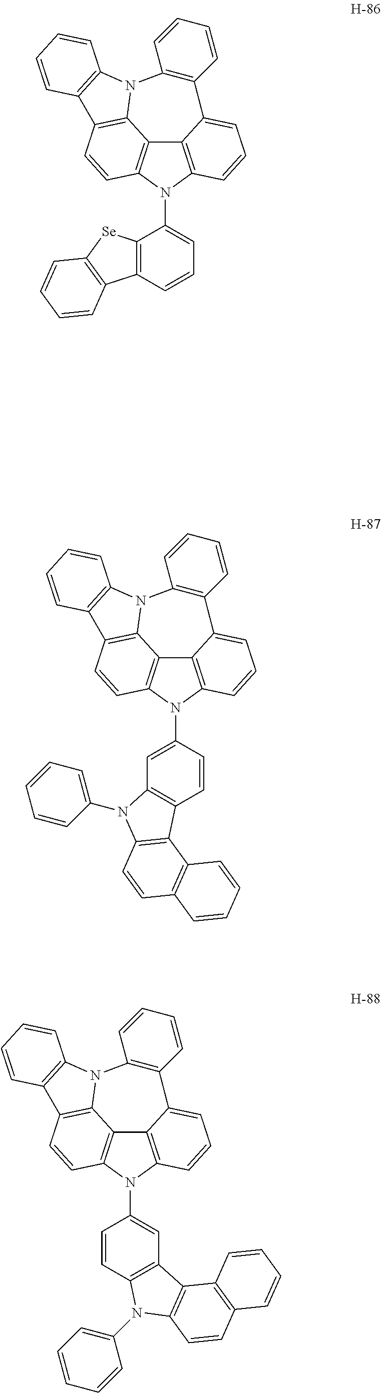 [SMILES: c1ccc2c(c1)[se]c1c(-n3c4cccc5c6ccccc6n6c7ccccc7c7ccc3c(c54)c76)cccc12 ; c1ccc(-n2c3cc(-n4c5cccc6c7ccccc7n7c8ccccc8c8ccc4c(c65)c87)ccc3c3c4ccccc4ccc32)cc1 ; c1ccc(-n2c3ccc(-n4c5cccc6c7ccccc7n7c8ccccc8c8ccc4c(c65)c87)cc3c3c4ccccc4ccc32)cc1]

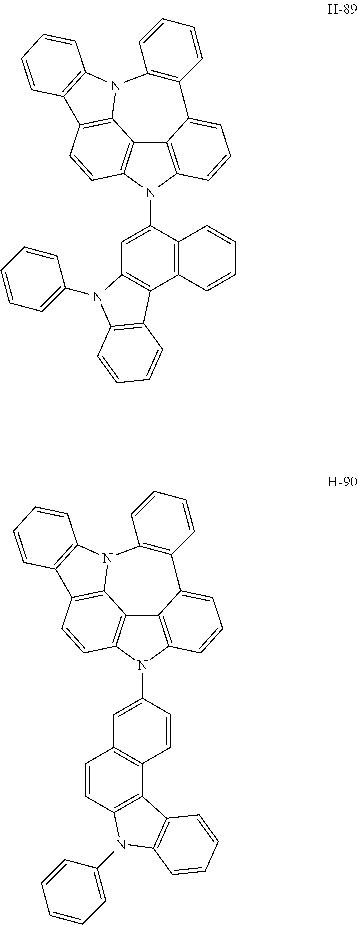 [SMILES: c1ccc(-n2c3ccccc3c3c4ccccc4c(-n4c5cccc6c7ccccc7n7c8ccccc8c8ccc4c(c65)c87)cc32)cc1 ; c1ccc(-n2c3ccccc3c3c4ccc(-n5c6cccc7c8ccccc8n8c9ccccc9c9ccc5c(c76)c98)cc4ccc32)cc1]

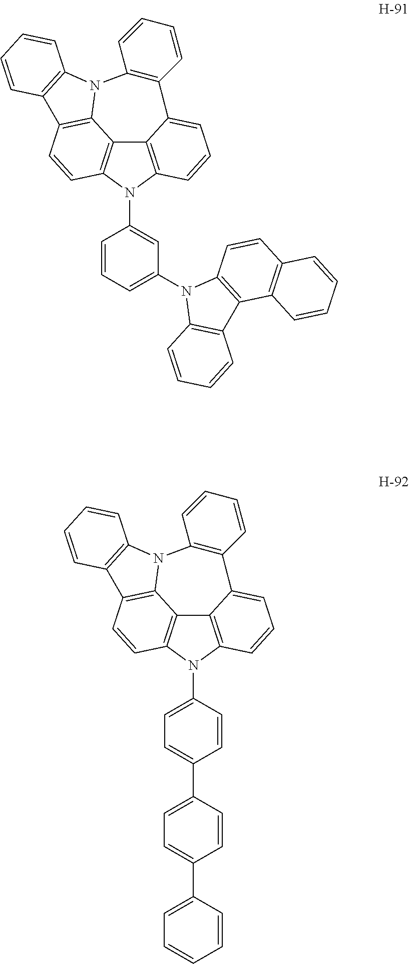 [SMILES: c1cc(-n2c3ccccc3c3c4ccccc4ccc32)cc(-n2c3cccc4c5ccccc5n5c6ccccc6c6ccc2c(c43)c65)c1 ; c1ccc(-c2ccc(-c3ccc(-n4c5cccc6c7ccccc7n7c8ccccc8c8ccc4c(c65)c87)cc3)cc2)cc1]

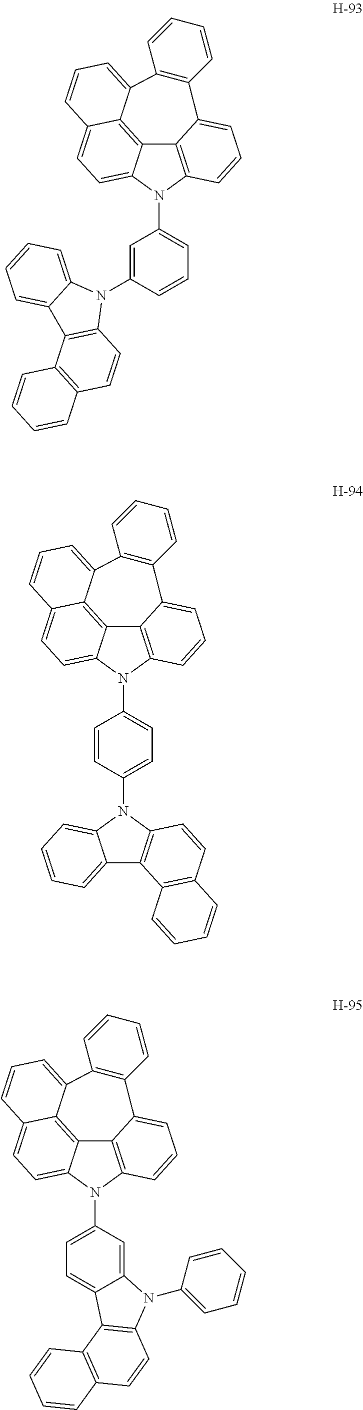 [SMILES: c1cc(-n2c3ccccc3c3c4ccccc4ccc32)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc(-n2c3ccccc3c3c4ccccc4ccc32)cc1 ; c1ccc(-n2c3cc(-n4c5cccc6c5c5c7c(cccc7ccc54)-c4ccccc4-6)ccc3c3c4ccccc4ccc32)cc1]

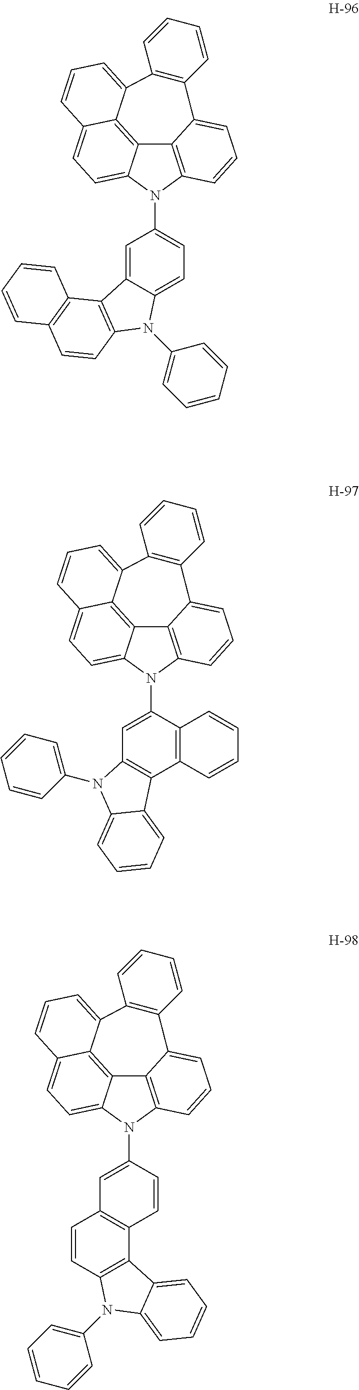 [SMILES: c1ccc(-n2c3ccc(-n4c5cccc6c5c5c7c(cccc7ccc54)-c4ccccc4-6)cc3c3c4ccccc4ccc32)cc1 ; c1ccc(-n2c3ccccc3c3c4ccccc4c(-n4c5cccc6c5c5c7c(cccc7ccc54)-c4ccccc4-6)cc32)cc1 ; c1ccc(-n2c3ccccc3c3c4ccc(-n5c6cccc7c6c6c8c(cccc8ccc65)-c5ccccc5-7)cc4ccc32)cc1]

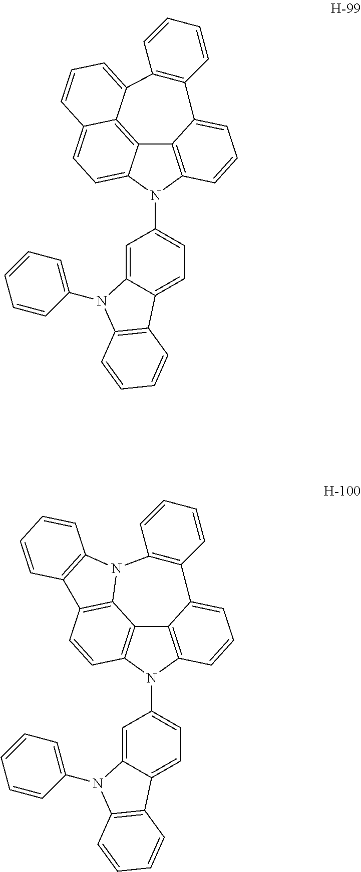 [SMILES: c1ccc(-n2c3ccccc3c3ccc(-n4c5cccc6c5c5c7c(cccc7ccc54)-c4ccccc4-6)cc32)cc1 ; c1ccc(-n2c3ccccc3c3ccc(-n4c5cccc6c7ccccc7n7c8ccccc8c8ccc4c(c65)c87)cc32)cc1]

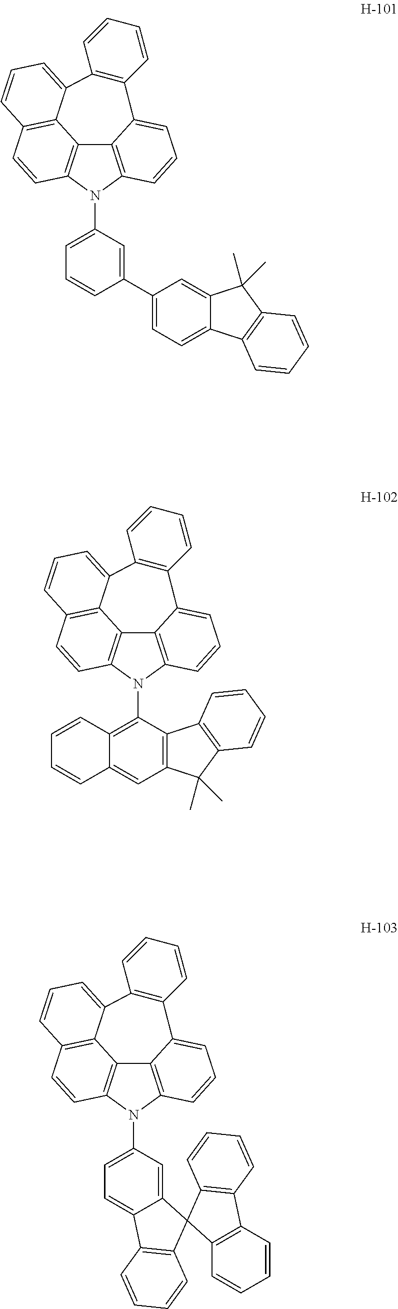 [SMILES: CC1(C)c2ccccc2-c2ccc(-c3cccc(-n4c5cccc6c5c5c7c(cccc7ccc54)-c4ccccc4-6)c3)cc21 ; CC1(C)c2ccccc2-c2c1cc1ccccc1c2-n1c2cccc3c2c2c4c(cccc4ccc21)-c1ccccc1-3 ; c1ccc2c(c1)-c1cccc3ccc4c(c13)c1c-2cccc1n4-c1ccc2c(c1)C1(c3ccccc3-c3ccccc31)c1ccccc1-2]

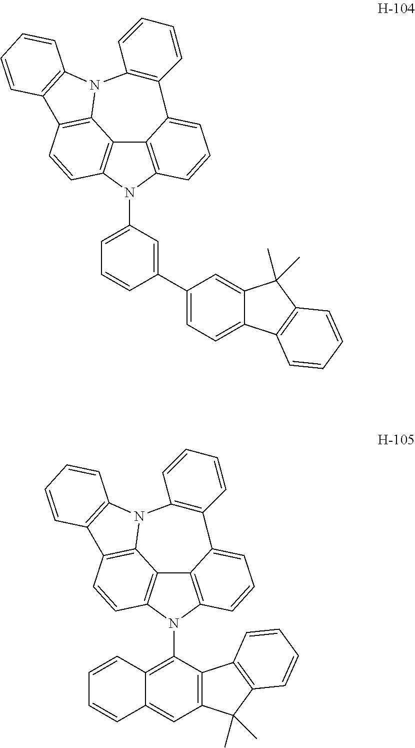 [SMILES: CC1(C)c2ccccc2-c2ccc(-c3cccc(-n4c5cccc6c7ccccc7n7c8ccccc8c8ccc4c(c65)c87)c3)cc21 ; CC1(C)c2ccccc2-c2c1cc1ccccc1c2-n1c2cccc3c4ccccc4n4c5ccccc5c5ccc1c(c32)c54]

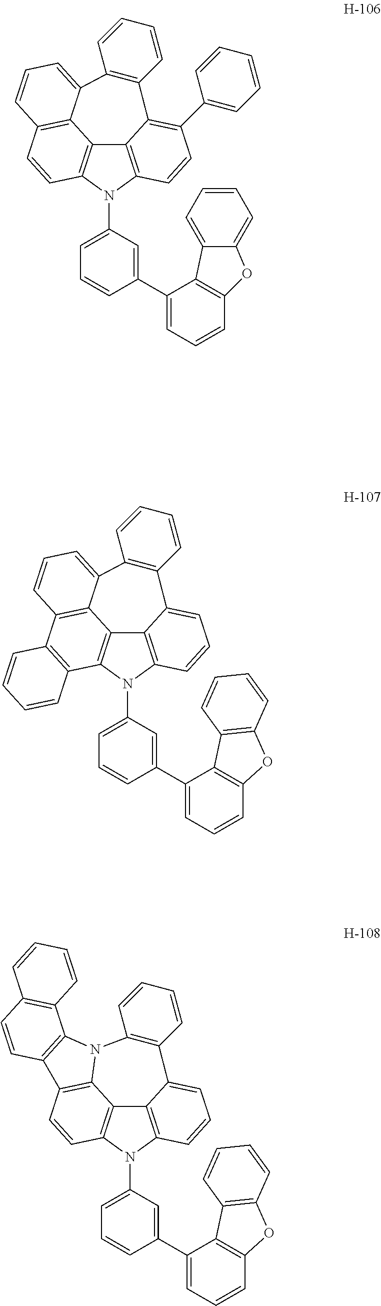 [SMILES: c1ccc(-c2ccc3c4c2-c2ccccc2-c2cccc5ccc(c4c25)n3-c2cccc(-c3cccc4oc5ccccc5c34)c2)cc1 ; c1cc(-c2cccc3oc4ccccc4c23)cc(-n2c3cccc4c3c3c5c(cccc5c5ccccc5c32)-c2ccccc2-4)c1 ; c1cc(-c2cccc3oc4ccccc4c23)cc(-n2c3cccc4c5ccccc5n5c6c7ccccc7ccc6c6ccc2c(c43)c65)c1]

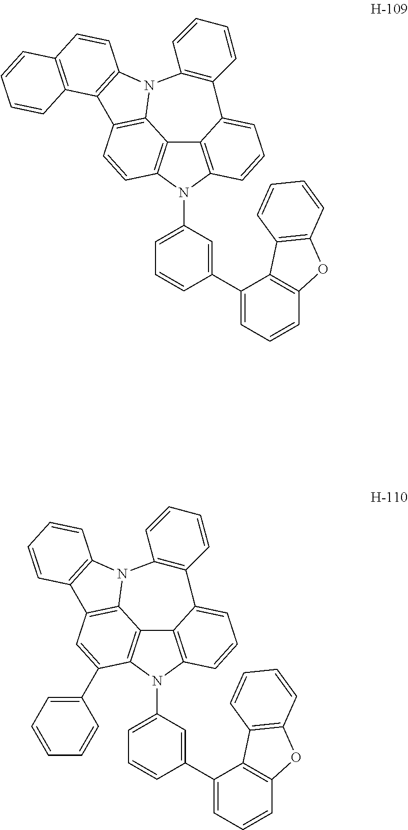 [SMILES: c1cc(-c2cccc3oc4ccccc4c23)cc(-n2c3cccc4c5ccccc5n5c6ccc7ccccc7c6c6ccc2c(c43)c65)c1 ; c1ccc(-c2cc3c4ccccc4n4c5ccccc5c5cccc6c5c(c2n6-c2cccc(-c5cccc6oc7ccccc7c56)c2)c34)cc1]

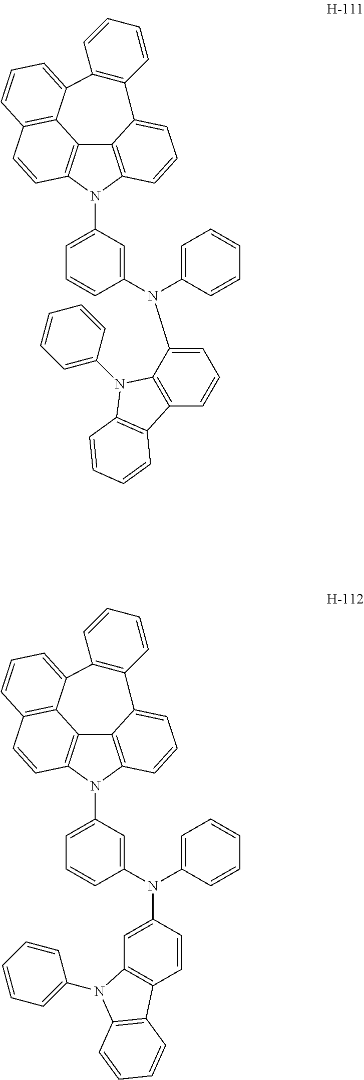 [SMILES: c1ccc(N(c2cccc(-n3c4cccc5c4c4c6c(cccc6ccc43)-c3ccccc3-5)c2)c2cccc3c4ccccc4n(-c4ccccc4)c23)cc1 ; c1ccc(N(c2cccc(-n3c4cccc5c4c4c6c(cccc6ccc43)-c3ccccc3-5)c2)c2ccc3c4ccccc4n(-c4ccccc4)c3c2)cc1]

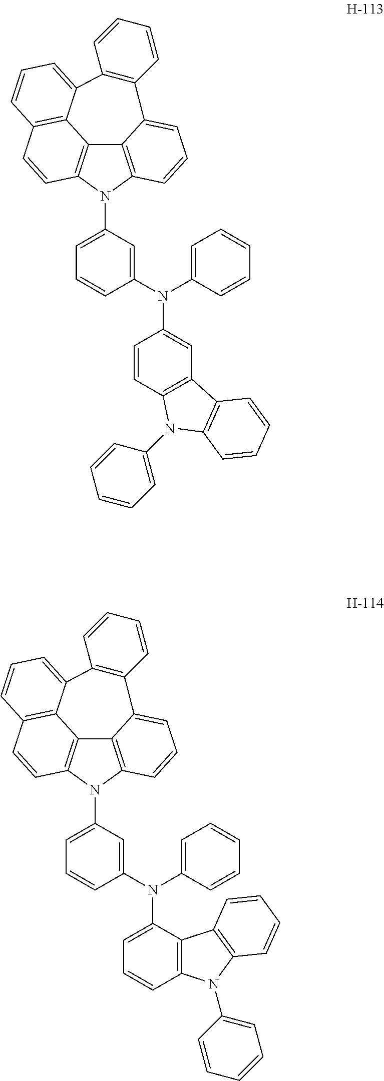 [SMILES: c1ccc(N(c2cccc(-n3c4cccc5c4c4c6c(cccc6ccc43)-c3ccccc3-5)c2)c2ccc3c(c2)c2ccccc2n3-c2ccccc2)cc1 ; c1ccc(N(c2cccc(-n3c4cccc5c4c4c6c(cccc6ccc43)-c3ccccc3-5)c2)c2cccc3c2c2ccccc2n3-c2ccccc2)cc1]

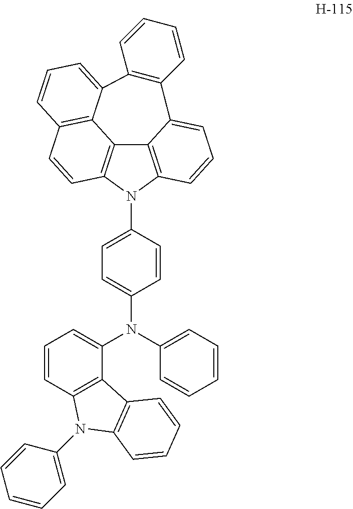 [SMILES: c1ccc(N(c2ccc(-n3c4cccc5c4c4c6c(cccc6ccc43)-c3ccccc3-5)cc2)c2cccc3c2c2ccccc2n3-c2ccccc2)cc1]

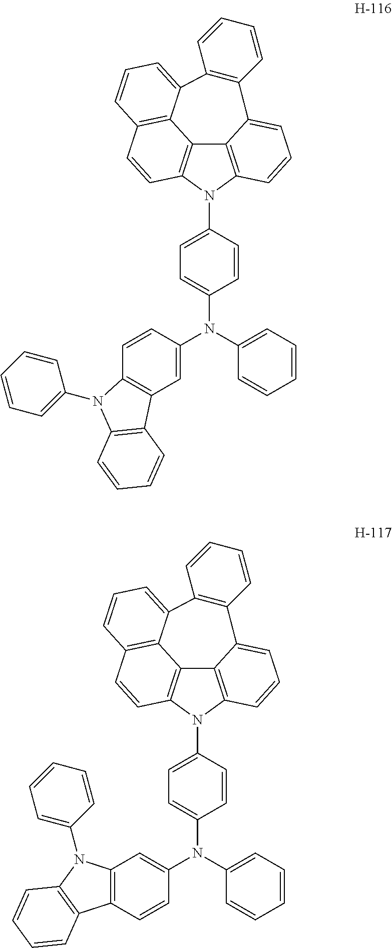 [SMILES: c1ccc(N(c2ccc(-n3c4cccc5c4c4c6c(cccc6ccc43)-c3ccccc3-5)cc2)c2ccc3c(c2)c2ccccc2n3-c2ccccc2)cc1 ; c1ccc(N(c2ccc(-n3c4cccc5c4c4c6c(cccc6ccc43)-c3ccccc3-5)cc2)c2ccc3c4ccccc4n(-c4ccccc4)c3c2)cc1]

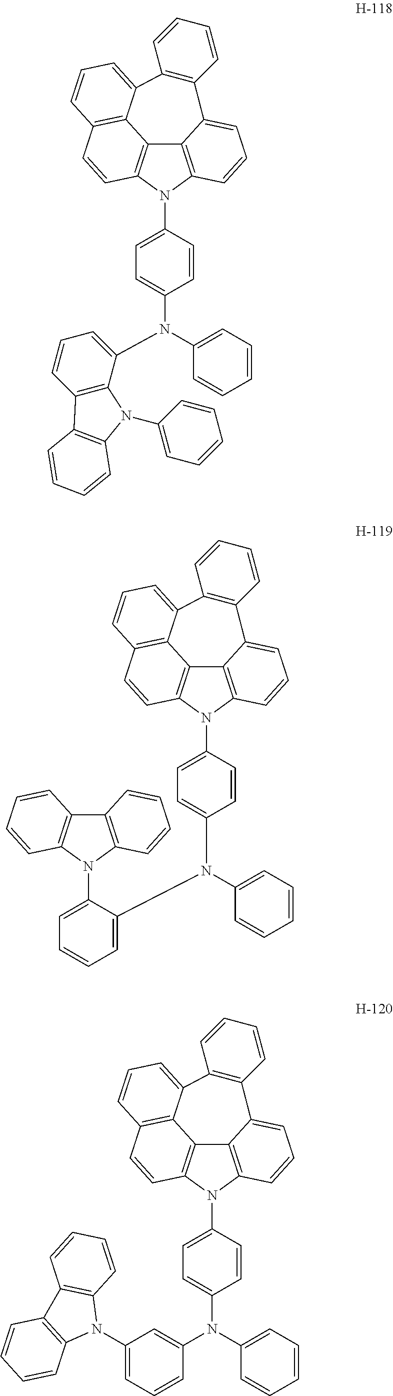 [SMILES: c1ccc(N(c2ccc(-n3c4cccc5c4c4c6c(cccc6ccc43)-c3ccccc3-5)cc2)c2cccc3c4ccccc4n(-c4ccccc4)c23)cc1 ; c1ccc(N(c2ccc(-n3c4cccc5c4c4c6c(cccc6ccc43)-c3ccccc3-5)cc2)c2ccccc2-n2c3ccccc3c3ccccc32)cc1 ; c1ccc(N(c2ccc(-n3c4cccc5c4c4c6c(cccc6ccc43)-c3ccccc3-5)cc2)c2cccc(-n3c4ccccc4c4ccccc43)c2)cc1]

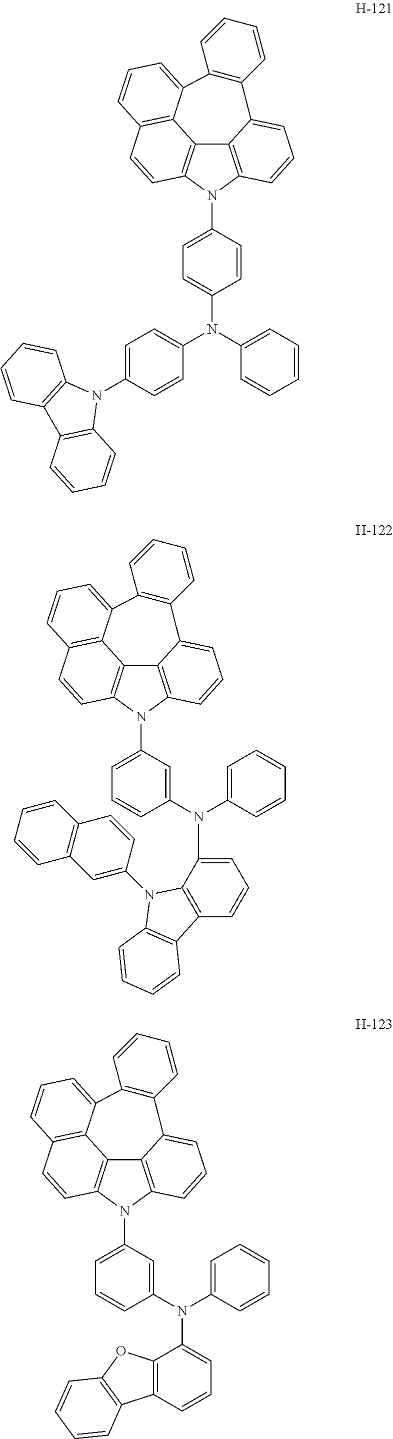

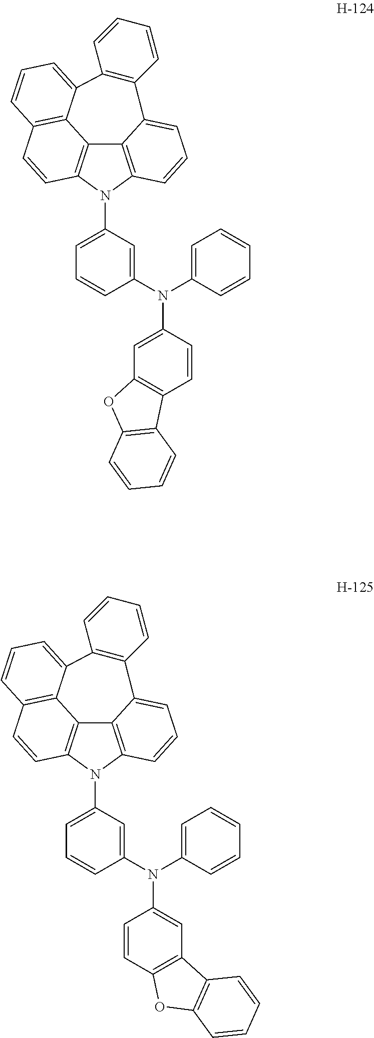 [SMILES: c1ccc(N(c2cccc(-n3c4cccc5c4c4c6c(cccc6ccc43)-c3ccccc3-5)c2)c2ccc3c(c2)oc2ccccc23)cc1 ; c1ccc(N(c2cccc(-n3c4cccc5c4c4c6c(cccc6ccc43)-c3ccccc3-5)c2)c2ccc3oc4ccccc4c3c2)cc1]

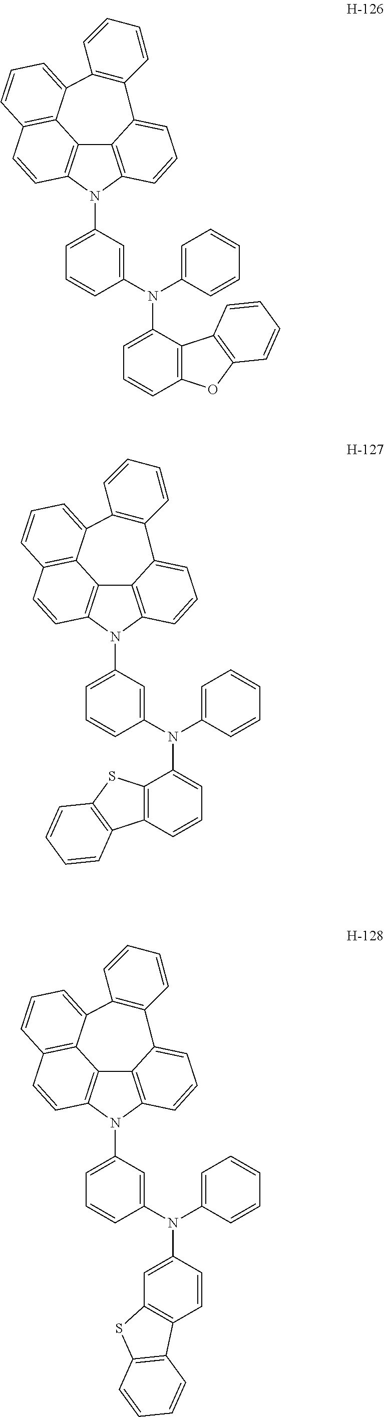 [SMILES: c1ccc(N(c2cccc(-n3c4cccc5c4c4c6c(cccc6ccc43)-c3ccccc3-5)c2)c2cccc3oc4ccccc4c23)cc1 ; c1ccc(N(c2cccc(-n3c4cccc5c4c4c6c(cccc6ccc43)-c3ccccc3-5)c2)c2cccc3c2sc2ccccc23)cc1 ; c1ccc(N(c2cccc(-n3c4cccc5c4c4c6c(cccc6ccc43)-c3ccccc3-5)c2)c2ccc3c(c2)sc2ccccc23)cc1]

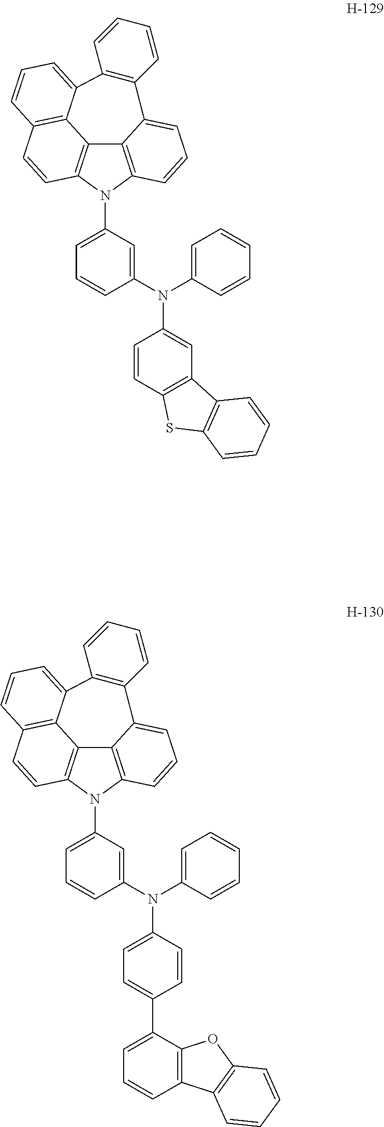 [SMILES: c1ccc(N(c2cccc(-n3c4cccc5c4c4c6c(cccc6ccc43)-c3ccccc3-5)c2)c2ccc3sc4ccccc4c3c2)cc1 ; c1ccc(N(c2ccc(-c3cccc4c3oc3ccccc34)cc2)c2cccc(-n3c4cccc5c4c4c6c(cccc6ccc43)-c3ccccc3-5)c2)cc1]

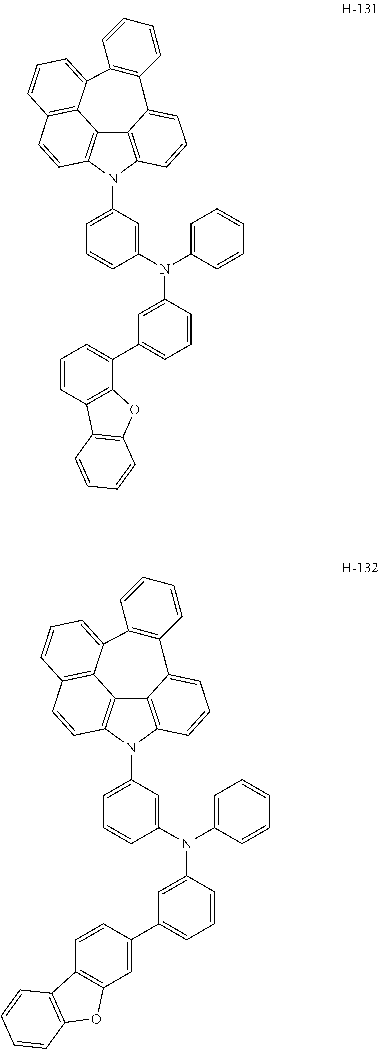 [SMILES: c1ccc(N(c2cccc(-c3cccc4c3oc3ccccc34)c2)c2cccc(-n3c4cccc5c4c4c6c(cccc6ccc43)-c3ccccc3-5)c2)cc1 ; c1ccc(N(c2cccc(-c3ccc4c(c3)oc3ccccc34)c2)c2cccc(-n3c4cccc5c4c4c6c(cccc6ccc43)-c3ccccc3-5)c2)cc1]

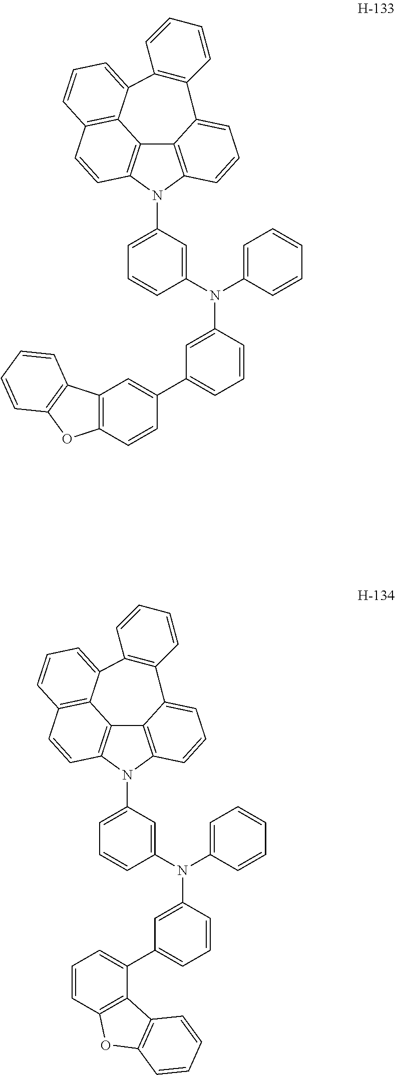 [SMILES: c1ccc(N(c2cccc(-c3ccc4oc5ccccc5c4c3)c2)c2cccc(-n3c4cccc5c4c4c6c(cccc6ccc43)-c3ccccc3-5)c2)cc1 ; c1ccc(N(c2cccc(-c3cccc4oc5ccccc5c34)c2)c2cccc(-n3c4cccc5c4c4c6c(cccc6ccc43)-c3ccccc3-5)c2)cc1]

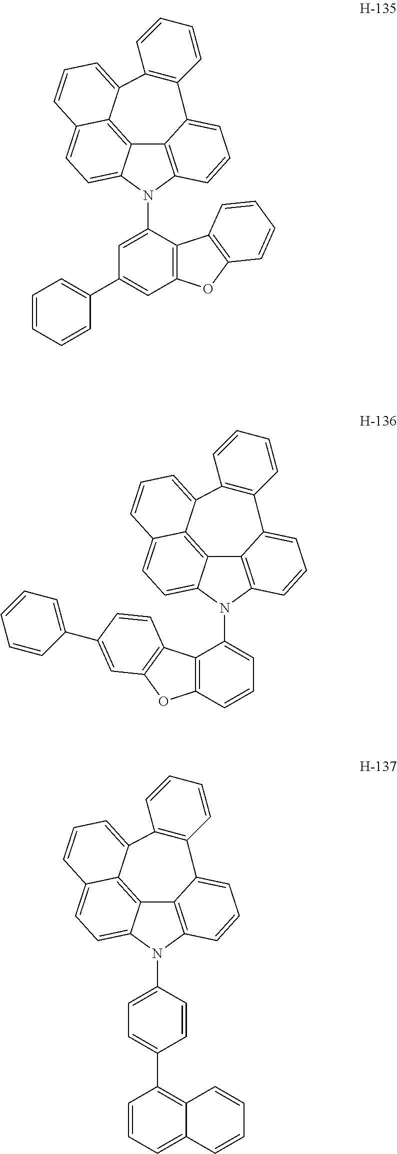 [SMILES: c1ccc(-c2cc(-n3c4cccc5c4c4c6c(cccc6ccc43)-c3ccccc3-5)c3c(c2)oc2ccccc23)cc1 ; c1ccc(-c2ccc3c(c2)oc2cccc(-n4c5cccc6c5c5c7c(cccc7ccc54)-c4ccccc4-6)c23)cc1 ; c1ccc2c(c1)-c1cccc3ccc4c(c13)c1c-2cccc1n4-c1ccc(-c2cccc3ccccc23)cc1]

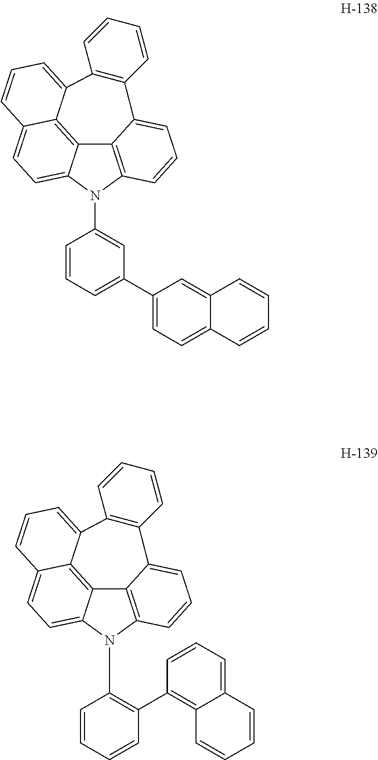 [SMILES: c1cc(-c2ccc3ccccc3c2)cc(-n2c3cccc4c3c3c5c(cccc5ccc32)-c2ccccc2-4)c1 ; c1ccc2c(c1)-c1cccc3ccc4c(c13)c1c-2cccc1n4-c1ccccc1-c1cccc2ccccc12]

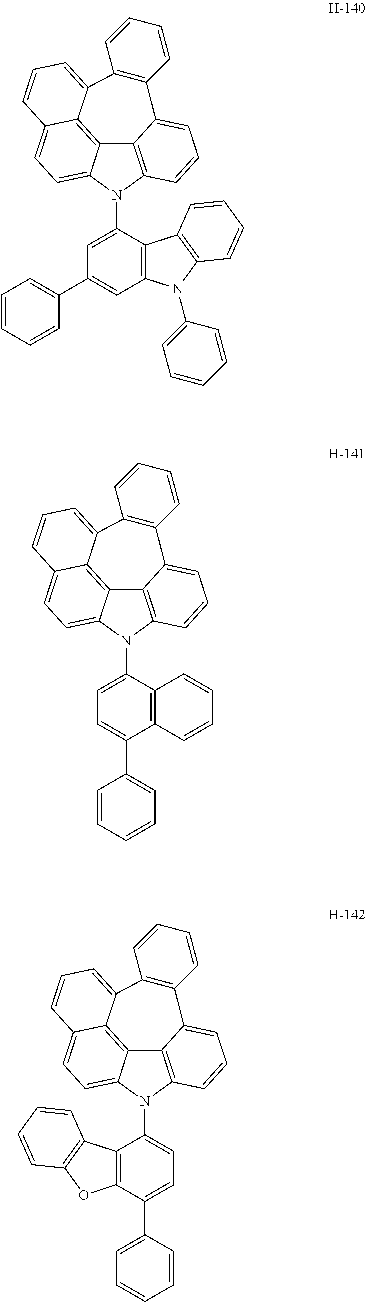 [SMILES: c1ccc(-c2cc(-n3c4cccc5c4c4c6c(cccc6ccc43)-c3ccccc3-5)c3c4ccccc4n(-c4ccccc4)c3c2)cc1 ; c1ccc(-c2ccc(-n3c4cccc5c4c4c6c(cccc6ccc43)-c3ccccc3-5)c3ccccc23)cc1 ; c1ccc(-c2ccc(-n3c4cccc5c4c4c6c(cccc6ccc43)-c3ccccc3-5)c3c2oc2ccccc23)cc1]

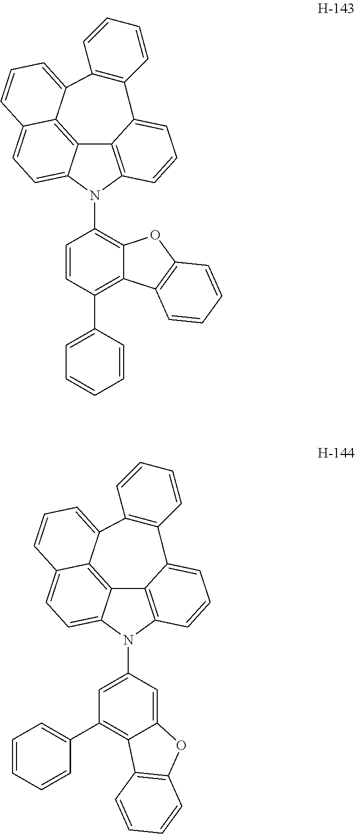 [SMILES: c1ccc(-c2ccc(-n3c4cccc5c4c4c6c(cccc6ccc43)-c3ccccc3-5)c3oc4ccccc4c23)cc1 ; c1ccc(-c2cc(-n3c4cccc5c4c4c6c(cccc6ccc43)-c3ccccc3-5)cc3oc4ccccc4c23)cc1]

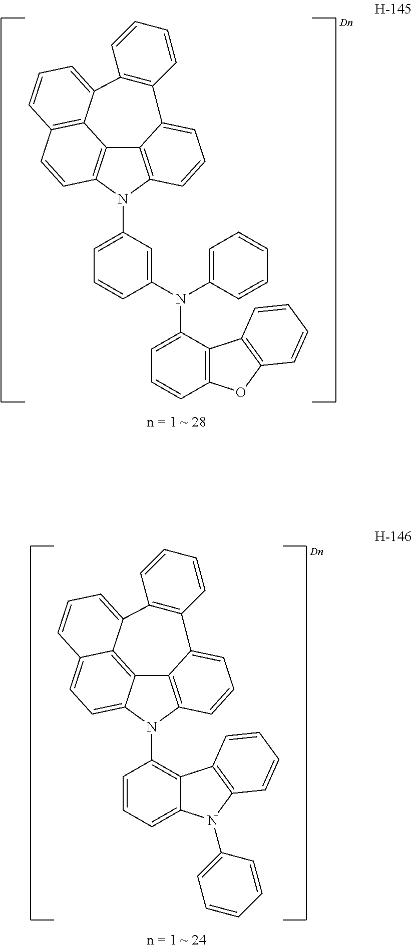

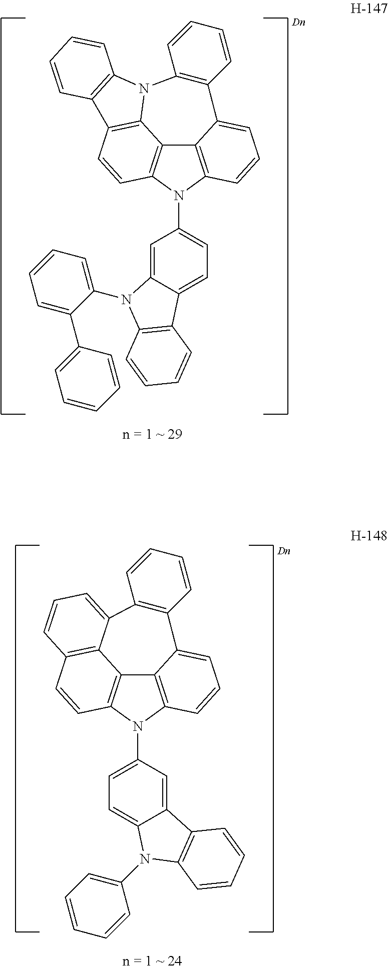

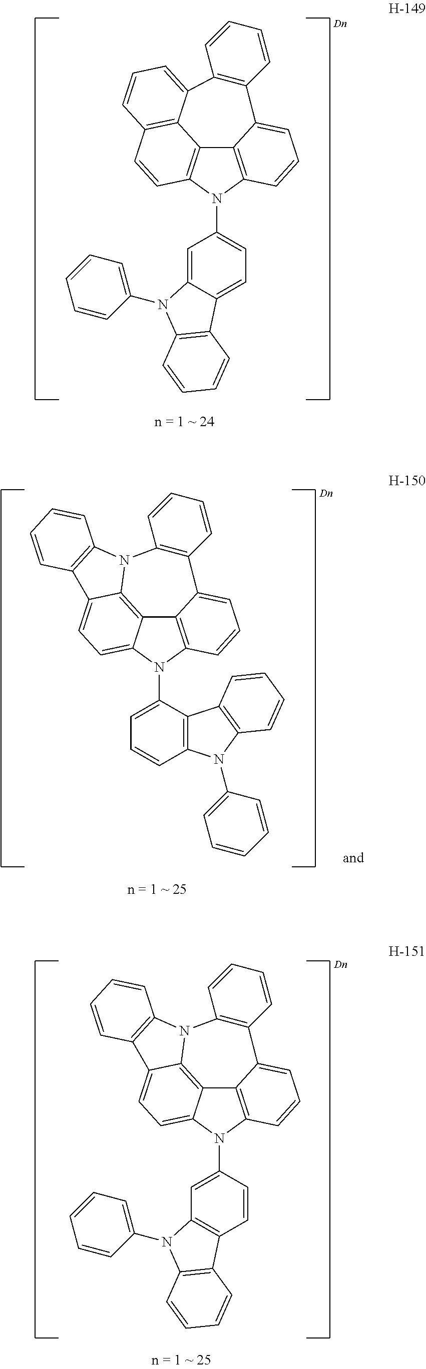

In the compounds above, Dn represents that n number of hydrogens are replaced with deuterium.

According to one embodiment of the present disclosure, the compound represented by Formula 2 may be selected from the group consisting of the following compounds, but is not limited thereto.

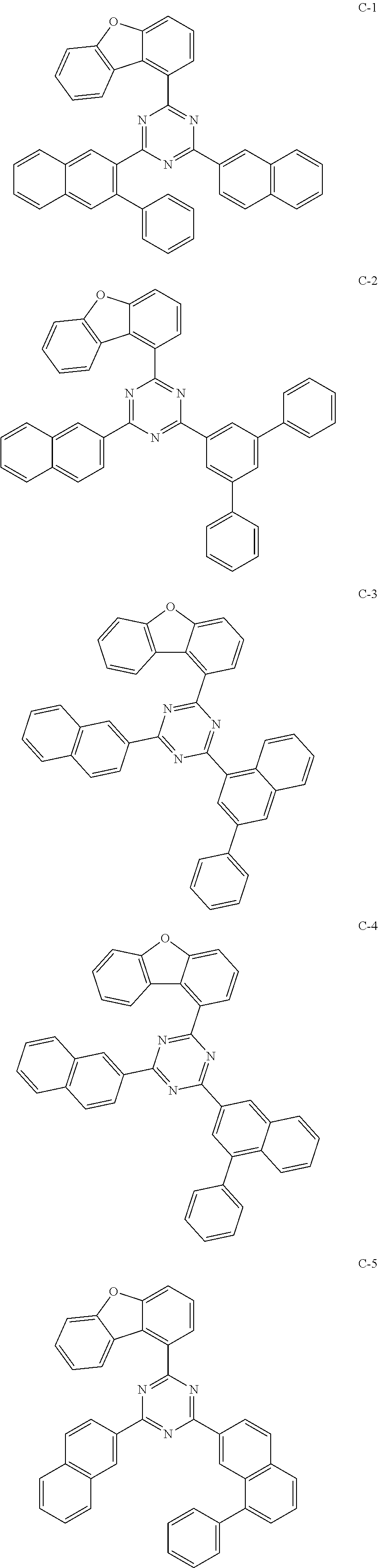

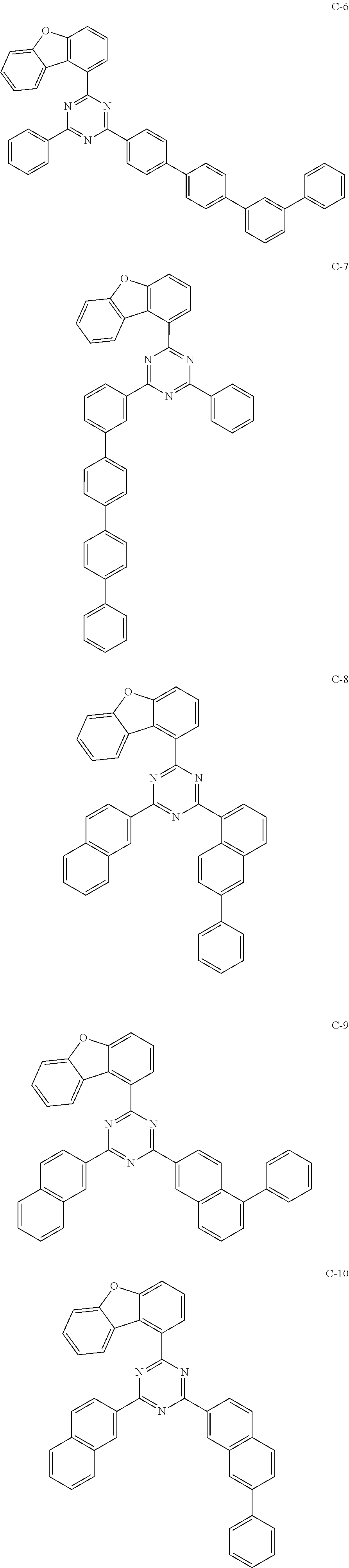 [SMILES: c1ccc(-c2cccc(-c3ccc(-c4ccc(-c5nc(-c6ccccc6)nc(-c6cccc7oc8ccccc8c67)n5)cc4)cc3)c2)cc1 ; c1ccc(-c2ccc(-c3ccc(-c4cccc(-c5nc(-c6ccccc6)nc(-c6cccc7oc8ccccc8c67)n5)c4)cc3)cc2)cc1 ; c1ccc(-c2ccc3c(-c4nc(-c5ccc6ccccc6c5)nc(-c5cccc6oc7ccccc7c56)n4)cccc3c2)cc1 ; c1ccc(-c2cccc3cc(-c4nc(-c5ccc6ccccc6c5)nc(-c5cccc6oc7ccccc7c56)n4)ccc23)cc1 ; c1ccc(-c2ccc3ccc(-c4nc(-c5ccc6ccccc6c5)nc(-c5cccc6oc7ccccc7c56)n4)cc3c2)cc1]

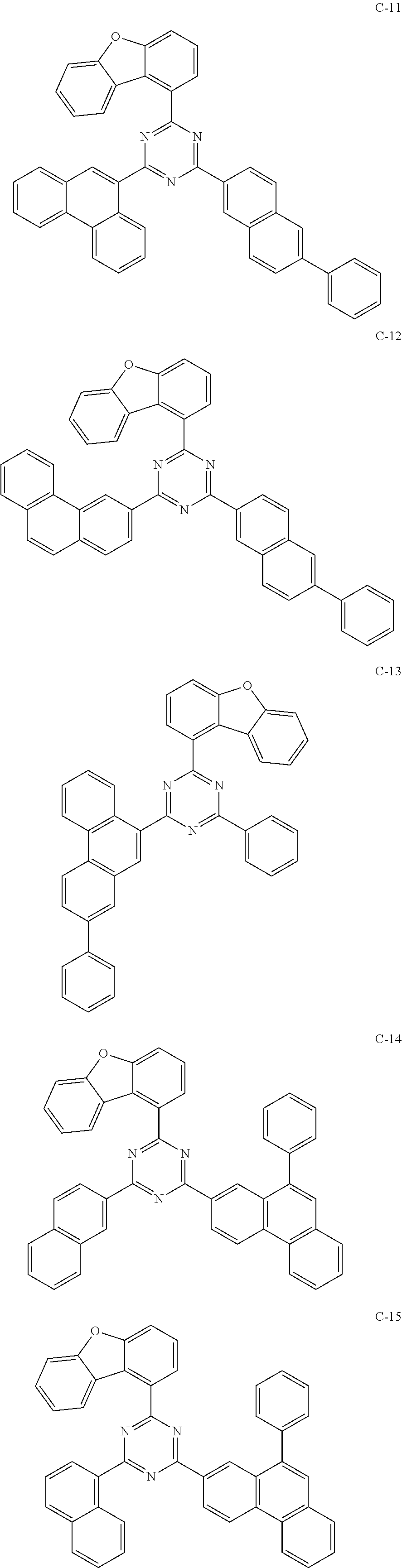 [SMILES: c1ccc(-c2ccc3cc(-c4nc(-c5cc6ccccc6c6ccccc56)nc(-c5cccc6oc7ccccc7c56)n4)ccc3c2)cc1 ; c1ccc(-c2ccc3cc(-c4nc(-c5ccc6ccc7ccccc7c6c5)nc(-c5cccc6oc7ccccc7c56)n4)ccc3c2)cc1 ; c1ccc(-c2ccc3c(c2)cc(-c2nc(-c4ccccc4)nc(-c4cccc5oc6ccccc6c45)n2)c2ccccc23)cc1 ; c1ccc(-c2cc3ccccc3c3ccc(-c4nc(-c5ccc6ccccc6c5)nc(-c5cccc6oc7ccccc7c56)n4)cc23)cc1 ; c1ccc(-c2cc3ccccc3c3ccc(-c4nc(-c5cccc6ccccc56)nc(-c5cccc6oc7ccccc7c56)n4)cc23)cc1]

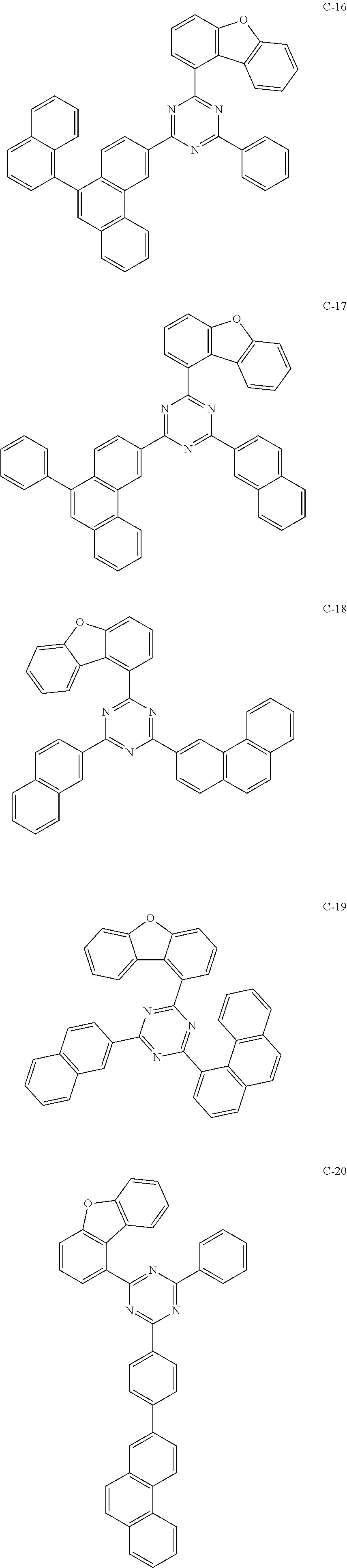 [SMILES: c1ccc(-c2nc(-c3ccc4c(-c5cccc6ccccc56)cc5ccccc5c4c3)nc(-c3cccc4oc5ccccc5c34)n2)cc1 ; c1ccc(-c2cc3ccccc3c3cc(-c4nc(-c5ccc6ccccc6c5)nc(-c5cccc6oc7ccccc7c56)n4)ccc23)cc1 ; c1ccc2cc(-c3nc(-c4ccc5ccc6ccccc6c5c4)nc(-c4cccc5oc6ccccc6c45)n3)ccc2c1 ; c1ccc2cc(-c3nc(-c4cccc5ccc6ccccc6c45)nc(-c4cccc5oc6ccccc6c45)n3)ccc2c1 ; c1ccc(-c2nc(-c3ccc(-c4ccc5c(ccc6ccccc65)c4)cc3)nc(-c3cccc4oc5ccccc5c34)n2)cc1]

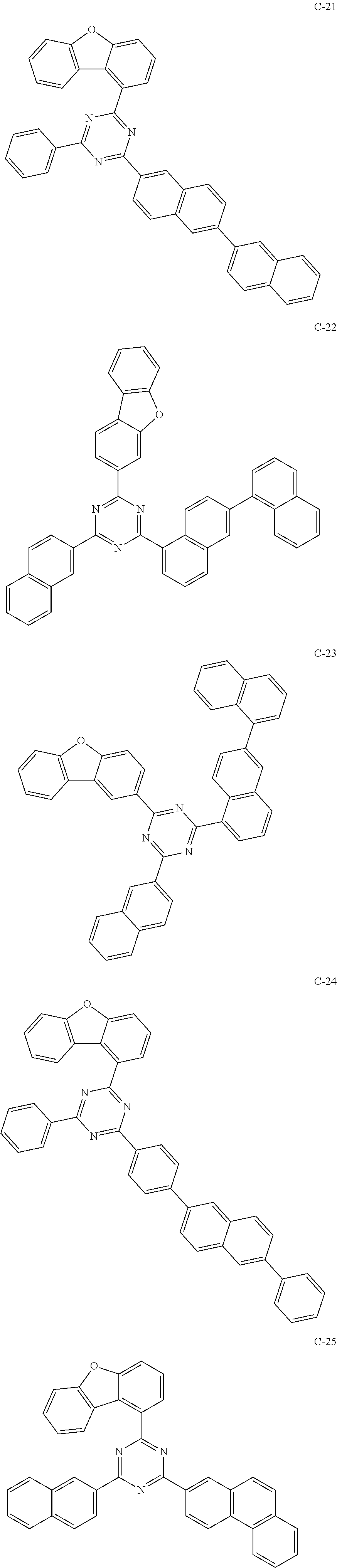 [SMILES: c1ccc(-c2nc(-c3ccc4cc(-c5ccc6ccccc6c5)ccc4c3)nc(-c3cccc4oc5ccccc5c34)n2)cc1 ; c1ccc2cc(-c3nc(-c4ccc5c(c4)oc4ccccc45)nc(-c4cccc5cc(-c6cccc7ccccc67)ccc45)n3)ccc2c1 ; c1ccc2cc(-c3nc(-c4ccc5oc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7oc8ccccc8c67)n5)cc4)ccc3c2)cc1 ; c1ccc2cc(-c3nc(-c4ccc5c(ccc6ccccc65)c4)nc(-c4cccc5oc6ccccc6c45)n3)ccc2c1]

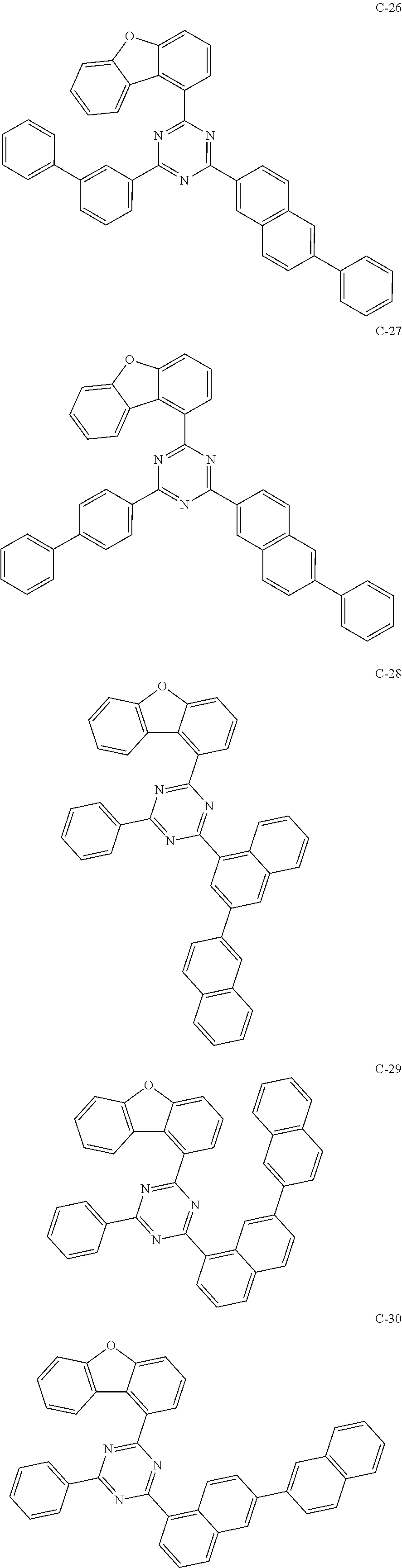 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc(-c3nc(-c4ccc5cc(-c6ccccc6)ccc5c4)nc(-c4cccc5oc6ccccc6c45)n3)cc2)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cccc4ccc(-c5ccc6ccccc6c5)cc34)nc(-c3cccc4oc5ccccc5c34)n2)cc1 ; c1ccc(-c2nc(-c3cccc4cc(-c5ccc6ccccc6c5)ccc34)nc(-c3cccc4oc5ccccc5c34)n2)cc1]

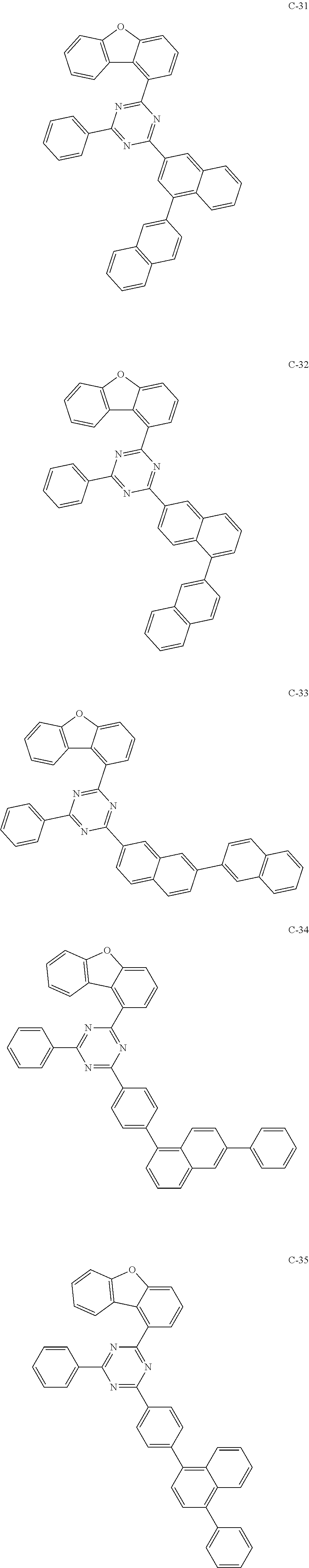 [SMILES: c1ccc(-c2nc(-c3cc(-c4ccc5ccccc5c4)c4ccccc4c3)nc(-c3cccc4oc5ccccc5c34)n2)cc1 ; c1ccc(-c2nc(-c3ccc4c(-c5ccc6ccccc6c5)cccc4c3)nc(-c3cccc4oc5ccccc5c34)n2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2ccc3c(-c4ccc(-c5nc(-c6ccccc6)nc(-c6cccc7oc8ccccc8c67)n5)cc4)cccc3c2)cc1 ; c1ccc(-c2nc(-c3ccc(-c4ccc(-c5ccccc5)c5ccccc45)cc3)nc(-c3cccc4oc5ccccc5c34)n2)cc1]

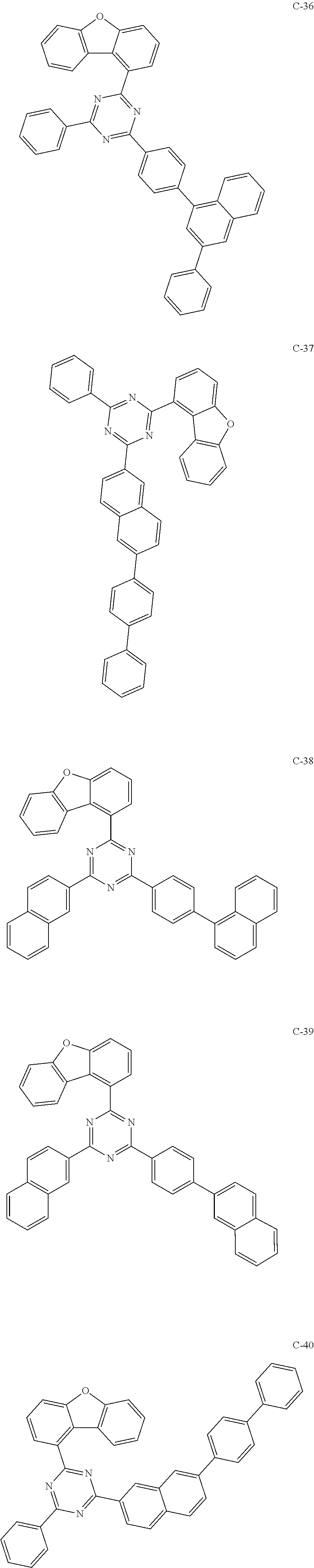 [SMILES: c1ccc(-c2cc(-c3ccc(-c4nc(-c5ccccc5)nc(-c5cccc6oc7ccccc7c56)n4)cc3)c3ccccc3c2)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc2cc(-c3nc(-c4ccc(-c5cccc6ccccc56)cc4)nc(-c4cccc5oc6ccccc6c45)n3)ccc2c1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3ccc4ccc(-c5nc(-c6ccccc6)nc(-c6cccc7oc8ccccc8c67)n5)cc4c3)cc2)cc1]

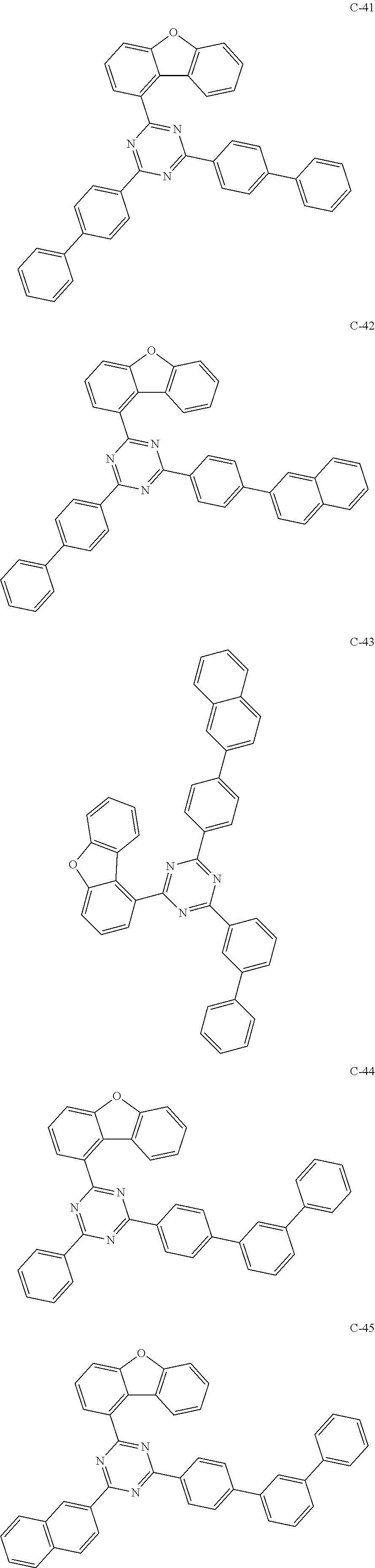 [SMILES: c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4ccc(-c5ccc6ccccc6c5)cc4)nc(-c4cccc5oc6ccccc6c45)n3)cc2)cc1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3ccc(-c4nc(-c5ccccc5)nc(-c5cccc6oc7ccccc7c56)n4)cc3)c2)cc1 ; c1ccc(-c2cccc(-c3ccc(-c4nc(-c5ccc6ccccc6c5)nc(-c5cccc6oc7ccccc7c56)n4)cc3)c2)cc1]

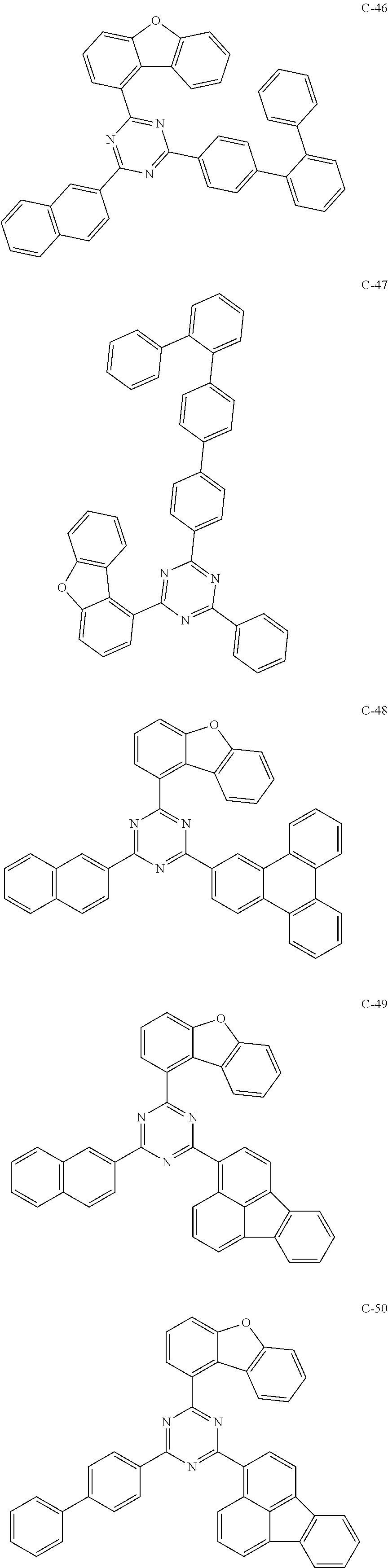 [SMILES: c1ccc(-c2ccccc2-c2ccc(-c3nc(-c4ccc5ccccc5c4)nc(-c4cccc5oc6ccccc6c45)n3)cc2)cc1 ; c1ccc(-c2nc(-c3ccc(-c4ccc(-c5ccccc5-c5ccccc5)cc4)cc3)nc(-c3cccc4oc5ccccc5c34)n2)cc1 ; c1ccc2cc(-c3nc(-c4ccc5c6ccccc6c6ccccc6c5c4)nc(-c4cccc5oc6ccccc6c45)n3)ccc2c1 ; c1ccc2c(c1)-c1cccc3c(-c4nc(-c5ccc6ccccc6c5)nc(-c5cccc6oc7ccccc7c56)n4)ccc-2c13 ; c1ccc(-c2ccc(-c3nc(-c4ccc5c6c(cccc46)-c4ccccc4-5)nc(-c4cccc5oc6ccccc6c45)n3)cc2)cc1]

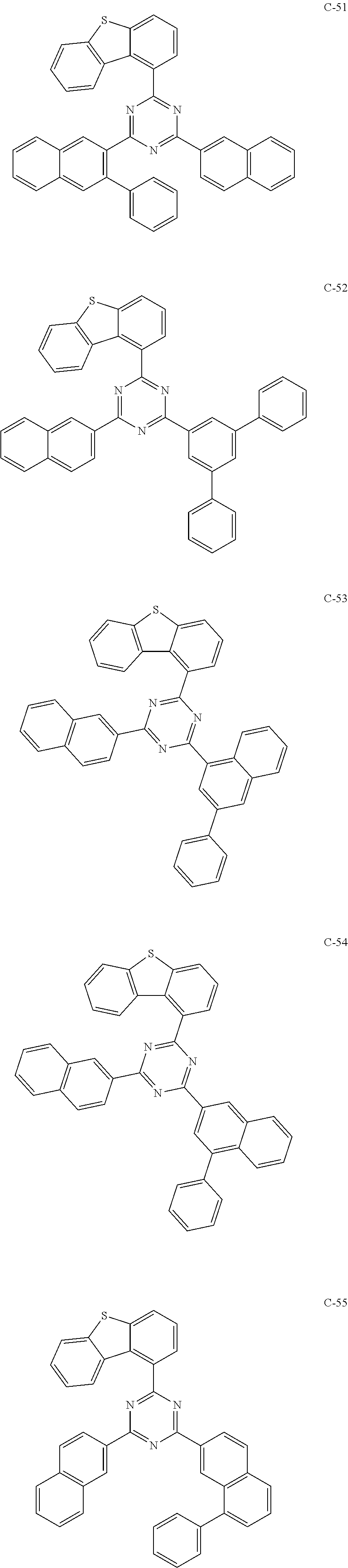 [SMILES: c1ccc(-c2cc3ccccc3cc2-c2nc(-c3ccc4ccccc4c3)nc(-c3cccc4sc5ccccc5c34)n2)cc1 ; c1ccc(-c2cc(-c3ccccc3)cc(-c3nc(-c4ccc5ccccc5c4)nc(-c4cccc5sc6ccccc6c45)n3)c2)cc1 ; c1ccc(-c2cc(-c3nc(-c4ccc5ccccc5c4)nc(-c4cccc5sc6ccccc6c45)n3)c3ccccc3c2)cc1 ; c1ccc(-c2cc(-c3nc(-c4ccc5ccccc5c4)nc(-c4cccc5sc6ccccc6c45)n3)cc3ccccc23)cc1 ; c1ccc(-c2cccc3ccc(-c4nc(-c5ccc6ccccc6c5)nc(-c5cccc6sc7ccccc7c56)n4)cc23)cc1]

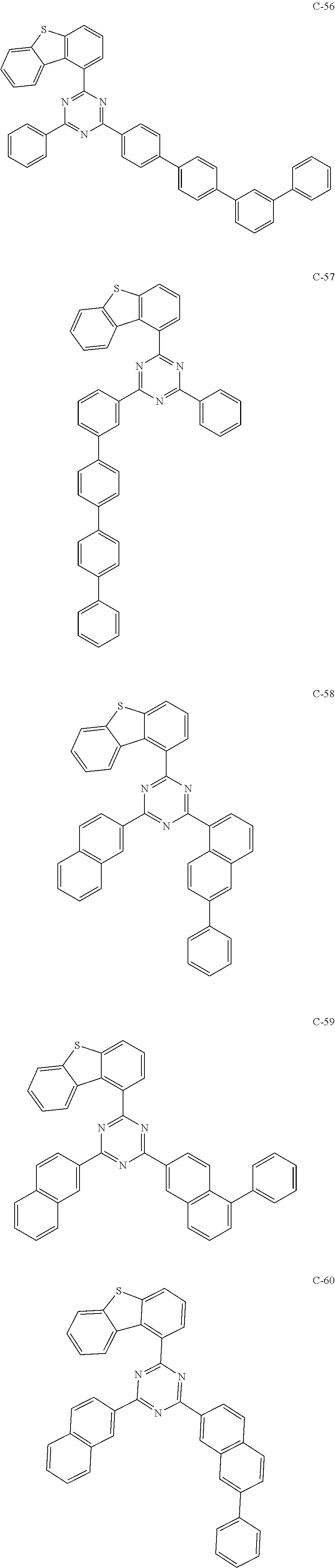 [SMILES: c1ccc(-c2cccc(-c3ccc(-c4ccc(-c5nc(-c6ccccc6)nc(-c6cccc7sc8ccccc8c67)n5)cc4)cc3)c2)cc1 ; c1ccc(-c2ccc(-c3ccc(-c4cccc(-c5nc(-c6ccccc6)nc(-c6cccc7sc8ccccc8c67)n5)c4)cc3)cc2)cc1 ; c1ccc(-c2ccc3c(-c4nc(-c5ccc6ccccc6c5)nc(-c5cccc6sc7ccccc7c56)n4)cccc3c2)cc1 ; c1ccc(-c2cccc3cc(-c4nc(-c5ccc6ccccc6c5)nc(-c5cccc6sc7ccccc7c56)n4)ccc23)cc1 ; c1ccc(-c2ccc3ccc(-c4nc(-c5ccc6ccccc6c5)nc(-c5cccc6sc7ccccc7c56)n4)cc3c2)cc1]

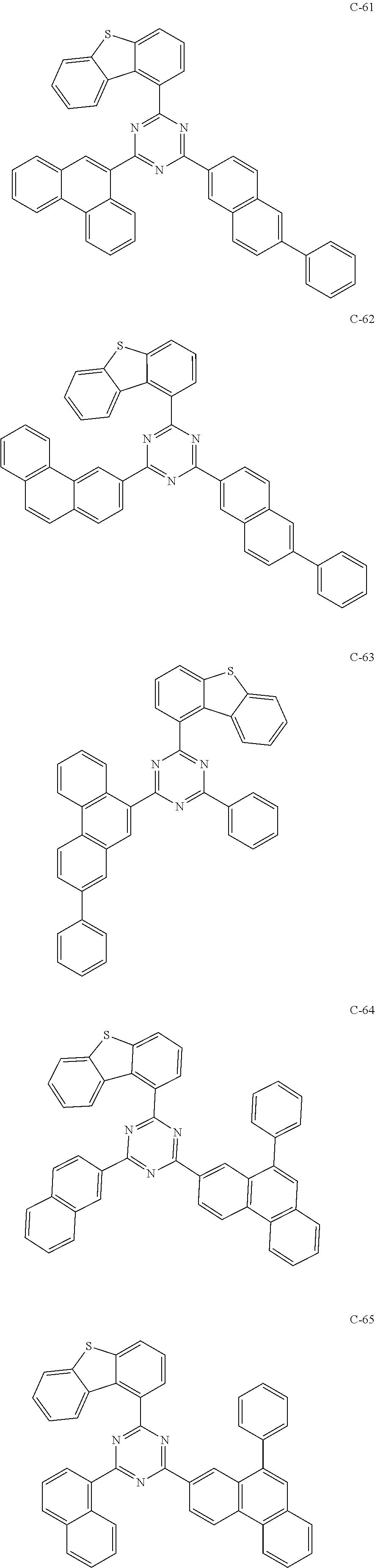 [SMILES: c1ccc(-c2ccc3cc(-c4nc(-c5cc6ccccc6c6ccccc56)nc(-c5cccc6sc7ccccc7c56)n4)ccc3c2)cc1 ; c1ccc(-c2ccc3cc(-c4nc(-c5ccc6ccc7ccccc7c6c5)nc(-c5cccc6sc7ccccc7c56)n4)ccc3c2)cc1 ; c1ccc(-c2ccc3c(c2)cc(-c2nc(-c4ccccc4)nc(-c4cccc5sc6ccccc6c45)n2)c2ccccc23)cc1 ; c1ccc(-c2cc3ccccc3c3ccc(-c4nc(-c5ccc6ccccc6c5)nc(-c5cccc6sc7ccccc7c56)n4)cc23)cc1 ; c1ccc(-c2cc3ccccc3c3ccc(-c4nc(-c5cccc6ccccc56)nc(-c5cccc6sc7ccccc7c56)n4)cc23)cc1]

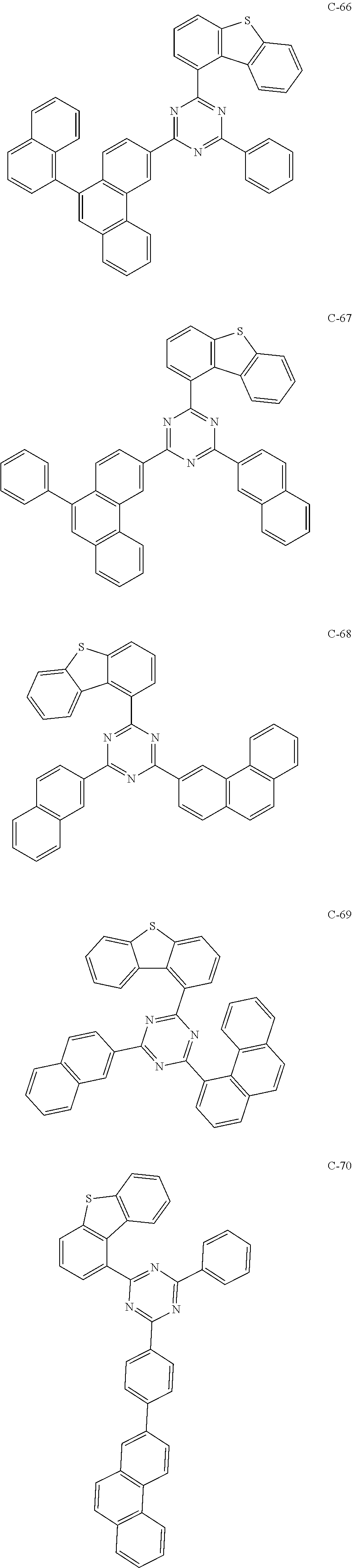 [SMILES: c1ccc(-c2nc(-c3ccc4c(-c5cccc6ccccc56)cc5ccccc5c4c3)nc(-c3cccc4sc5ccccc5c34)n2)cc1 ; c1ccc(-c2cc3ccccc3c3cc(-c4nc(-c5ccc6ccccc6c5)nc(-c5cccc6sc7ccccc7c56)n4)ccc23)cc1 ; c1ccc2cc(-c3nc(-c4ccc5ccc6ccccc6c5c4)nc(-c4cccc5sc6ccccc6c45)n3)ccc2c1 ; c1ccc2cc(-c3nc(-c4cccc5ccc6ccccc6c45)nc(-c4cccc5sc6ccccc6c45)n3)ccc2c1 ; c1ccc(-c2nc(-c3ccc(-c4ccc5c(ccc6ccccc65)c4)cc3)nc(-c3cccc4sc5ccccc5c34)n2)cc1]

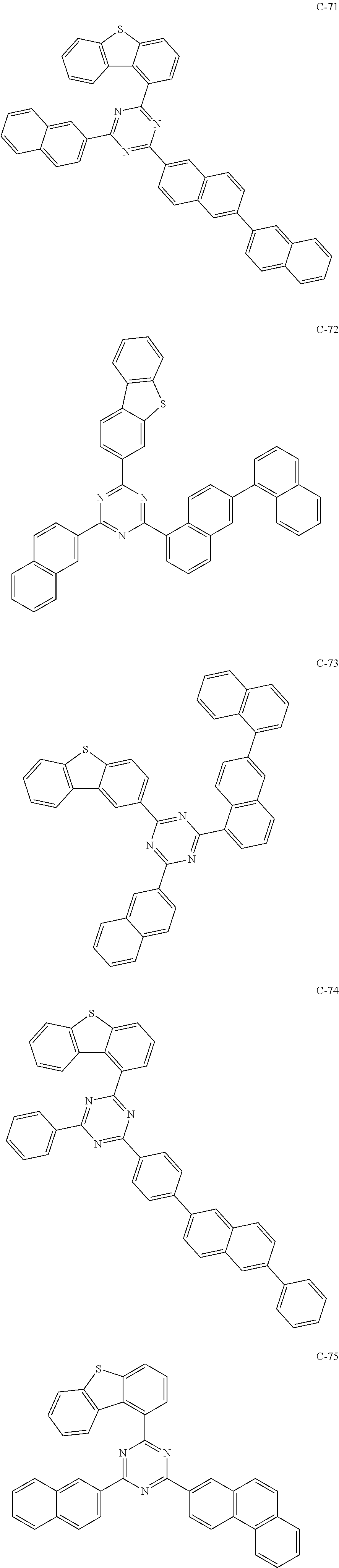 [SMILES: c1ccc2cc(-c3ccc4cc(-c5nc(-c6ccc7ccccc7c6)nc(-c6cccc7sc8ccccc8c67)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1 ; c1ccc2cc(-c3nc(-c4ccc5sc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7sc8ccccc8c67)n5)cc4)ccc3c2)cc1 ; c1ccc2cc(-c3nc(-c4ccc5c(ccc6ccccc65)c4)nc(-c4cccc5sc6ccccc6c45)n3)ccc2c1]

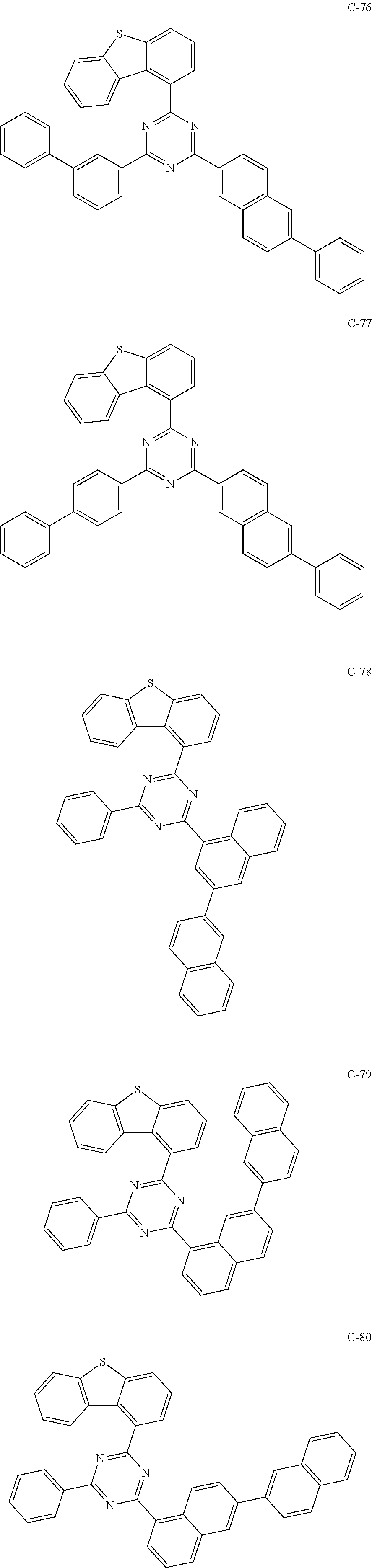 [SMILES: c1ccc(-c2cccc(-c3nc(-c4ccc5cc(-c6ccccc6)ccc5c4)nc(-c4cccc5sc6ccccc6c45)n3)c2)cc1 ; c1ccc(-c2ccc(-c3nc(-c4ccc5cc(-c6ccccc6)ccc5c4)nc(-c4cccc5sc6ccccc6c45)n3)cc2)cc1 ; c1ccc(-c2nc(-c3cc(-c4ccc5ccccc5c4)cc4ccccc34)nc(-c3cccc4sc5ccccc5c34)n2)cc1 ; c1ccc(-c2nc(-c3cccc4ccc(-c5ccc6ccccc6c5)cc34)nc(-c3cccc4sc5ccccc5c34)n2)cc1 ; c1ccc(-c2nc(-c3cccc4cc(-c5ccc6ccccc6c5)ccc34)nc(-c3cccc4sc5ccccc5c34)n2)cc1]

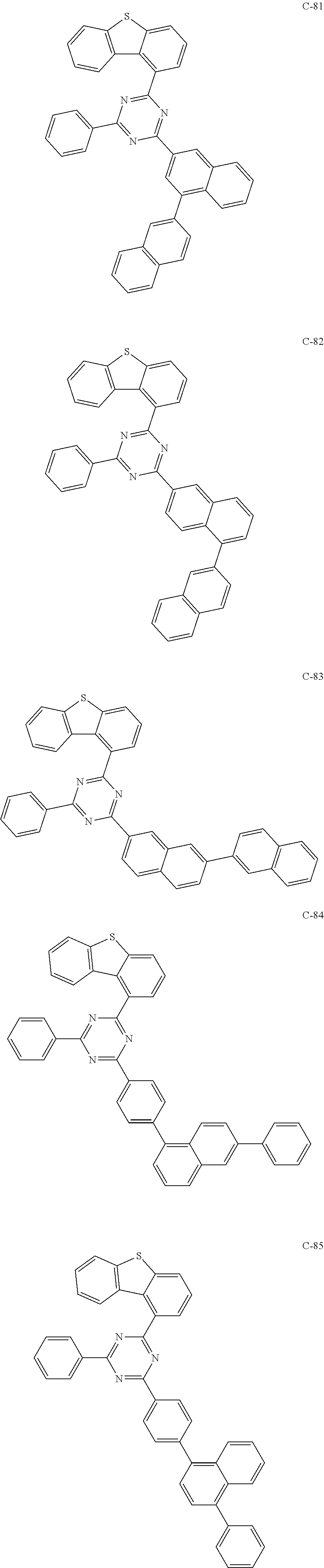 [SMILES: c1ccc(-c2nc(-c3cc(-c4ccc5ccccc5c4)c4ccccc4c3)nc(-c3cccc4sc5ccccc5c34)n2)cc1 ; c1ccc(-c2nc(-c3ccc4c(-c5ccc6ccccc6c5)cccc4c3)nc(-c3cccc4sc5ccccc5c34)n2)cc1 ; c1ccc(-c2nc(-c3ccc4ccc(-c5ccc6ccccc6c5)cc4c3)nc(-c3cccc4sc5ccccc5c34)n2)cc1 ; c1ccc(-c2ccc3c(-c4ccc(-c5nc(-c6ccccc6)nc(-c6cccc7sc8ccccc8c67)n5)cc4)cccc3c2)cc1 ; c1ccc(-c2nc(-c3ccc(-c4ccc(-c5ccccc5)c5ccccc45)cc3)nc(-c3cccc4sc5ccccc5c34)n2)cc1]

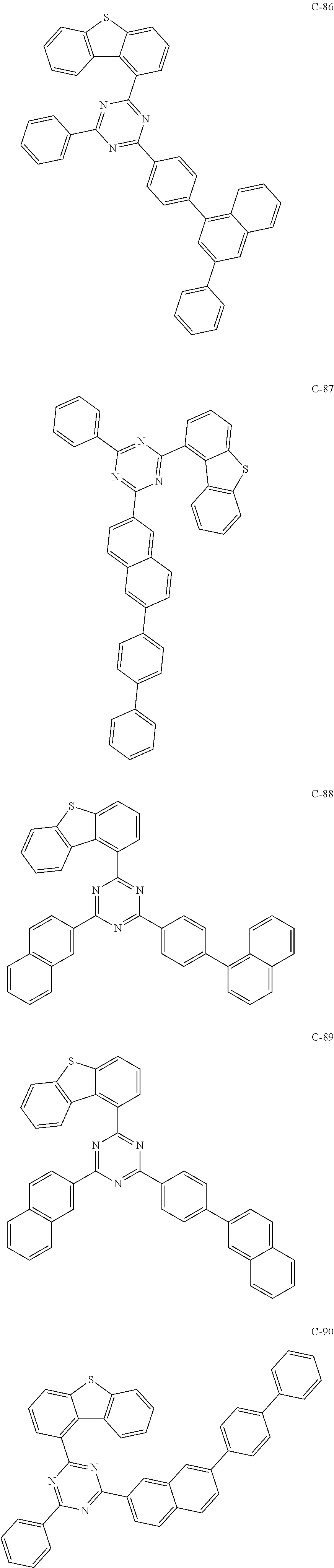 [SMILES: c1ccc(-c2cc(-c3ccc(-c4nc(-c5ccccc5)nc(-c5cccc6sc7ccccc7c56)n4)cc3)c3ccccc3c2)cc1 ; c1ccc(-c2ccc(-c3ccc4cc(-c5nc(-c6ccccc6)nc(-c6cccc7sc8ccccc8c67)n5)ccc4c3)cc2)cc1 ; c1ccc2cc(-c3nc(-c4ccc(-c5cccc6ccccc56)cc4)nc(-c4cccc5sc6ccccc6c45)n3)ccc2c1 ; c1ccc2cc(-c3ccc(-c4nc(-c5ccc6ccccc6c5)nc(-c5cccc6sc7ccccc7c56)n4)cc3)ccc2c1 ; c1ccc(-c2ccc(-c3ccc4ccc(-c5nc(-c6ccccc6)nc(-c6cccc7sc8ccccc8c67)n5)cc4c3)cc2)cc1]

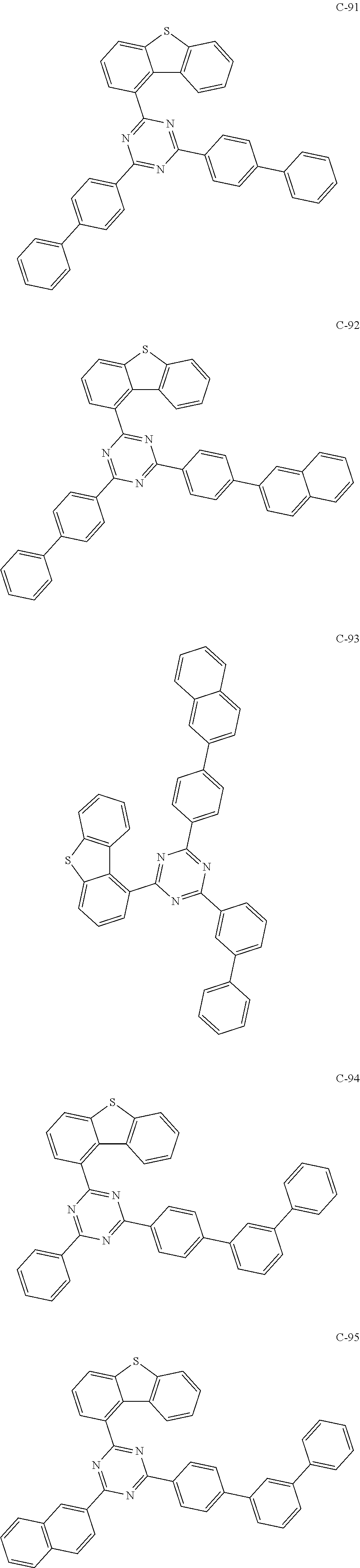 [SMILES: c1ccc(-c2ccc(-c3nc(-c4ccc(-c5ccccc5)cc4)nc(-c4cccc5sc6ccccc6c45)n3)cc2)cc1 ; c1ccc(-c2ccc(-c3nc(-c4ccc(-c5ccc6ccccc6c5)cc4)nc(-c4cccc5sc6ccccc6c45)n3)cc2)cc1 ; c1ccc(-c2cccc(-c3nc(-c4ccc(-c5ccc6ccccc6c5)cc4)nc(-c4cccc5sc6ccccc6c45)n3)c2)cc1 ; c1ccc(-c2cccc(-c3ccc(-c4nc(-c5ccccc5)nc(-c5cccc6sc7ccccc7c56)n4)cc3)c2)cc1 ; c1ccc(-c2cccc(-c3ccc(-c4nc(-c5ccc6ccccc6c5)nc(-c5cccc6sc7ccccc7c56)n4)cc3)c2)cc1]

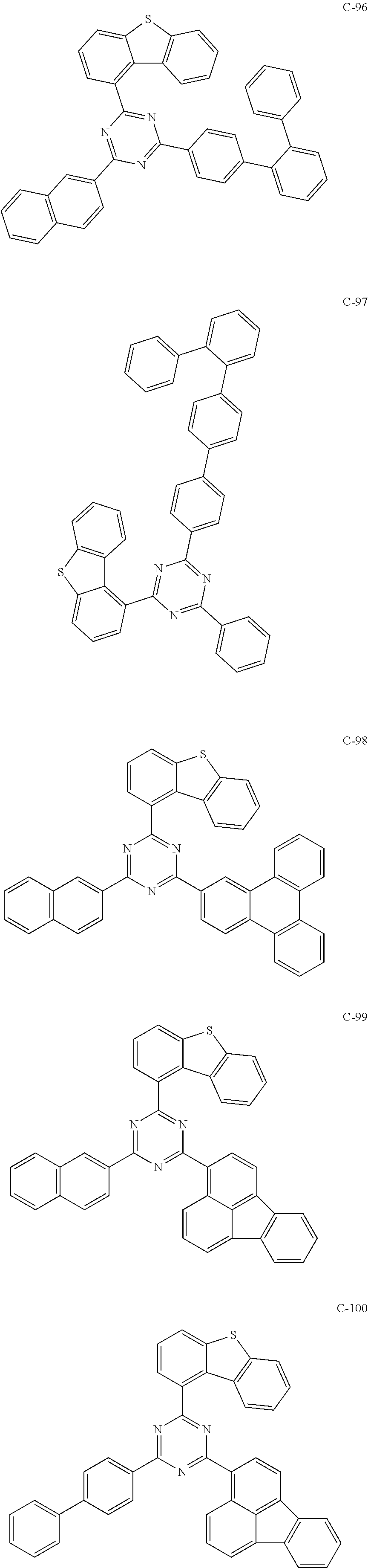 [SMILES: c1ccc(-c2ccccc2-c2ccc(-c3nc(-c4ccc5ccccc5c4)nc(-c4cccc5sc6ccccc6c45)n3)cc2)cc1 ; c1ccc(-c2nc(-c3ccc(-c4ccc(-c5ccccc5-c5ccccc5)cc4)cc3)nc(-c3cccc4sc5ccccc5c34)n2)cc1 ; c1ccc2cc(-c3nc(-c4ccc5c6ccccc6c6ccccc6c5c4)nc(-c4cccc5sc6ccccc6c45)n3)ccc2c1 ; c1ccc2c(c1)-c1cccc3c(-c4nc(-c5ccc6ccccc6c5)nc(-c5cccc6sc7ccccc7c56)n4)ccc-2c13 ; c1ccc(-c2ccc(-c3nc(-c4ccc5c6c(cccc46)-c4ccccc4-5)nc(-c4cccc5sc6ccccc6c45)n3)cc2)cc1]

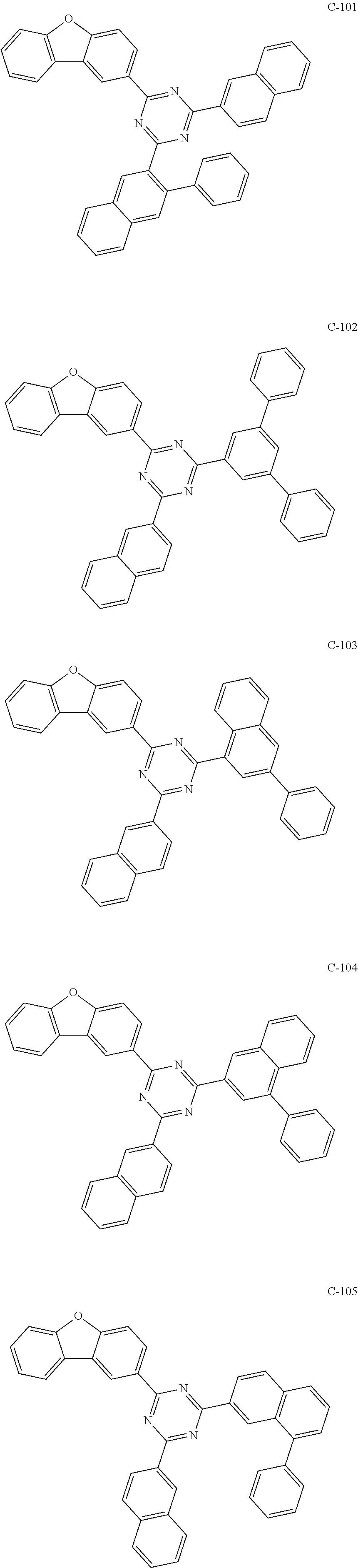 [SMILES: c1ccc(-c2cc3ccccc3cc2-c2nc(-c3ccc4ccccc4c3)nc(-c3ccc4oc5ccccc5c4c3)n2)cc1 ; c1ccc(-c2cc(-c3ccccc3)cc(-c3nc(-c4ccc5ccccc5c4)nc(-c4ccc5oc6ccccc6c5c4)n3)c2)cc1 ; c1ccc(-c2cc(-c3nc(-c4ccc5ccccc5c4)nc(-c4ccc5oc6ccccc6c5c4)n3)c3ccccc3c2)cc1 ; c1ccc(-c2cc(-c3nc(-c4ccc5ccccc5c4)nc(-c4ccc5oc6ccccc6c5c4)n3)cc3ccccc23)cc1 ; c1ccc(-c2cccc3ccc(-c4nc(-c5ccc6ccccc6c5)nc(-c5ccc6oc7ccccc7c6c5)n4)cc23)cc1]

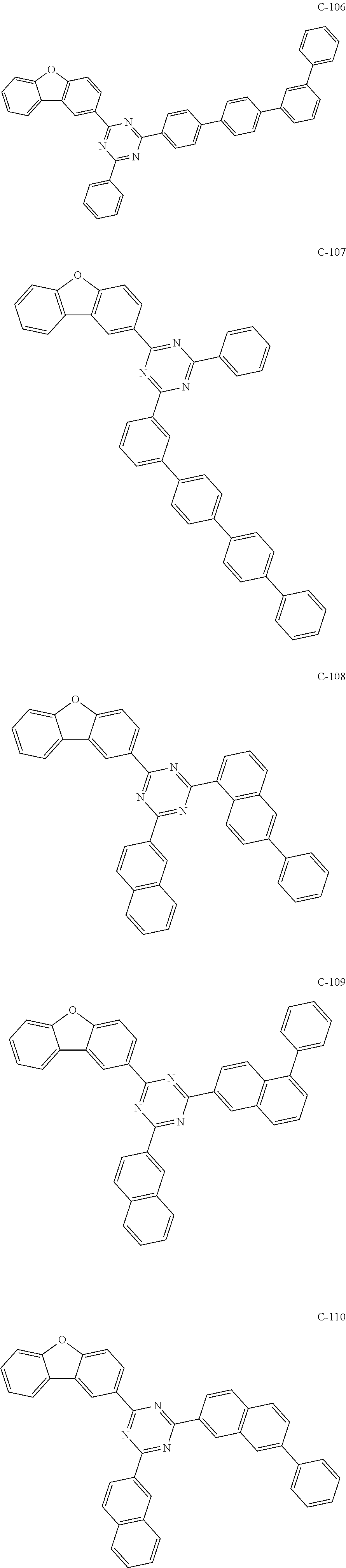 [SMILES: c1ccc(-c2cccc(-c3ccc(-c4ccc(-c5nc(-c6ccccc6)nc(-c6ccc7oc8ccccc8c7c6)n5)cc4)cc3)c2)cc1 ; c1ccc(-c2ccc(-c3ccc(-c4cccc(-c5nc(-c6ccccc6)nc(-c6ccc7oc8ccccc8c7c6)n5)c4)cc3)cc2)cc1 ; c1ccc(-c2ccc3c(-c4nc(-c5ccc6ccccc6c5)nc(-c5ccc6oc7ccccc7c6c5)n4)cccc3c2)cc1 ; c1ccc(-c2cccc3cc(-c4nc(-c5ccc6ccccc6c5)nc(-c5ccc6oc7ccccc7c6c5)n4)ccc23)cc1 ; c1ccc(-c2ccc3ccc(-c4nc(-c5ccc6ccccc6c5)nc(-c5ccc6oc7ccccc7c6c5)n4)cc3c2)cc1]

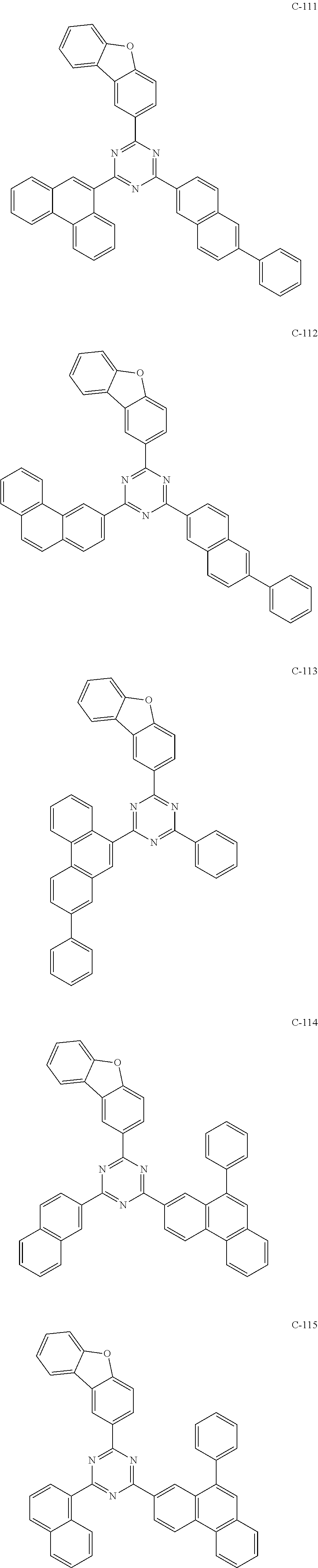 [SMILES: c1ccc(-c2ccc3cc(-c4nc(-c5ccc6oc7ccccc7c6c5)nc(-c5cc6ccccc6c6ccccc56)n4)ccc3c2)cc1 ; c1ccc(-c2ccc3cc(-c4nc(-c5ccc6ccc7ccccc7c6c5)nc(-c5ccc6oc7ccccc7c6c5)n4)ccc3c2)cc1 ; c1ccc(-c2ccc3c(c2)cc(-c2nc(-c4ccccc4)nc(-c4ccc5oc6ccccc6c5c4)n2)c2ccccc23)cc1 ; c1ccc(-c2cc3ccccc3c3ccc(-c4nc(-c5ccc6ccccc6c5)nc(-c5ccc6oc7ccccc7c6c5)n4)cc23)cc1 ; c1ccc(-c2cc3ccccc3c3ccc(-c4nc(-c5ccc6oc7ccccc7c6c5)nc(-c5cccc6ccccc56)n4)cc23)cc1]

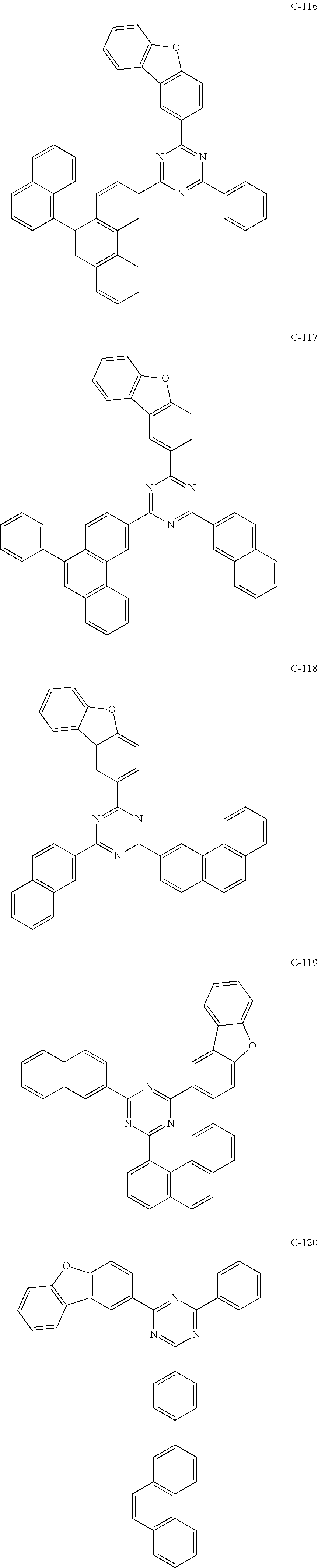 [SMILES: c1ccc(-c2nc(-c3ccc4oc5ccccc5c4c3)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6oc7ccccc7c6c5)n4)ccc23)cc1 ; c1ccc2cc(-c3nc(-c4ccc5ccc6ccccc6c5c4)nc(-c4ccc5oc6ccccc6c5c4)n3)ccc2c1 ; c1ccc2cc(-c3nc(-c4ccc5oc6ccccc6c5c4)nc(-c4cccc5ccc6ccccc6c45)n3)ccc2c1 ; c1ccc(-c2nc(-c3ccc(-c4ccc5c(ccc6ccccc65)c4)cc3)nc(-c3ccc4oc5ccccc5c4c3)n2)cc1]

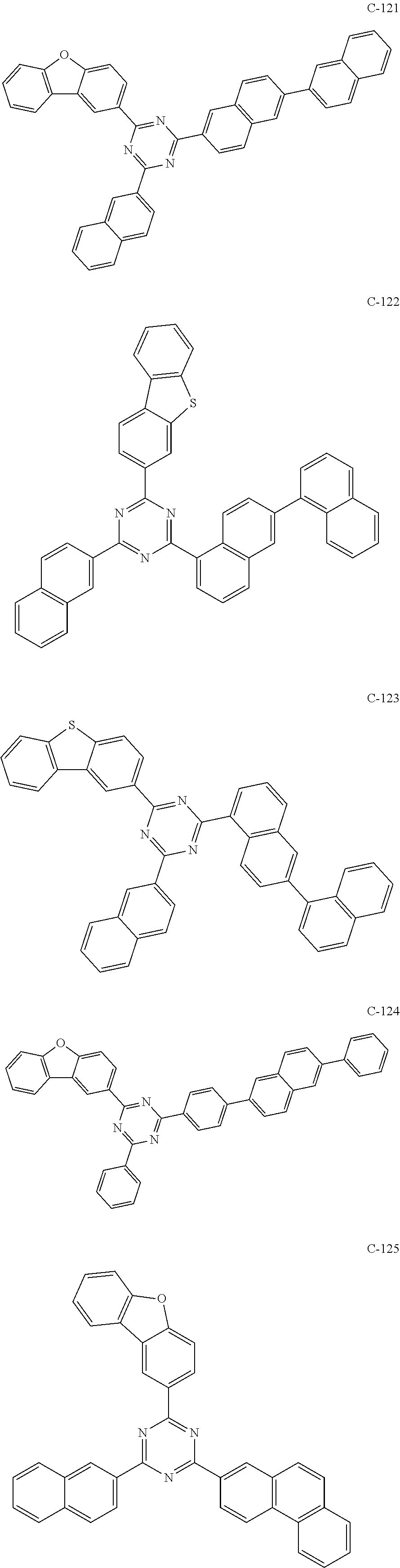 [SMILES: c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1 ; c1ccc2cc(-c3nc(-c4ccc5sc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6ccc7oc8ccccc8c7c6)n5)cc4)ccc3c2)cc1 ; c1ccc2cc(-c3nc(-c4ccc5c(ccc6ccccc65)c4)nc(-c4ccc5oc6ccccc6c5c4)n3)ccc2c1]

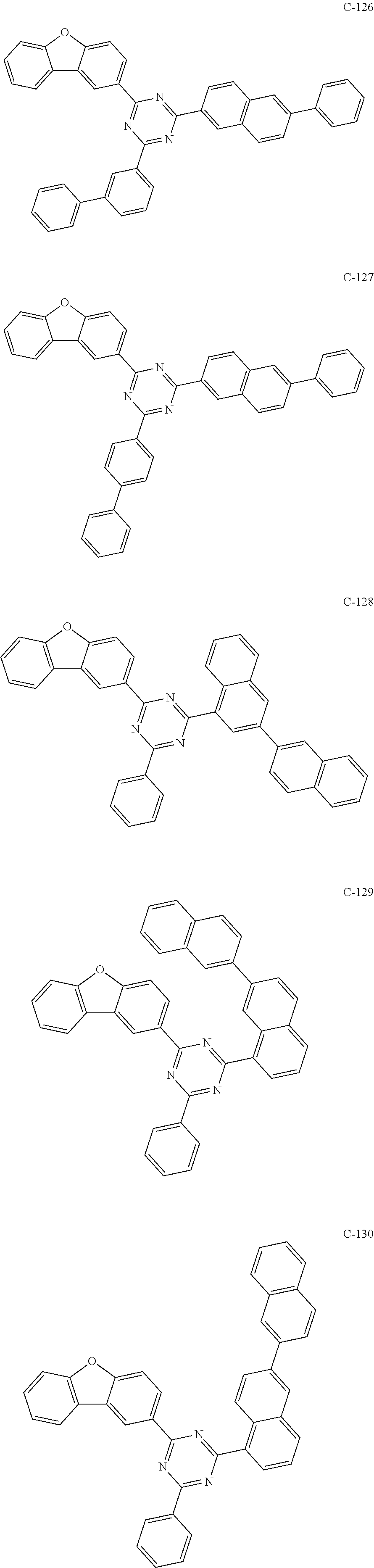 [SMILES: c1ccc(-c2cccc(-c3nc(-c4ccc5cc(-c6ccccc6)ccc5c4)nc(-c4ccc5oc6ccccc6c5c4)n3)c2)cc1 ; c1ccc(-c2ccc(-c3nc(-c4ccc5cc(-c6ccccc6)ccc5c4)nc(-c4ccc5oc6ccccc6c5c4)n3)cc2)cc1 ; c1ccc(-c2nc(-c3ccc4oc5ccccc5c4c3)nc(-c3cc(-c4ccc5ccccc5c4)cc4ccccc34)n2)cc1 ; c1ccc(-c2nc(-c3ccc4oc5ccccc5c4c3)nc(-c3cccc4ccc(-c5ccc6ccccc6c5)cc34)n2)cc1 ; c1ccc(-c2nc(-c3ccc4oc5ccccc5c4c3)nc(-c3cccc4cc(-c5ccc6ccccc6c5)ccc34)n2)cc1]

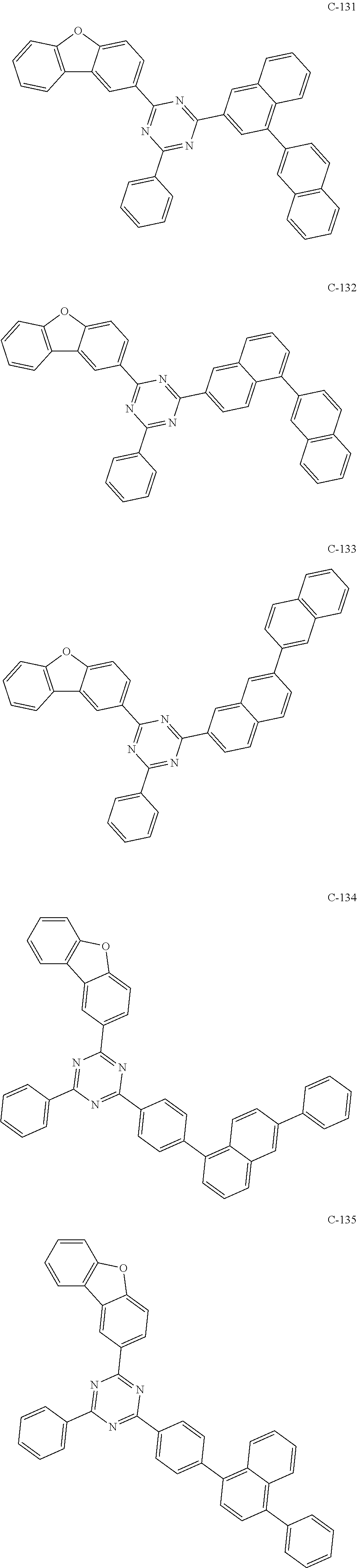 [SMILES: c1ccc(-c2nc(-c3cc(-c4ccc5ccccc5c4)c4ccccc4c3)nc(-c3ccc4oc5ccccc5c4c3)n2)cc1 ; c1ccc(-c2nc(-c3ccc4c(-c5ccc6ccccc6c5)cccc4c3)nc(-c3ccc4oc5ccccc5c4c3)n2)cc1 ; c1ccc(-c2nc(-c3ccc4ccc(-c5ccc6ccccc6c5)cc4c3)nc(-c3ccc4oc5ccccc5c4c3)n2)cc1 ; c1ccc(-c2ccc3c(-c4ccc(-c5nc(-c6ccccc6)nc(-c6ccc7oc8ccccc8c7c6)n5)cc4)cccc3c2)cc1 ; c1ccc(-c2nc(-c3ccc(-c4ccc(-c5ccccc5)c5ccccc45)cc3)nc(-c3ccc4oc5ccccc5c4c3)n2)cc1]

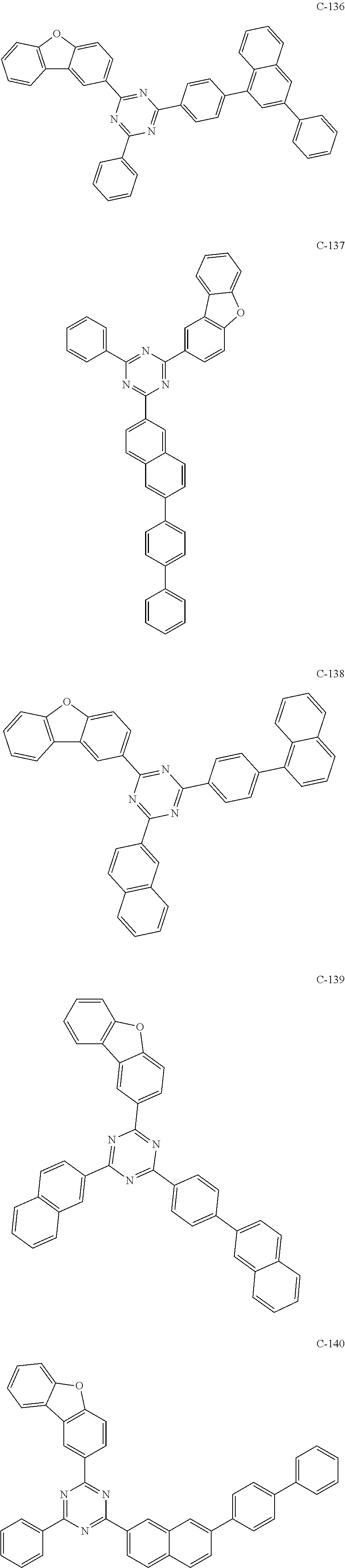 [SMILES: c1ccc(-c2cc(-c3ccc(-c4nc(-c5ccccc5)nc(-c5ccc6oc7ccccc7c6c5)n4)cc3)c3ccccc3c2)cc1 ; c1ccc(-c2ccc(-c3ccc4cc(-c5nc(-c6ccccc6)nc(-c6ccc7oc8ccccc8c7c6)n5)ccc4c3)cc2)cc1 ; c1ccc2cc(-c3nc(-c4ccc(-c5cccc6ccccc56)cc4)nc(-c4ccc5oc6ccccc6c5c4)n3)ccc2c1 ; c1ccc2cc(-c3ccc(-c4nc(-c5ccc6ccccc6c5)nc(-c5ccc6oc7ccccc7c6c5)n4)cc3)ccc2c1 ; c1ccc(-c2ccc(-c3ccc4ccc(-c5nc(-c6ccccc6)nc(-c6ccc7oc8ccccc8c7c6)n5)cc4c3)cc2)cc1]

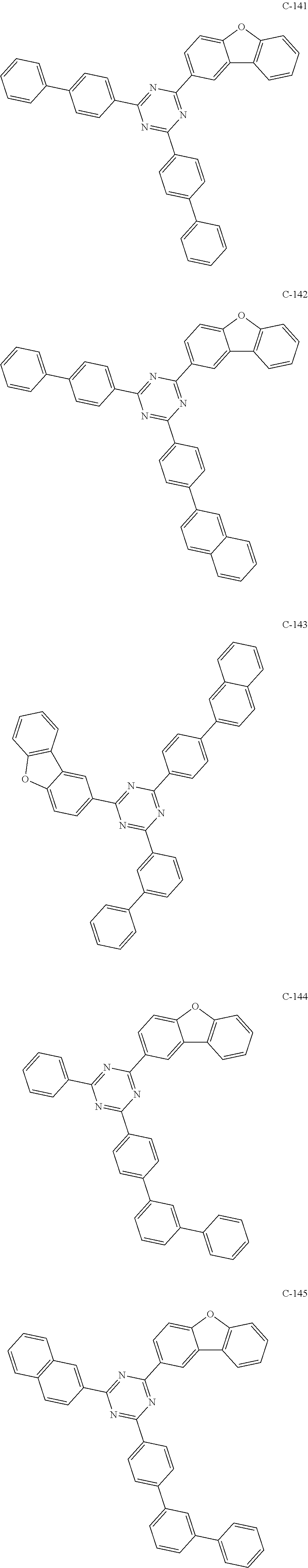 [SMILES: c1ccc(-c2ccc(-c3nc(-c4ccc(-c5ccccc5)cc4)nc(-c4ccc5oc6ccccc6c5c4)n3)cc2)cc1 ; c1ccc(-c2ccc(-c3nc(-c4ccc(-c5ccc6ccccc6c5)cc4)nc(-c4ccc5oc6ccccc6c5c4)n3)cc2)cc1 ; c1ccc(-c2cccc(-c3nc(-c4ccc(-c5ccc6ccccc6c5)cc4)nc(-c4ccc5oc6ccccc6c5c4)n3)c2)cc1 ; c1ccc(-c2cccc(-c3ccc(-c4nc(-c5ccccc5)nc(-c5ccc6oc7ccccc7c6c5)n4)cc3)c2)cc1 ; c1ccc(-c2cccc(-c3ccc(-c4nc(-c5ccc6ccccc6c5)nc(-c5ccc6oc7ccccc7c6c5)n4)cc3)c2)cc1]

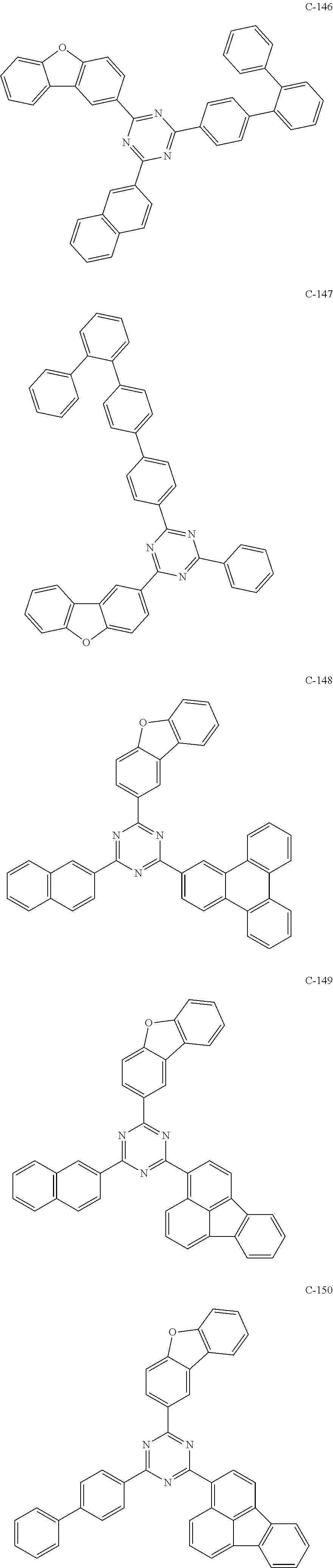 [SMILES: c1ccc(-c2ccccc2-c2ccc(-c3nc(-c4ccc5ccccc5c4)nc(-c4ccc5oc6ccccc6c5c4)n3)cc2)cc1 ; c1ccc(-c2nc(-c3ccc(-c4ccc(-c5ccccc5-c5ccccc5)cc4)cc3)nc(-c3ccc4oc5ccccc5c4c3)n2)cc1 ; c1ccc2cc(-c3nc(-c4ccc5oc6ccccc6c5c4)nc(-c4ccc5c6ccccc6c6ccccc6c5c4)n3)ccc2c1 ; c1ccc2c(c1)-c1cccc3c(-c4nc(-c5ccc6ccccc6c5)nc(-c5ccc6oc7ccccc7c6c5)n4)ccc-2c13 ; c1ccc(-c2ccc(-c3nc(-c4ccc5oc6ccccc6c5c4)nc(-c4ccc5c6c(cccc46)-c4ccccc4-5)n3)cc2)cc1]

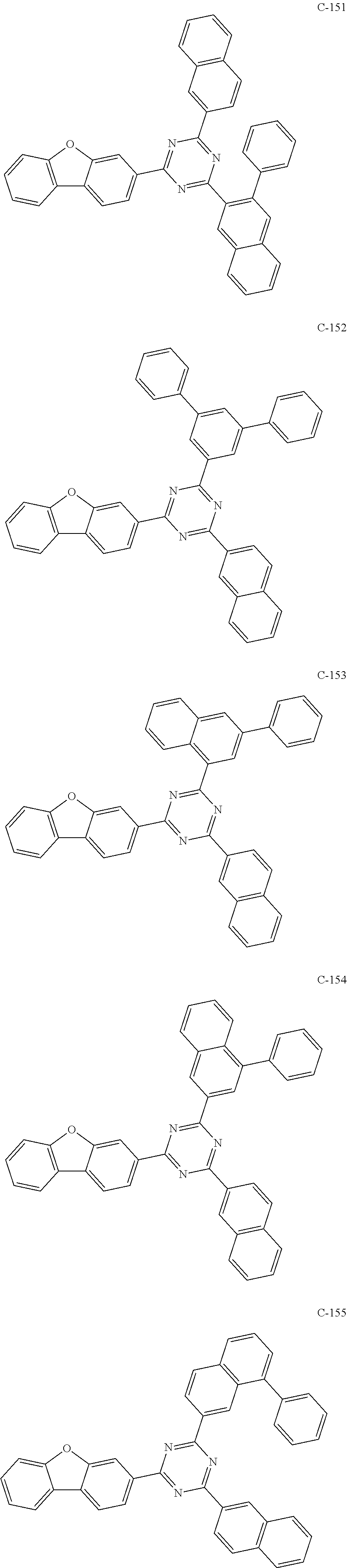 [SMILES: c1ccc(-c2cc3ccccc3cc2-c2nc(-c3ccc4ccccc4c3)nc(-c3ccc4c(c3)oc3ccccc34)n2)cc1 ; c1ccc(-c2cc(-c3ccccc3)cc(-c3nc(-c4ccc5ccccc5c4)nc(-c4ccc5c(c4)oc4ccccc45)n3)c2)cc1 ; c1ccc(-c2cc(-c3nc(-c4ccc5ccccc5c4)nc(-c4ccc5c(c4)oc4ccccc45)n3)c3ccccc3c2)cc1 ; c1ccc(-c2cc(-c3nc(-c4ccc5ccccc5c4)nc(-c4ccc5c(c4)oc4ccccc45)n3)cc3ccccc23)cc1 ; c1ccc(-c2cccc3ccc(-c4nc(-c5ccc6ccccc6c5)nc(-c5ccc6c(c5)oc5ccccc56)n4)cc23)cc1]

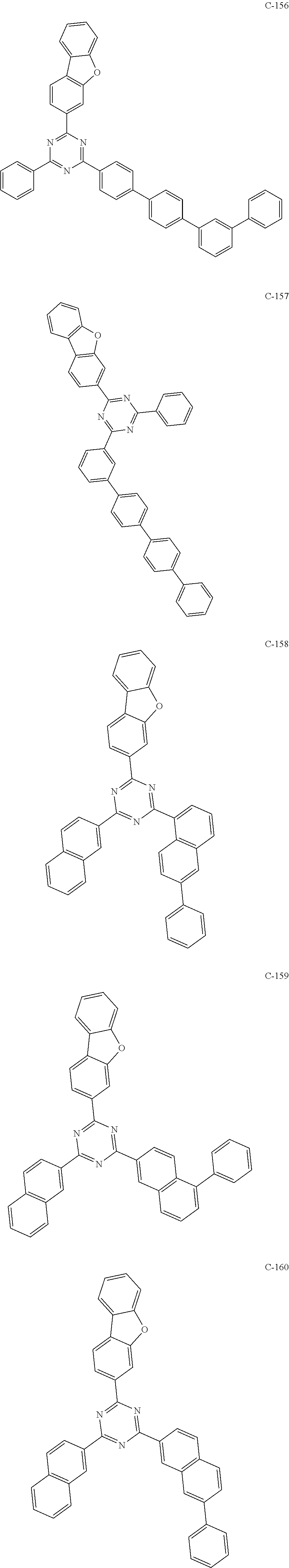 [SMILES: c1ccc(-c2cccc(-c3ccc(-c4ccc(-c5nc(-c6ccccc6)nc(-c6ccc7c(c6)oc6ccccc67)n5)cc4)cc3)c2)cc1 ; c1ccc(-c2ccc(-c3ccc(-c4cccc(-c5nc(-c6ccccc6)nc(-c6ccc7c(c6)oc6ccccc67)n5)c4)cc3)cc2)cc1 ; c1ccc(-c2ccc3c(-c4nc(-c5ccc6ccccc6c5)nc(-c5ccc6c(c5)oc5ccccc56)n4)cccc3c2)cc1 ; c1ccc(-c2cccc3cc(-c4nc(-c5ccc6ccccc6c5)nc(-c5ccc6c(c5)oc5ccccc56)n4)ccc23)cc1 ; c1ccc(-c2ccc3ccc(-c4nc(-c5ccc6ccccc6c5)nc(-c5ccc6c(c5)oc5ccccc56)n4)cc3c2)cc1]

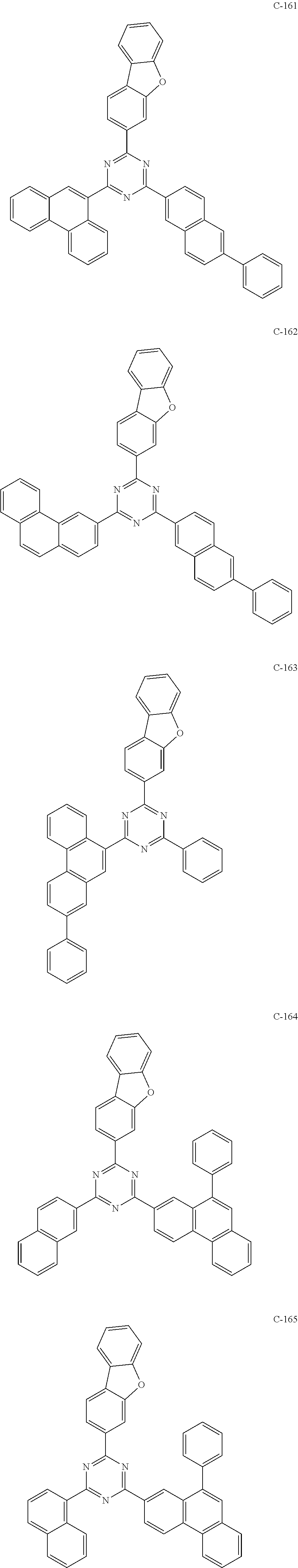 [SMILES: c1ccc(-c2ccc3cc(-c4nc(-c5ccc6c(c5)oc5ccccc56)nc(-c5cc6ccccc6c6ccccc56)n4)ccc3c2)cc1 ; c1ccc(-c2ccc3cc(-c4nc(-c5ccc6c(c5)oc5ccccc56)nc(-c5ccc6ccc7ccccc7c6c5)n4)ccc3c2)cc1 ; c1ccc(-c2ccc3c(c2)cc(-c2nc(-c4ccccc4)nc(-c4ccc5c(c4)oc4ccccc45)n2)c2ccccc23)cc1 ; c1ccc(-c2cc3ccccc3c3ccc(-c4nc(-c5ccc6ccccc6c5)nc(-c5ccc6c(c5)oc5ccccc56)n4)cc23)cc1 ; c1ccc(-c2cc3ccccc3c3ccc(-c4nc(-c5ccc6c(c5)oc5ccccc56)nc(-c5cccc6ccccc56)n4)cc23)cc1]

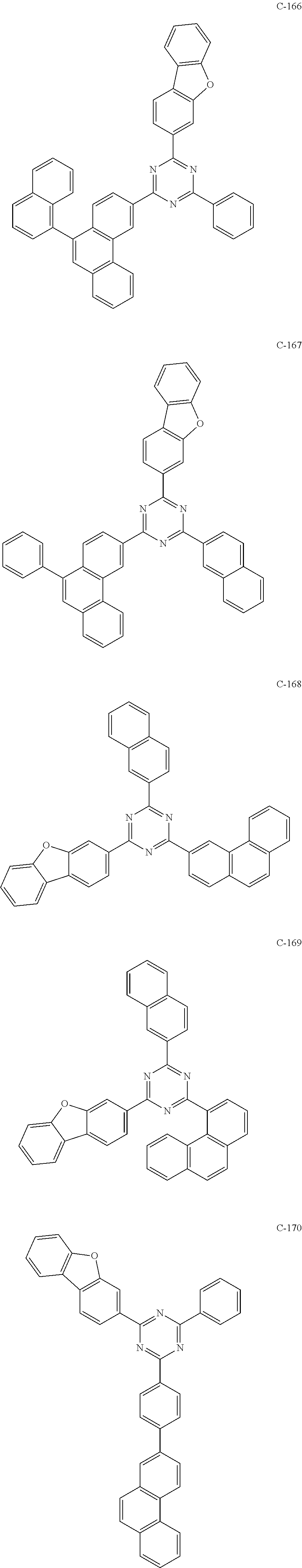 [SMILES: c1ccc(-c2nc(-c3ccc4c(c3)oc3ccccc34)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6c(c5)oc5ccccc56)n4)ccc23)cc1 ; c1ccc2cc(-c3nc(-c4ccc5c(c4)oc4ccccc45)nc(-c4ccc5ccc6ccccc6c5c4)n3)ccc2c1 ; c1ccc2cc(-c3nc(-c4ccc5c(c4)oc4ccccc45)nc(-c4cccc5ccc6ccccc6c45)n3)ccc2c1 ; c1ccc(-c2nc(-c3ccc(-c4ccc5c(ccc6ccccc65)c4)cc3)nc(-c3ccc4c(c3)oc3ccccc34)n2)cc1]

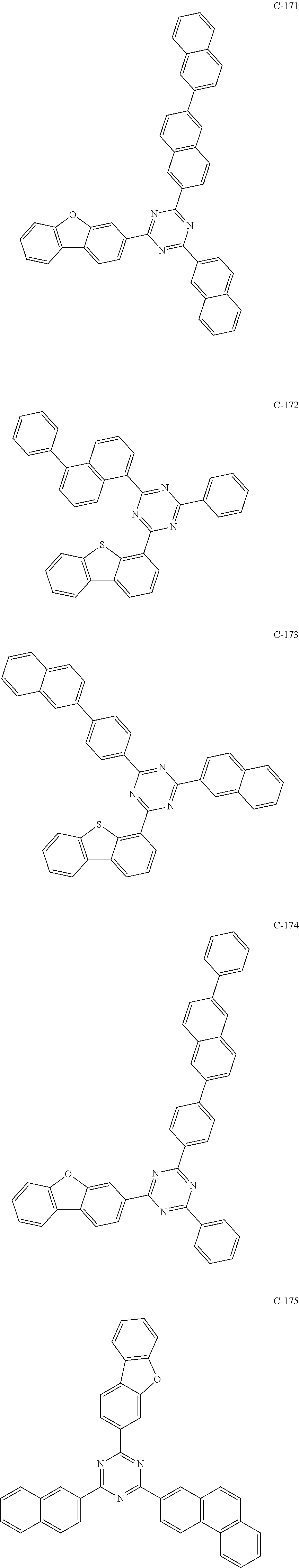 [SMILES: c1ccc2cc(-c3ccc4cc(-c5nc(-c6ccc7ccccc7c6)nc(-c6ccc7c(c6)oc6ccccc67)n5)ccc4c3)ccc2c1 ; c1ccc(-c2nc(-c3cccc4c(-c5ccccc5)cccc34)nc(-c3cccc4c3sc3ccccc34)n2)cc1 ; c1ccc2cc(-c3ccc(-c4nc(-c5ccc6ccccc6c5)nc(-c5cccc6c5sc5ccccc56)n4)cc3)ccc2c1 ; c1ccc(-c2ccc3cc(-c4ccc(-c5nc(-c6ccccc6)nc(-c6ccc7c(c6)oc6ccccc67)n5)cc4)ccc3c2)cc1 ; c1ccc2cc(-c3nc(-c4ccc5c(ccc6ccccc65)c4)nc(-c4ccc5c(c4)oc4ccccc45)n3)ccc2c1]

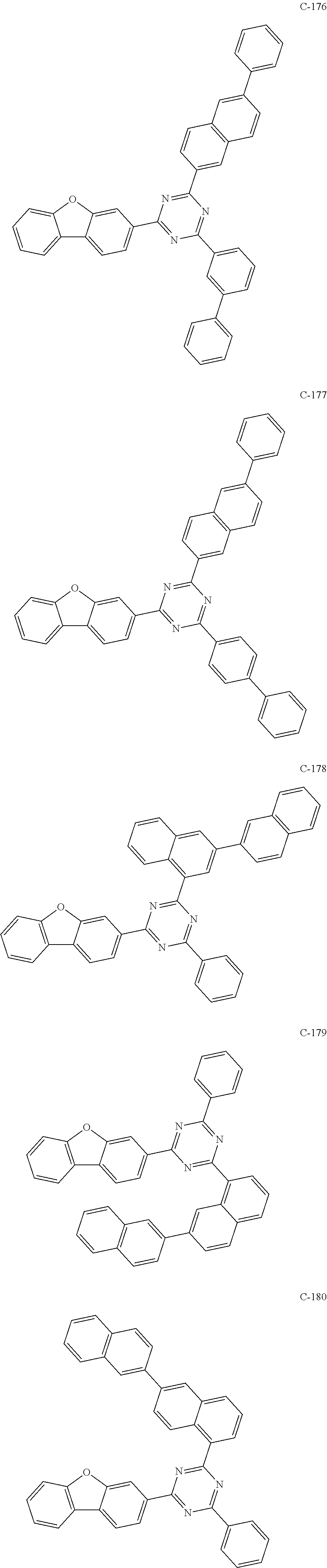 [SMILES: c1ccc(-c2cccc(-c3nc(-c4ccc5cc(-c6ccccc6)ccc5c4)nc(-c4ccc5c(c4)oc4ccccc45)n3)c2)cc1 ; c1ccc(-c2ccc(-c3nc(-c4ccc5cc(-c6ccccc6)ccc5c4)nc(-c4ccc5c(c4)oc4ccccc45)n3)cc2)cc1 ; c1ccc(-c2nc(-c3ccc4c(c3)oc3ccccc34)nc(-c3cc(-c4ccc5ccccc5c4)cc4ccccc34)n2)cc1 ; c1ccc(-c2nc(-c3ccc4c(c3)oc3ccccc34)nc(-c3cccc4ccc(-c5ccc6ccccc6c5)cc34)n2)cc1 ; c1ccc(-c2nc(-c3ccc4c(c3)oc3ccccc34)nc(-c3cccc4cc(-c5ccc6ccccc6c5)ccc34)n2)cc1]

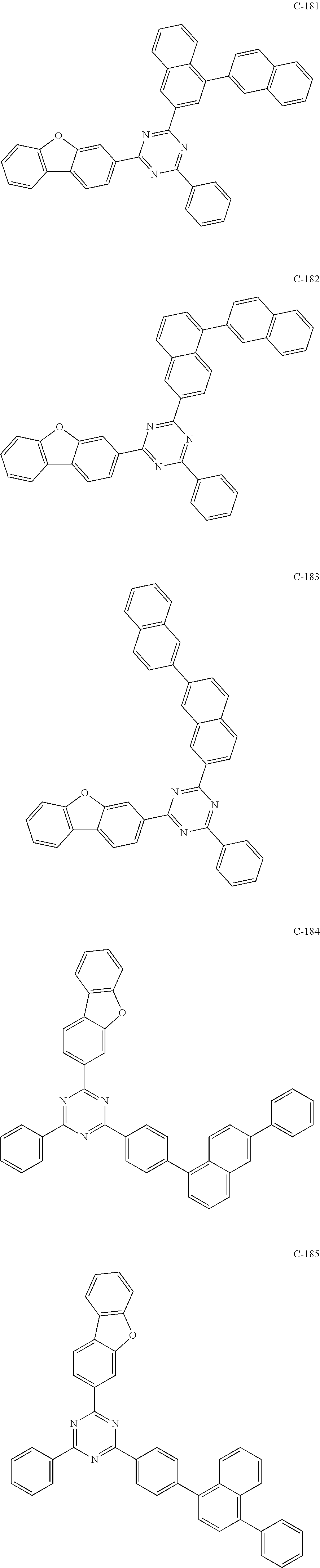 [SMILES: c1ccc(-c2nc(-c3cc(-c4ccc5ccccc5c4)c4ccccc4c3)nc(-c3ccc4c(c3)oc3ccccc34)n2)cc1 ; c1ccc(-c2nc(-c3ccc4c(-c5ccc6ccccc6c5)cccc4c3)nc(-c3ccc4c(c3)oc3ccccc34)n2)cc1 ; c1ccc(-c2nc(-c3ccc4ccc(-c5ccc6ccccc6c5)cc4c3)nc(-c3ccc4c(c3)oc3ccccc34)n2)cc1 ; c1ccc(-c2ccc3c(-c4ccc(-c5nc(-c6ccccc6)nc(-c6ccc7c(c6)oc6ccccc67)n5)cc4)cccc3c2)cc1 ; c1ccc(-c2nc(-c3ccc(-c4ccc(-c5ccccc5)c5ccccc45)cc3)nc(-c3ccc4c(c3)oc3ccccc34)n2)cc1]

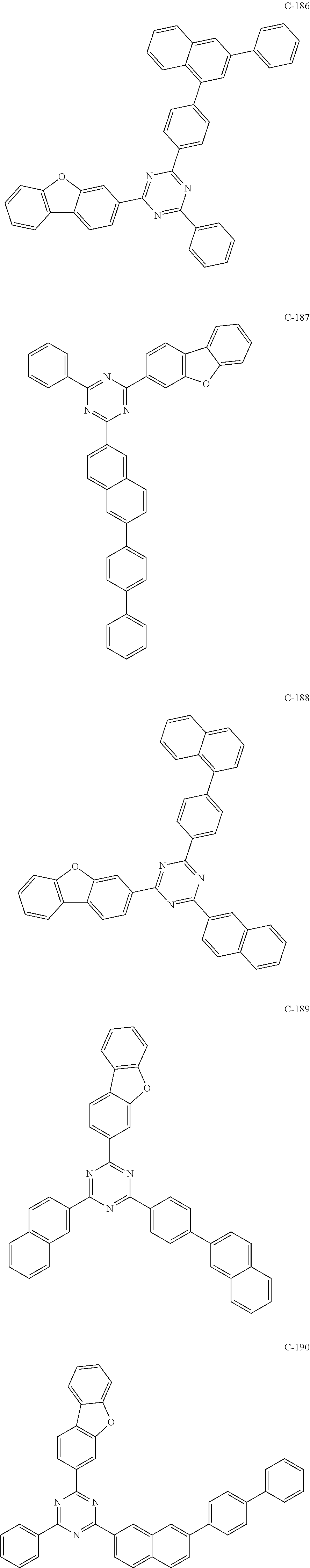 [SMILES: c1ccc(-c2cc(-c3ccc(-c4nc(-c5ccccc5)nc(-c5ccc6c(c5)oc5ccccc56)n4)cc3)c3ccccc3c2)cc1 ; c1ccc(-c2ccc(-c3ccc4cc(-c5nc(-c6ccccc6)nc(-c6ccc7c(c6)oc6ccccc67)n5)ccc4c3)cc2)cc1 ; c1ccc2cc(-c3nc(-c4ccc(-c5cccc6ccccc56)cc4)nc(-c4ccc5c(c4)oc4ccccc45)n3)ccc2c1 ; c1ccc2cc(-c3ccc(-c4nc(-c5ccc6ccccc6c5)nc(-c5ccc6c(c5)oc5ccccc56)n4)cc3)ccc2c1 ; c1ccc(-c2ccc(-c3ccc4ccc(-c5nc(-c6ccccc6)nc(-c6ccc7c(c6)oc6ccccc67)n5)cc4c3)cc2)cc1]

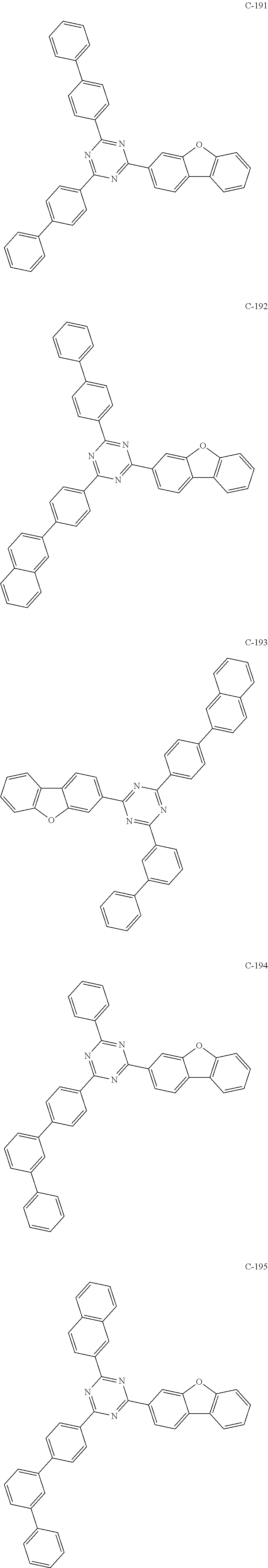 [SMILES: c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2cccc(-c3nc(-c4ccc(-c5ccc6ccccc6c5)cc4)nc(-c4ccc5c(c4)oc4ccccc45)n3)c2)cc1 ; c1ccc(-c2cccc(-c3ccc(-c4nc(-c5ccccc5)nc(-c5ccc6c(c5)oc5ccccc56)n4)cc3)c2)cc1 ; c1ccc(-c2cccc(-c3ccc(-c4nc(-c5ccc6ccccc6c5)nc(-c5ccc6c(c5)oc5ccccc56)n4)cc3)c2)cc1]

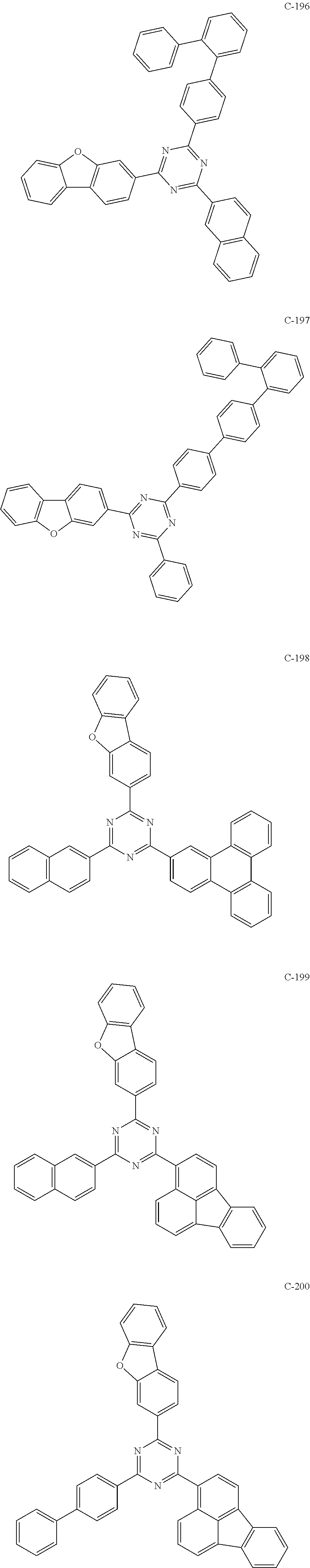 [SMILES: c1ccc(-c2ccccc2-c2ccc(-c3nc(-c4ccc5ccccc5c4)nc(-c4ccc5c(c4)oc4ccccc45)n3)cc2)cc1 ; c1ccc(-c2nc(-c3ccc(-c4ccc(-c5ccccc5-c5ccccc5)cc4)cc3)nc(-c3ccc4c(c3)oc3ccccc34)n2)cc1 ; c1ccc2cc(-c3nc(-c4ccc5c(c4)oc4ccccc45)nc(-c4ccc5c6ccccc6c6ccccc6c5c4)n3)ccc2c1 ; c1ccc2c(c1)-c1cccc3c(-c4nc(-c5ccc6ccccc6c5)nc(-c5ccc6c(c5)oc5ccccc56)n4)ccc-2c13 ; c1ccc(-c2ccc(-c3nc(-c4ccc5c(c4)oc4ccccc45)nc(-c4ccc5c6c(cccc46)-c4ccccc4-5)n3)cc2)cc1]

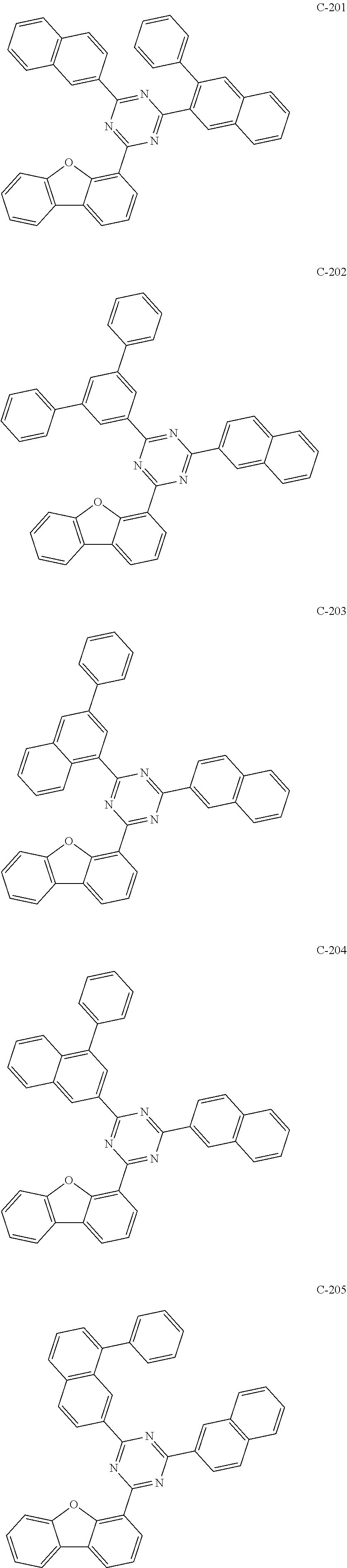 [SMILES: c1ccc(-c2cc3ccccc3cc2-c2nc(-c3ccc4ccccc4c3)nc(-c3cccc4c3oc3ccccc34)n2)cc1 ; c1ccc(-c2cc(-c3ccccc3)cc(-c3nc(-c4ccc5ccccc5c4)nc(-c4cccc5c4oc4ccccc45)n3)c2)cc1 ; c1ccc(-c2cc(-c3nc(-c4ccc5ccccc5c4)nc(-c4cccc5c4oc4ccccc45)n3)c3ccccc3c2)cc1 ; c1ccc(-c2cc(-c3nc(-c4ccc5ccccc5c4)nc(-c4cccc5c4oc4ccccc45)n3)cc3ccccc23)cc1 ; c1ccc(-c2cccc3ccc(-c4nc(-c5ccc6ccccc6c5)nc(-c5cccc6c5oc5ccccc56)n4)cc23)cc1]

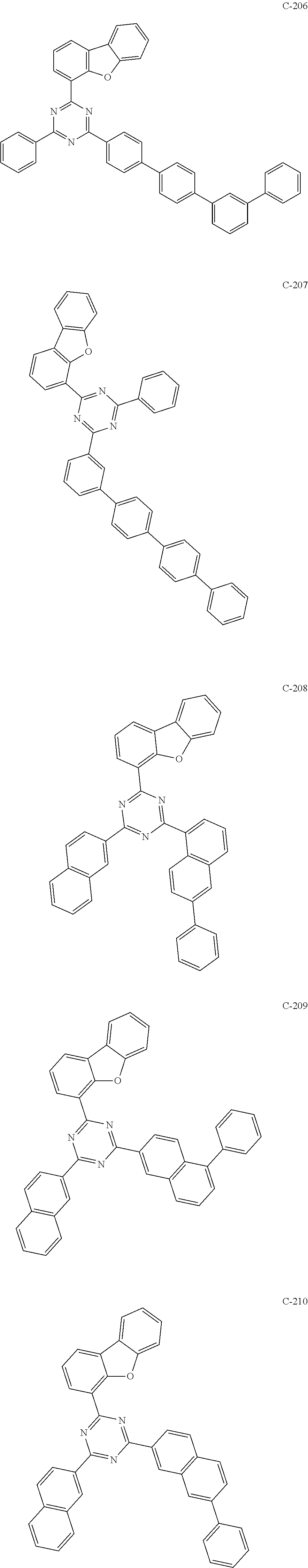 [SMILES: c1ccc(-c2cccc(-c3ccc(-c4ccc(-c5nc(-c6ccccc6)nc(-c6cccc7c6oc6ccccc67)n5)cc4)cc3)c2)cc1 ; c1ccc(-c2ccc(-c3ccc(-c4cccc(-c5nc(-c6ccccc6)nc(-c6cccc7c6oc6ccccc67)n5)c4)cc3)cc2)cc1 ; c1ccc(-c2ccc3c(-c4nc(-c5ccc6ccccc6c5)nc(-c5cccc6c5oc5ccccc56)n4)cccc3c2)cc1 ; c1ccc(-c2cccc3cc(-c4nc(-c5ccc6ccccc6c5)nc(-c5cccc6c5oc5ccccc56)n4)ccc23)cc1 ; c1ccc(-c2ccc3ccc(-c4nc(-c5ccc6ccccc6c5)nc(-c5cccc6c5oc5ccccc56)n4)cc3c2)cc1]

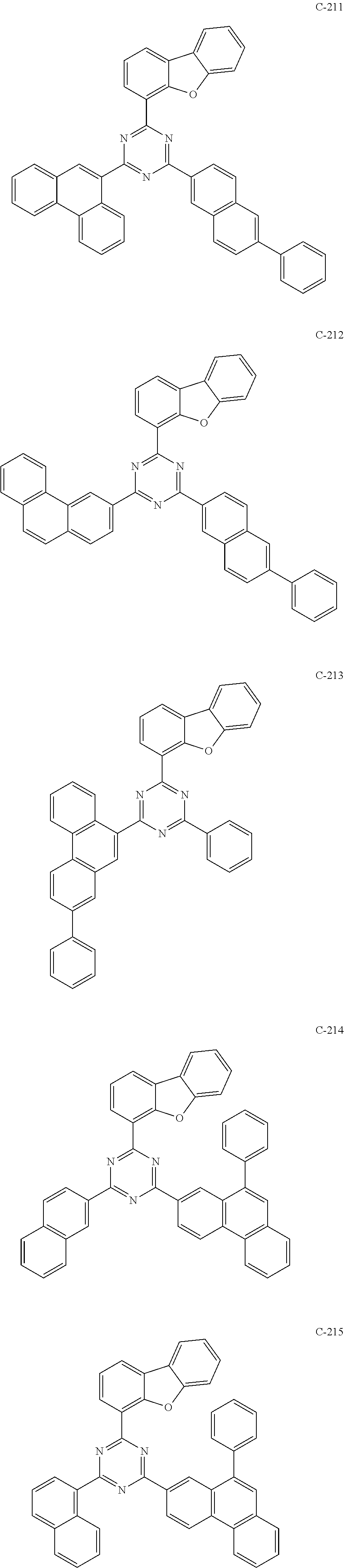 [SMILES: c1ccc(-c2ccc3cc(-c4nc(-c5cc6ccccc6c6ccccc56)nc(-c5cccc6c5oc5ccccc56)n4)ccc3c2)cc1 ; c1ccc(-c2ccc3cc(-c4nc(-c5ccc6ccc7ccccc7c6c5)nc(-c5cccc6c5oc5ccccc56)n4)ccc3c2)cc1 ; c1ccc(-c2ccc3c(c2)cc(-c2nc(-c4ccccc4)nc(-c4cccc5c4oc4ccccc45)n2)c2ccccc23)cc1 ; c1ccc(-c2cc3ccccc3c3ccc(-c4nc(-c5ccc6ccccc6c5)nc(-c5cccc6c5oc5ccccc56)n4)cc23)cc1 ; c1ccc(-c2cc3ccccc3c3ccc(-c4nc(-c5cccc6ccccc56)nc(-c5cccc6c5oc5ccccc56)n4)cc23)cc1]

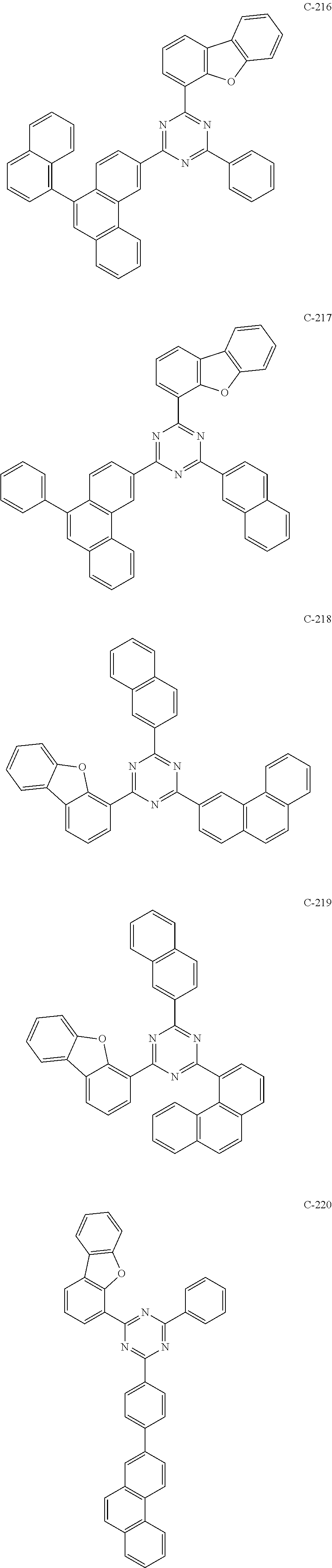 [SMILES: c1ccc(-c2nc(-c3ccc4c(-c5cccc6ccccc56)cc5ccccc5c4c3)nc(-c3cccc4c3oc3ccccc34)n2)cc1 ; c1ccc(-c2cc3ccccc3c3cc(-c4nc(-c5ccc6ccccc6c5)nc(-c5cccc6c5oc5ccccc56)n4)ccc23)cc1 ; c1ccc2cc(-c3nc(-c4ccc5ccc6ccccc6c5c4)nc(-c4cccc5c4oc4ccccc45)n3)ccc2c1 ; c1ccc2cc(-c3nc(-c4cccc5c4oc4ccccc45)nc(-c4cccc5ccc6ccccc6c45)n3)ccc2c1 ; c1ccc(-c2nc(-c3ccc(-c4ccc5c(ccc6ccccc65)c4)cc3)nc(-c3cccc4c3oc3ccccc34)n2)cc1]

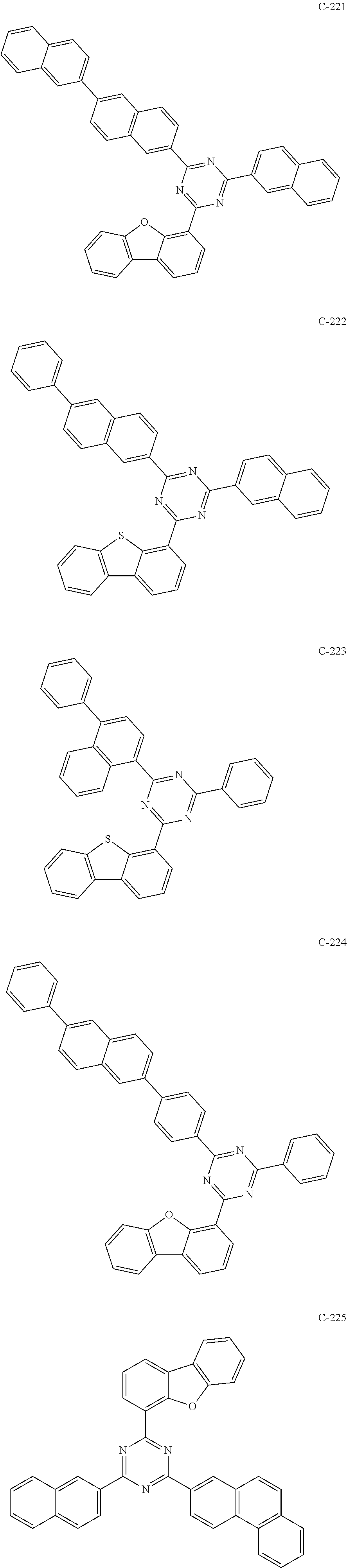 [SMILES: c1ccc2cc(-c3ccc4cc(-c5nc(-c6ccc7ccccc7c6)nc(-c6cccc7c6oc6ccccc67)n5)ccc4c3)ccc2c1 ; c1ccc(-c2ccc3cc(-c4nc(-c5ccc6ccccc6c5)nc(-c5cccc6c5sc5ccccc56)n4)ccc3c2)cc1 ; c1ccc(-c2nc(-c3ccc(-c4ccccc4)c4ccccc34)nc(-c3cccc4c3sc3ccccc34)n2)cc1 ; c1ccc(-c2ccc3cc(-c4ccc(-c5nc(-c6ccccc6)nc(-c6cccc7c6oc6ccccc67)n5)cc4)ccc3c2)cc1 ; c1ccc2cc(-c3nc(-c4ccc5c(ccc6ccccc65)c4)nc(-c4cccc5c4oc4ccccc45)n3)ccc2c1]

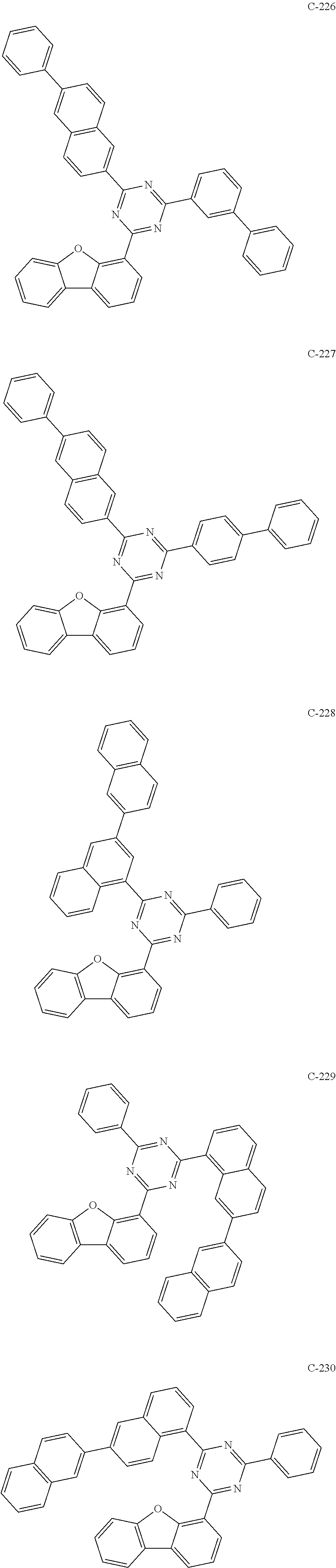 [SMILES: c1ccc(-c2cccc(-c3nc(-c4ccc5cc(-c6ccccc6)ccc5c4)nc(-c4cccc5c4oc4ccccc45)n3)c2)cc1 ; c1ccc(-c2ccc(-c3nc(-c4ccc5cc(-c6ccccc6)ccc5c4)nc(-c4cccc5c4oc4ccccc45)n3)cc2)cc1 ; c1ccc(-c2nc(-c3cc(-c4ccc5ccccc5c4)cc4ccccc34)nc(-c3cccc4c3oc3ccccc34)n2)cc1 ; c1ccc(-c2nc(-c3cccc4ccc(-c5ccc6ccccc6c5)cc34)nc(-c3cccc4c3oc3ccccc34)n2)cc1 ; c1ccc(-c2nc(-c3cccc4cc(-c5ccc6ccccc6c5)ccc34)nc(-c3cccc4c3oc3ccccc34)n2)cc1]

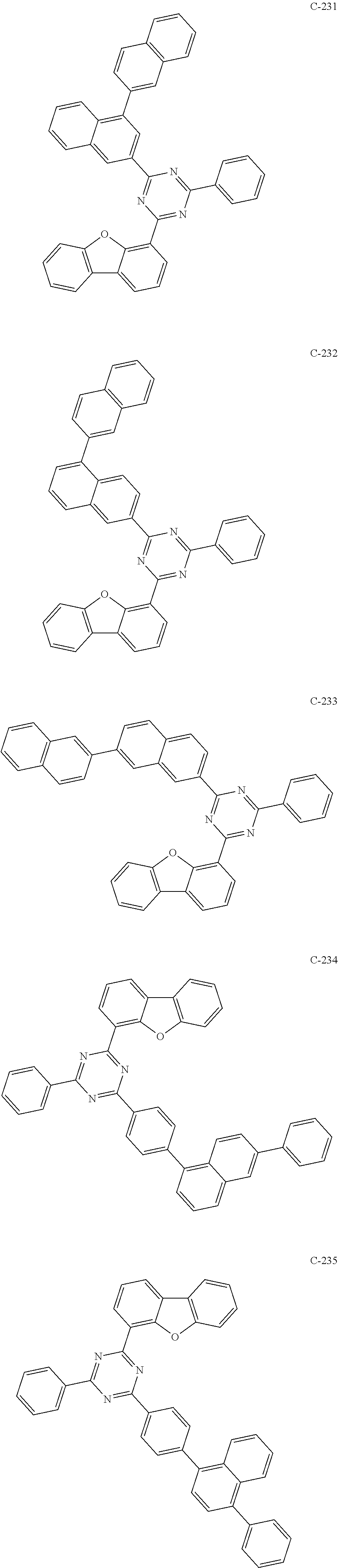 [SMILES: c1ccc(-c2nc(-c3cc(-c4ccc5ccccc5c4)c4ccccc4c3)nc(-c3cccc4c3oc3ccccc34)n2)cc1 ; c1ccc(-c2nc(-c3ccc4c(-c5ccc6ccccc6c5)cccc4c3)nc(-c3cccc4c3oc3ccccc34)n2)cc1 ; c1ccc(-c2nc(-c3ccc4ccc(-c5ccc6ccccc6c5)cc4c3)nc(-c3cccc4c3oc3ccccc34)n2)cc1 ; c1ccc(-c2ccc3c(-c4ccc(-c5nc(-c6ccccc6)nc(-c6cccc7c6oc6ccccc67)n5)cc4)cccc3c2)cc1 ; c1ccc(-c2nc(-c3ccc(-c4ccc(-c5ccccc5)c5ccccc45)cc3)nc(-c3cccc4c3oc3ccccc34)n2)cc1]

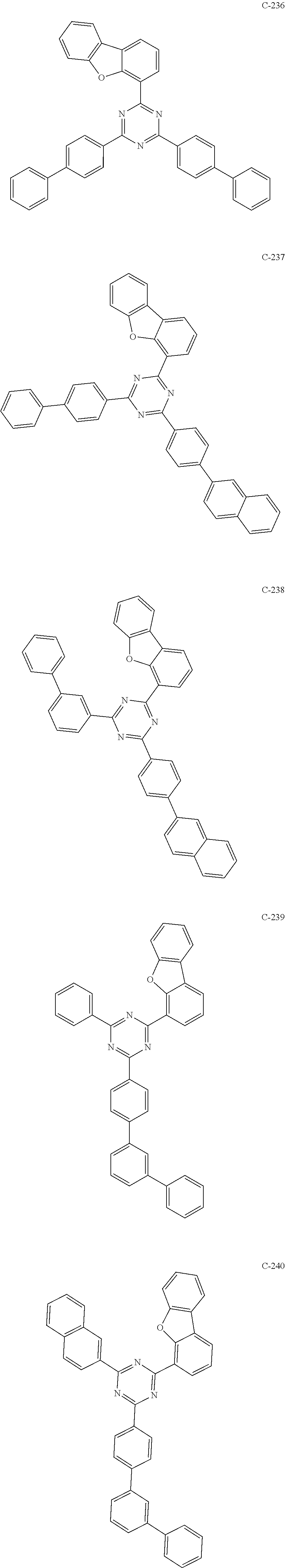 [SMILES: c1ccc(-c2ccc(-c3nc(-c4ccc(-c5ccccc5)cc4)nc(-c4cccc5c4oc4ccccc45)n3)cc2)cc1 ; c1ccc(-c2ccc(-c3nc(-c4ccc(-c5ccc6ccccc6c5)cc4)nc(-c4cccc5c4oc4ccccc45)n3)cc2)cc1 ; c1ccc(-c2cccc(-c3nc(-c4ccc(-c5ccc6ccccc6c5)cc4)nc(-c4cccc5c4oc4ccccc45)n3)c2)cc1 ; c1ccc(-c2cccc(-c3ccc(-c4nc(-c5ccccc5)nc(-c5cccc6c5oc5ccccc56)n4)cc3)c2)cc1 ; c1ccc(-c2cccc(-c3ccc(-c4nc(-c5ccc6ccccc6c5)nc(-c5cccc6c5oc5ccccc56)n4)cc3)c2)cc1]

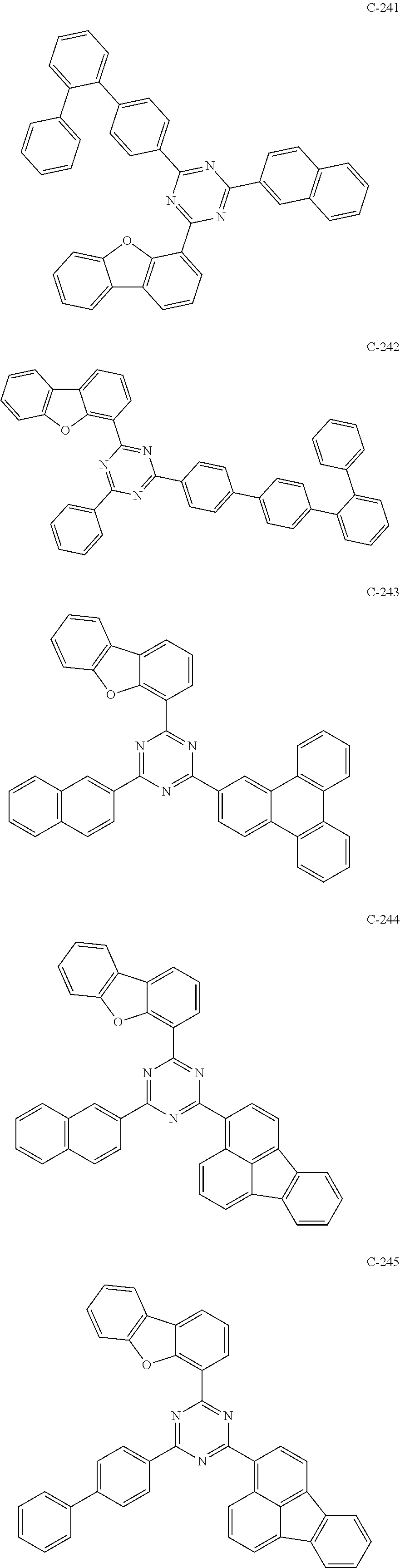 [SMILES: c1ccc(-c2ccccc2-c2ccc(-c3nc(-c4ccc5ccccc5c4)nc(-c4cccc5c4oc4ccccc45)n3)cc2)cc1 ; c1ccc(-c2nc(-c3ccc(-c4ccc(-c5ccccc5-c5ccccc5)cc4)cc3)nc(-c3cccc4c3oc3ccccc34)n2)cc1 ; c1ccc2cc(-c3nc(-c4ccc5c6ccccc6c6ccccc6c5c4)nc(-c4cccc5c4oc4ccccc45)n3)ccc2c1 ; c1ccc2c(c1)-c1cccc3c(-c4nc(-c5ccc6ccccc6c5)nc(-c5cccc6c5oc5ccccc56)n4)ccc-2c13 ; c1ccc(-c2ccc(-c3nc(-c4ccc5c6c(cccc46)-c4ccccc4-5)nc(-c4cccc5c4oc4ccccc45)n3)cc2)cc1]

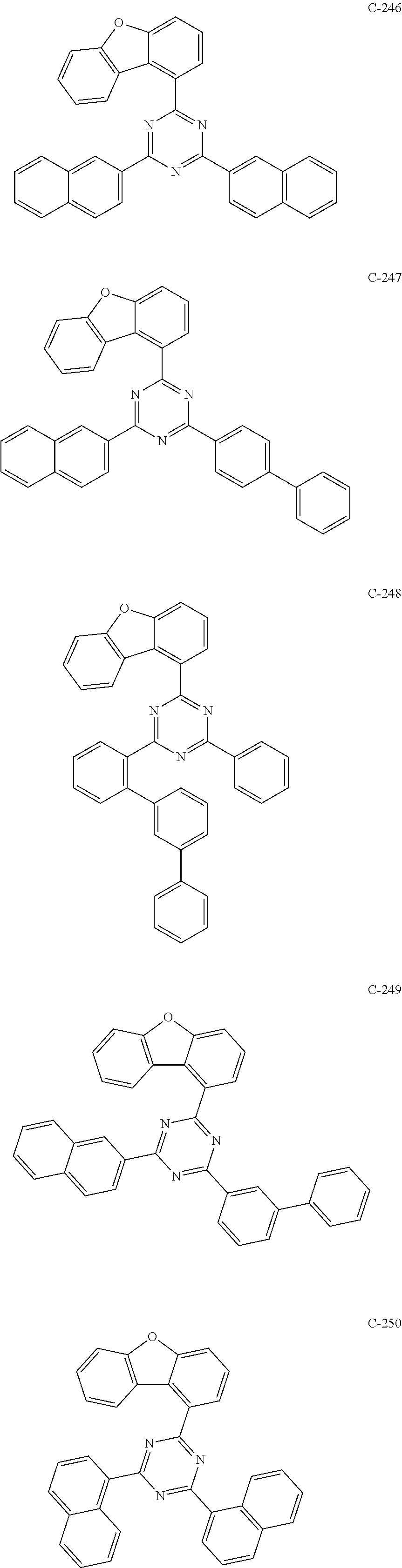 [SMILES: c1ccc2cc(-c3nc(-c4ccc5ccccc5c4)nc(-c4cccc5oc6ccccc6c45)n3)ccc2c1 ; c1ccc(-c2ccc(-c3nc(-c4ccc5ccccc5c4)nc(-c4cccc5oc6ccccc6c45)n3)cc2)cc1 ; c1ccc(-c2cccc(-c3ccccc3-c3nc(-c4ccccc4)nc(-c4cccc5oc6ccccc6c45)n3)c2)cc1 ; c1ccc(-c2cccc(-c3nc(-c4ccc5ccccc5c4)nc(-c4cccc5oc6ccccc6c45)n3)c2)cc1 ; c1ccc2c(-c3nc(-c4cccc5ccccc45)nc(-c4cccc5oc6ccccc6c45)n3)cccc2c1]

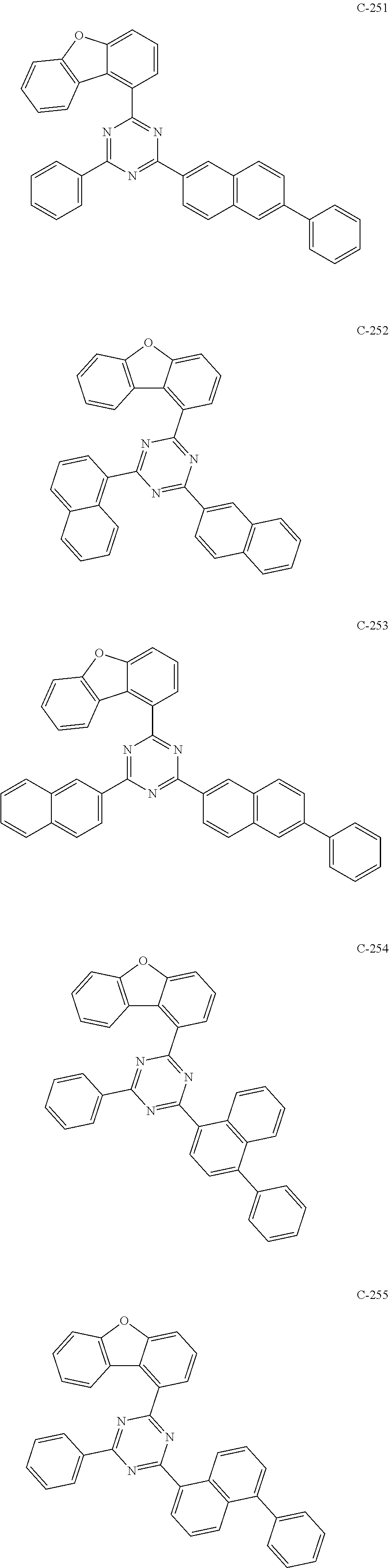 [SMILES: c1ccc(-c2ccc3cc(-c4nc(-c5ccccc5)nc(-c5cccc6oc7ccccc7c56)n4)ccc3c2)cc1 ; c1ccc2cc(-c3nc(-c4cccc5ccccc45)nc(-c4cccc5oc6ccccc6c45)n3)ccc2c1 ; c1ccc(-c2ccc3cc(-c4nc(-c5ccc6ccccc6c5)nc(-c5cccc6oc7ccccc7c56)n4)ccc3c2)cc1 ; c1ccc(-c2nc(-c3ccc(-c4ccccc4)c4ccccc34)nc(-c3cccc4oc5ccccc5c34)n2)cc1 ; c1ccc(-c2nc(-c3cccc4c(-c5ccccc5)cccc34)nc(-c3cccc4oc5ccccc5c34)n2)cc1]

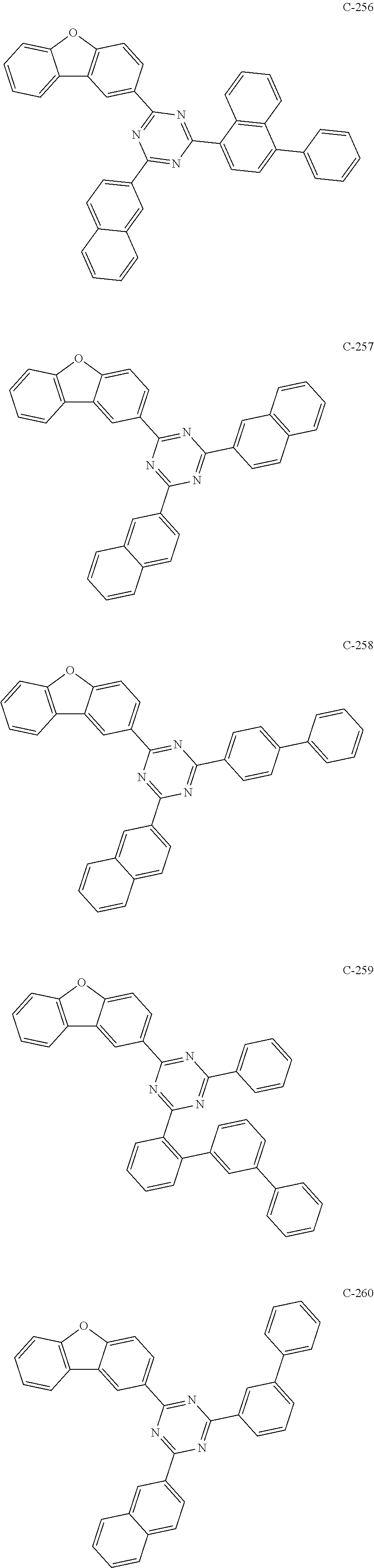 [SMILES: c1ccc(-c2ccc(-c3nc(-c4ccc5ccccc5c4)nc(-c4ccc5oc6ccccc6c5c4)n3)c3ccccc23)cc1 ; c1ccc2cc(-c3nc(-c4ccc5ccccc5c4)nc(-c4ccc5oc6ccccc6c5c4)n3)ccc2c1 ; c1ccc(-c2ccc(-c3nc(-c4ccc5ccccc5c4)nc(-c4ccc5oc6ccccc6c5c4)n3)cc2)cc1 ; c1ccc(-c2cccc(-c3ccccc3-c3nc(-c4ccccc4)nc(-c4ccc5oc6ccccc6c5c4)n3)c2)cc1 ; c1ccc(-c2cccc(-c3nc(-c4ccc5ccccc5c4)nc(-c4ccc5oc6ccccc6c5c4)n3)c2)cc1]

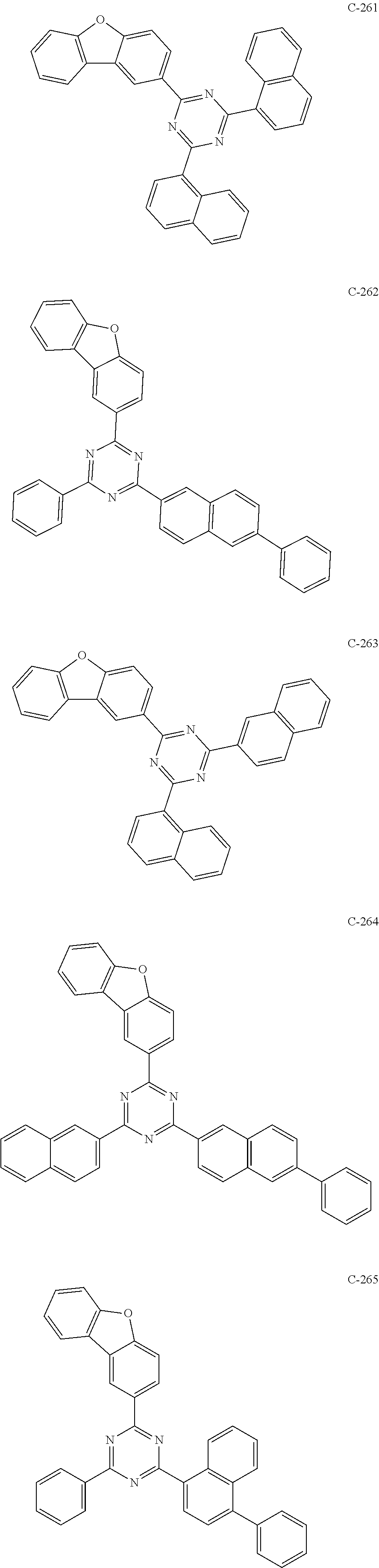 [SMILES: c1ccc2c(-c3nc(-c4ccc5oc6ccccc6c5c4)nc(-c4cccc5ccccc45)n3)cccc2c1 ; c1ccc(-c2ccc3cc(-c4nc(-c5ccccc5)nc(-c5ccc6oc7ccccc7c6c5)n4)ccc3c2)cc1 ; c1ccc2cc(-c3nc(-c4ccc5oc6ccccc6c5c4)nc(-c4cccc5ccccc45)n3)ccc2c1 ; c1ccc(-c2ccc3cc(-c4nc(-c5ccc6ccccc6c5)nc(-c5ccc6oc7ccccc7c6c5)n4)ccc3c2)cc1 ; c1ccc(-c2nc(-c3ccc4oc5ccccc5c4c3)nc(-c3ccc(-c4ccccc4)c4ccccc34)n2)cc1]

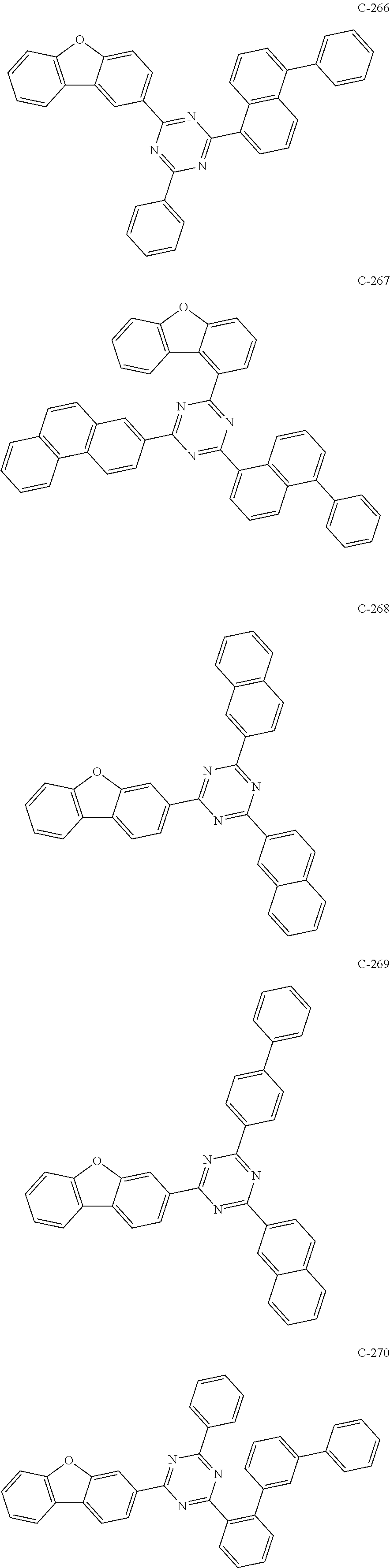 [SMILES: c1ccc(-c2nc(-c3ccc4oc5ccccc5c4c3)nc(-c3cccc4c(-c5ccccc5)cccc34)n2)cc1 ; c1ccc(-c2cccc3c(-c4nc(-c5ccc6c(ccc7ccccc76)c5)nc(-c5cccc6oc7ccccc7c56)n4)cccc23)cc1 ; c1ccc2cc(-c3nc(-c4ccc5ccccc5c4)nc(-c4ccc5c(c4)oc4ccccc45)n3)ccc2c1 ; c1ccc(-c2ccc(-c3nc(-c4ccc5ccccc5c4)nc(-c4ccc5c(c4)oc4ccccc45)n3)cc2)cc1 ; c1ccc(-c2cccc(-c3ccccc3-c3nc(-c4ccccc4)nc(-c4ccc5c(c4)oc4ccccc45)n3)c2)cc1]

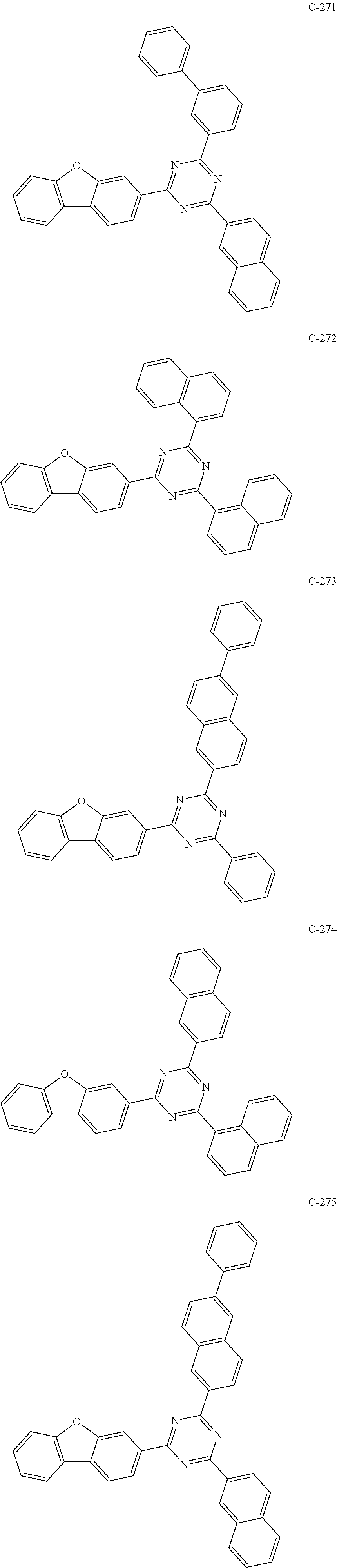 [SMILES: c1ccc(-c2cccc(-c3nc(-c4ccc5ccccc5c4)nc(-c4ccc5c(c4)oc4ccccc45)n3)c2)cc1 ; c1ccc2c(-c3nc(-c4ccc5c(c4)oc4ccccc45)nc(-c4cccc5ccccc45)n3)cccc2c1 ; c1ccc(-c2ccc3cc(-c4nc(-c5ccccc5)nc(-c5ccc6c(c5)oc5ccccc56)n4)ccc3c2)cc1 ; c1ccc2cc(-c3nc(-c4ccc5c(c4)oc4ccccc45)nc(-c4cccc5ccccc45)n3)ccc2c1 ; c1ccc(-c2ccc3cc(-c4nc(-c5ccc6ccccc6c5)nc(-c5ccc6c(c5)oc5ccccc56)n4)ccc3c2)cc1]

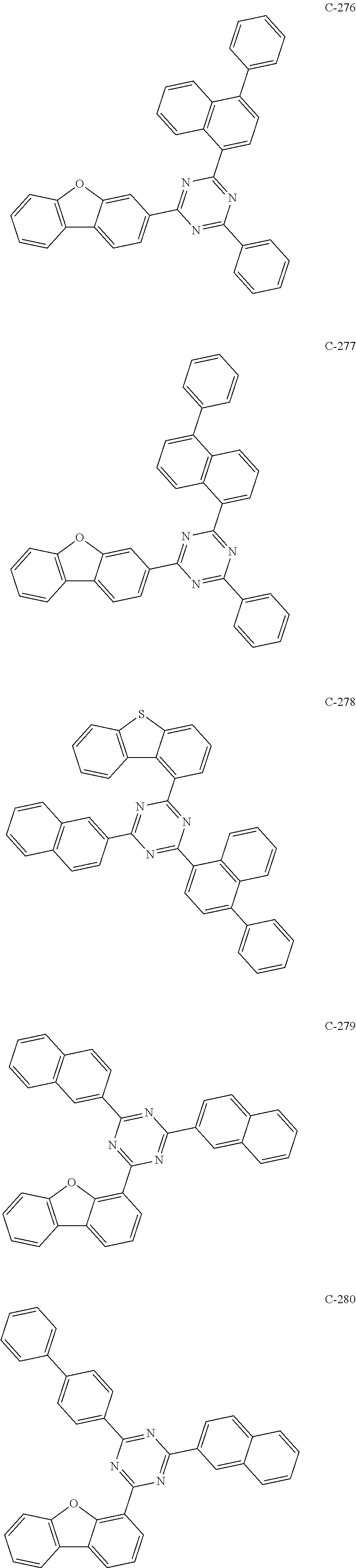 [SMILES: c1ccc(-c2nc(-c3ccc4c(c3)oc3ccccc34)nc(-c3ccc(-c4ccccc4)c4ccccc34)n2)cc1 ; c1ccc(-c2nc(-c3ccc4c(c3)oc3ccccc34)nc(-c3cccc4c(-c5ccccc5)cccc34)n2)cc1 ; c1ccc(-c2ccc(-c3nc(-c4ccc5ccccc5c4)nc(-c4cccc5sc6ccccc6c45)n3)c3ccccc23)cc1 ; c1ccc2cc(-c3nc(-c4ccc5ccccc5c4)nc(-c4cccc5c4oc4ccccc45)n3)ccc2c1 ; c1ccc(-c2ccc(-c3nc(-c4ccc5ccccc5c4)nc(-c4cccc5c4oc4ccccc45)n3)cc2)cc1]

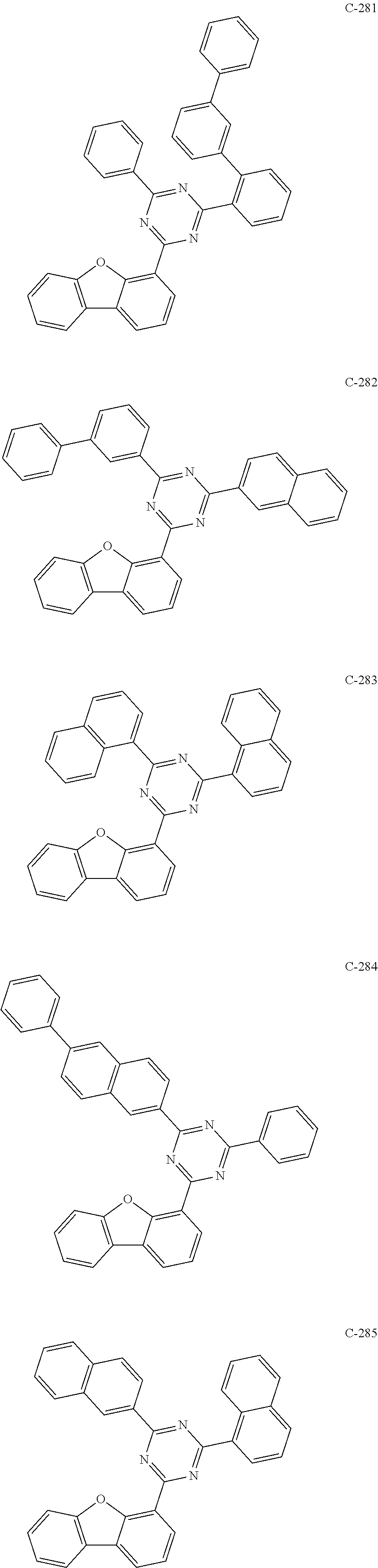 [SMILES: c1ccc(-c2cccc(-c3ccccc3-c3nc(-c4ccccc4)nc(-c4cccc5c4oc4ccccc45)n3)c2)cc1 ; c1ccc(-c2cccc(-c3nc(-c4ccc5ccccc5c4)nc(-c4cccc5c4oc4ccccc45)n3)c2)cc1 ; c1ccc2c(-c3nc(-c4cccc5ccccc45)nc(-c4cccc5c4oc4ccccc45)n3)cccc2c1 ; c1ccc(-c2ccc3cc(-c4nc(-c5ccccc5)nc(-c5cccc6c5oc5ccccc56)n4)ccc3c2)cc1 ; c1ccc2cc(-c3nc(-c4cccc5ccccc45)nc(-c4cccc5c4oc4ccccc45)n3)ccc2c1]

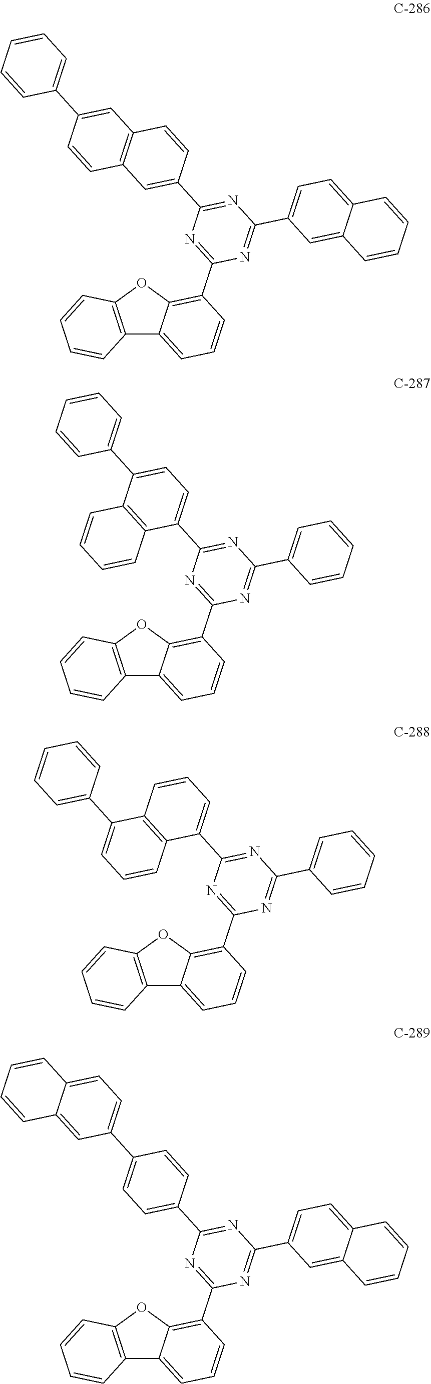

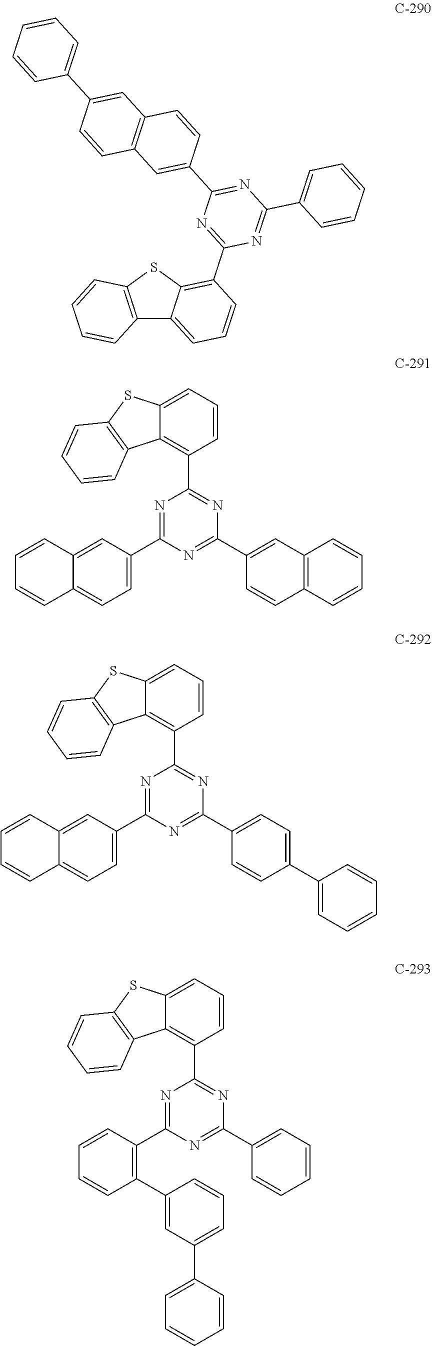

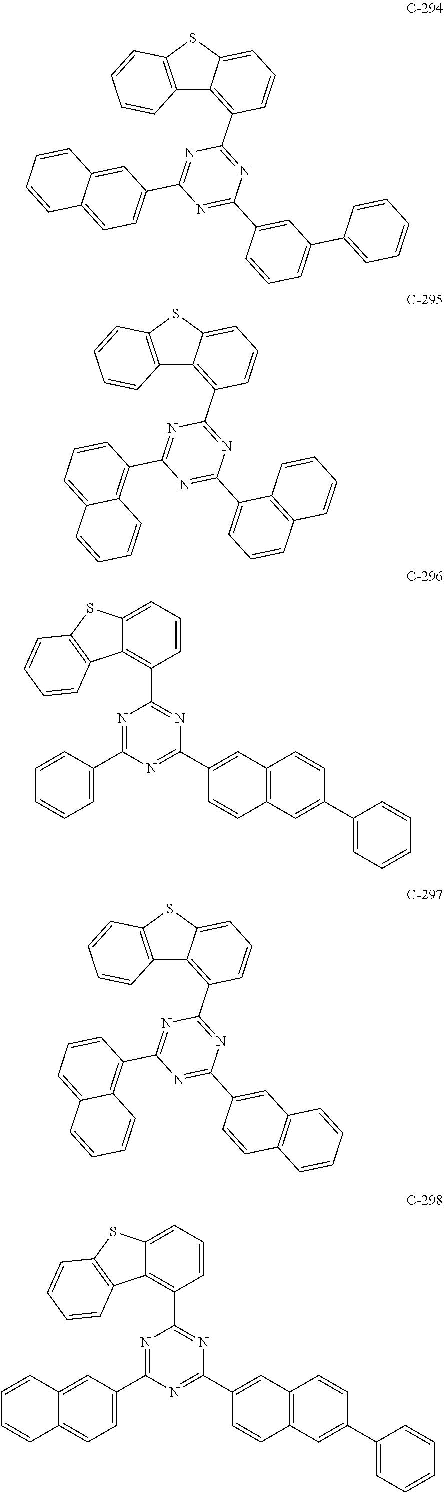

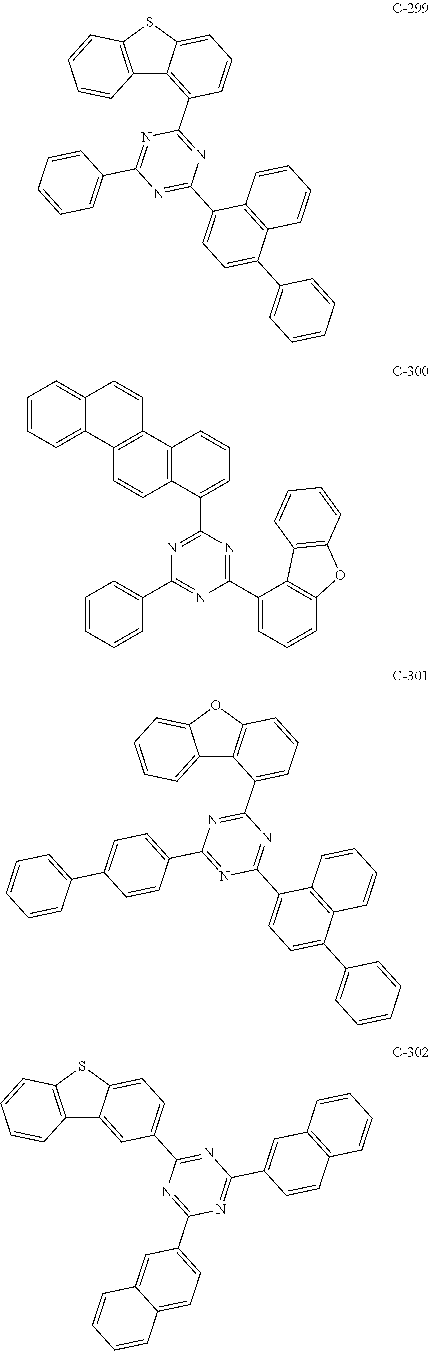 [SMILES: c1ccc(-c2nc(-c3ccc(-c4ccccc4)c4ccccc34)nc(-c3cccc4sc5ccccc5c34)n2)cc1 ; c1ccc(-c2nc(-c3cccc4c3ccc3c5ccccc5ccc43)nc(-c3cccc4oc5ccccc5c34)n2)cc1 ; c1ccc(-c2ccc(-c3nc(-c4ccc(-c5ccccc5)c5ccccc45)nc(-c4cccc5oc6ccccc6c45)n3)cc2)cc1 ; c1ccc2cc(-c3nc(-c4ccc5ccccc5c4)nc(-c4ccc5sc6ccccc6c5c4)n3)ccc2c1]

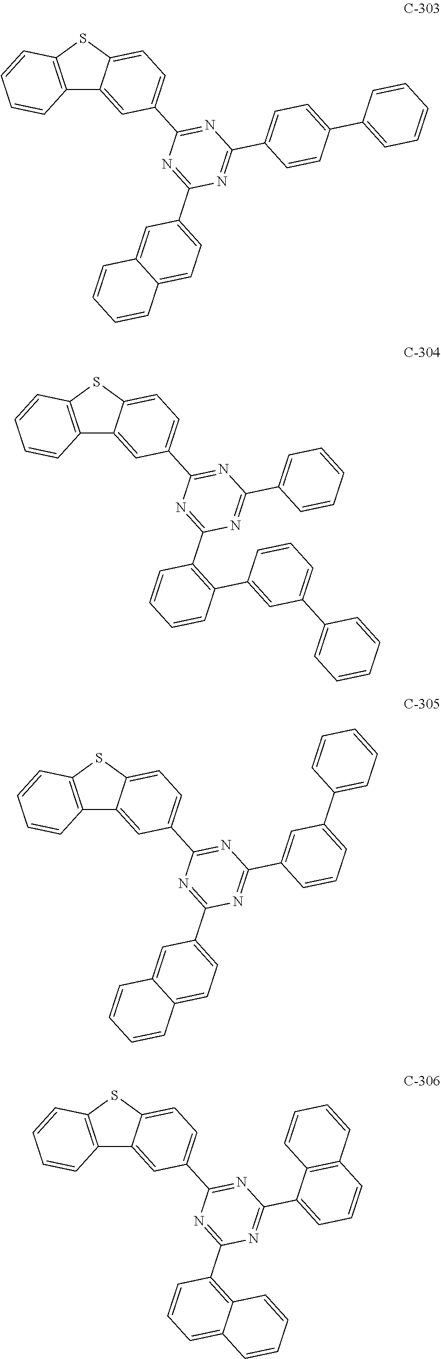 [SMILES: c1ccc(-c2ccc(-c3nc(-c4ccc5ccccc5c4)nc(-c4ccc5sc6ccccc6c5c4)n3)cc2)cc1 ; c1ccc(-c2cccc(-c3ccccc3-c3nc(-c4ccccc4)nc(-c4ccc5sc6ccccc6c5c4)n3)c2)cc1 ; c1ccc(-c2cccc(-c3nc(-c4ccc5ccccc5c4)nc(-c4ccc5sc6ccccc6c5c4)n3)c2)cc1 ; c1ccc2c(-c3nc(-c4ccc5sc6ccccc6c5c4)nc(-c4cccc5ccccc45)n3)cccc2c1]

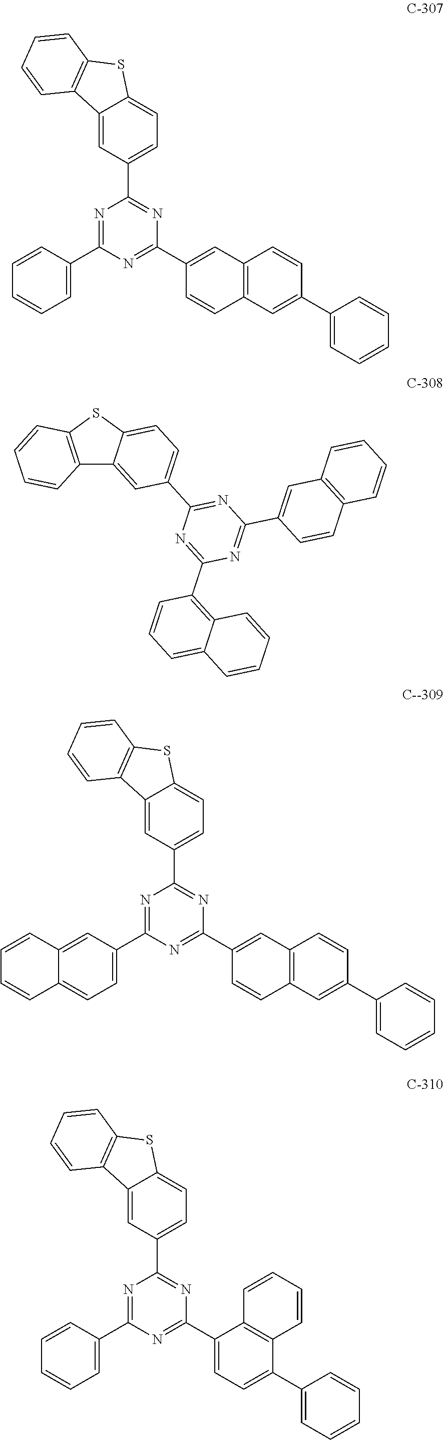 [SMILES: c1ccc(-c2ccc3cc(-c4nc(-c5ccccc5)nc(-c5ccc6sc7ccccc7c6c5)n4)ccc3c2)cc1 ; c1ccc2cc(-c3nc(-c4ccc5sc6ccccc6c5c4)nc(-c4cccc5ccccc45)n3)ccc2c1 ; c1ccc(-c2ccc3cc(-c4nc(-c5ccc6ccccc6c5)nc(-c5ccc6sc7ccccc7c6c5)n4)ccc3c2)cc1 ; c1ccc(-c2nc(-c3ccc4sc5ccccc5c4c3)nc(-c3ccc(-c4ccccc4)c4ccccc34)n2)cc1]

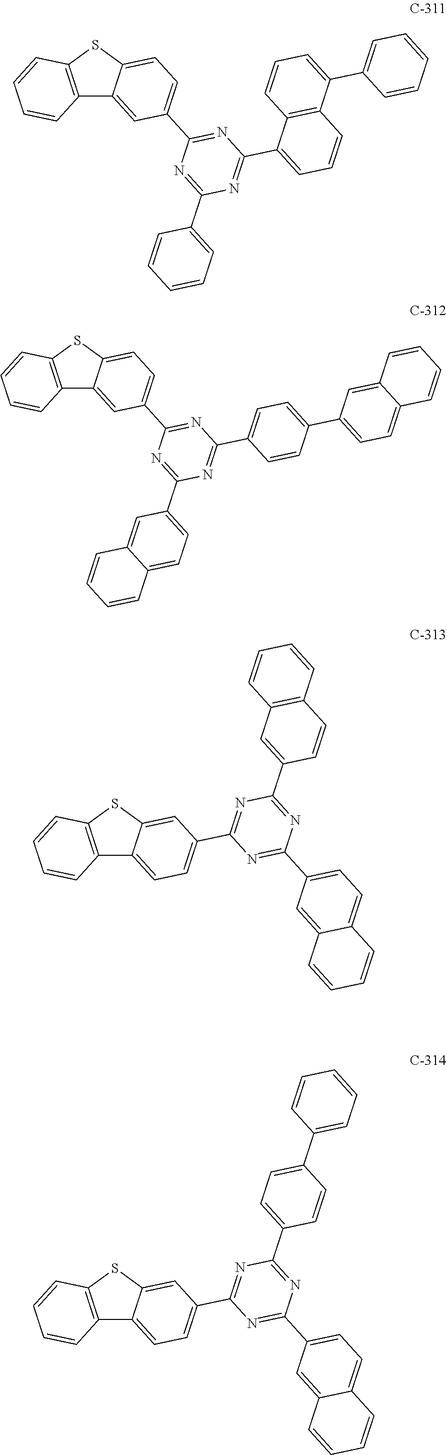 [SMILES: c1ccc(-c2nc(-c3ccc4sc5ccccc5c4c3)nc(-c3cccc4c(-c5ccccc5)cccc34)n2)cc1 ; c1ccc2cc(-c3ccc(-c4nc(-c5ccc6ccccc6c5)nc(-c5ccc6sc7ccccc7c6c5)n4)cc3)ccc2c1 ; c1ccc2cc(-c3nc(-c4ccc5ccccc5c4)nc(-c4ccc5c(c4)sc4ccccc45)n3)ccc2c1 ; c1ccc(-c2ccc(-c3nc(-c4ccc5ccccc5c4)nc(-c4ccc5c(c4)sc4ccccc45)n3)cc2)cc1]

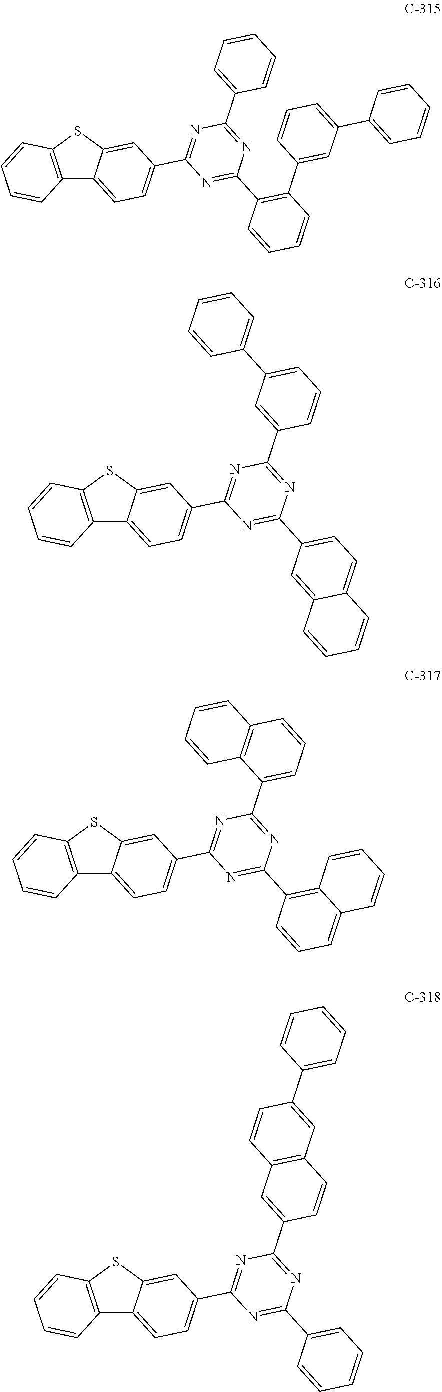 [SMILES: c1ccc(-c2cccc(-c3ccccc3-c3nc(-c4ccccc4)nc(-c4ccc5c(c4)sc4ccccc45)n3)c2)cc1 ; c1ccc(-c2cccc(-c3nc(-c4ccc5ccccc5c4)nc(-c4ccc5c(c4)sc4ccccc45)n3)c2)cc1 ; c1ccc2c(-c3nc(-c4ccc5c(c4)sc4ccccc45)nc(-c4cccc5ccccc45)n3)cccc2c1 ; c1ccc(-c2ccc3cc(-c4nc(-c5ccccc5)nc(-c5ccc6c(c5)sc5ccccc56)n4)ccc3c2)cc1]

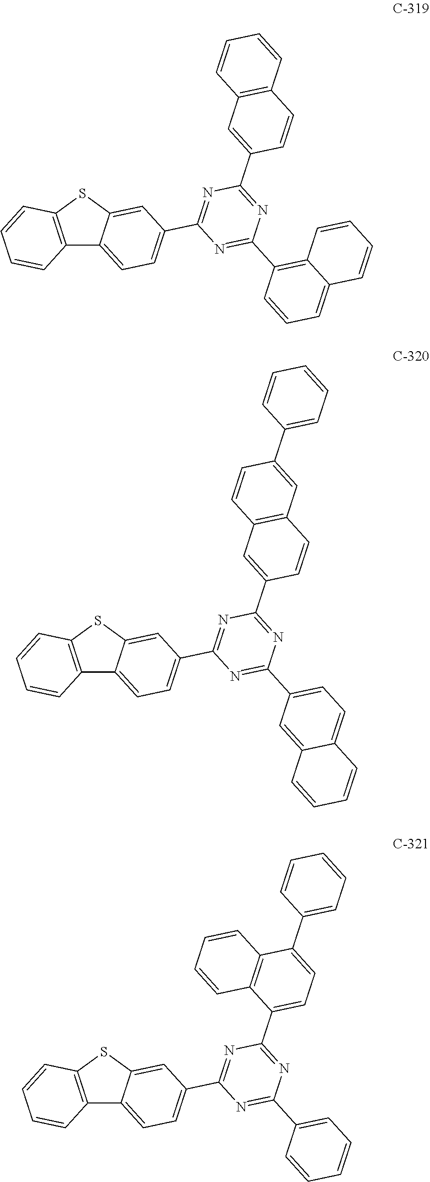

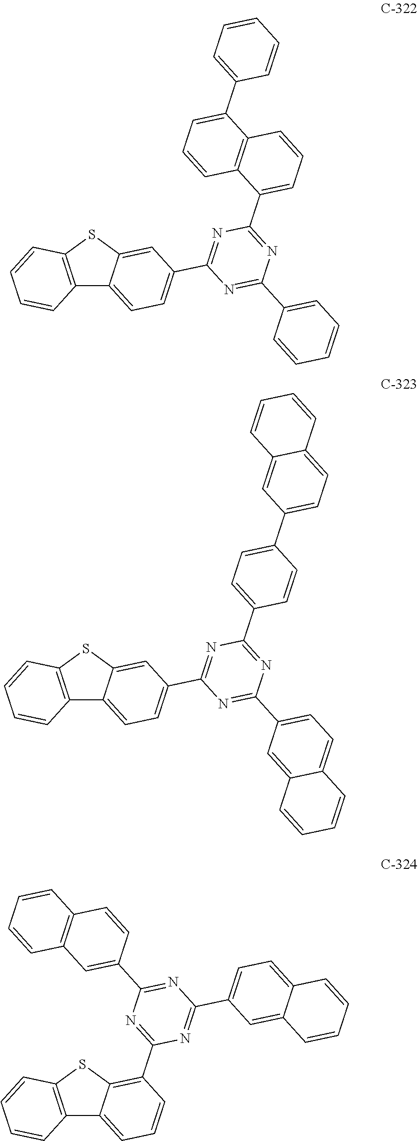

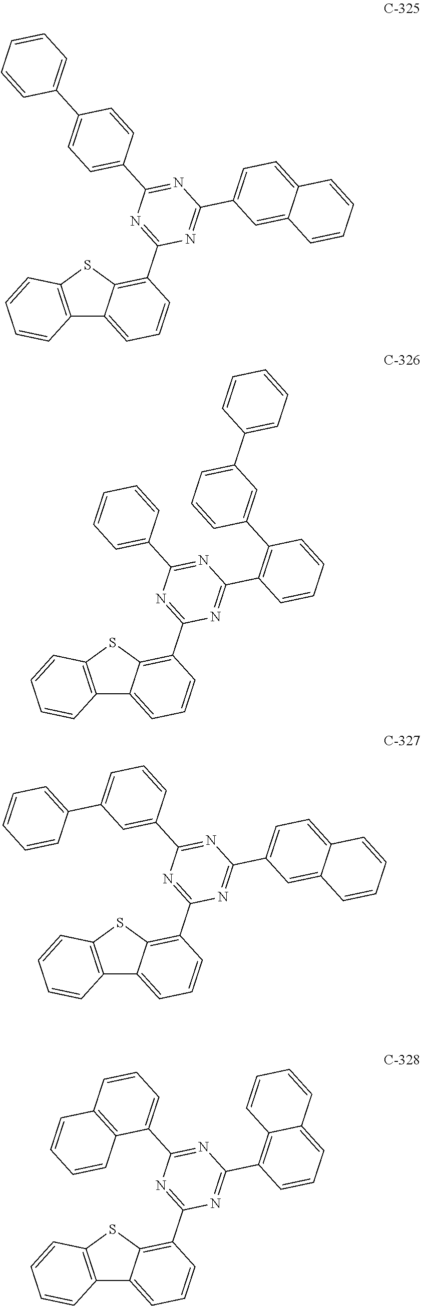 [SMILES: c1ccc(-c2ccc(-c3nc(-c4ccc5ccccc5c4)nc(-c4cccc5c4sc4ccccc45)n3)cc2)cc1 ; c1ccc(-c2cccc(-c3ccccc3-c3nc(-c4ccccc4)nc(-c4cccc5c4sc4ccccc45)n3)c2)cc1 ; c1ccc(-c2cccc(-c3nc(-c4ccc5ccccc5c4)nc(-c4cccc5c4sc4ccccc45)n3)c2)cc1 ; c1ccc2c(-c3nc(-c4cccc5ccccc45)nc(-c4cccc5c4sc4ccccc45)n3)cccc2c1]

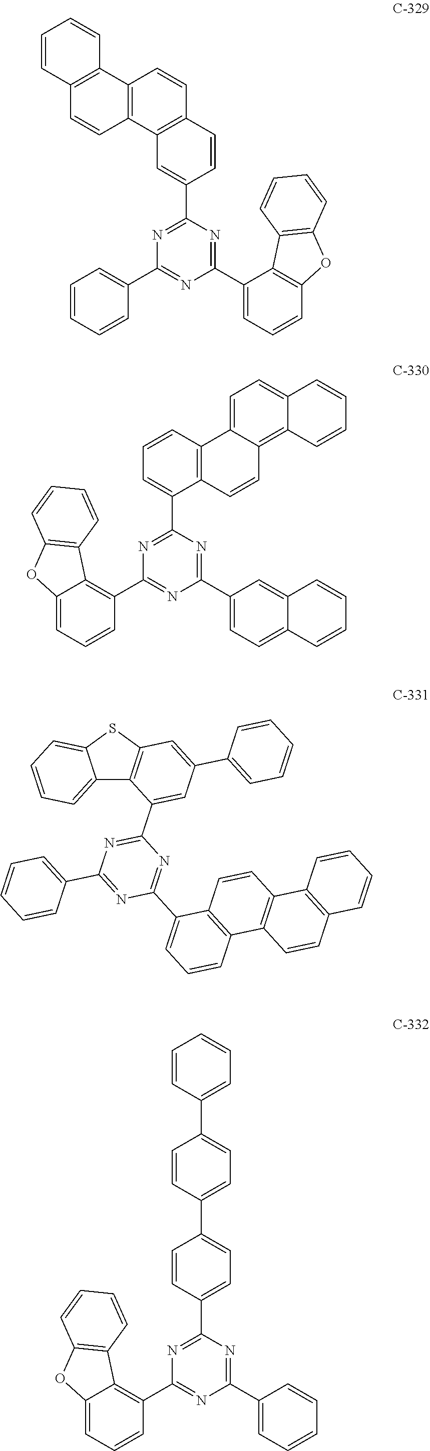 [SMILES: c1ccc(-c2nc(-c3ccc4ccc5c6ccccc6ccc5c4c3)nc(-c3cccc4oc5ccccc5c34)n2)cc1 ; c1ccc2cc(-c3nc(-c4cccc5c4ccc4c6ccccc6ccc54)nc(-c4cccc5oc6ccccc6c45)n3)ccc2c1 ; c1ccc(-c2cc(-c3nc(-c4ccccc4)nc(-c4cccc5c4ccc4c6ccccc6ccc54)n3)c3c(c2)sc2ccccc23)cc1 ; c1ccc(-c2ccc(-c3ccc(-c4nc(-c5ccccc5)nc(-c5cccc6oc7ccccc7c56)n4)cc3)cc2)cc1]

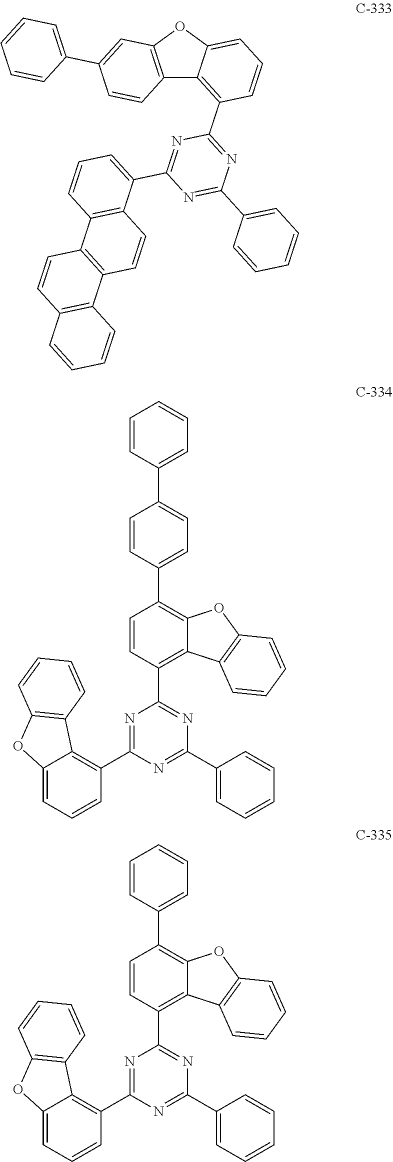 [SMILES: c1ccc(-c2ccc3c(c2)oc2cccc(-c4nc(-c5ccccc5)nc(-c5cccc6c5ccc5c7ccccc7ccc65)n4)c23)cc1 ; c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1]

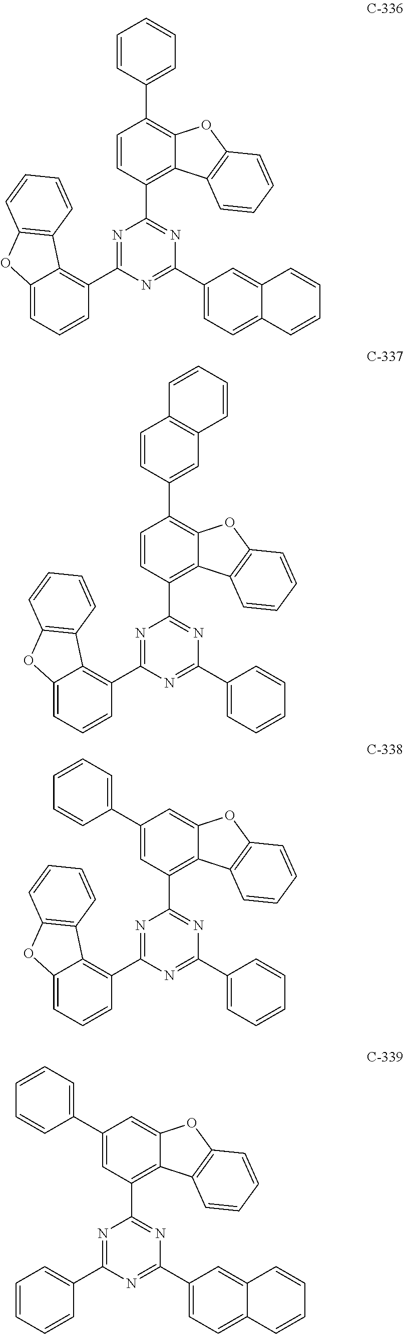

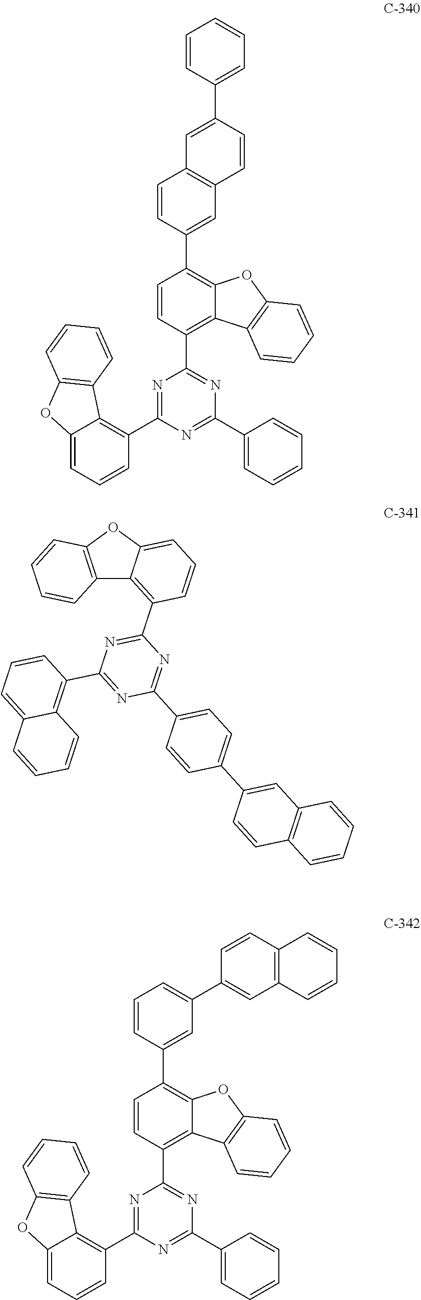

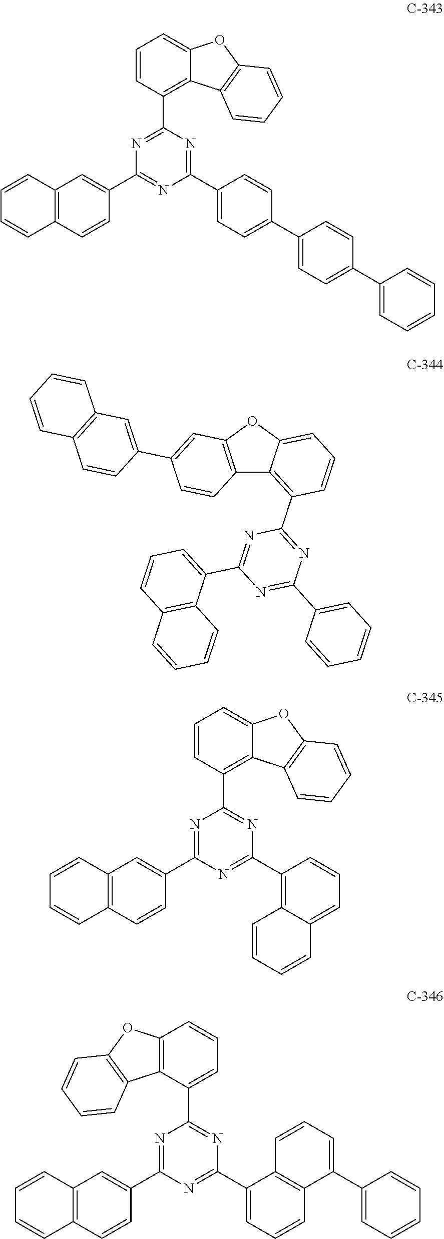 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2nc(-c3cccc4ccccc34)nc(-c3cccc4oc5cc(-c6ccc7ccccc7c6)ccc5c34)n2)cc1 ; c1ccc2cc(-c3nc(-c4cccc5ccccc45)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1]

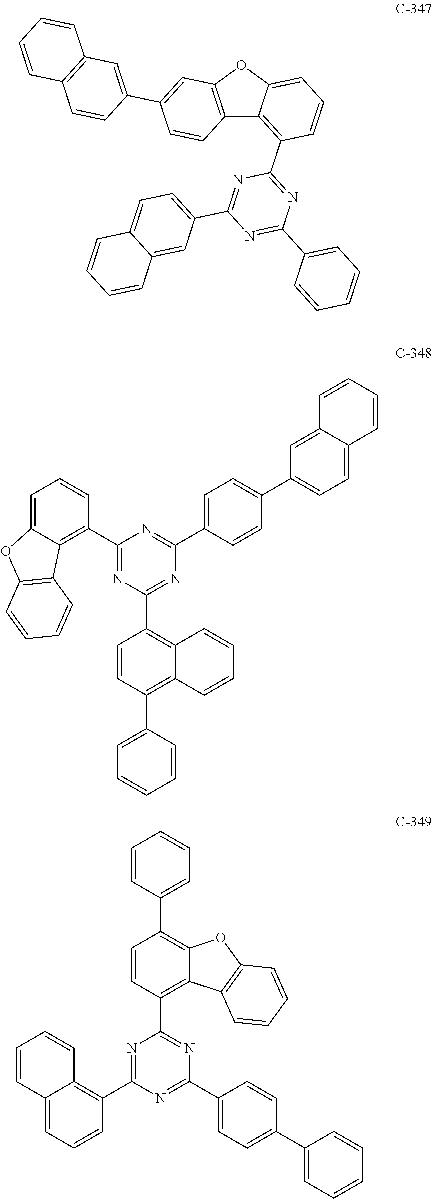

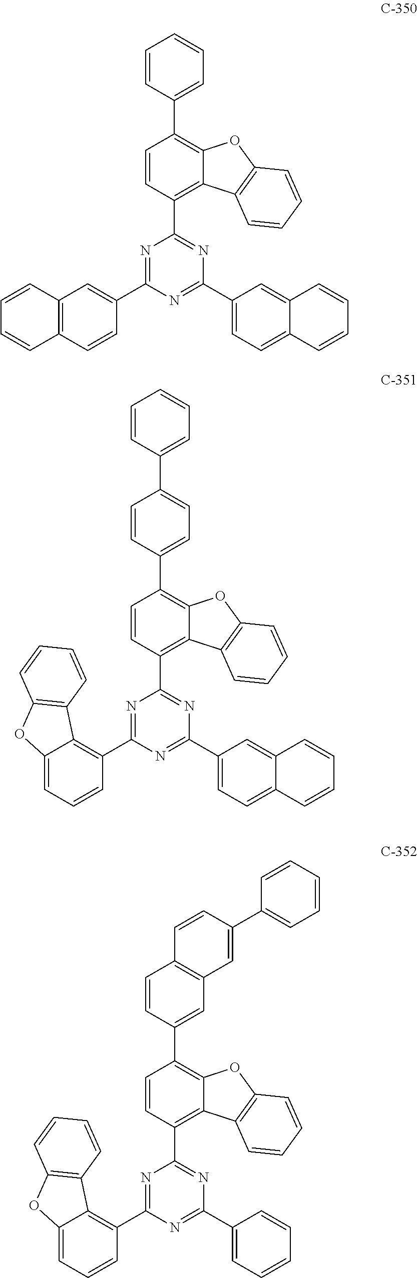 [SMILES: c1ccc(-c2ccc(-c3nc(-c4ccc5ccccc5c4)nc(-c4ccc5ccccc5c4)n3)c3c2oc2ccccc23)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1 ; c1ccc(-c2ccc3ccc(-c4ccc(-c5nc(-c6ccccc6)nc(-c6cccc7oc8ccccc8c67)n5)c5c4oc4ccccc45)cc3c2)cc1]

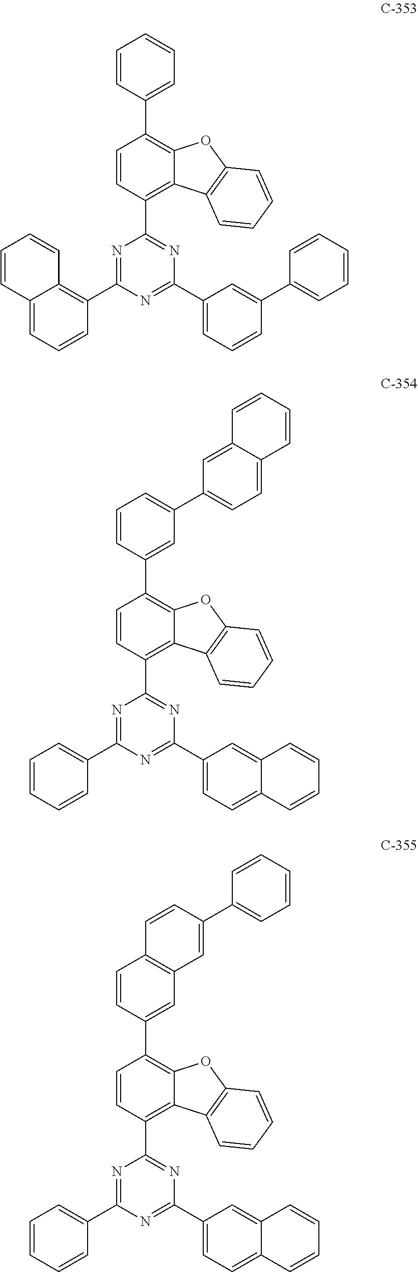 [SMILES: c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccc7ccccc7c6)n5)c5c4oc4ccccc45)cc3c2)cc1]

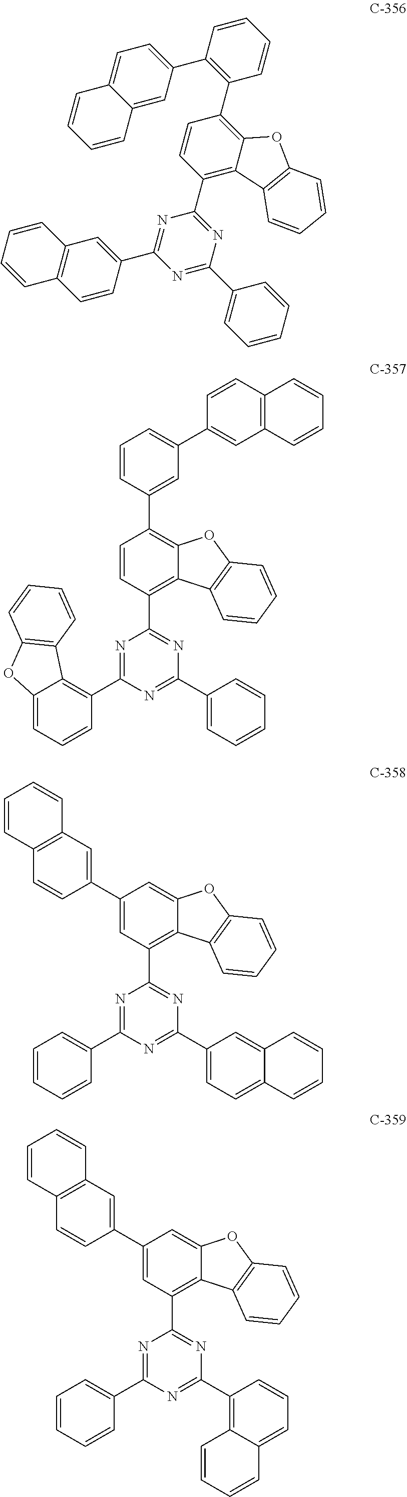

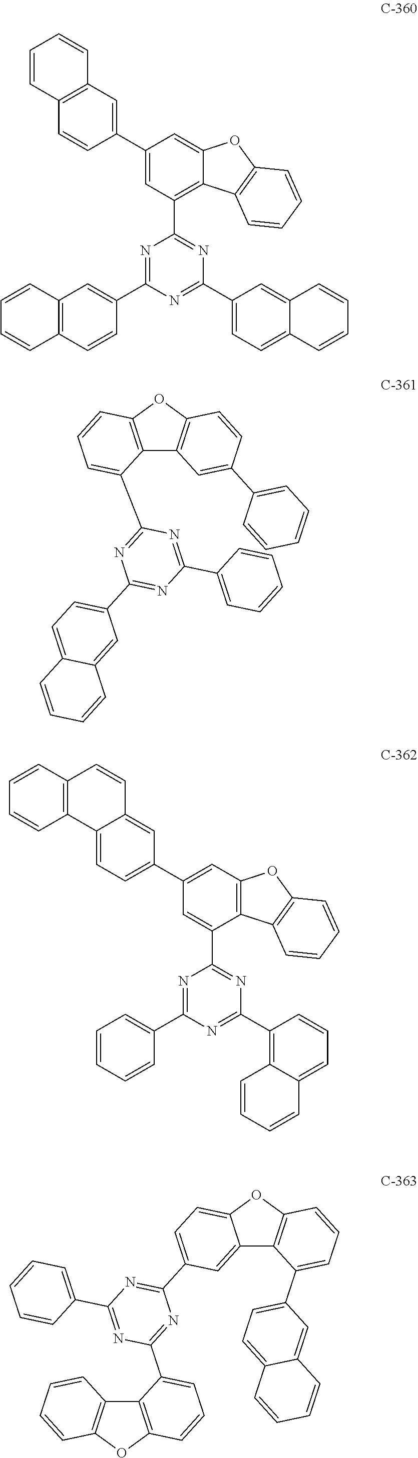

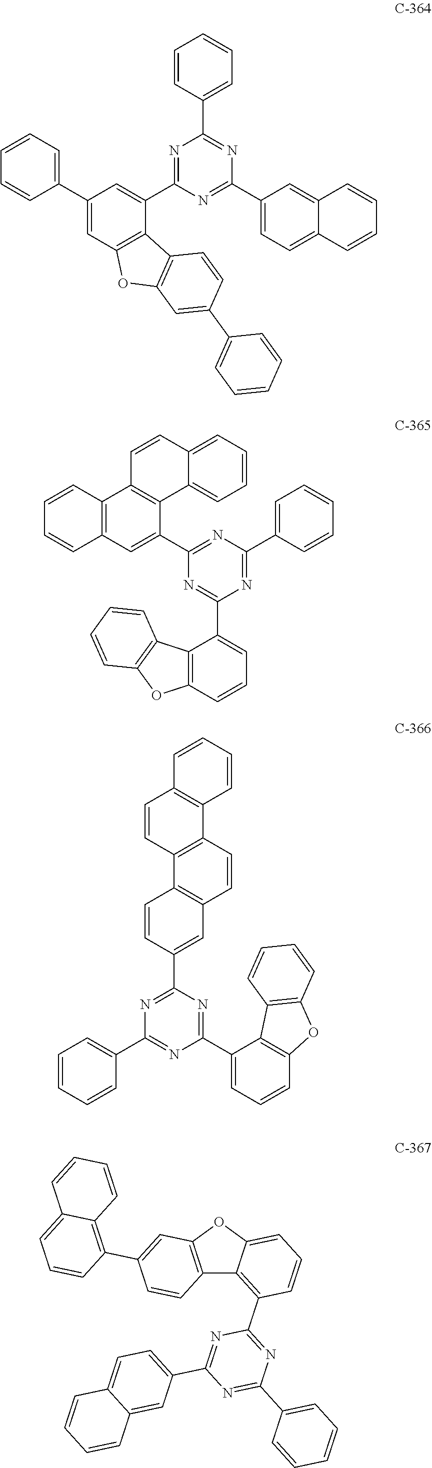

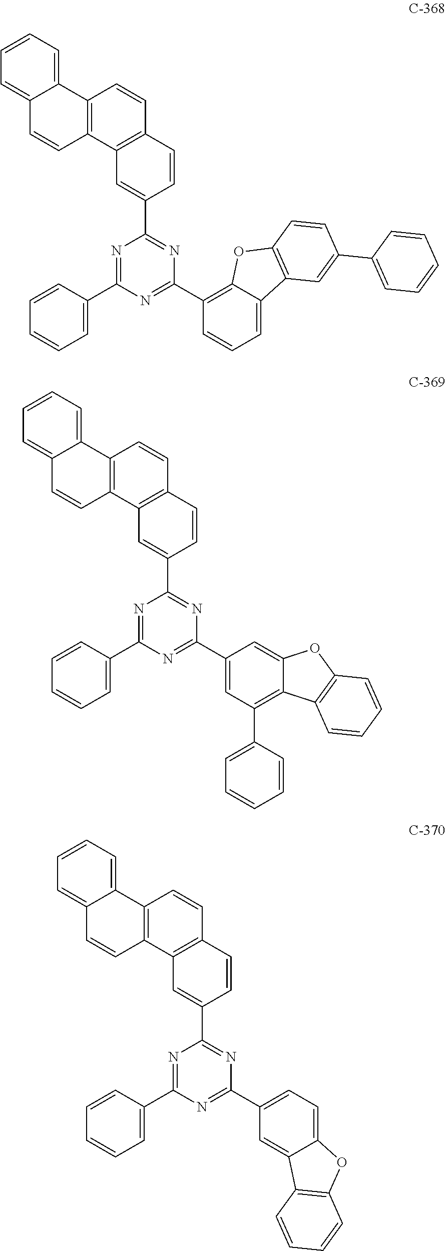

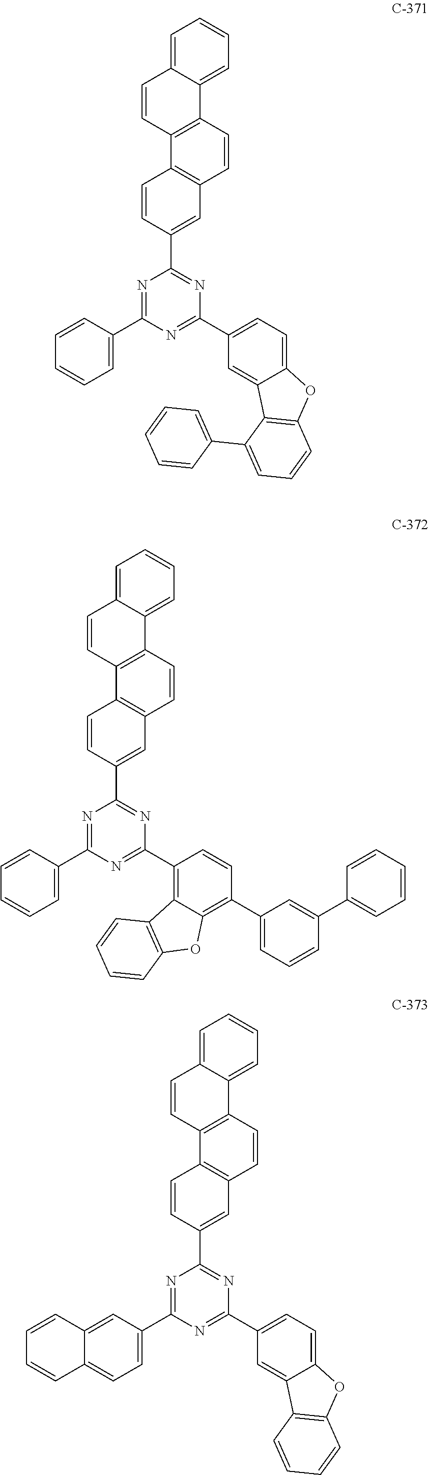 [SMILES: c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4ccc5oc6ccccc6c5c4)n3)ccc2c1]

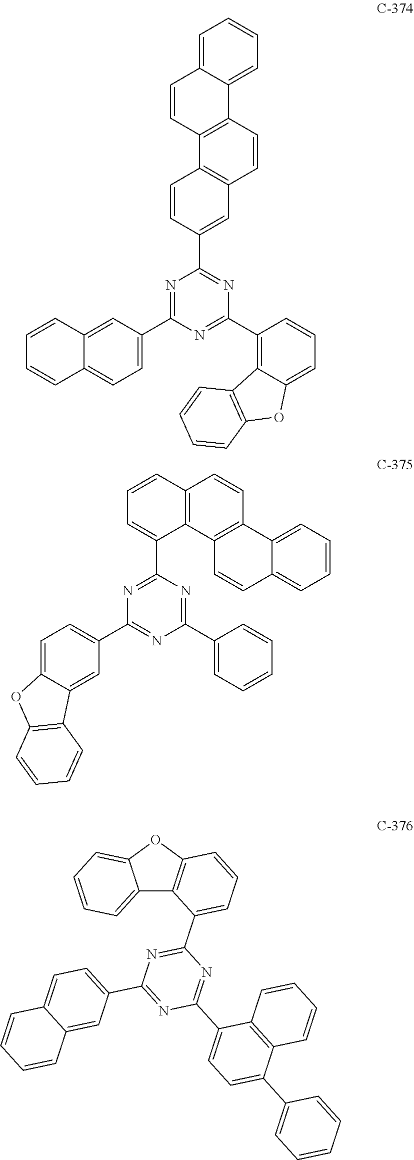

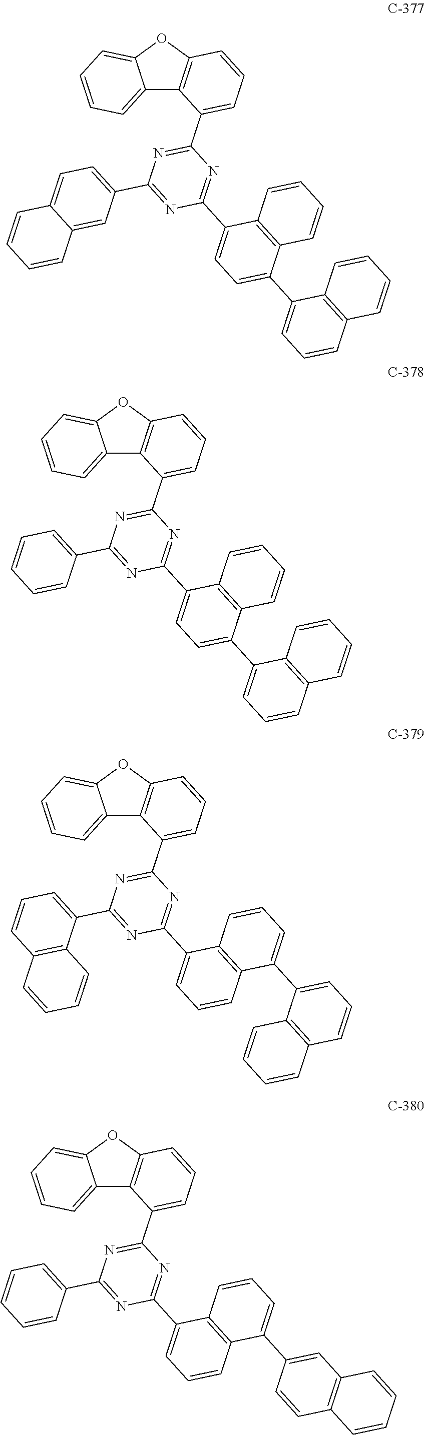 [SMILES: c1ccc2cc(-c3nc(-c4ccc(-c5cccc6ccccc56)c5ccccc45)nc(-c4cccc5oc6ccccc6c45)n3)ccc2c1 ; c1ccc(-c2nc(-c3ccc(-c4cccc5ccccc45)c4ccccc34)nc(-c3cccc4oc5ccccc5c34)n2)cc1 ; c1ccc2c(-c3nc(-c4cccc5c(-c6cccc7ccccc67)cccc45)nc(-c4cccc5oc6ccccc6c45)n3)cccc2c1 ; c1ccc(-c2nc(-c3cccc4c(-c5ccc6ccccc6c5)cccc34)nc(-c3cccc4oc5ccccc5c34)n2)cc1]

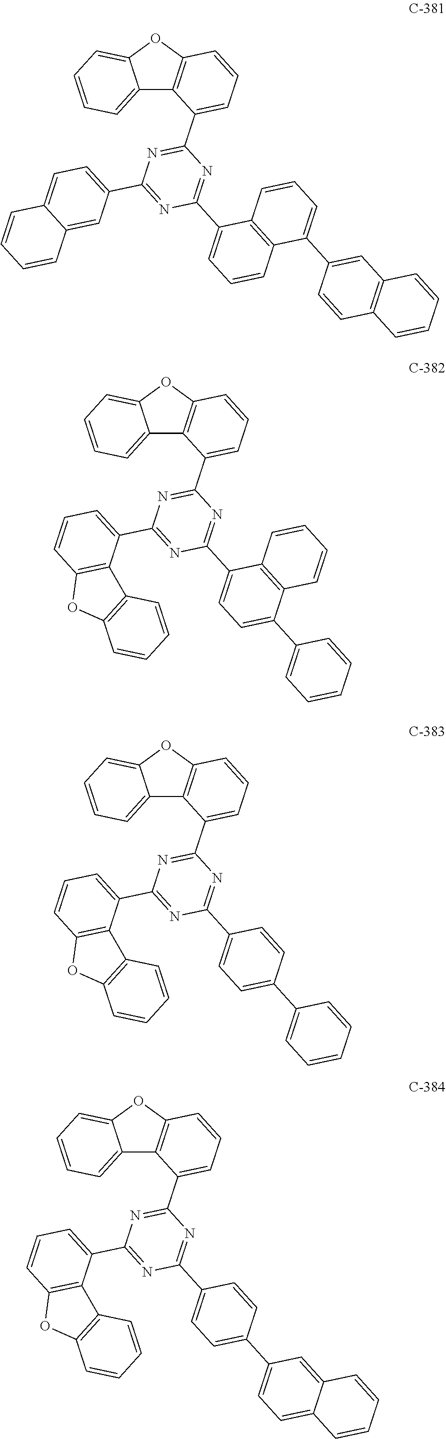 [SMILES: c1ccc2cc(-c3nc(-c4cccc5c(-c6ccc7ccccc7c6)cccc45)nc(-c4cccc5oc6ccccc6c45)n3)ccc2c1 ; c1ccc(-c2ccc(-c3nc(-c4cccc5oc6ccccc6c45)nc(-c4cccc5oc6ccccc6c45)n3)c3ccccc23)cc1 ; c1ccc(-c2ccc(-c3nc(-c4cccc5oc6ccccc6c45)nc(-c4cccc5oc6ccccc6c45)n3)cc2)cc1 ; c1ccc2cc(-c3ccc(-c4nc(-c5cccc6oc7ccccc7c56)nc(-c5cccc6oc7ccccc7c56)n4)cc3)ccc2c1]

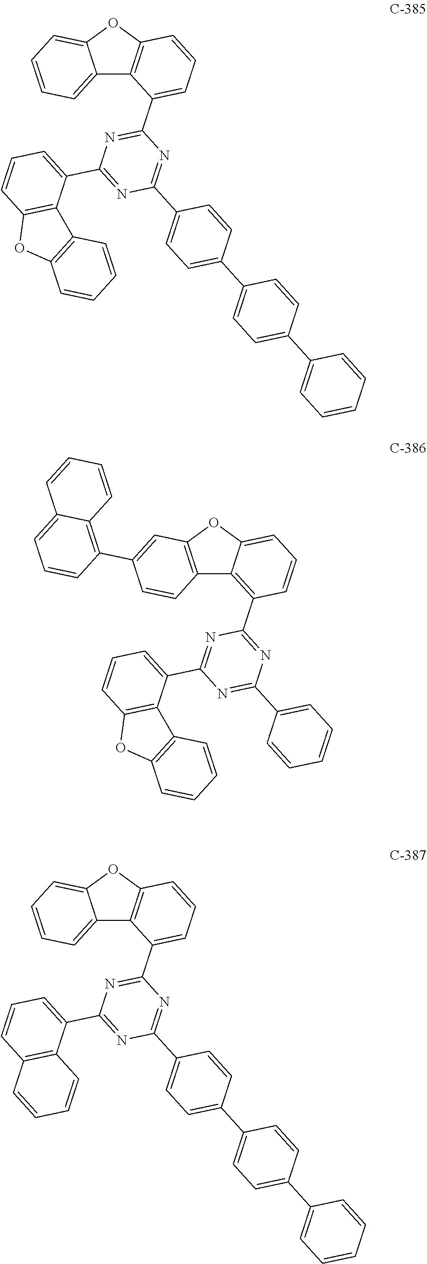 [SMILES: c1ccc(-c2ccc(-c3ccc(-c4nc(-c5cccc6oc7ccccc7c56)nc(-c5cccc6oc7ccccc7c56)n4)cc3)cc2)cc1 ; c1ccc(-c2nc(-c3cccc4oc5ccccc5c34)nc(-c3cccc4oc5cc(-c6cccc7ccccc67)ccc5c34)n2)cc1 ; c1ccc(-c2ccc(-c3ccc(-c4nc(-c5cccc6ccccc56)nc(-c5cccc6oc7ccccc7c56)n4)cc3)cc2)cc1]

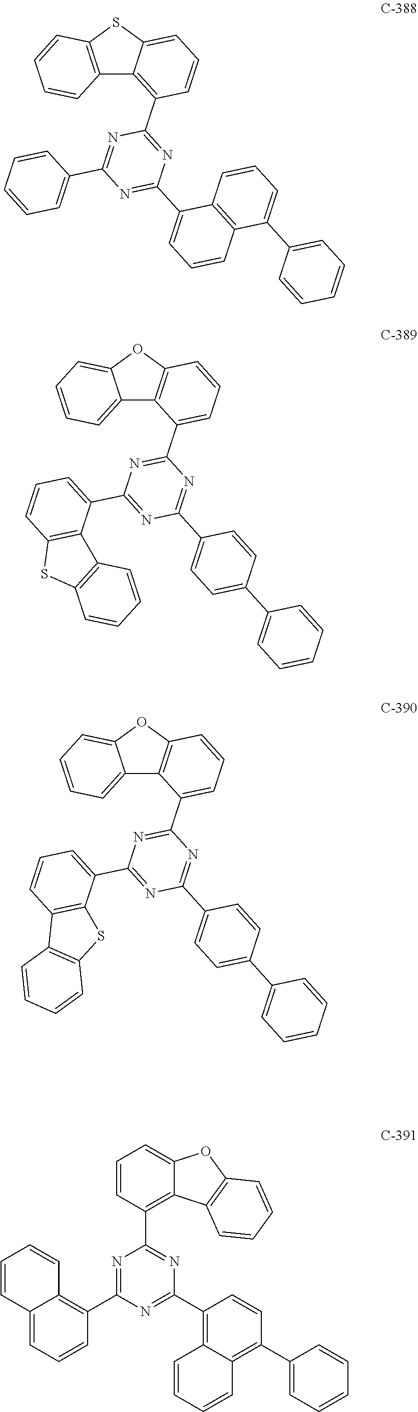 [SMILES: c1ccc(-c2nc(-c3cccc4c(-c5ccccc5)cccc34)nc(-c3cccc4sc5ccccc5c34)n2)cc1 ; c1ccc(-c2ccc(-c3nc(-c4cccc5oc6ccccc6c45)nc(-c4cccc5sc6ccccc6c45)n3)cc2)cc1 ; c1ccc(-c2ccc(-c3nc(-c4cccc5c4sc4ccccc45)nc(-c4cccc5oc6ccccc6c45)n3)cc2)cc1 ; c1ccc(-c2ccc(-c3nc(-c4cccc5ccccc45)nc(-c4cccc5oc6ccccc6c45)n3)c3ccccc23)cc1]

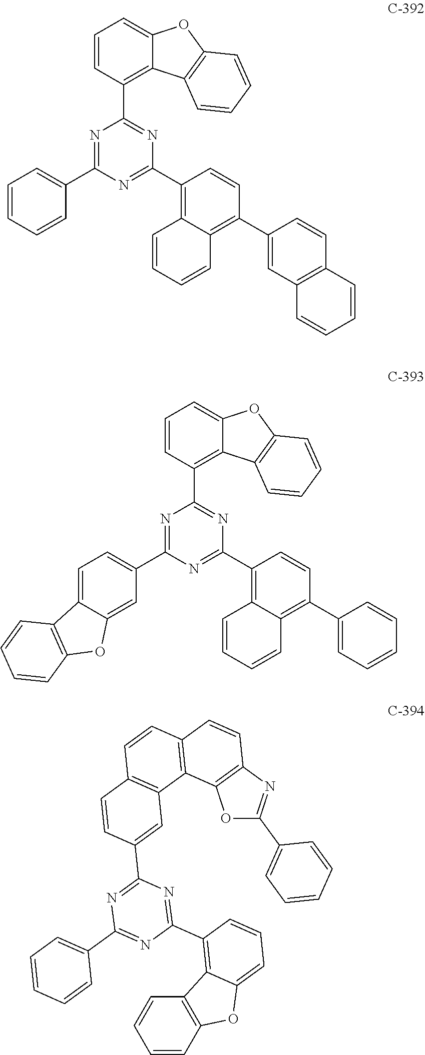 [SMILES: c1ccc(-c2nc(-c3ccc(-c4ccc5ccccc5c4)c4ccccc34)nc(-c3cccc4oc5ccccc5c34)n2)cc1 ; c1ccc(-c2ccc(-c3nc(-c4ccc5c(c4)oc4ccccc45)nc(-c4cccc5oc6ccccc6c45)n3)c3ccccc23)cc1 ; c1ccc(-c2nc(-c3ccc4ccc5ccc6nc(-c7ccccc7)oc6c5c4c3)nc(-c3cccc4oc5ccccc5c34)n2)cc1]

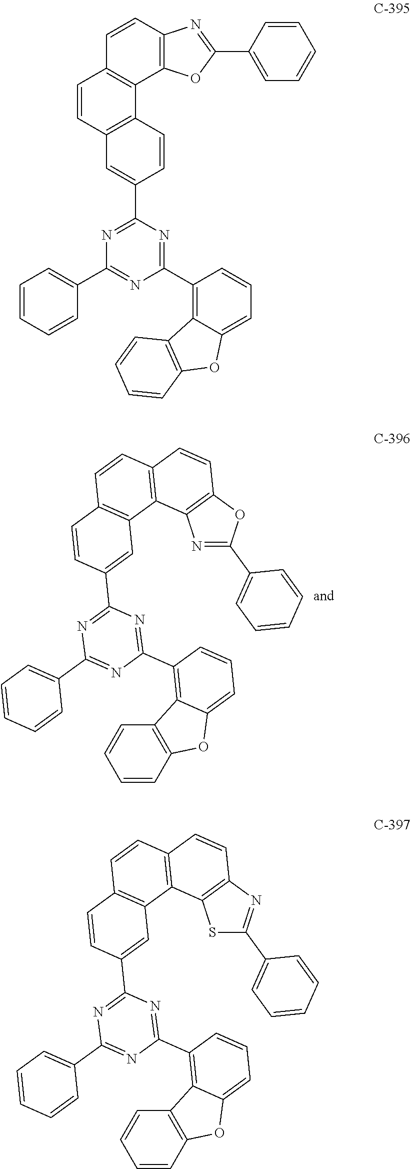 [SMILES: c1ccc(-c2nc(-c3ccc4c(ccc5ccc6nc(-c7ccccc7)oc6c54)c3)nc(-c3cccc4oc5ccccc5c34)n2)cc1 ; c1ccc(-c2nc(-c3ccc4ccc5ccc6oc(-c7ccccc7)nc6c5c4c3)nc(-c3cccc4oc5ccccc5c34)n2)cc1 ; c1ccc(-c2nc(-c3ccc4ccc5ccc6nc(-c7ccccc7)sc6c5c4c3)nc(-c3cccc4oc5ccccc5c34)n2)cc1]

The combination of at least one of compounds H1-1 to H1-151 and at least one of compounds C-1 to C-397 may be used in an organic electroluminescent device.

The compound represented by Formula 1 according to the present disclosure, specifically the compound represented by Formula 1-1 may be synthesized by referring to the method disclosed in Korean Patent Application No. 2018-0021961 (filed on Feb. 23, 2018), and the compound represented by Formula 1-2 may be synthesized by referring to the method disclosed in Korean Patent Application Laid-open No. 2018-0012709 (published on Feb. 6, 2018), but these are not limited thereto, may also be prepared by way of other synthetic methods known to one skilled in the art.

The compound represented by Formula 2 may be produced by way of synthetic methods known to one skilled in the art; for example, it may be produced by referring to the following Reaction Scheme 1, but is not limited thereto.

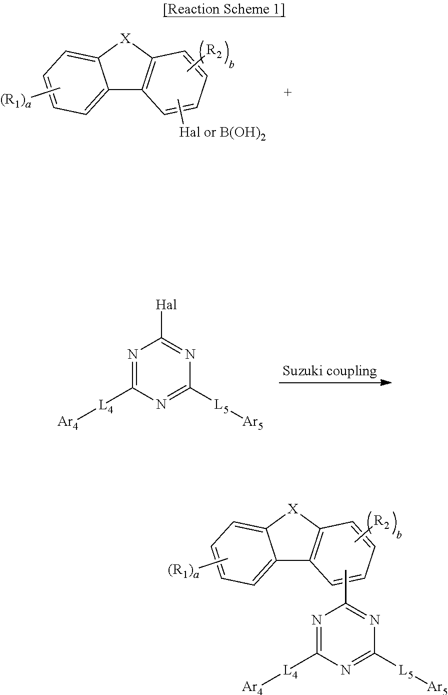

In Reaction Scheme 1, X, R₁, R₂, L₄, L₅, Ar₄, Ar₅, a, and b have the same definition as defined in Formula 2, and Hal represents halogen.

Although illustrative synthesis examples of the compound represented by Formulas 1 and 2 are described above, one skilled in the art will be able to readily understand that all of these are based on a Buchwald-Hartwig cross-coupling reaction, an N-arylation reaction, an H-mont-mediated etherification reaction, a Miyaura borylation reaction, a Suzuki cross-coupling reaction, an intramolecular acid-induced cyclization reaction, a Pd(II)-catalyzed oxidative cyclization reaction, a Grignard reaction, a Heck reaction, a cyclic dehydration reaction, an SN₁ substitution reaction, an SN₂ substitution reaction, a phosphine-mediated reductive cyclization reaction, etc., and the above reaction proceeds even when substituents which are defined in Formulas 1 and 2, but not specified in the specific synthesis example, are bonded.

Hereinafter, an organic electroluminescent device using the plurality of host materials described above will be described in more detail.

The present disclosure provides an organic electroluminescent device comprising an anode; a cathode; and at least one light-emitting layer between the anode and the cathode, wherein at least one light-emitting layer comprises the plurality of host materials according to the present disclosure. The first host material and the second host material of the present disclosure may be comprised in one light-emitting layer or may be respectively comprised in different light-emitting layers among a plurality of light-emitting layers. The plurality of host materials of the present disclosure may comprise the compound represented by Formula 1 and the compound represented by Formula 2 at a ratio of about 1:99 to about 99:1, preferably about 10:90 to about 90:10, more preferably about 30:70 to about 70:30. In addition, the compound represented by Formula 1 and the compound represented by Formula 2 in a desired ratio may be combined by mixing them in a shaker, by dissolving them in a glass tube by way of heat, or by dissolving them in a solvent, etc.

According to one embodiment of the present disclosure, the doping concentration of the dopant compound with respect to the host compound of the light-emitting layer may be less than 20 wt %. The dopants comprised in the organic electroluminescent device of the present disclosure may be at least one phosphorescent or fluorescent dopant, and is preferably a phosphorescent dopant. The phosphorescent dopant materials applied to the organic electroluminescent device according to the present disclosure are not particularly limited, but may be a metallated complex compound of a metal atom selected from iridium (Ir), osmium (Os), copper (Cu), and platinum (Pt), preferably a ortho-metallated complex compound of a metal atom selected from iridium (Ir), osmium (Os), copper (Cu), and platinum (Pt), and more preferably an ortho-metallated iridium complex compound.

The dopant comprised in the organic electroluminescent device of the present disclosure may be a compound represented by the following Formula 101, but is not limited thereto.

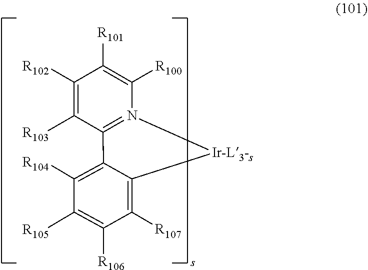

In Formula 101,
L′ is any one selected from the following Structures 1 to 3,

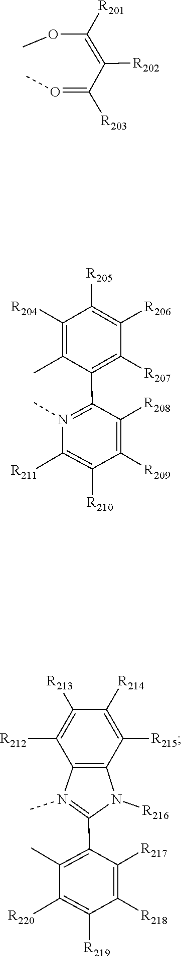

R₁₀₀ to R₁₀₃ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline, together with pyridine;
R₁₀₄ to R₁₀₇ each independently represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;
R₂₀₁ to R₂₂₀ each independently represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and
s represents an integer of 1 to 3.

The specific examples of the dopant compound are as follows, but are not limited thereto.

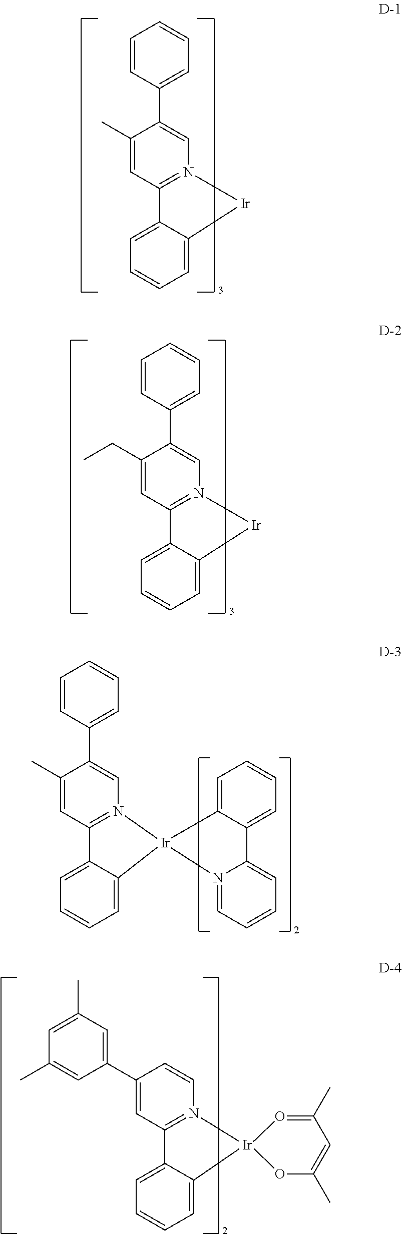

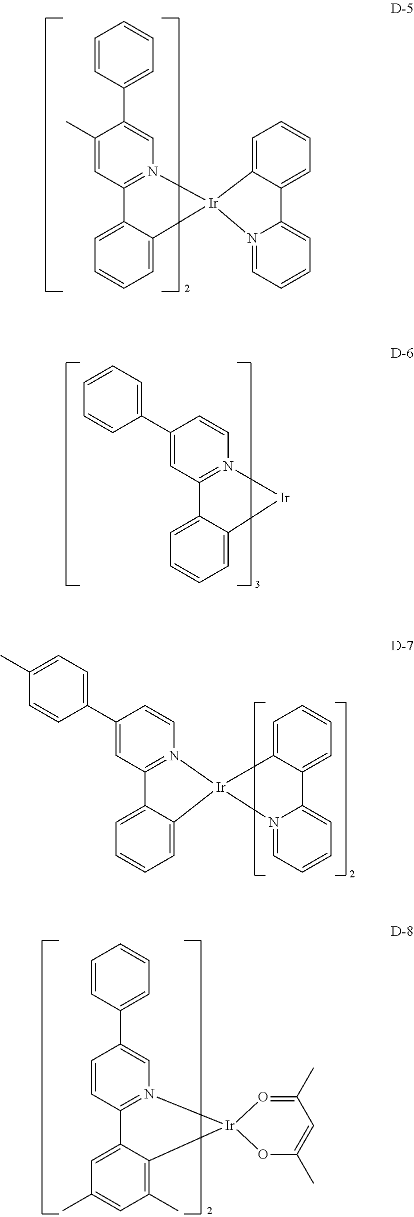

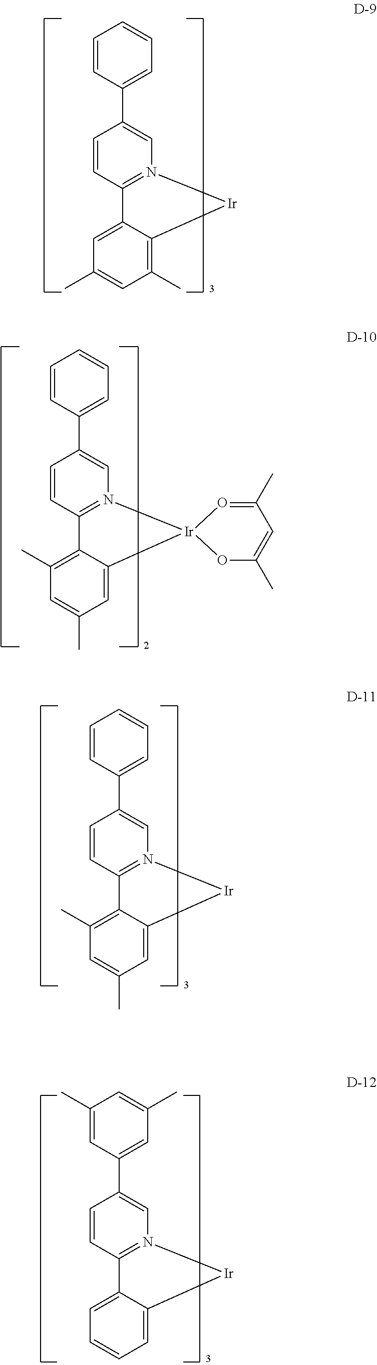

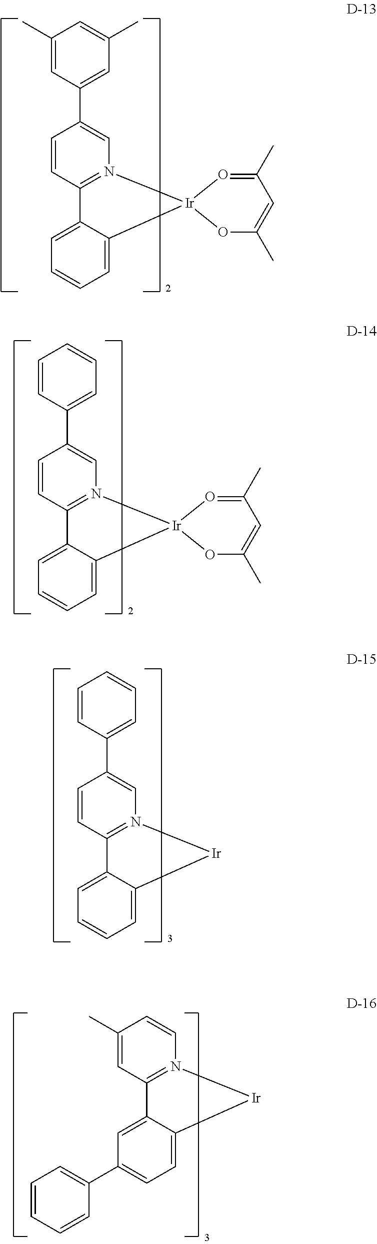

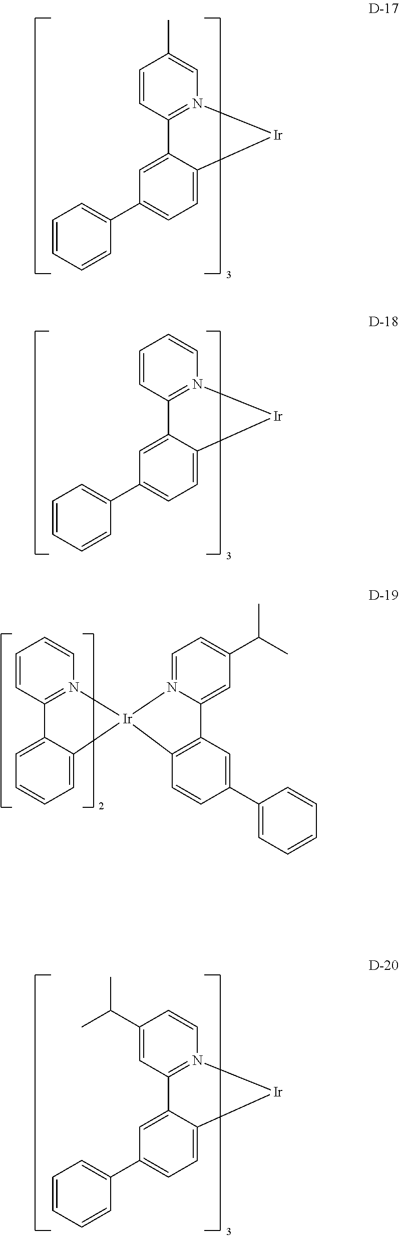

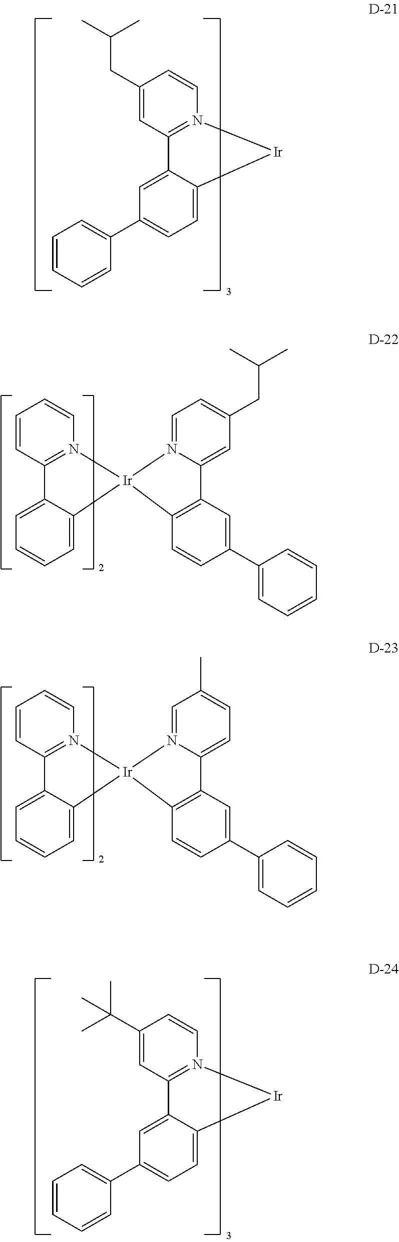

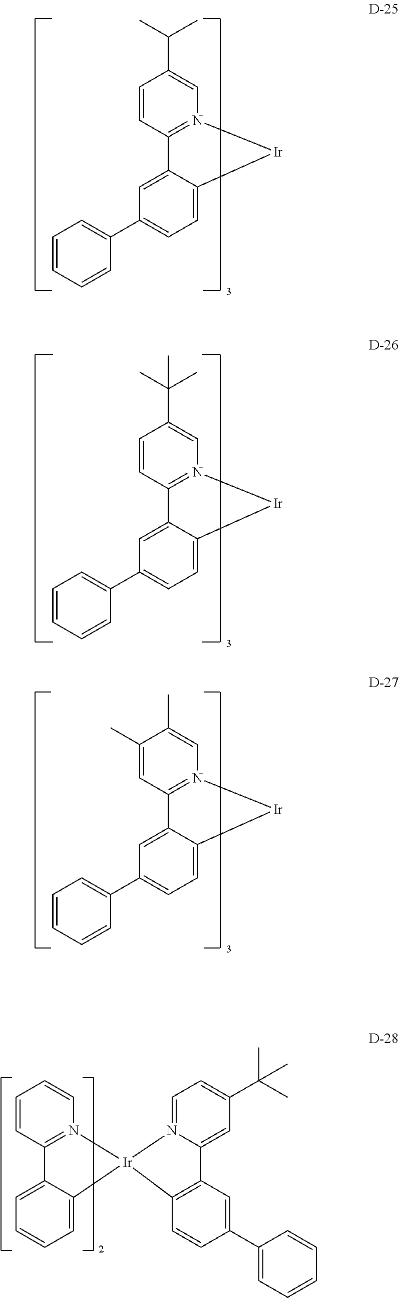

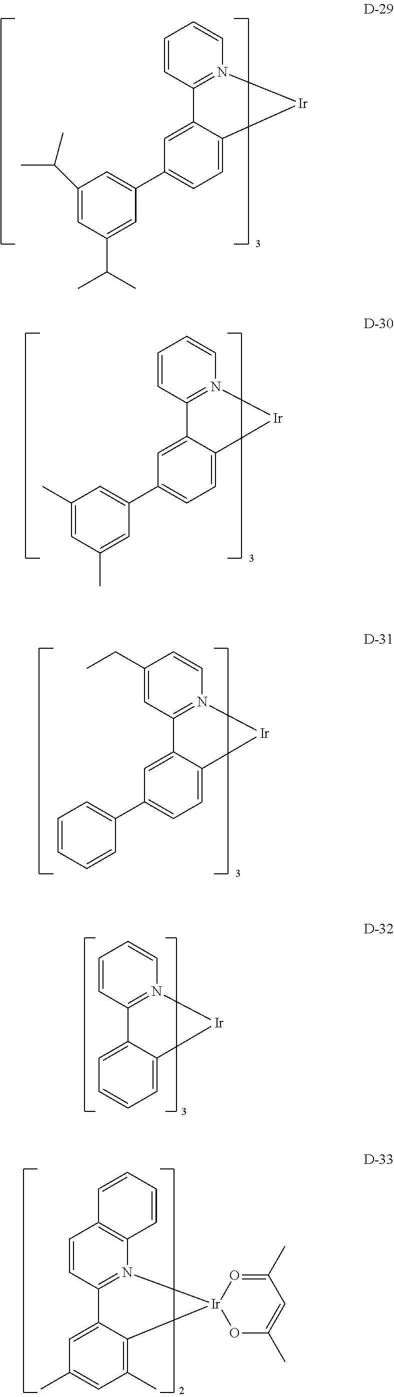

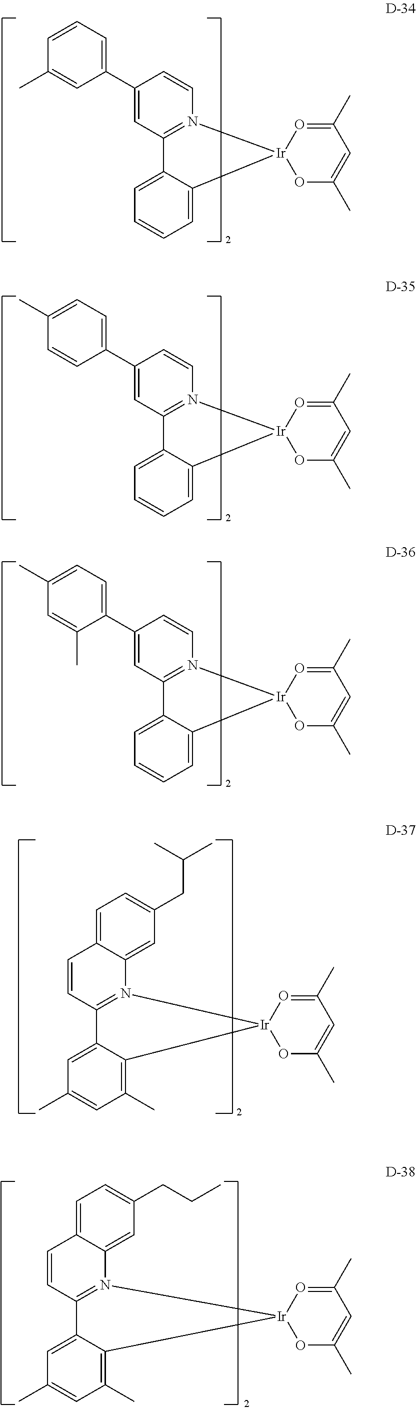

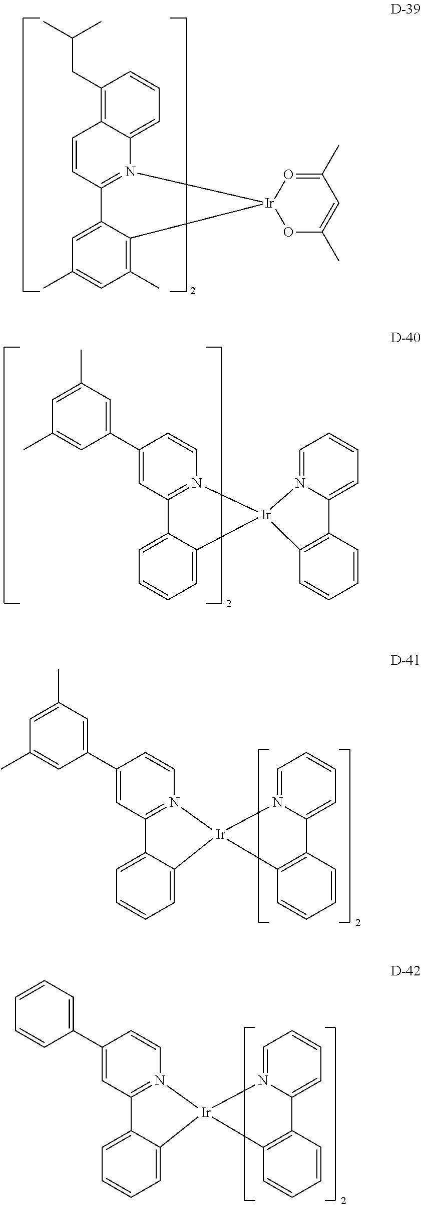

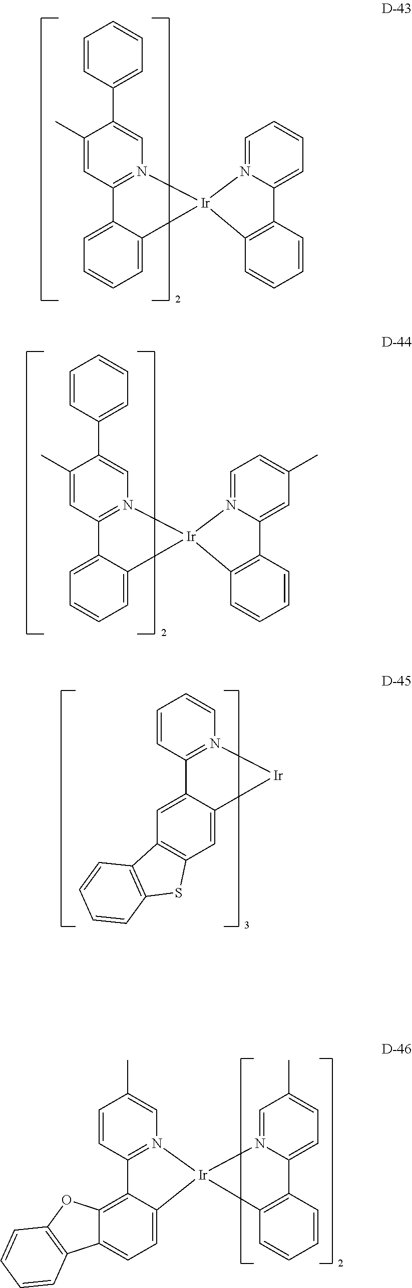

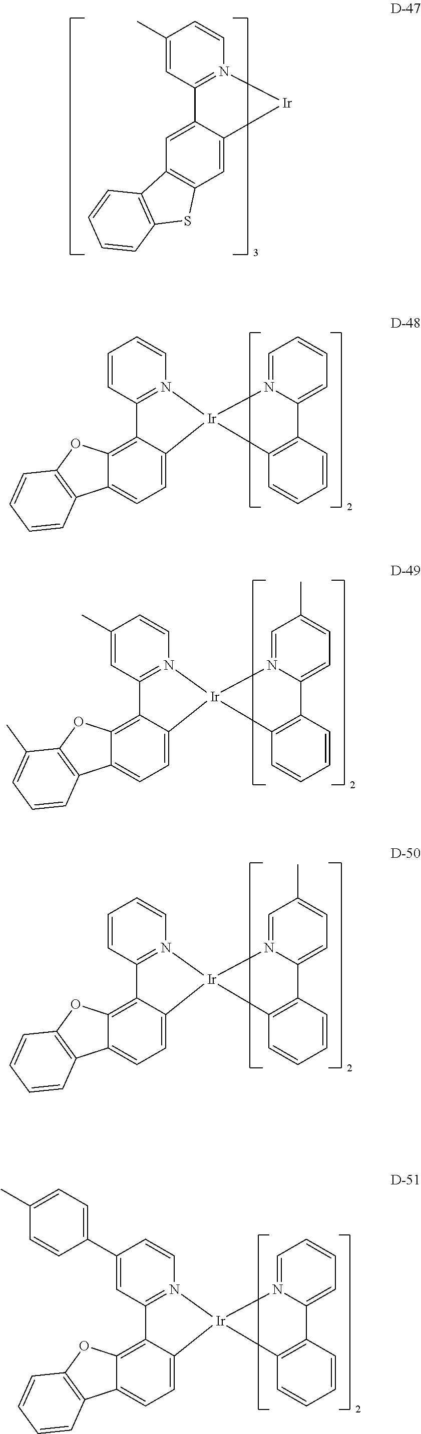

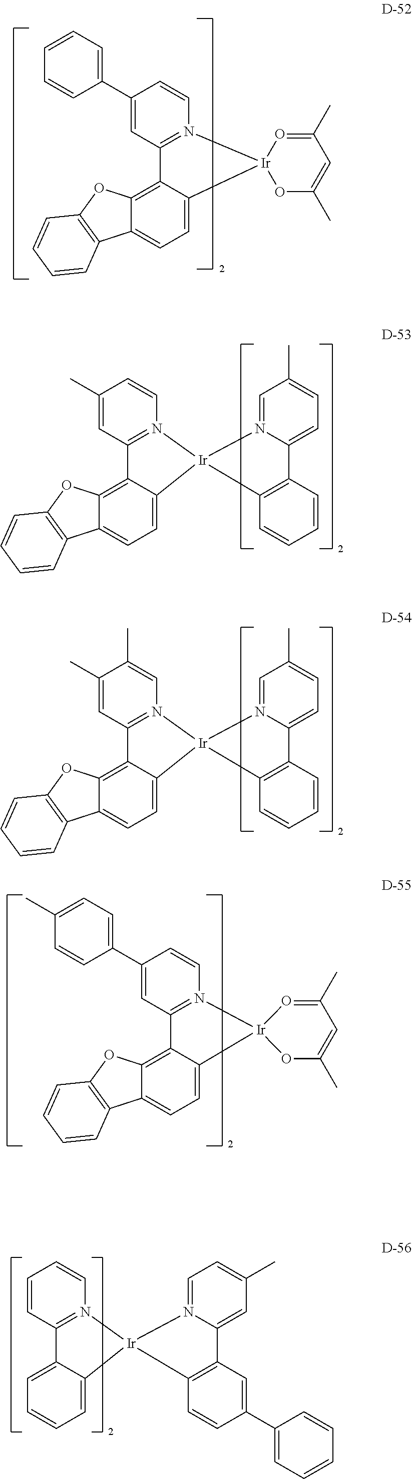

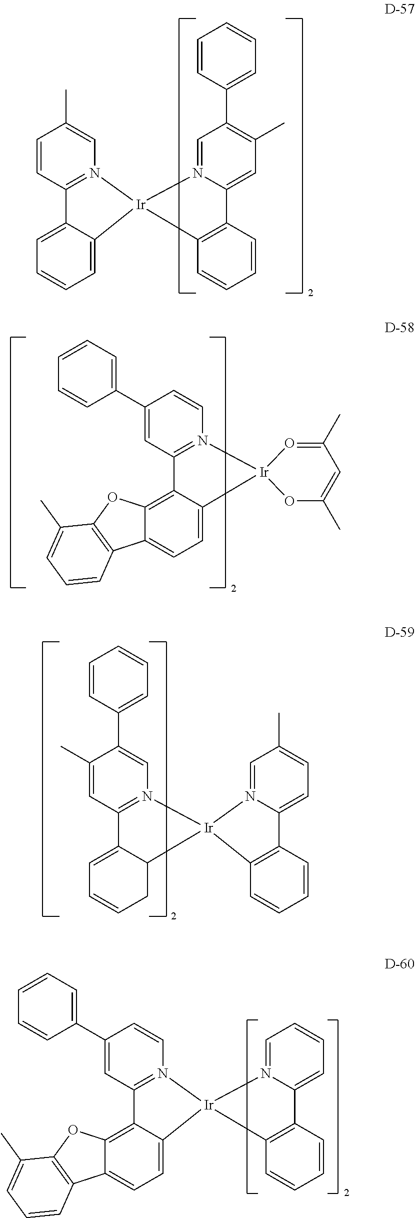

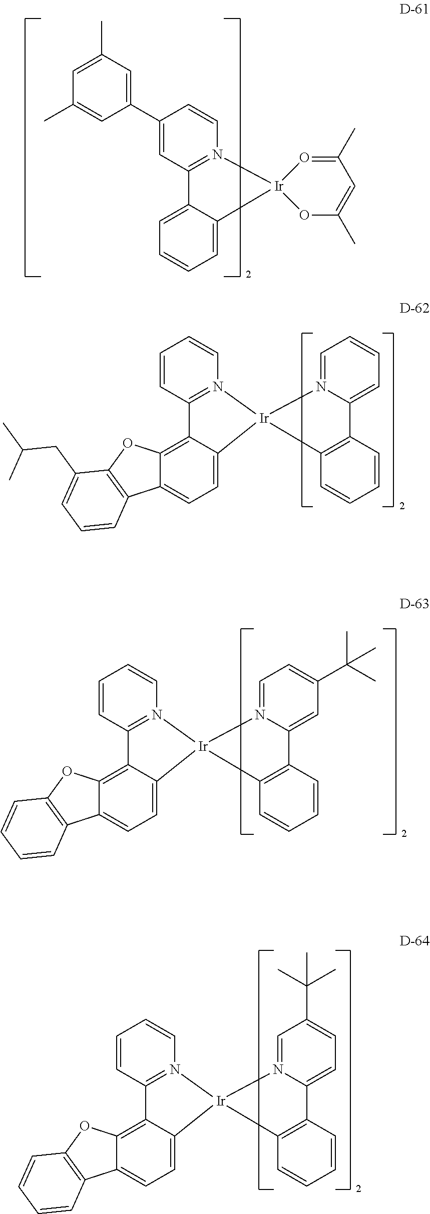

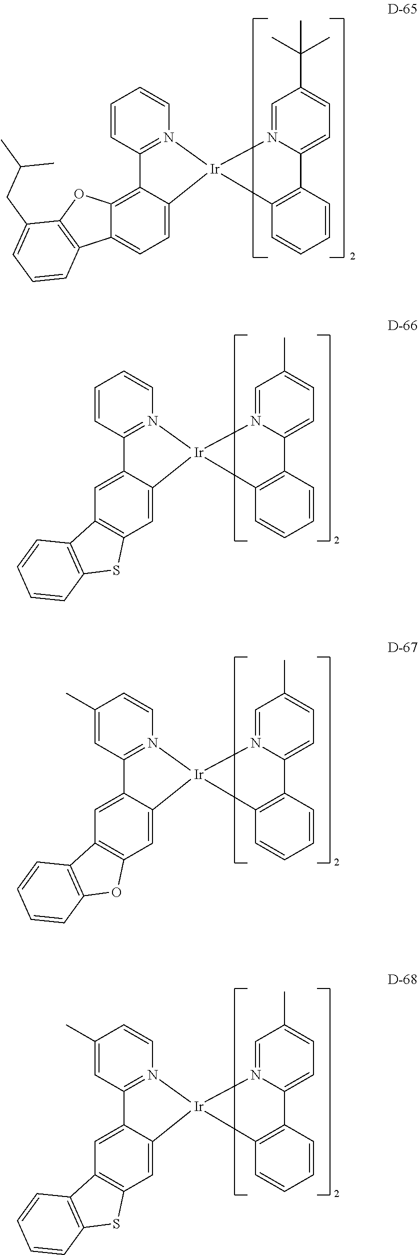

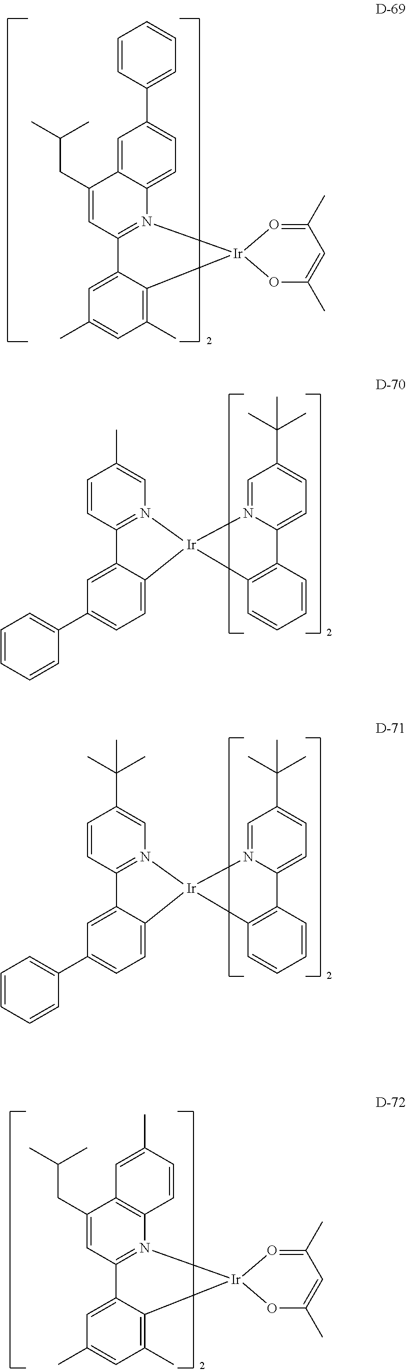

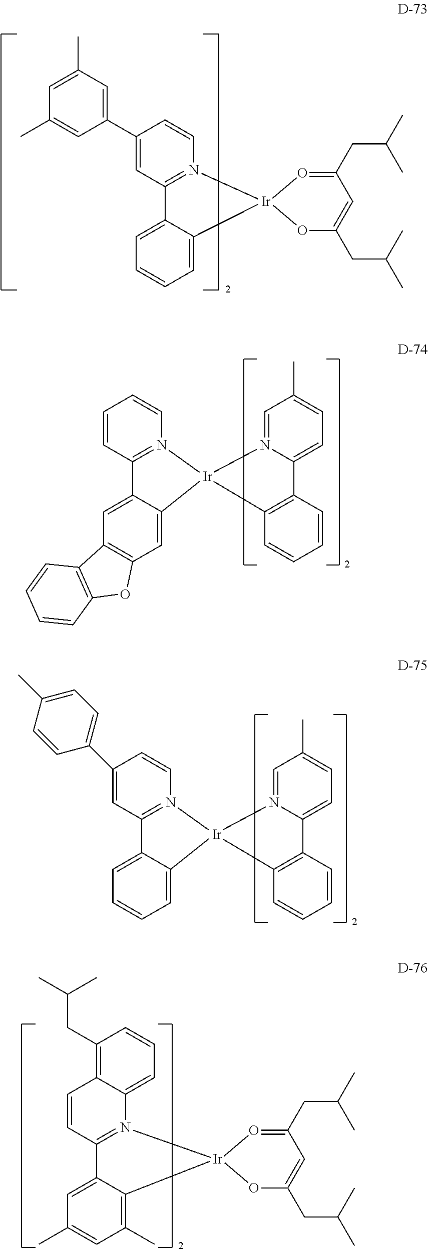

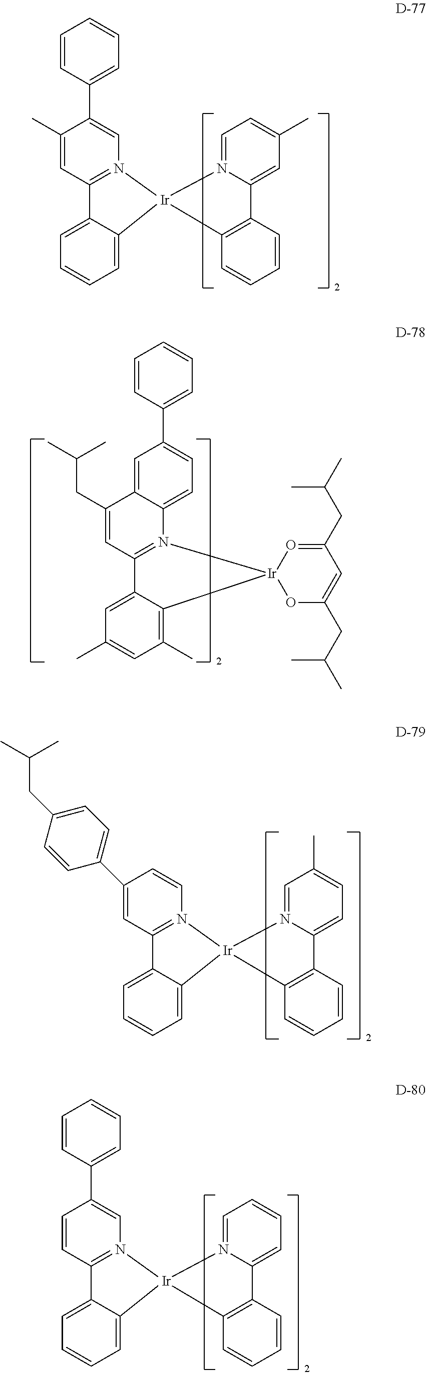

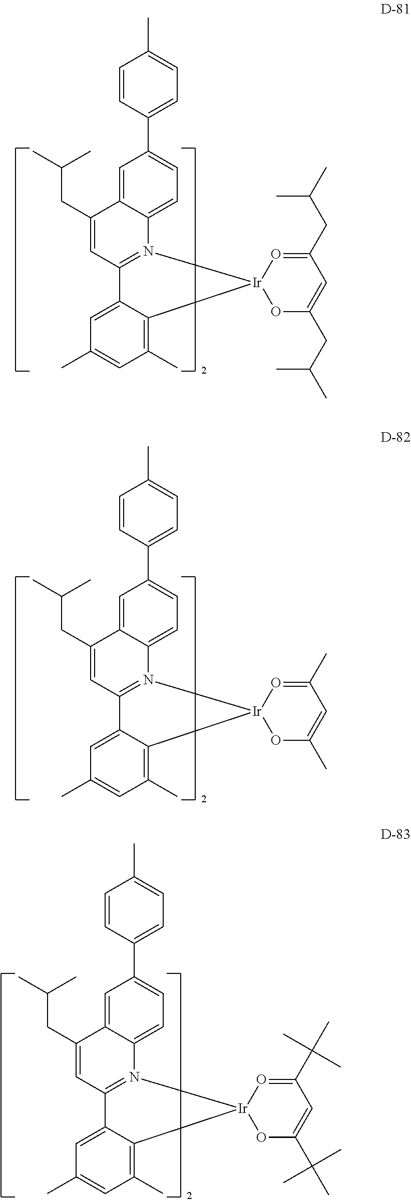

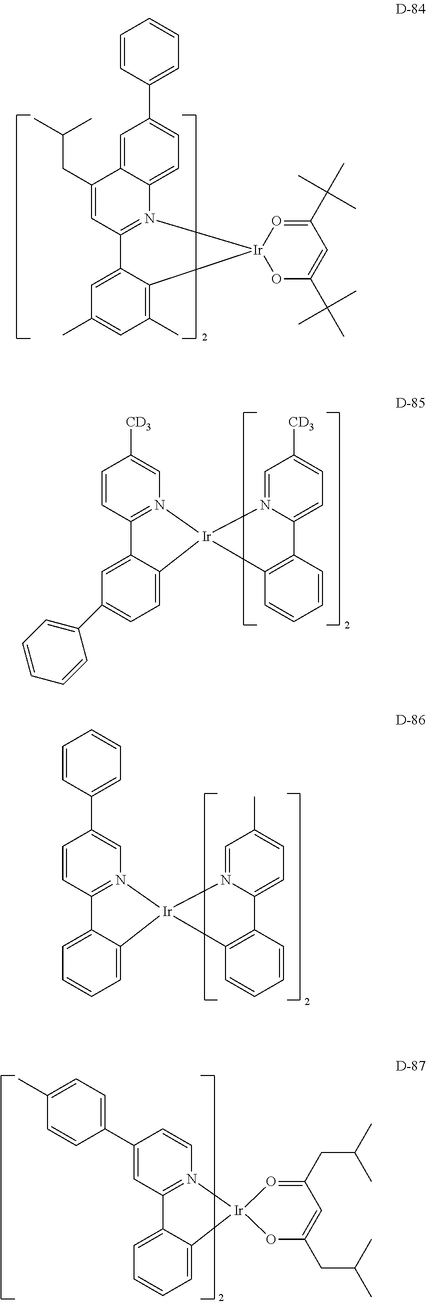

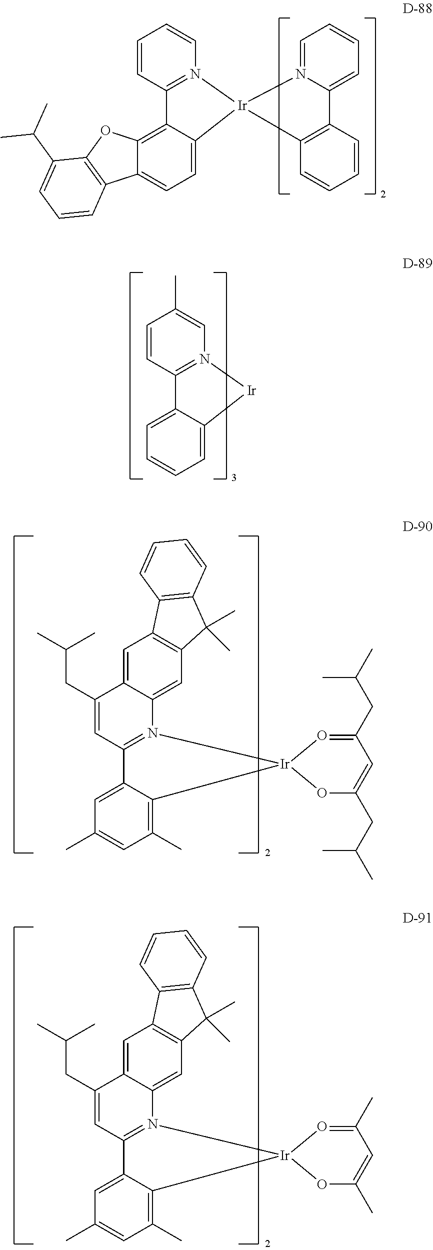

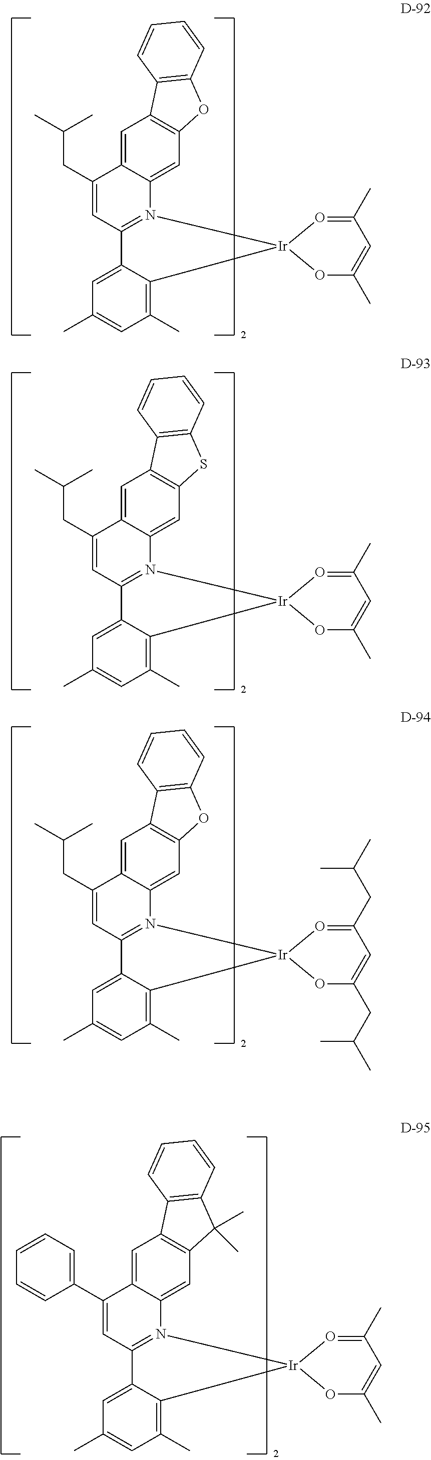

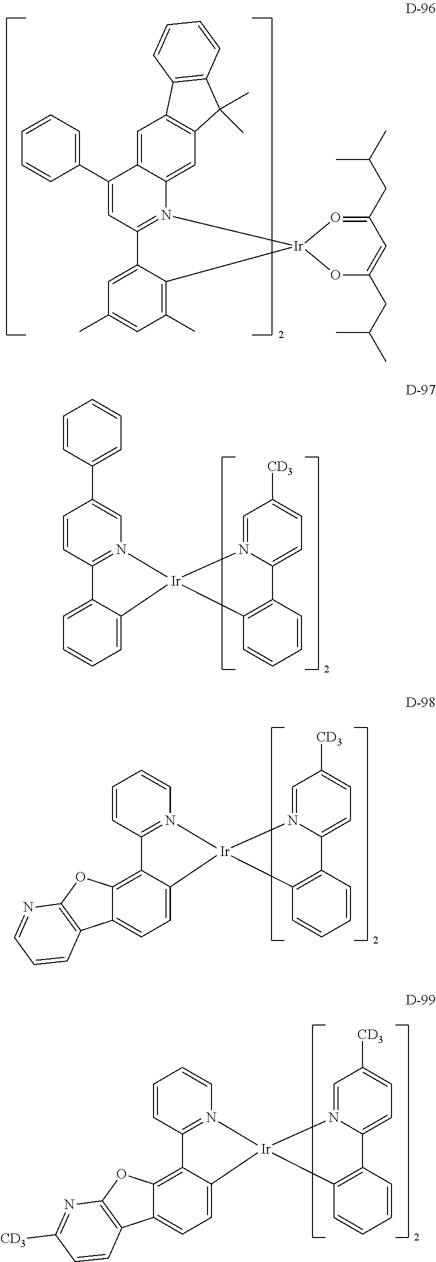

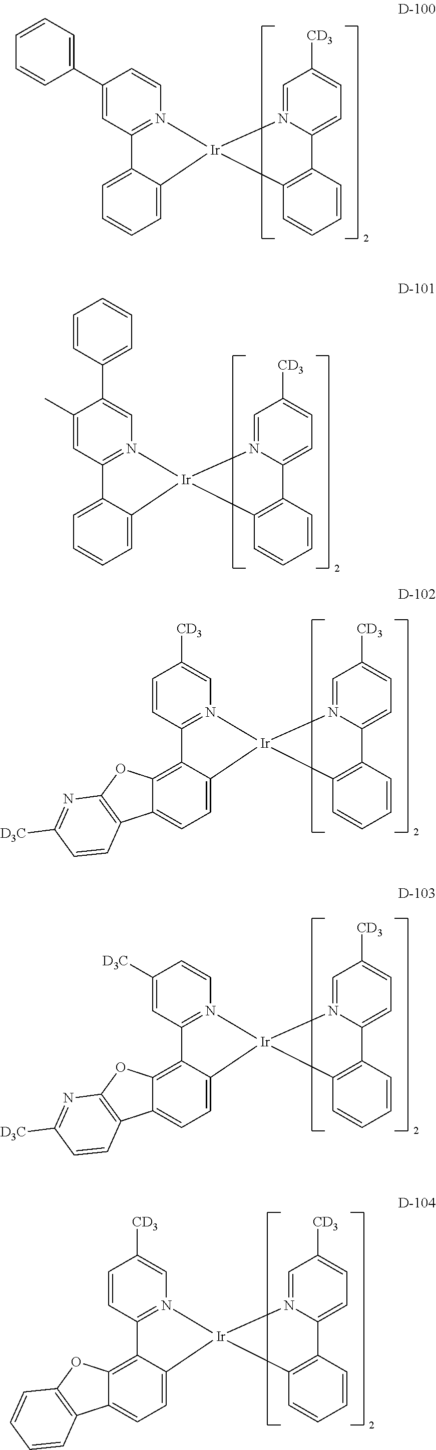

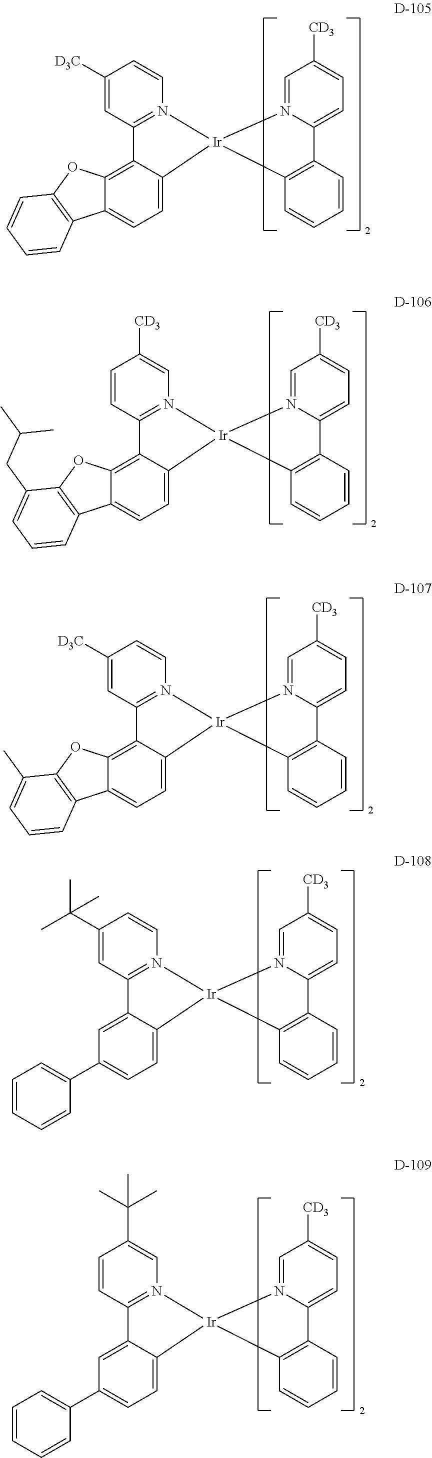

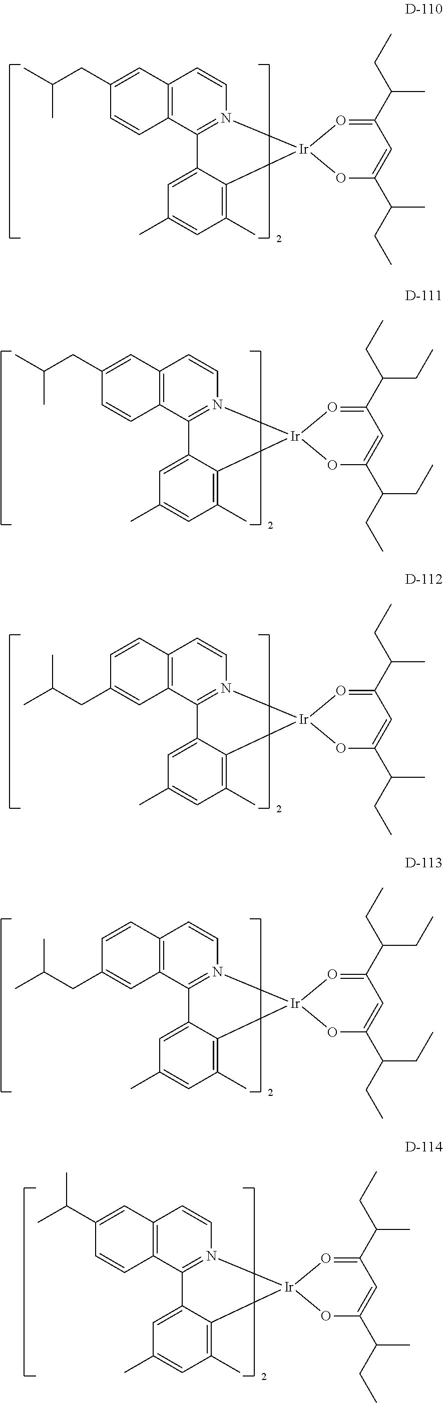

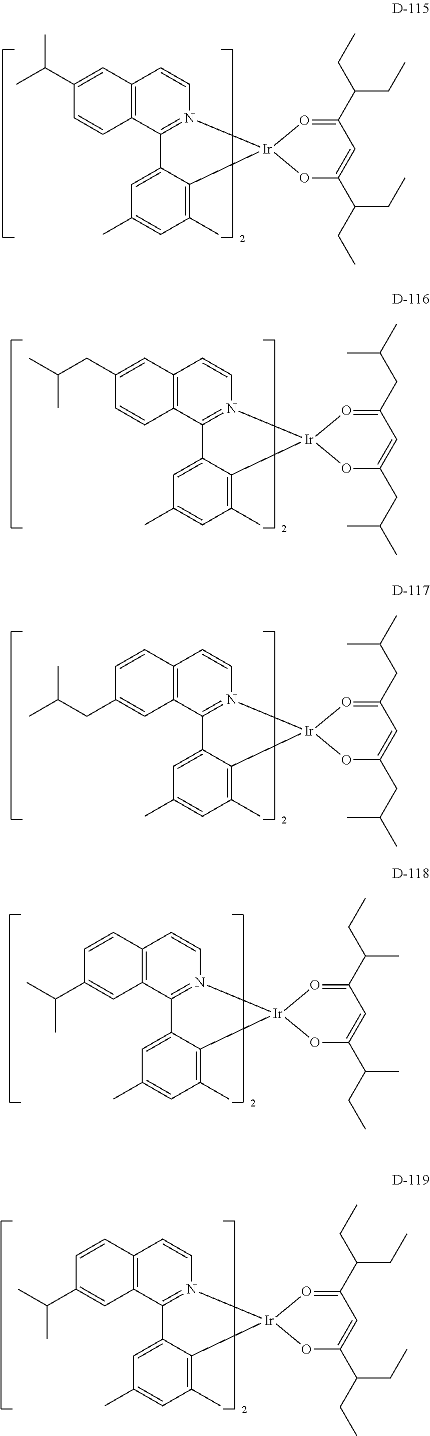

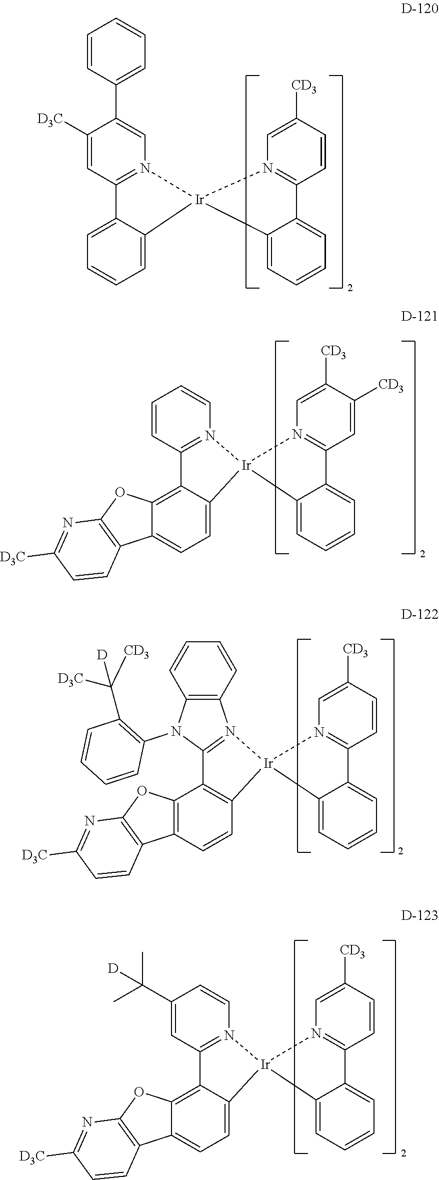

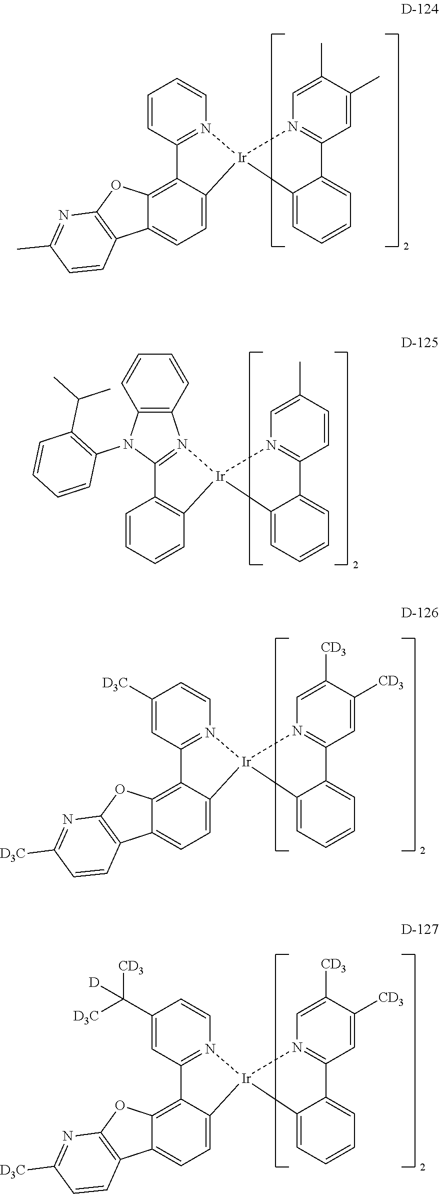

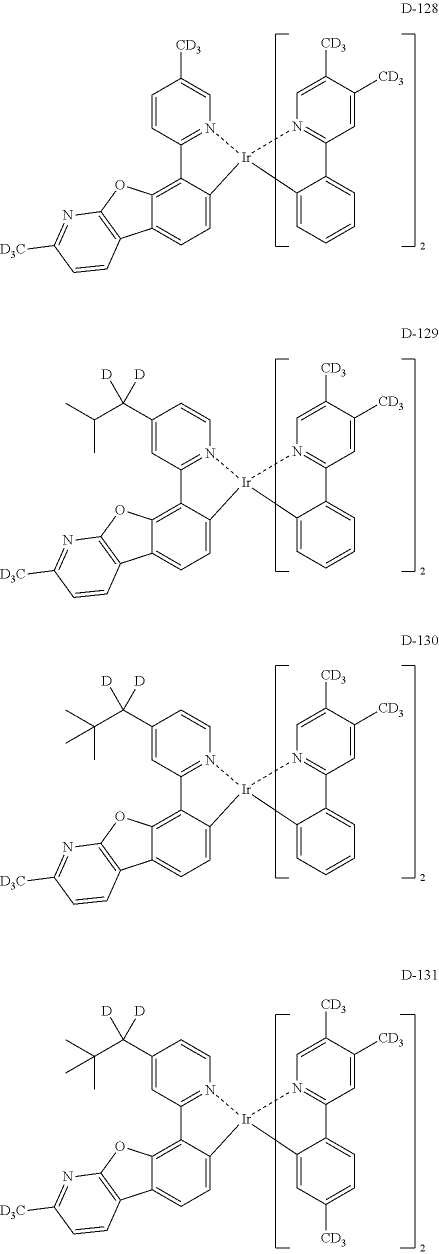

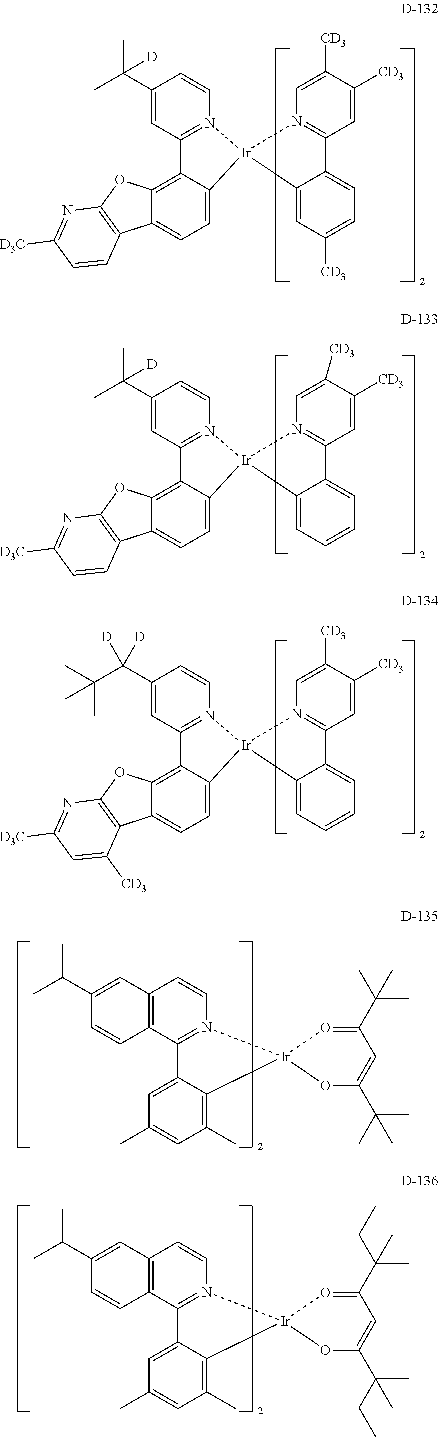

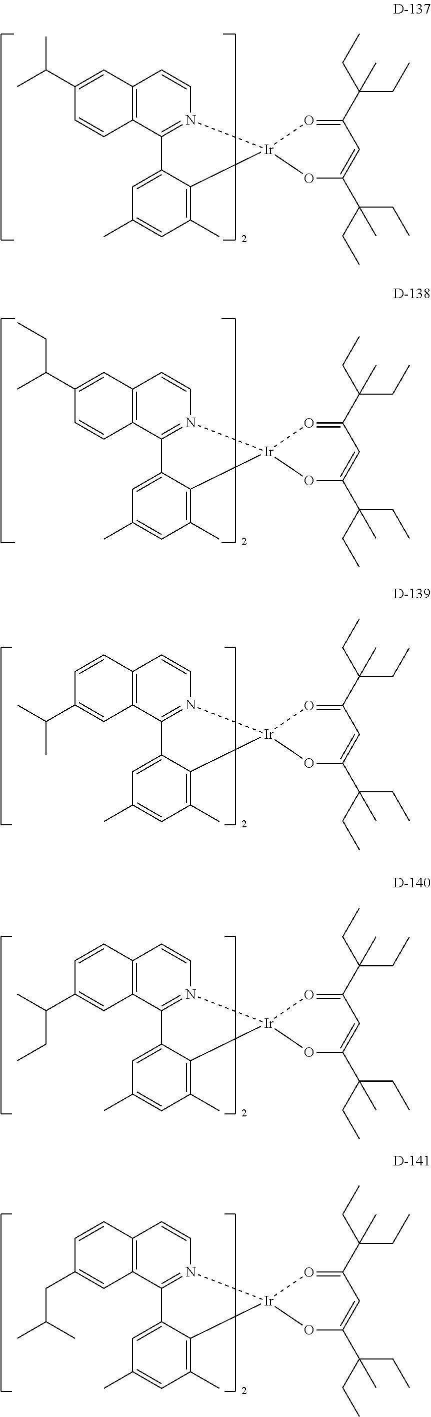

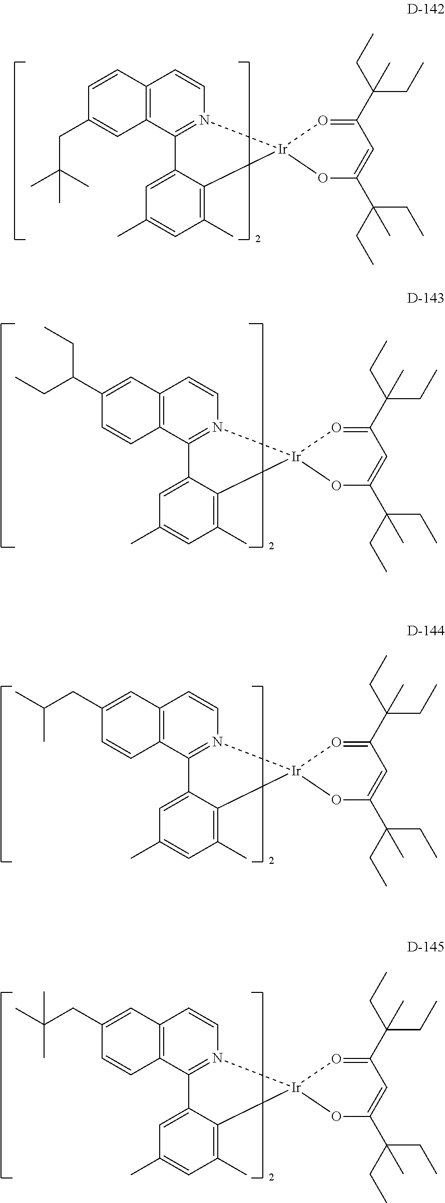

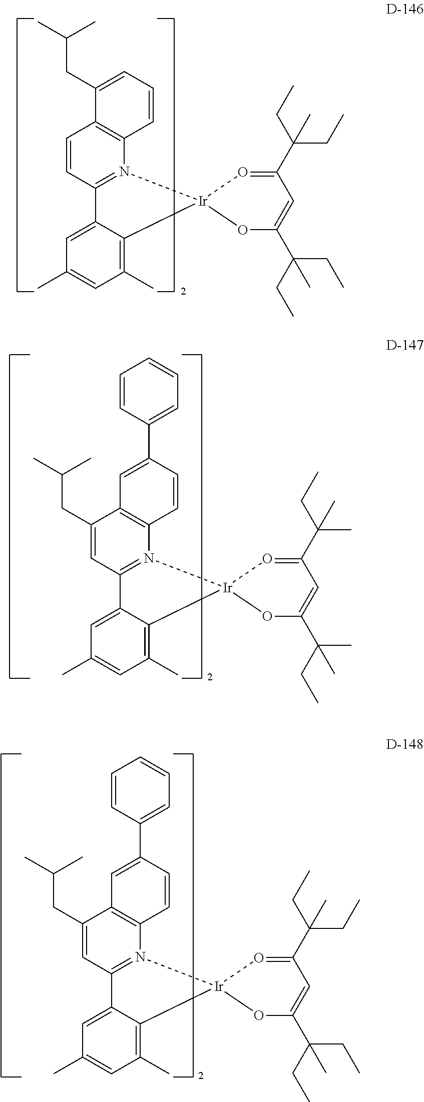

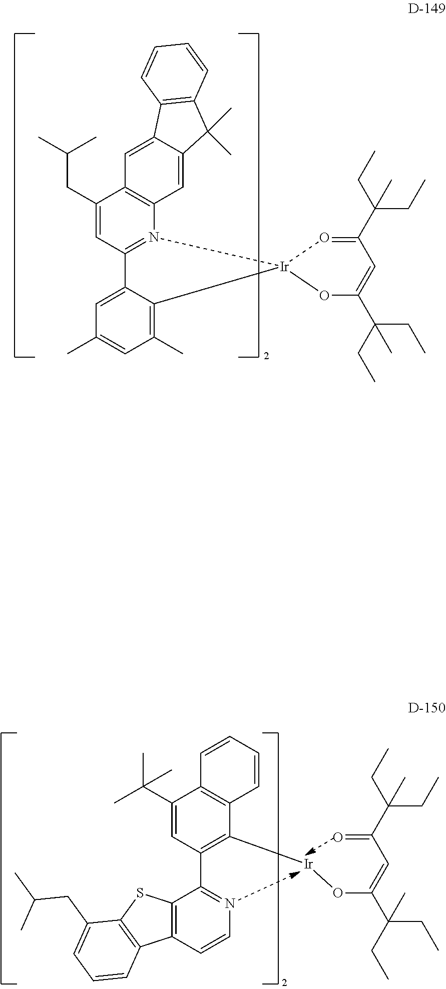

The organic electroluminescent device according to the present disclosure comprises an anode; a cathode; and at least one organic layer between the anode and the cathode. The organic layer comprises a light-emitting layer, and may further comprise at least one layer selected from any of the following: a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may be further configured as a plurality of layers.

Each of the anode and the cathode may be formed of a transparent conductive material or a transflective or reflective conductive material. Depending on the type of material forming the anode and the cathode, the organic electroluminescent device may be a top light-emitting type, a bottom light-emitting type, or a double side light-emitting type. In addition, the hole injection layer may be further doped with a p-dopant(s), and the electron injection layer may be further doped with an n-dopant(s).

At least one compound(s) selected from the group consisting of arylamine-based compounds and styrylarylamine-based compounds may be further comprised in the organic layer. In addition, the organic material layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, red, or green light-emitting compound known in the art, besides the compound of the present disclosure. In addition, if necessary, it may further comprise a yellow or orange light-emitting layer.

In the organic electroluminescent device of the present disclosure, it is preferable to dispose at least one layer selected from a chalcogenide layer, a metal halide layer, and a metal oxide layer (hereinafter referred to as a “surface layer”) on at least one inner surface of a pair of electrodes. Specifically, a chalcogenide (including an oxide) layer of silicon and aluminum is preferably placed on an anode surface of an electroluminescent medium layer side, and a metal halide layer or a metal oxide layer is preferably placed on a cathode surface of an electroluminescent medium layer side. Driving stabilization of the organic electroluminescent device can be obtained by the surface layer. Preferred examples of the chalcogenide include SiO_{X}(1≤X≤2), AlO_{X}(1≤X≤1.5), SiON, SiAlON, etc., preferred examples of the metal halide include LiF, MgF₂, CaF₂, a rare earth metal fluoride, etc, and preferred examples of the metal oxide include Cs₂O, Li₂O, MgO, SrO, BaO, CaO, etc.

A hole injection layer, a hole transport layer, an electron blocking layer, or a combination thereof may be used between an anode and a light-emitting layer. The hole injection layer may be multi-layered in order to lower the hole injection barrier (or hole injection voltage) from the anode to the hole transport layer or the electron blocking layer, wherein two compounds may be simultaneously used in each of the multi-layers. The hole transport layer or the electron blocking layer may be multi-layered.

An electron buffer layer, a hole blocking layer, an electron transport layer, an electron injection layer, or a combination thereof may be used between a light-emitting layer and a cathode. The electron buffer layer may be multi-layered in order to control electron injection and improve interfacial properties between the light-emitting layer and the electron injection layer, wherein two compounds may be simultaneously used in each of the multi-layers. The hole blocking layer or the electron transport layer may be multi-layered, wherein a plurality of compounds may be used in each of the multi-layers.

A light-emitting auxiliary layer may be a layer placed between an anode and a light-emitting layer, or between a cathode and a light-emitting layer. When placed between the anode and the light-emitting layer, the light-emitting auxiliary layer may be used to facilitate hole injection and/or hole transport or to block the overflow of electrons. When placed between the cathode and the light-emitting layer, the light-emitting auxiliary layer may be used to facilitate electron injection and/or electron transport or to block the overflow of holes. In addition, the hole auxiliary layer may be placed between the hole transport layer (or hole injection layer) and the light-emitting layer, and may exhibit an effect of facilitating or blocking the hole transport rate (or hole injection rate), and accordingly may adjust the charge balance. In addition, the electron blocking layer may be placed between the hole transport layer (or hole injection layer) and the light-emitting layer, and may block the overflow of electrons from the light-emitting layer and confine the excitons in the light-emitting layer to prevent light leakage. When an organic electroluminescent device includes two or more hole transport layers, the hole transport layer, which is further included, may be used as a hole auxiliary layer or an electron blocking layer. The light-emitting auxiliary layer, the hole auxiliary layer, or the electron blocking layer may have an effect of improving the efficiency and/or lifetime of the organic electroluminescent device.

In addition, in an organic electroluminescent device of the present disclosure, a mixed region of an electron transport compound and a reductive dopant, or a mixed region of a hole transport compound and an oxidative dopant, may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferred oxidative dopants include various Lewis acids and acceptor compounds, and preferred reductive dopants include alkali metals, alkali metal compounds, alkaline earth metals, rare earth metals, and mixtures thereof. In addition, an organic electroluminescent device having at least two light-emitting layers and emitting white light may be manufactured by using the reductive dopant layer as a charge-generating layer.

The organic electroluminescent material according to one embodiment of the present disclosure may be used as a light-emitting material for a white organic light-emitting device. The white organic light-emitting device has been suggested to have various structures such as a side-by-side arrangement method, a stacking arrangement method, or a color conversion material (CCM) method, etc., according to the arrangement of R (Red), G (Green) or YG (Yellowish Green), and B (Blue) light-emitting units. In addition, the organic electroluminescent material according to one embodiment of the present disclosure may also be used in an organic electroluminescent device comprising a quantum dot (QD).

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When forming a film of the first host compound and the second host compound of the present disclosure, co-deposition or mixed deposition is performed.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

In addition, it is possible to produce a display system, e.g. a display system for smartphones, tablets, notebooks, PCs, TVs, or cars; or a lighting system, e.g. an outdoor or indoor lighting system, using the organic electroluminescent device of the present disclosure.

Hereinafter, the preparation method of the compound of the present disclosure, the properties thereof, and the driving voltage and the luminous efficiency of the organic electroluminescent device (OLED) comprising a plurality of host materials according to the present disclosure, will be explained in detail with reference to the representative compounds of the present disclosure. However, the following examples only describe the properties of the compound according to the present disclosure and the OLED comprising the same, and the present disclosure is not limited to the following examples.

### Example 1: Preparation of Compound H-99

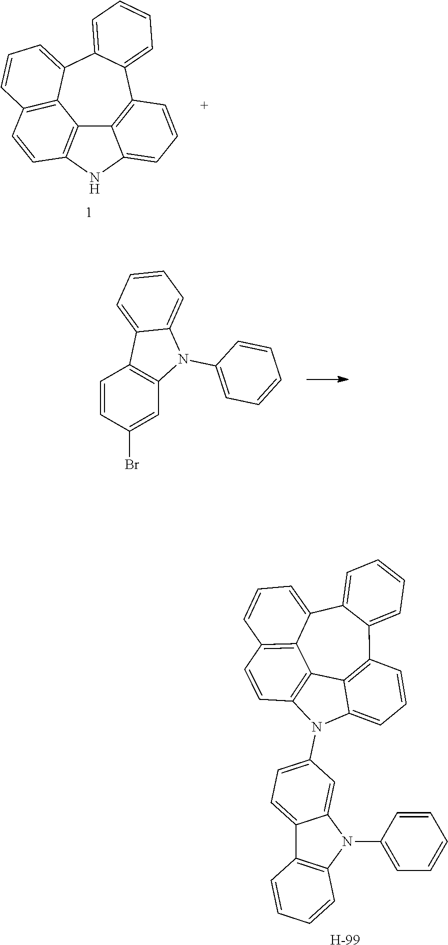

In a flask, compound 1 (35 g, 120.13 mmol), 2-bromo-9-phenyl-9H-carbazole (58 g, 180.19 mmol), CuI (11 g, 60.06 mmol), ethylenediamine (8 mL, 120.13 mmol), and cesium carbonate (Cs₂CO₃) (78 g, 240.26 mmol) were dissolved in 600 mL of o-xylene, and the mixture was stirred under reflux for 6 hours. The mixture was cooled to room temperature, and H₂O was added to the solid reaction product, and this was stirred for 30 minutes, filtered, and separated by way of column chromatography to obtain compound H-99 (29.5 g, yield: 46.8%).

| | MW | M.P. |
|---|---|---|
| H-99 | 532.65 | 278.6° C. |

### Example 2: Preparation of Compound H-1

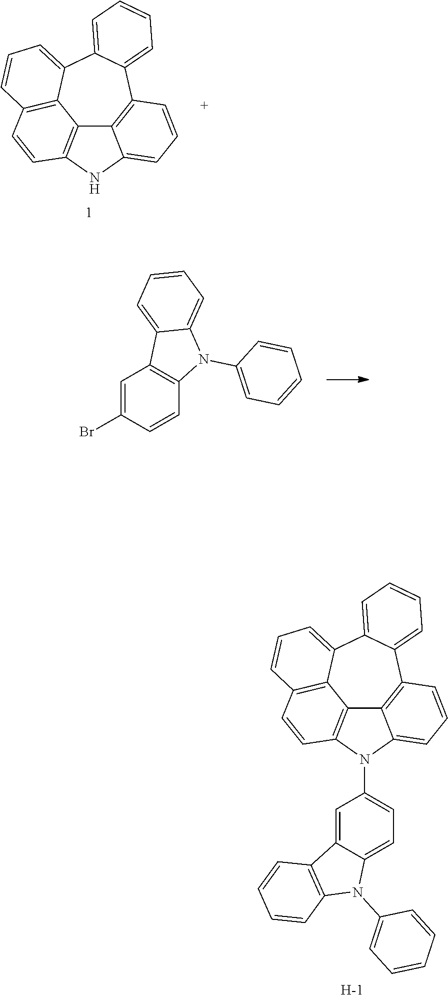

In a flask, compound 1 (10 g, 34.322 mmol), 3-bromo-9-phenyl-9H-carbazole (16.5 g, 51.484 mmol), CuI (3.2 g, 17.162 mmol), ethylenediamine (2.2 mL, 34.322 mmol), and cesium carbonate (Cs₂CO₃) (22.4 g, 68.646 mmol) were dissolved in 171 mL of o-xylene, and the mixture was stirred under reflux for 6 hours. The mixture was cooled to room temperature, and H₂O was added to the solid reaction product, and this was stirred for 30 minutes, filtered, and separated by way of column chromatography to obtain compound H-1 (11.4 g, yield: 63%).

| | MW | M.P. |
|---|---|---|
| H-1 | 532.65 | 167.1° C. |

### Example 3: Preparation of Compound H-2

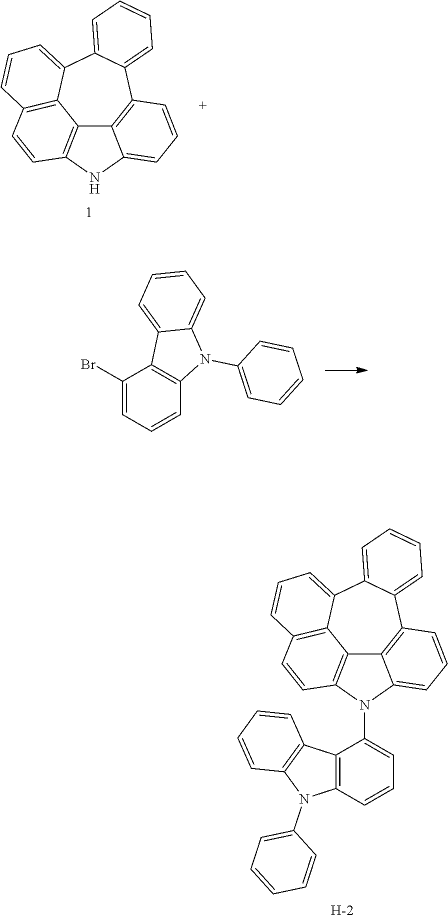

In a flask, compound 1 (9 g, 30.8 mmol), 4-bromo-9-phenyl-9H-carbazole (15 g, 46.5 mmol), Cu₂SO₄ (2.5 g, 15.6 mmol), and potassium carbonate (K₂CO₃) (8.5 g, 61.5 mmol) were dissolved in 200 mL of o-dichlorobenzene, and the mixture was stirred under reflux for 4 hours. The mixture was cooled to room temperature, and H₂O was added to the solid reaction product, and this was stirred for 30 minutes, filtered, and separated by way of column chromatography to obtain compound H-2 (4 g, yield: 24%).

| | MW | M.P. |
|---|---|---|
| H-2 | 532.65 | 248.8° C. |

### Example 4: Preparation of Compound H-50

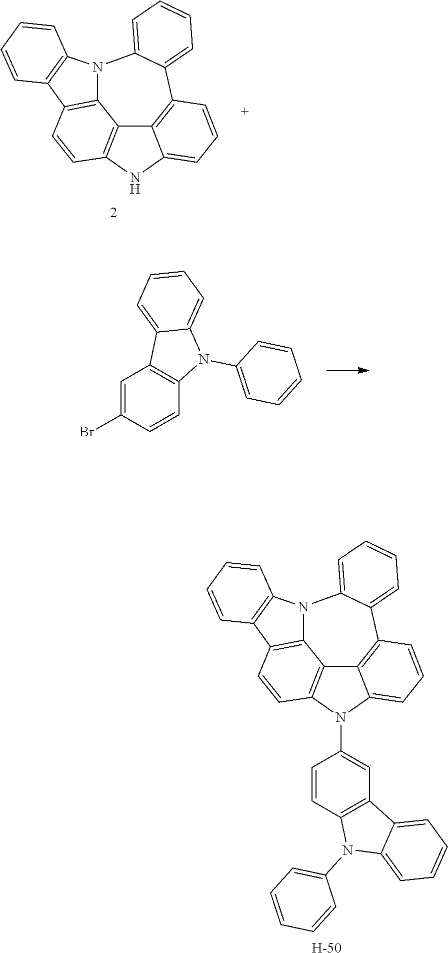

In a flask, compound 2 (5 g, 15.1 mmol), 3-bromo-9-phenyl-9H-carbazole (5.3 g, 16.6 mmol), tris(dibenzylideneacetone)dipalladium (Pd₂dba₃) (0.69 g, 0.75 mmol), 2-dicyclohexylphosphino-2′, 4′, 6′-triisopropylbiphenyl (XPhos) (0.72 g, 1.51 mmol), and sodium tert-butoxide (3.6 g, 37.8 mmol) were dissolved in 75 mL of o-xylene, and the mixture was stirred under reflux for 1.5 hours. The mixture was cooled to room temperature, filtered through silica, and separated by way of column chromatography to obtain compound H-50 (7.3 g, yield: 85%).

| | MW | M.P. |
|---|---|---|
| H-50 | 571.68 | 216° C. |

### Example 5: Preparation of Compound H-100

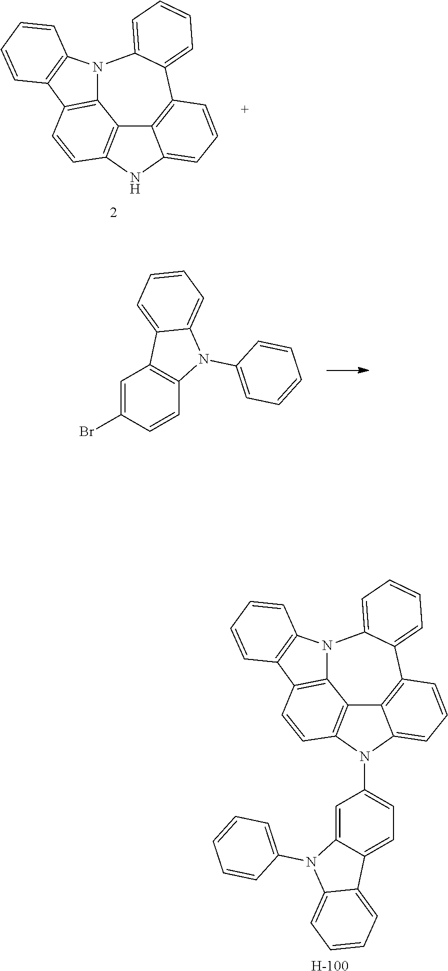

In a flask, compound 2 (7 g, 21.187 mmol), 2-bromo-9-phenyl-9H-carbazole (7.5 g, 23.31 mmol), tris(dibenzylideneacetone)dipalladium (Pd₂dba₃) (1.0 g, 1.093 mmol), 2-dicyclohexylphosphino-2′, 4′, 6′-triisopropylbiphenyl (XPhos) (1.0 g, 2.118 mmol), and sodium tert-butoxide (3.0 g, 31.78 mmol) were dissolved in 105 mL of o-xylene, and the mixture was stirred under reflux for 1.5 hours. The mixture was cooled to room temperature, filtered through silica, and separated by way of column chromatography to obtain compound H-100 (6.5 q, yield: 54%).

| | MW | M.P. |
|---|---|---|
| H-100 | 571.68 | 204.2° C. |

### Example 6: Preparation of Compound H-51

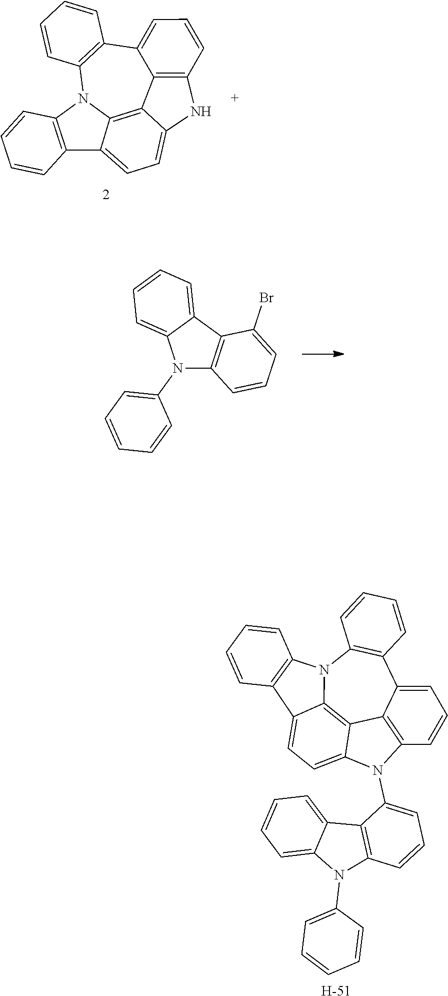

In a flask, compound 2 (10 g, 30.2 mmol), 4-bromo-9-phenyl-9H-carbazole (10.7 g, 32 mmol), tris(dibenzylideneacetone)dipalladium (Pd₂dba₃) (1.4 g, 1.5 mmol), 2-dicyclohexylphosphino-2′, 4′, 6′-triisopropylbiphenyl (XPhos) (1.45 g, 3 mmol), and sodium tert-butoxide (7.3 g, 75.96 mmol) were dissolved in 150 mL of o-xylene, and the mixture was stirred under reflux for 1 hour. After completion of the reaction, the mixture was extracted with ethyl acetate and separated by way of column chromatography to obtain compound H-51 (8 g, yield: 46.8%).

| | MW | M.P. |
|---|---|---|
| H-51 | 571.68 | 240° C. |

### Example 7: Preparation of Compound H-111

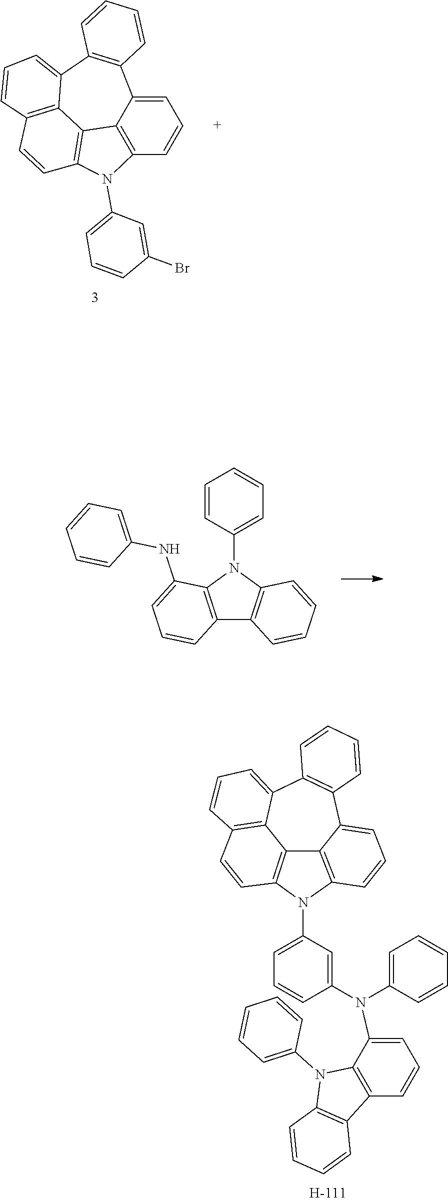 [SMILES: Brc1cccc(-n2c3cccc4c3c3c5c(cccc5ccc32)-c2ccccc2-4)c1 ; c1ccc(Nc2cccc3c4ccccc4n(-c4ccccc4)c23)cc1 ; c1ccc(N(c2cccc(-n3c4cccc5c4c4c6c(cccc6ccc43)-c3ccccc3-5)c2)c2cccc3c4ccccc4n(-c4ccccc4)c23)cc1]

In a flask, compound 3 (10 g, 22.4 mmol), N,9-diphenyl-9H-carbazole-1-amine (9 g, 26 mmol), tris(dibenzylideneacetone)dipalladium (Pd₂dba₃) (1.0 g, 1.1 mmol), 2-dicyclohexylphosphino-2′, 6′-dimethoxybiphenyl (SPhos) (900 mg, 2.2 mmol), and sodium tert-butoxide (3.17 g, 33 mmol) were dissolved in 110 mL of xylene, and the mixture was stirred at 160° C. for 3 hours. The mixture was cooled to room temperature and filtered through celite to make a solid, and then separated by way of column chromatography to obtain compound H-111 (7.1 g, yield: 45.3%).

| | MW | M.P. |
|---|---|---|
| H-111 | 699.84 | 211.4° C. |

### Example 8: Preparation of Compound H-6

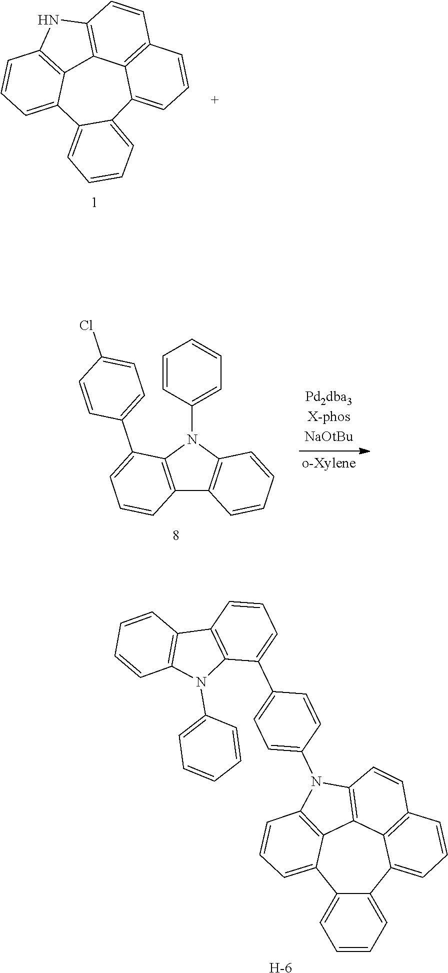

In a flask, compound 1 (5.0 g, 17.213 mmol), compound 8 (6.7 g, 18.93 mmol), tris(dibenzylideneacetone)dipalladium (Pd₂dba₃) (0.78 g, 0.86 mmol), sodium tert-butoxide (2.5 g, 25.819 mmol), and 2-dicyclohexylphosphino-2′, 4′, 6′-triisopropylbiphenyl (XPhos) (0.82 g, 1.721 mmol) were dissolved in 100 mL of xylene, and the mixture was stirred under reflux for 1 hour. After completion of the reaction, the mixture was cooled to room temperature, and filtered through celite to make a solid, and then separated by way of column chromatography to obtain compound H-6 (7.8 g, yield: 75%).

### Device Examples 1 to 5: Producing OLEDs by Co-Depositing First Host Compound and Second Host Compound According to Present Disclosure

An OLED according to the present disclosure was produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and was then stored in isopropyl alcohol. The ITO substrate was mounted on a substrate holder of a vacuum vapor deposition apparatus. Compound HI-1, shown in Table 2, was introduced into a cell of the vacuum vapor deposition apparatus, and compound HT-1 was introduced into another cell. The two materials were evaporated at different rates, and compound HI-1 was deposited in a doping amount of 3 wt % based to the total amount of compound HI-1 and compound HT-1 to form a first hole injection layer with a thickness of 10 nm. Subsequently, compound HT-1 was deposited on the first hole injection layer to form a first hole transport layer with a thickness of 80 nm. Next, compound HT-2 was introduced into another cell of the vacuum vapor deposition apparatus and was evaporated by applying an electric current to the cell, thereby depositing a second hole transport layer with a thickness of 60 nm on the first hole transport layer. After forming the hole injection layer and the hole transport layers, a light-emitting layer was deposited thereon as follows: each of the first host compound and the second host compound shown in Table 1 below were introduced into two cells of the vacuum vapor deposition apparatus as hosts, and compound D-39 was introduced into another cell as a dopant. The two host materials were evaporated at a rate of 1:1, the dopant material was simultaneously evaporated at a different rate, and the dopant was deposited in a doping amount of 3 wt % based on the total amount of the hosts and dopant to form a light-emitting layer with a thickness of 40 nm on the second hole transport layer. Then, compound ET-1 and compound E1-1 were deposited at a weight ratio of 50:50 as an electron transport material to form an electron transport layer having a thickness of 35 nm on the light-emitting layer. After depositing compound E1-1 as an electron injection layer with a thickness of 2 nm on the electron transport layer, an Al cathode was deposited with a thickness of 80 nm on the electron injection layer by using another vacuum vapor deposition apparatus to thereby produce an OLED. All the materials used for producing the OLED were purified by vacuum sublimation at 10⁻⁶ Torr.

### Comparative Examples 1 to 5: Producing OLED Comprising Comparative Compound as Host

An OLED was produced in the same manner as in Device Examples 1 to 5, except that the second host compound shown in Table 1 below was used as a host of a light-emitting layer.

Table 1, below, shows the driving voltage, luminous efficiency, light-emitting color at a luminance of 5,000 nit, and time taken for luminance to reduce from 100% to 95% at a luminance of 10,000 nit (lifespan: T95) for the OLEDs of Device Examples 1 to 5, and Comparative Examples 1 to 5 produced as described above.

TABLE 1

| | | | Driving | Luminous | Light- | |
|---|---|---|---|---|---|---|
| | First | Second | Voltage | Efficiency | Emitting | Lifespan |
| | Host | Host | (V) | (cd/A) | Color | T95(hr) |
| Device | H-99 | C-300 | 3.0 | 30.9 | Red | 264 |
| Example 1 | | | | | | |
| Comparative | H-99 | T-1 | 2.8 | 28.2 | Red | 186 |
| Example 1 | | | | | | |
| Device | H-2 | C-300 | 3.1 | 30.7 | Red | 269 |
| Example 2 | | | | | | |
| Comparative | H-2 | T-1 | 2.9 | 27.8 | Red | 125 |
| Example 2 | | | | | | |
| Device | H-1 | C-300 | 3.0 | 32.3 | Red | 283 |
| Example 3 | | | | | | |
| Comparative | H-1 | T-1 | 2.9 | 30.6 | Red | 176 |
| Example 3 | | | | | | |
| Device | H-6 | C-300 | 3.1 | 32.4 | Red | 174 |
| Example 4 | | | | | | |
| Comparative | H-6 | T-1 | 3.0 | 29.8 | Red | 64 |
| Example 4 | | | | | | |
| Device | H-50 | C-300 | 3.0 | 34.0 | Red | 316 |
| Example 5 | | | | | | |
| Comparative | H-50 | T-1 | 3.0 | 32.0 | Red | 195 |
| Example 5 | | | | | | |

From Table 1 above, it can be confirmed that the OLEDs using the plurality of host materials according to the present disclosure exhibit a driving voltage and luminous efficiency of a level at least the same as or similar to that of the organic electroluminescent device comprising the conventional combination of hosts as well as exhibiting excellent lifetime properties.

The compounds used in the Device Examples and the Comparative Examples are shown in Table 2 below.

TABLE 2

| | |
|---|---|
| Hole Injection Layer/ Hole Transport Layer | 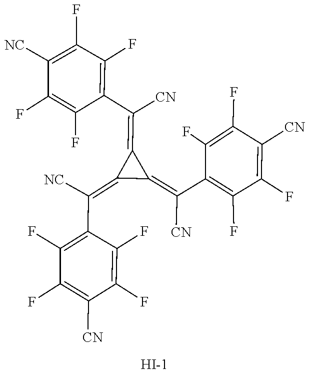 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 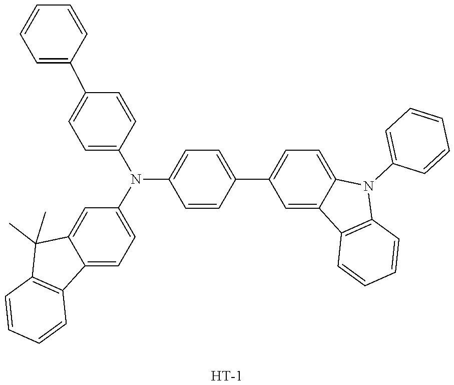 |
| | 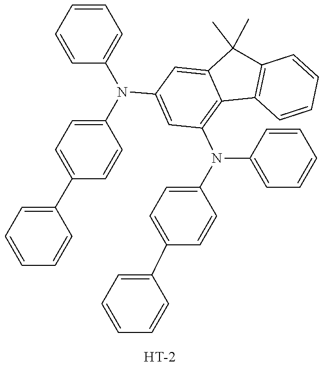 |
| Light-Emitting Layer/ Electron Buffer Layer | 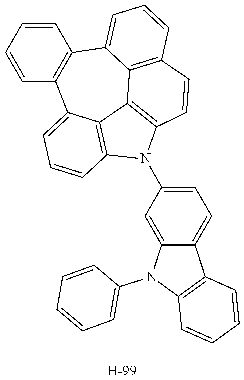 |
| | 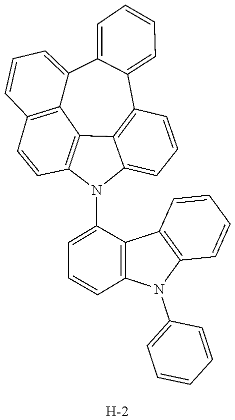 |
| | 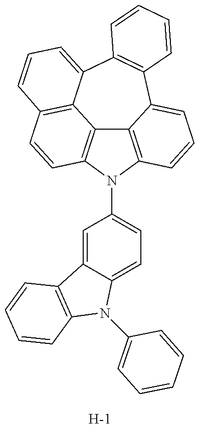 |
| | 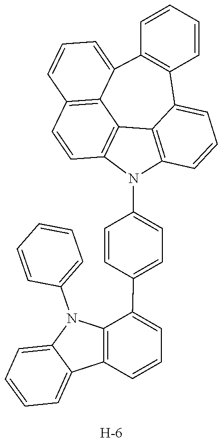 |
| | 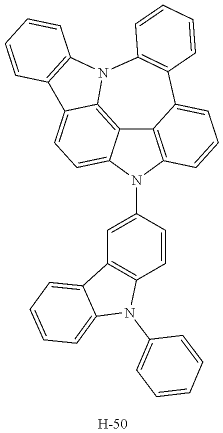 |
| | 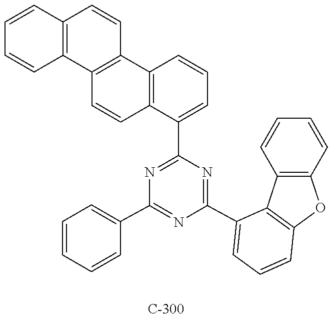 [SMILES: c1ccc(-c2nc(-c3cccc4c3ccc3c5ccccc5ccc43)nc(-c3cccc4oc5ccccc5c34)n2)cc1] |
| | 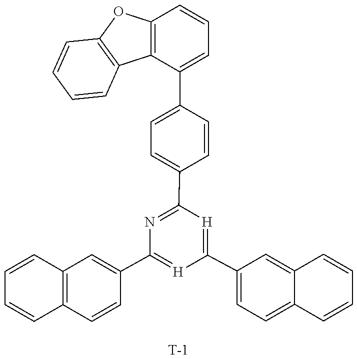 |
| | 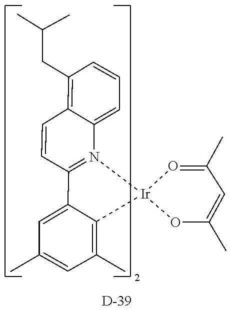 |
| Electron Transport Layer/ Electron Injection Layer | 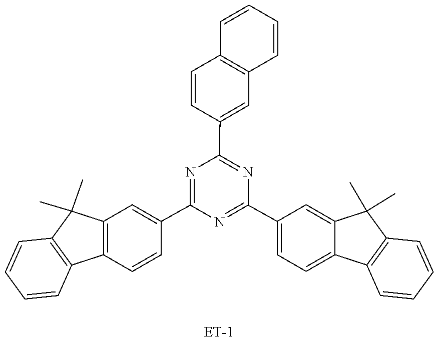 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 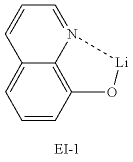 |

1. A plurality of host materials comprising a first host material comprising a compound represented by the following Formula 1, and a second host material comprising a compound represented by the following Formula 2: 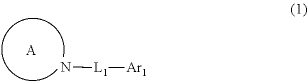
In Formula 1,
L₁ represents a single bond, a substituted or unsubstituted (C6-C30)arylene, or a substituted or unsubstituted (3- to 30-membered)heteroarylene;
Ar₁ represents hydrogen, deuterium, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, —N-(L₂-Ar₂)(L₃-Ar₃), a substituted or unsubstituted (C6-C30)aryl, or a substituted or unsubstituted (3- to 30-membered)heteroaryl;
L₂ and L₃ each independently represent a single bond, a substituted or unsubstituted (C6-C30)arylene, or a substituted or unsubstituted (3- to 30-membered)heteroarylene;
Ar₂ and Ar₃ each independently represent a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; 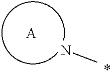
is represented by the following Formula 1-1 or 1-2; 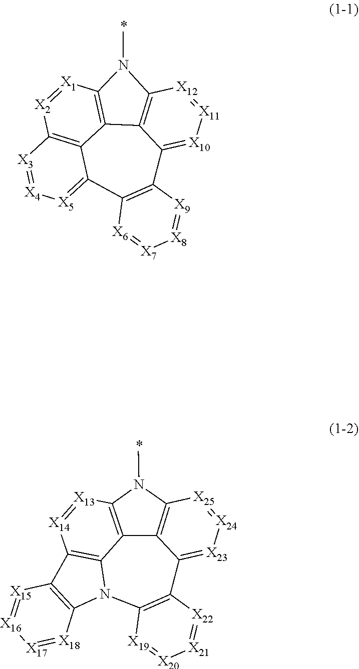
In Formulas 1-1 and 1-2,
X₁ to X₂₅ each independently represent N or CRₐ;
Rₐ each independently represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a ring(s);
* represents a site linked with L₁; 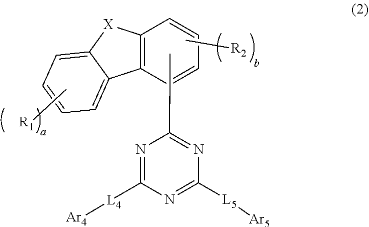
In Formula 2,
X represents O or S;
L₄ and L₅ each independently represent a single bond, a substituted or unsubstituted (C6-C30)arylene, or a substituted or unsubstituted (3- to 30-membered)heteroarylene;
Ar₄ and Ar₅ each independently represent a substituted or unsubstituted (C6-C30)aryl, or a substituted or unsubstituted (3- to 30-membered)heteroaryl;
R₁ and R₂ each independently represent hydrogen, deuterium, a substituted or unsubstituted (C1-C30)alkyl, a substituted or unsubstituted (C6-C30)aryl, a substituted or unsubstituted (3- to 30-membered)heteroaryl, or —SiR₁₁R₁₂R₁₃;
a represents an integer of 1 to 4, b represents an integer of 1 to 3, and if a and b each represent an integer of 2 or more, each of R₁ and each of R₂ may be the same as or different from each other.

2. The plurality of host materials according to claim 1, wherein Formula 1-1 is represented by the following Formula 1-1-1: 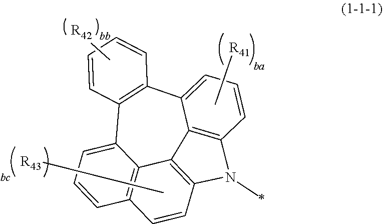
In formula 1-1-1,
R₄₁ to R₄₃ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a ring(s);
ba represents an integer of 1 to 3, bb represents an integer of 1 to 4, bc represents an integer of 1 to 5, and if ba, bb, and bc each represent an integer of 2 or more, each of R₄₁, each of R₄₂, and each of R₄₃, may be the same as or different from one another.

3. The plurality of host materials according to claim 1, wherein Formula 1-2 is represented by the following Formula 1-2-1: 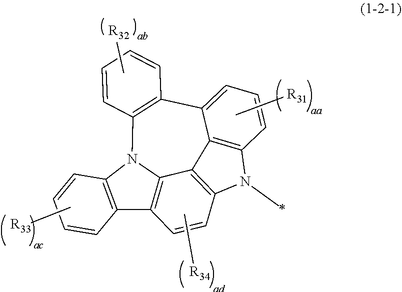
In Formula 1-2-1,
R₃₁ to R₃₄ each independently represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or may be linked to an adjacent substituent(s) to form a ring(s);
aa represents an integer of 1 to 3, ab and ac each independently represents an integer of 1 to 4, ad represents an integer of 1 or 2, and if aa, ab, ac, and ad each represent an integer of 2 or more, each of R₃₁, each of R₃₂, each of R₃₃, and each of R₃₄ may be the same as or different from one another.

4. The plurality of host materials according to claim 1, wherein at least one of Ar₂ and Ar₃ in Formula 1 is represented by any one of the following Formulas 1-3-1 to 1-3-4: 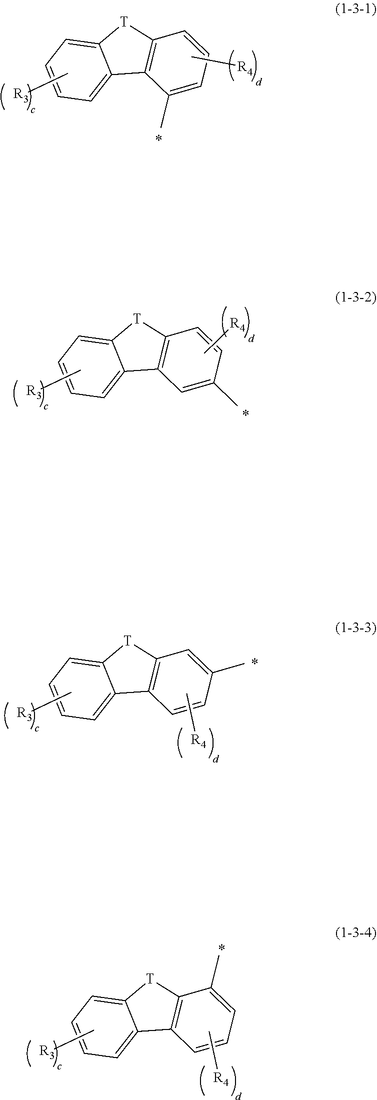
In Formulas 1-3-1 to 1-3-4,
T represents O, S, CR₅R₆, NR₇, or Se,
R₃ to R₇ have the same definition as Rₐ in claim 1, c represents an integer of 1 to 4, d represents an integer of 1 to 3, and if c and d each represent an integer of 2 or more, each of R₃ and each of R₄ may be the same as or different from each other.

5. The plurality of host materials according to claim 1, wherein Formula 2 is represented by any one of the following Formulas 2-1 to 2-4: 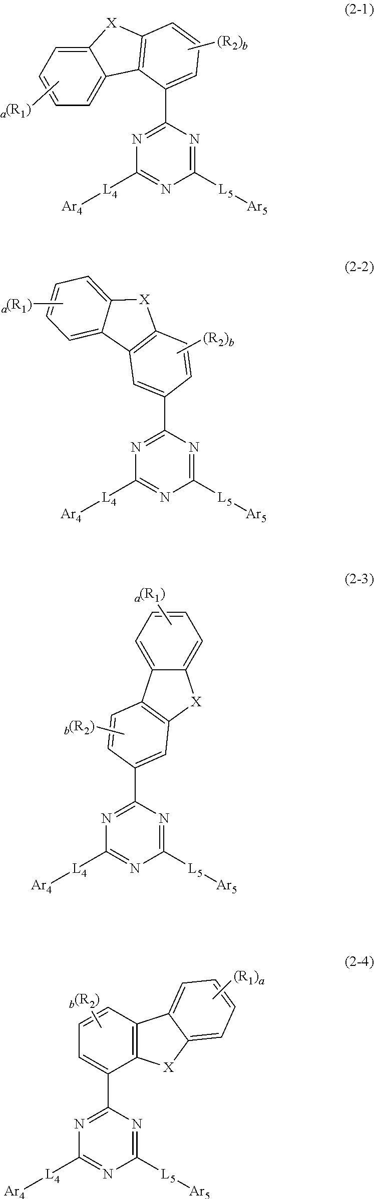
In Formulas 2-1 to 2-4,
X, R₁, R₂, L₄, L₅, Ar₄, Ar₅, a, and b have the same definitions as defined in claim 1.

6. The plurality of host materials according to claim 1, wherein the compound represented by Formula 1 is at least one selected from the following compounds. 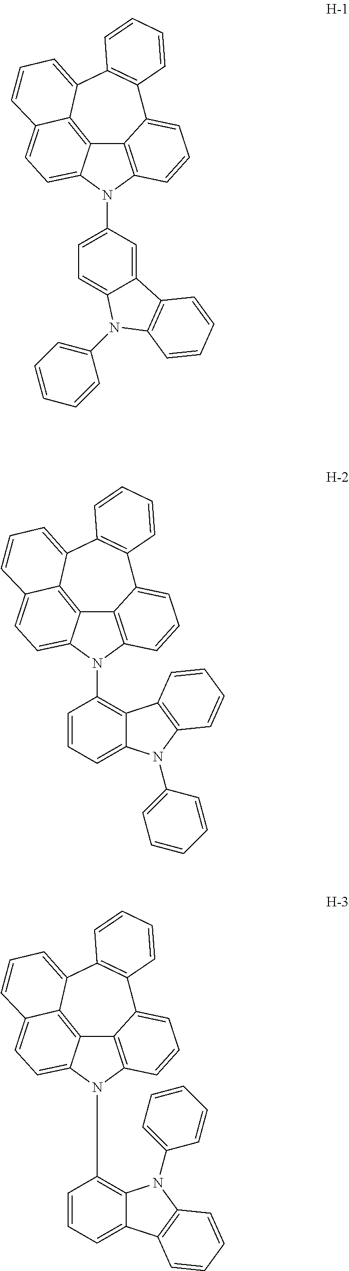 [SMILES: c1ccc(-n2c3ccccc3c3cc(-n4c5cccc6c5c5c7c(cccc7ccc54)-c4ccccc4-6)ccc32)cc1 ; c1ccc(-n2c3ccccc3c3c(-n4c5cccc6c5c5c7c(cccc7ccc54)-c4ccccc4-6)cccc32)cc1 ; c1ccc(-n2c3ccccc3c3cccc(-n4c5cccc6c5c5c7c(cccc7ccc54)-c4ccccc4-6)c32)cc1] 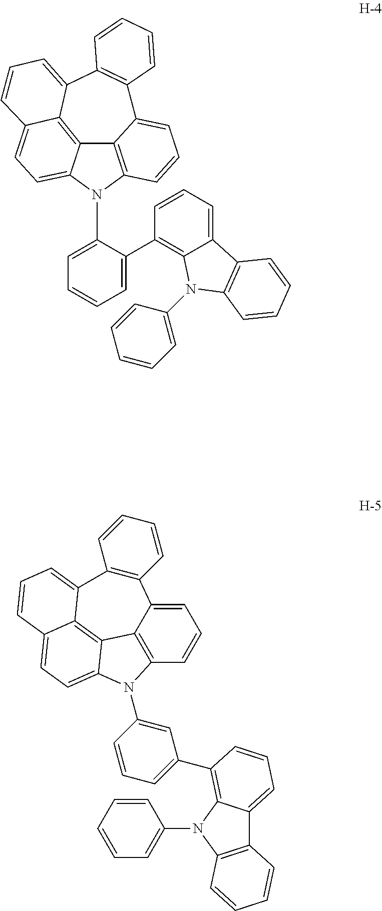 [SMILES: c1ccc(-n2c3ccccc3c3cccc(-c4ccccc4-n4c5cccc6c5c5c7c(cccc7ccc54)-c4ccccc4-6)c32)cc1 ; c1ccc(-n2c3ccccc3c3cccc(-c4cccc(-n5c6cccc7c6c6c8c(cccc8ccc65)-c5ccccc5-7)c4)c32)cc1] 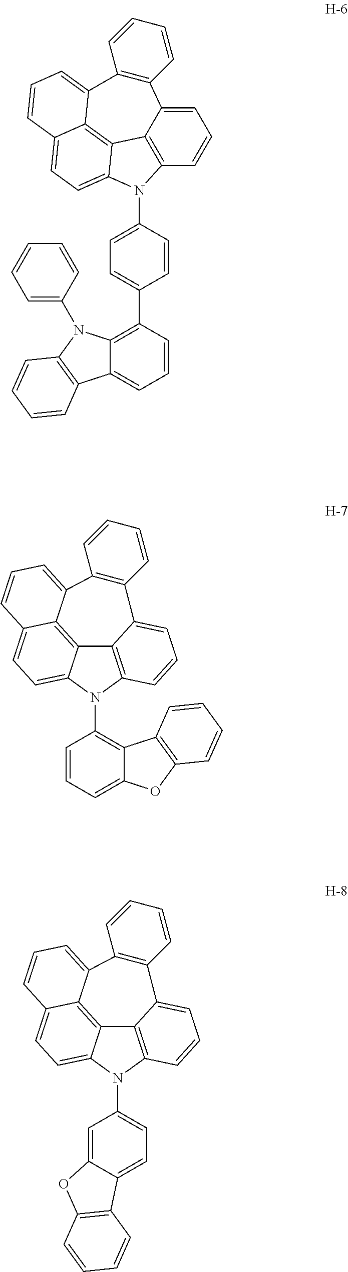 [SMILES: c1ccc(-n2c3ccccc3c3cccc(-c4ccc(-n5c6cccc7c6c6c8c(cccc8ccc65)-c5ccccc5-7)cc4)c32)cc1 ; c1ccc2c(c1)-c1cccc3ccc4c(c13)c1c-2cccc1n4-c1cccc2oc3ccccc3c12 ; c1ccc2c(c1)-c1cccc3ccc4c(c13)c1c-2cccc1n4-c1ccc2c(c1)oc1ccccc12] 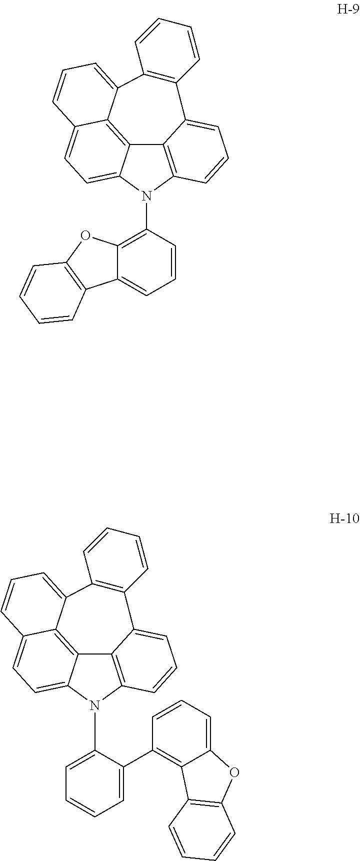 [SMILES: c1ccc2c(c1)-c1cccc3ccc4c(c13)c1c-2cccc1n4-c1cccc2c1oc1ccccc12 ; c1ccc2c(c1)-c1cccc3ccc4c(c13)c1c-2cccc1n4-c1ccccc1-c1cccc2oc3ccccc3c12] 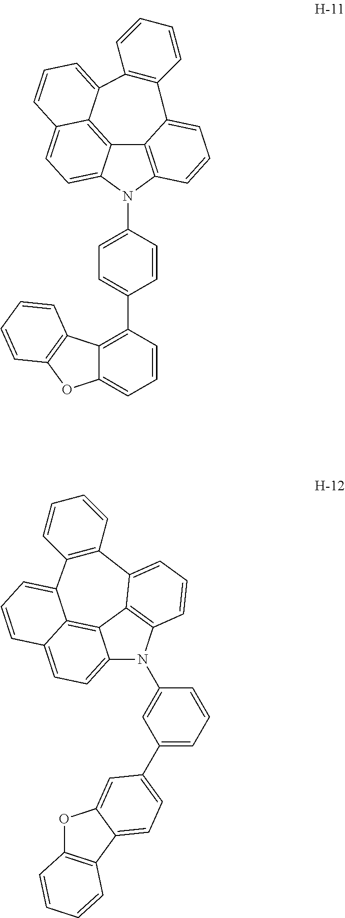 [SMILES: c1ccc2c(c1)-c1cccc3ccc4c(c13)c1c-2cccc1n4-c1ccc(-c2cccc3oc4ccccc4c23)cc1 ; c1cc(-c2ccc3c(c2)oc2ccccc23)cc(-n2c3cccc4c3c3c5c(cccc5ccc32)-c2ccccc2-4)c1] 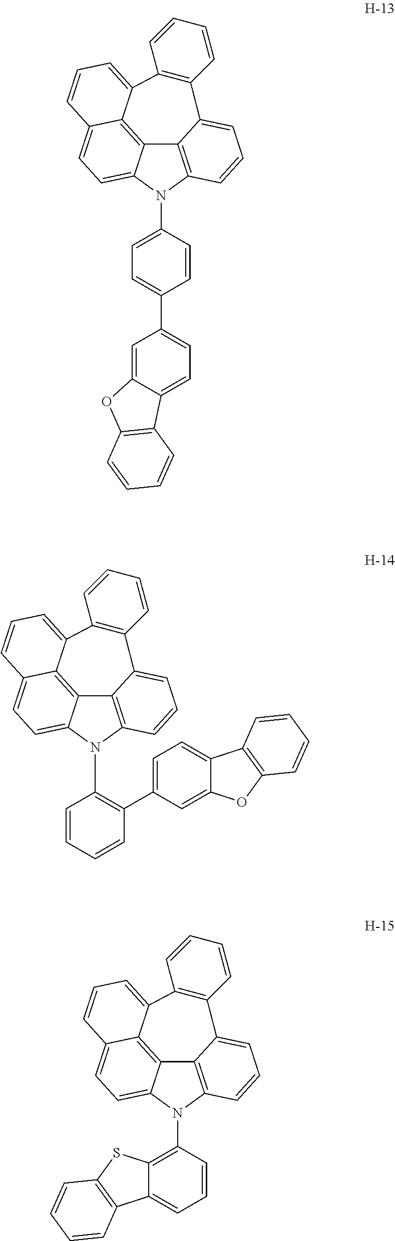 [SMILES: c1ccc2c(c1)-c1cccc3ccc4c(c13)c1c-2cccc1n4-c1ccc(-c2ccc3c(c2)oc2ccccc23)cc1 ; c1ccc2c(c1)-c1cccc3ccc4c(c13)c1c-2cccc1n4-c1ccccc1-c1ccc2c(c1)oc1ccccc12 ; c1ccc2c(c1)-c1cccc3ccc4c(c13)c1c-2cccc1n4-c1cccc2c1sc1ccccc12] 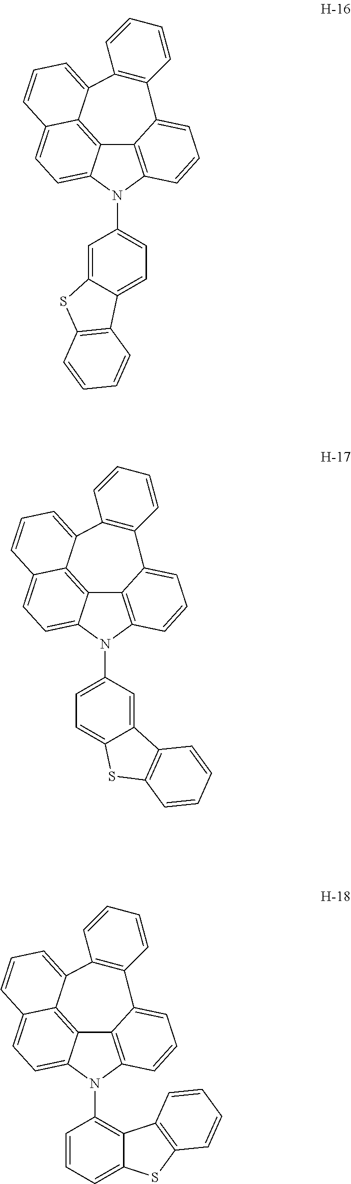 [SMILES: c1ccc2c(c1)-c1cccc3ccc4c(c13)c1c-2cccc1n4-c1ccc2c(c1)sc1ccccc12 ; c1ccc2c(c1)-c1cccc3ccc4c(c13)c1c-2cccc1n4-c1ccc2sc3ccccc3c2c1 ; c1ccc2c(c1)-c1cccc3ccc4c(c13)c1c-2cccc1n4-c1cccc2sc3ccccc3c12] 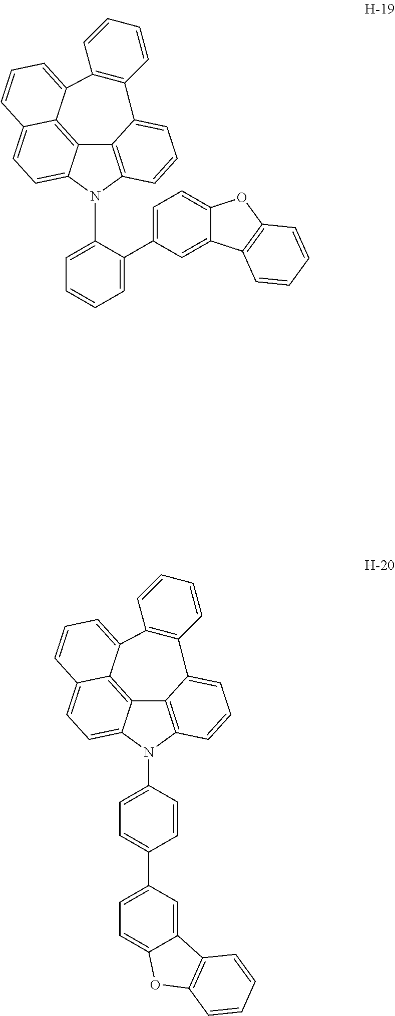 [SMILES: c1ccc2c(c1)-c1cccc3ccc4c(c13)c1c-2cccc1n4-c1ccccc1-c1ccc2oc3ccccc3c2c1 ; c1ccc2c(c1)-c1cccc3ccc4c(c13)c1c-2cccc1n4-c1ccc(-c2ccc3oc4ccccc4c3c2)cc1] 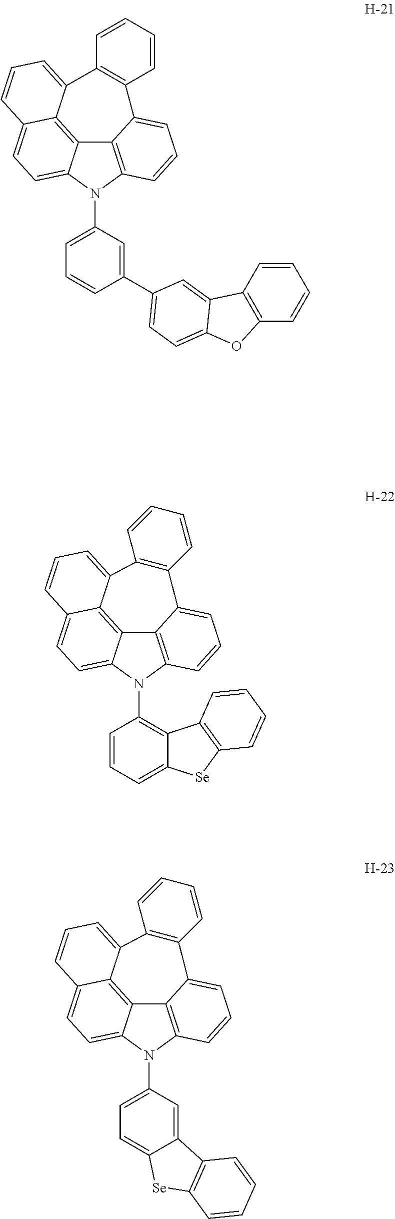 [SMILES: c1cc(-c2ccc3oc4ccccc4c3c2)cc(-n2c3cccc4c3c3c5c(cccc5ccc32)-c2ccccc2-4)c1 ; c1ccc2c(c1)-c1cccc3ccc4c(c13)c1c-2cccc1n4-c1cccc2[se]c3ccccc3c12 ; c1ccc2c(c1)-c1cccc3ccc4c(c13)c1c-2cccc1n4-c1ccc2[se]c3ccccc3c2c1] 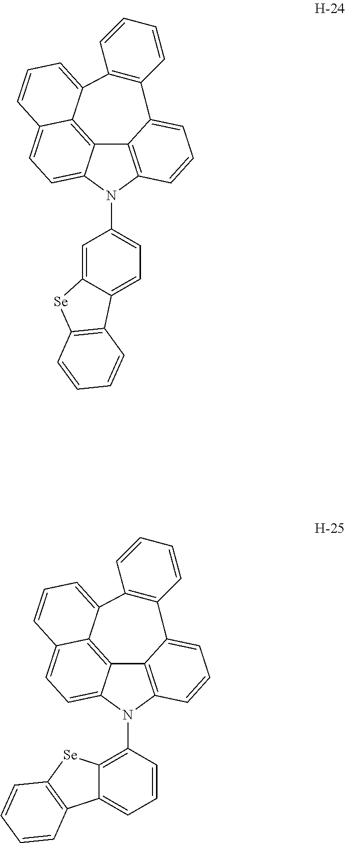 [SMILES: c1ccc2c(c1)-c1cccc3ccc4c(c13)c1c-2cccc1n4-c1ccc2c(c1)[se]c1ccccc12 ; c1ccc2c(c1)-c1cccc3ccc4c(c13)c1c-2cccc1n4-c1cccc2c1[se]c1ccccc12] 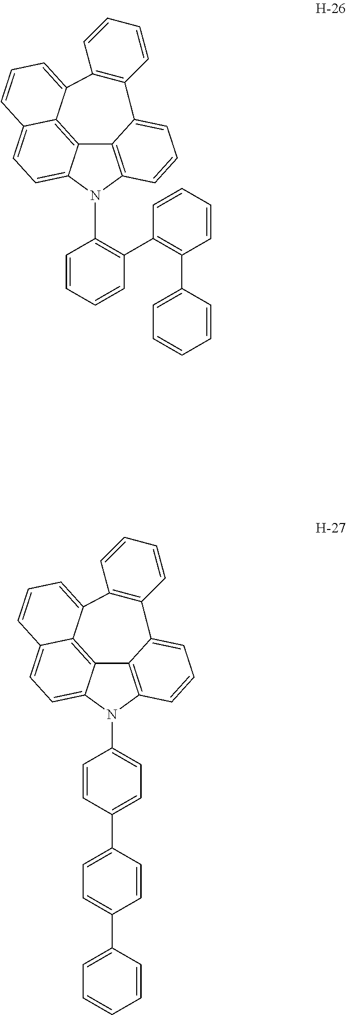 [SMILES: c1ccc(-c2ccccc2-c2ccccc2-n2c3cccc4c3c3c5c(cccc5ccc32)-c2ccccc2-4)cc1 ; c1ccc(-c2ccc(-c3ccc(-n4c5cccc6c5c5c7c(cccc7ccc54)-c4ccccc4-6)cc3)cc2)cc1] 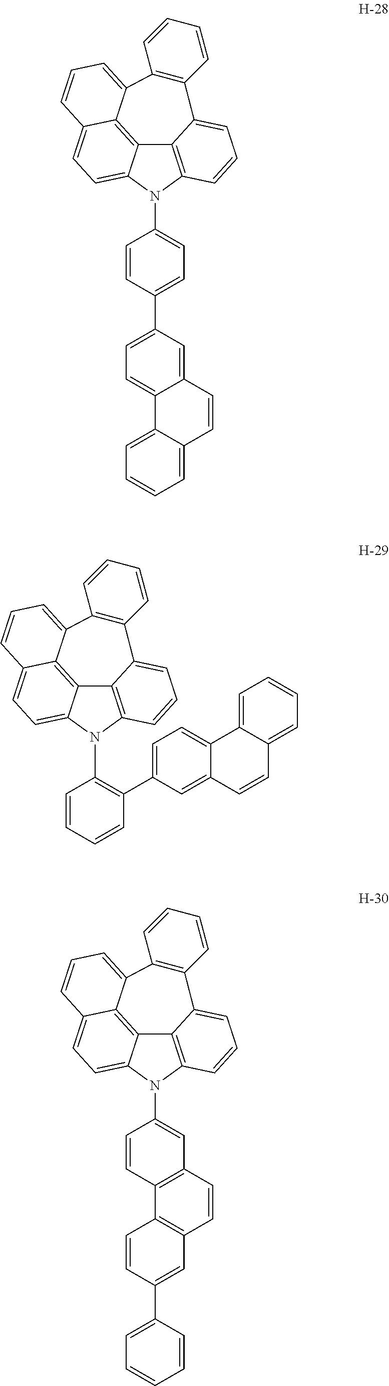 [SMILES: c1ccc2c(c1)-c1cccc3ccc4c(c13)c1c-2cccc1n4-c1ccc(-c2ccc3c(ccc4ccccc43)c2)cc1 ; c1ccc2c(c1)-c1cccc3ccc4c(c13)c1c-2cccc1n4-c1ccccc1-c1ccc2c(ccc3ccccc32)c1 ; c1ccc(-c2ccc3c(ccc4cc(-n5c6cccc7c6c6c8c(cccc8ccc65)-c5ccccc5-7)ccc43)c2)cc1] 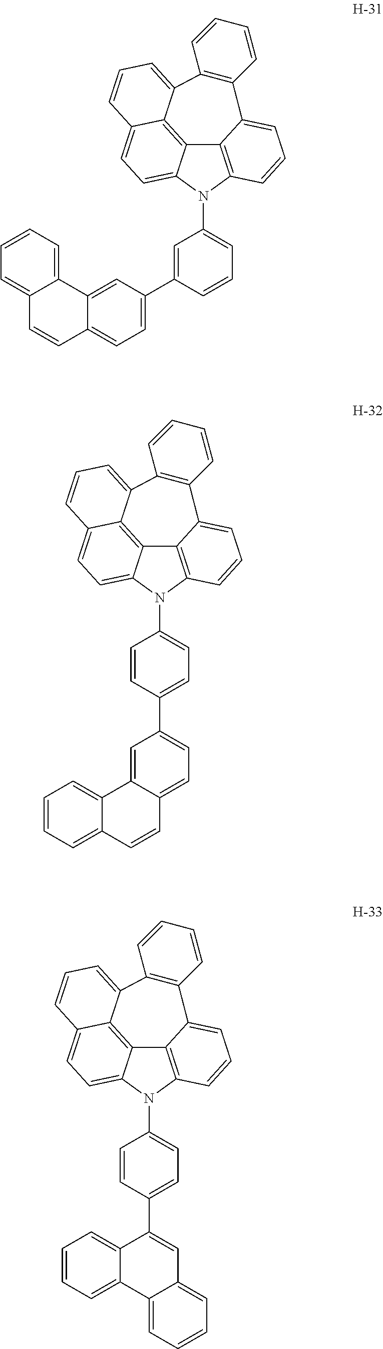 [SMILES: c1cc(-c2ccc3ccc4ccccc4c3c2)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc(-c2ccc3ccc4ccccc4c3c2)cc1 ; c1ccc2c(c1)-c1cccc3ccc4c(c13)c1c-2cccc1n4-c1ccc(-c2cc3ccccc3c3ccccc23)cc1] 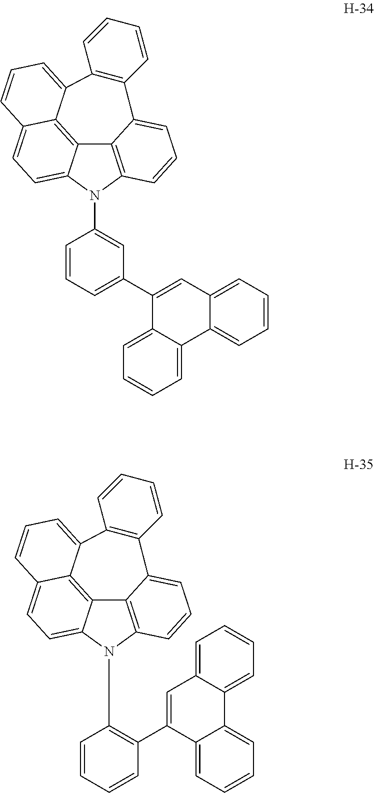 [SMILES: c1cc(-c2cc3ccccc3c3ccccc23)cc(-n2c3cccc4c3c3c5c(cccc5ccc32)-c2ccccc2-4)c1 ; c1ccc2c(c1)-c1cccc3ccc4c(c13)c1c-2cccc1n4-c1ccccc1-c1cc2ccccc2c2ccccc12] 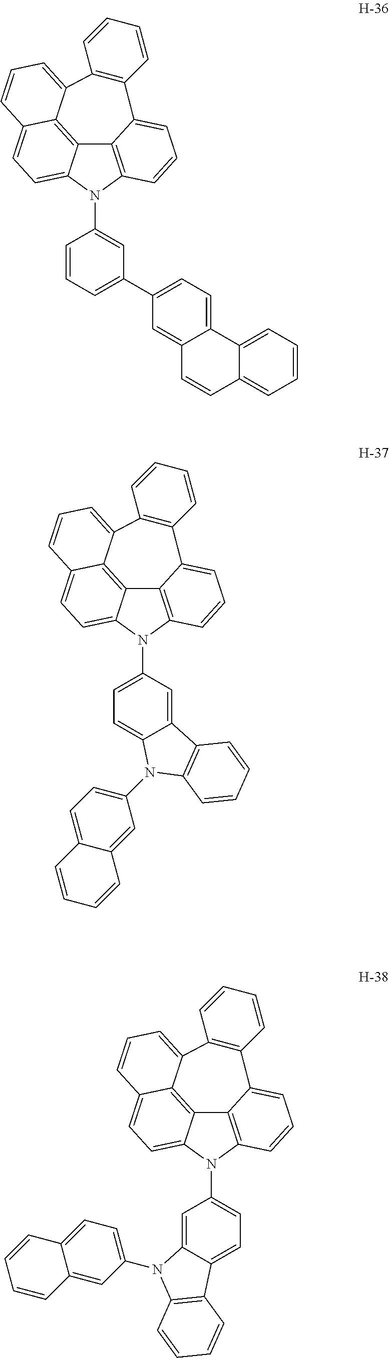 [SMILES: c1cc(-c2ccc3c(ccc4ccccc43)c2)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc2c(c1)c1ccccc1n2-c1ccc2ccccc2c1 ; c1ccc2c(c1)-c1cccc3ccc4c(c13)c1c-2cccc1n4-c1ccc2c3ccccc3n(-c3ccc4ccccc4c3)c2c1] 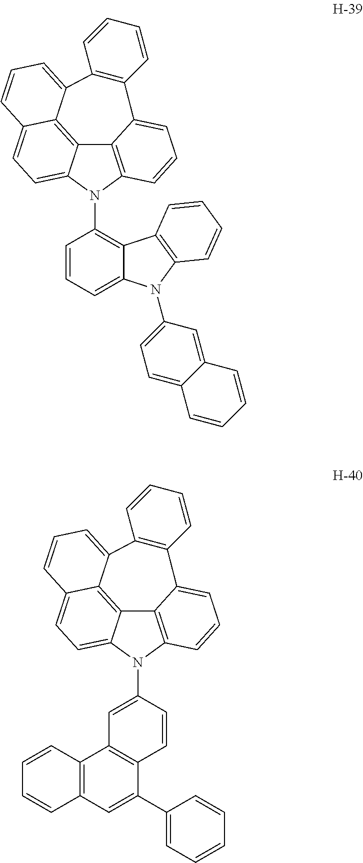 [SMILES: c1ccc2c(c1)-c1cccc3ccc4c(c13)c1c-2cccc1n4-c1cccc2c1c1ccccc1n2-c1ccc2ccccc2c1 ; c1ccc(-c2cc3ccccc3c3cc(-n4c5cccc6c5c5c7c(cccc7ccc54)-c4ccccc4-6)ccc23)cc1] 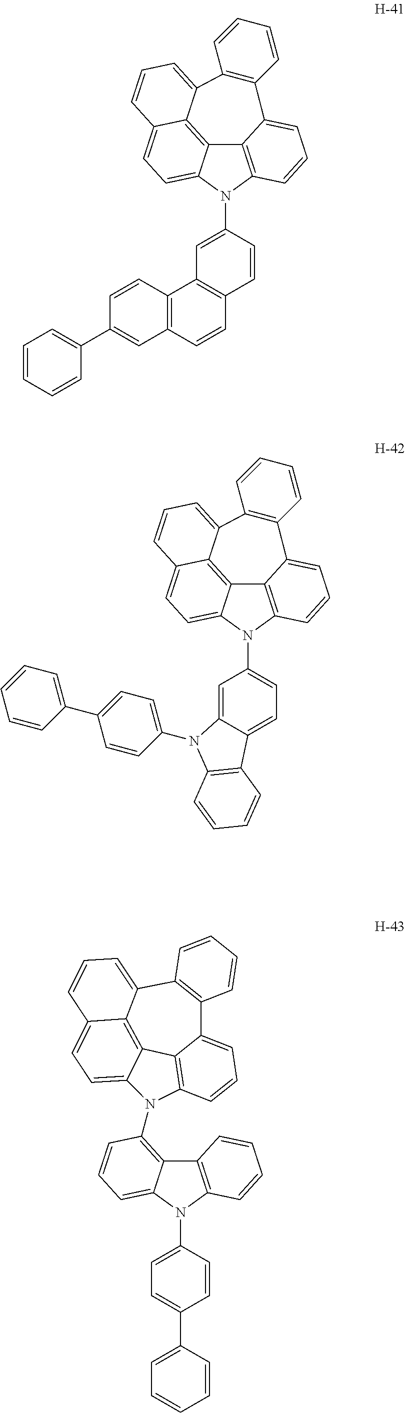 [SMILES: c1ccc(-c2ccc3c(ccc4ccc(-n5c6cccc7c6c6c8c(cccc8ccc65)-c5ccccc5-7)cc43)c2)cc1 ; c1ccc(-c2ccc(-n3c4ccccc4c4ccc(-n5c6cccc7c6c6c8c(cccc8ccc65)-c5ccccc5-7)cc43)cc2)cc1 ; c1ccc(-c2ccc(-n3c4ccccc4c4c(-n5c6cccc7c6c6c8c(cccc8ccc65)-c5ccccc5-7)cccc43)cc2)cc1] 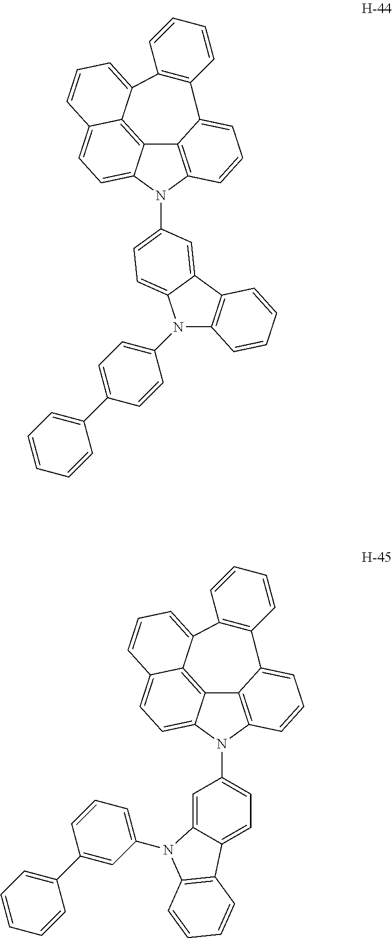 [SMILES: c1ccc(-c2ccc(-n3c4ccccc4c4cc(-n5c6cccc7c6c6c8c(cccc8ccc65)-c5ccccc5-7)ccc43)cc2)cc1 ; c1ccc(-c2cccc(-n3c4ccccc4c4ccc(-n5c6cccc7c6c6c8c(cccc8ccc65)-c5ccccc5-7)cc43)c2)cc1] 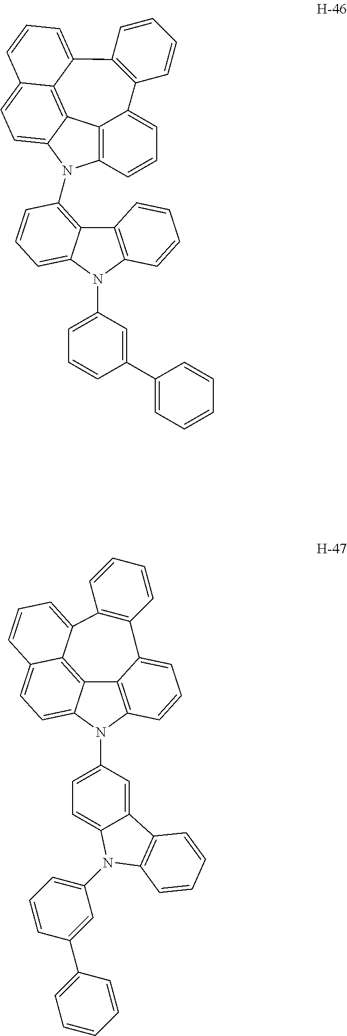 [SMILES: c1ccc(-c2cccc(-n3c4ccccc4c4c(-n5c6cccc7c6c6c8c(cccc8ccc65)-c5ccccc5-7)cccc43)c2)cc1 ; c1ccc(-c2cccc(-n3c4ccccc4c4cc(-n5c6cccc7c6c6c8c(cccc8ccc65)-c5ccccc5-7)ccc43)c2)cc1] 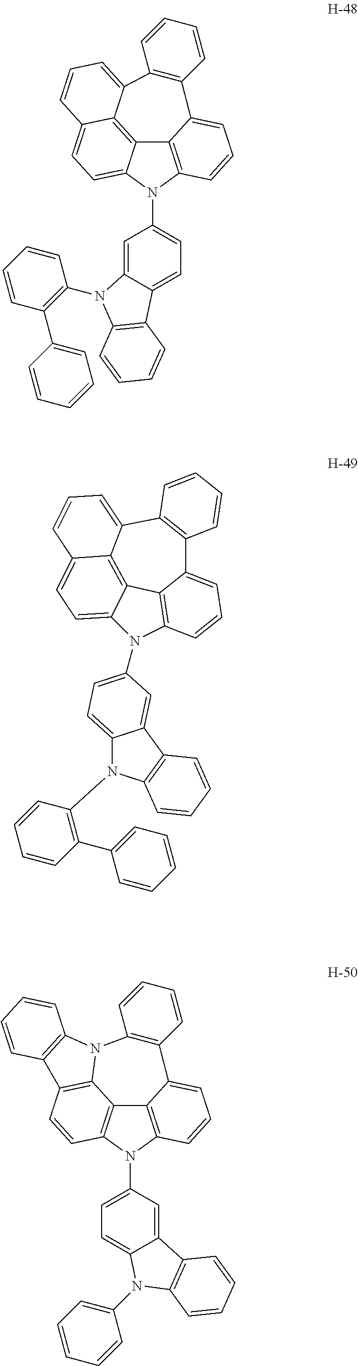 [SMILES: c1ccc(-c2ccccc2-n2c3ccccc3c3ccc(-n4c5cccc6c5c5c7c(cccc7ccc54)-c4ccccc4-6)cc32)cc1 ; c1ccc(-c2ccccc2-n2c3ccccc3c3cc(-n4c5cccc6c5c5c7c(cccc7ccc54)-c4ccccc4-6)ccc32)cc1 ; c1ccc(-n2c3ccccc3c3cc(-n4c5cccc6c7ccccc7n7c8ccccc8c8ccc4c(c65)c87)ccc32)cc1] 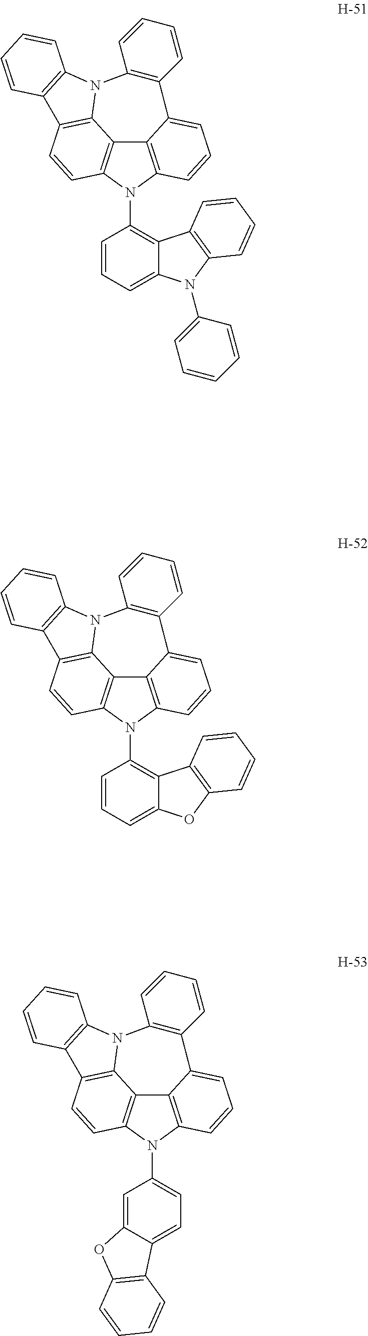 [SMILES: c1ccc(-n2c3ccccc3c3c(-n4c5cccc6c7ccccc7n7c8ccccc8c8ccc4c(c65)c87)cccc32)cc1 ; c1ccc2c(c1)oc1cccc(-n3c4cccc5c6ccccc6n6c7ccccc7c7ccc3c(c54)c76)c12 ; c1ccc2c(c1)oc1cc(-n3c4cccc5c6ccccc6n6c7ccccc7c7ccc3c(c54)c76)ccc12] 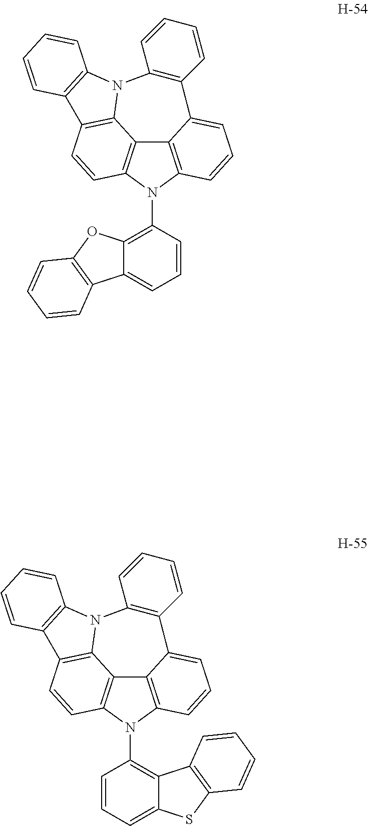 [SMILES: c1ccc2c(c1)oc1c(-n3c4cccc5c6ccccc6n6c7ccccc7c7ccc3c(c54)c76)cccc12 ; c1ccc2c(c1)sc1cccc(-n3c4cccc5c6ccccc6n6c7ccccc7c7ccc3c(c54)c76)c12] 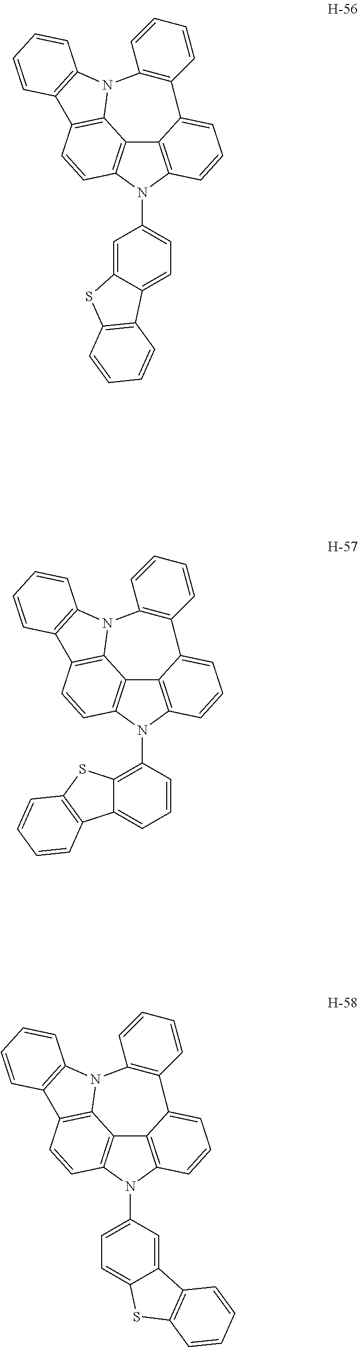 [SMILES: c1ccc2c(c1)sc1cc(-n3c4cccc5c6ccccc6n6c7ccccc7c7ccc3c(c54)c76)ccc12 ; c1ccc2c(c1)sc1c(-n3c4cccc5c6ccccc6n6c7ccccc7c7ccc3c(c54)c76)cccc12 ; c1ccc2c(c1)sc1ccc(-n3c4cccc5c6ccccc6n6c7ccccc7c7ccc3c(c54)c76)cc12] 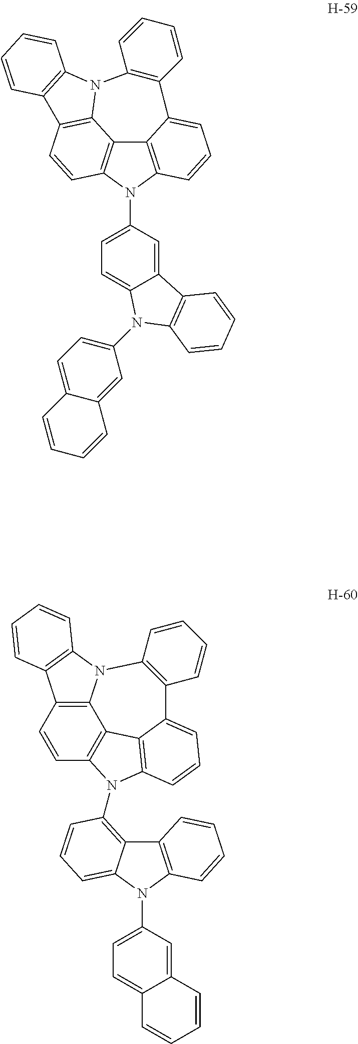 [SMILES: c1ccc2cc(-n3c4ccccc4c4cc(-n5c6cccc7c8ccccc8n8c9ccccc9c9ccc5c(c76)c98)ccc43)ccc2c1 ; c1ccc2cc(-n3c4ccccc4c4c(-n5c6cccc7c8ccccc8n8c9ccccc9c9ccc5c(c76)c98)cccc43)ccc2c1] 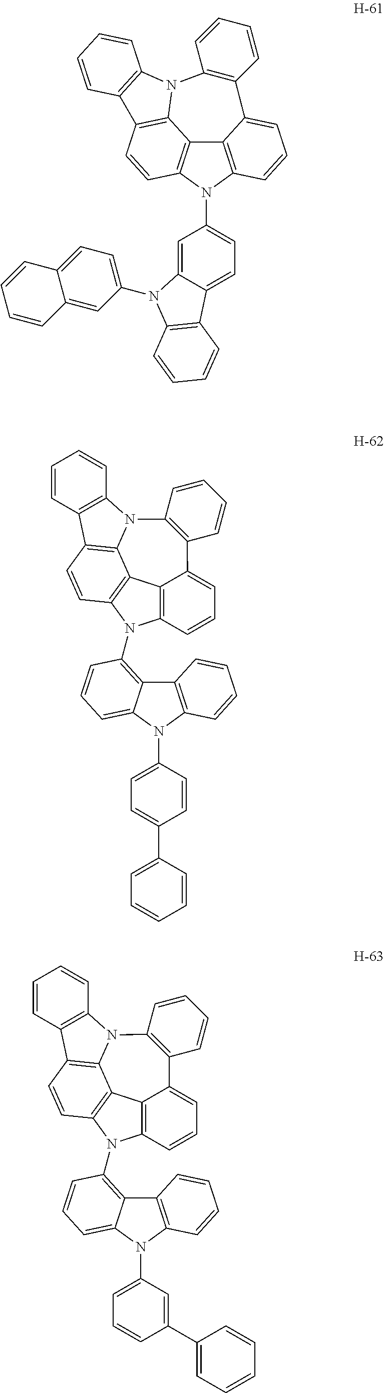 [SMILES: c1ccc2cc(-n3c4ccccc4c4ccc(-n5c6cccc7c8ccccc8n8c9ccccc9c9ccc5c(c76)c98)cc43)ccc2c1 ; c1ccc(-c2ccc(-n3c4ccccc4c4c(-n5c6cccc7c8ccccc8n8c9ccccc9c9ccc5c(c76)c98)cccc43)cc2)cc1 ; c1ccc(-c2cccc(-n3c4ccccc4c4c(-n5c6cccc7c8ccccc8n8c9ccccc9c9ccc5c(c76)c98)cccc43)c2)cc1] 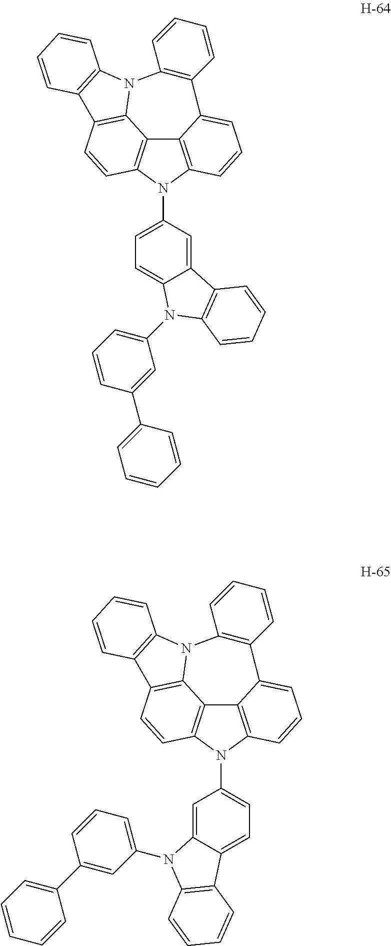 [SMILES: c1ccc(-c2cccc(-n3c4ccccc4c4cc(-n5c6cccc7c8ccccc8n8c9ccccc9c9ccc5c(c76)c98)ccc43)c2)cc1 ; c1ccc(-c2cccc(-n3c4ccccc4c4ccc(-n5c6cccc7c8ccccc8n8c9ccccc9c9ccc5c(c76)c98)cc43)c2)cc1] 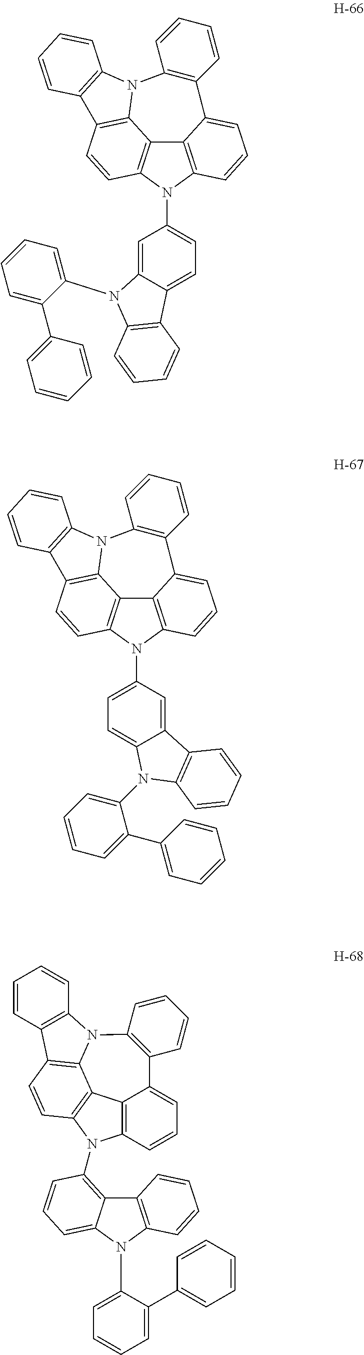 [SMILES: c1ccc(-c2ccccc2-n2c3ccccc3c3ccc(-n4c5cccc6c7ccccc7n7c8ccccc8c8ccc4c(c65)c87)cc32)cc1 ; c1ccc(-c2ccccc2-n2c3ccccc3c3cc(-n4c5cccc6c7ccccc7n7c8ccccc8c8ccc4c(c65)c87)ccc32)cc1 ; c1ccc(-c2ccccc2-n2c3ccccc3c3c(-n4c5cccc6c7ccccc7n7c8ccccc8c8ccc4c(c65)c87)cccc32)cc1] 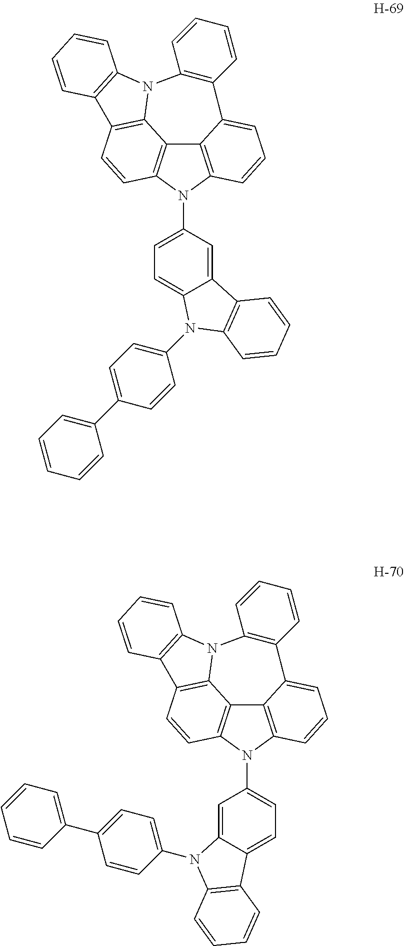 [SMILES: c1ccc(-c2ccc(-n3c4ccccc4c4cc(-n5c6cccc7c8ccccc8n8c9ccccc9c9ccc5c(c76)c98)ccc43)cc2)cc1 ; c1ccc(-c2ccc(-n3c4ccccc4c4ccc(-n5c6cccc7c8ccccc8n8c9ccccc9c9ccc5c(c76)c98)cc43)cc2)cc1] 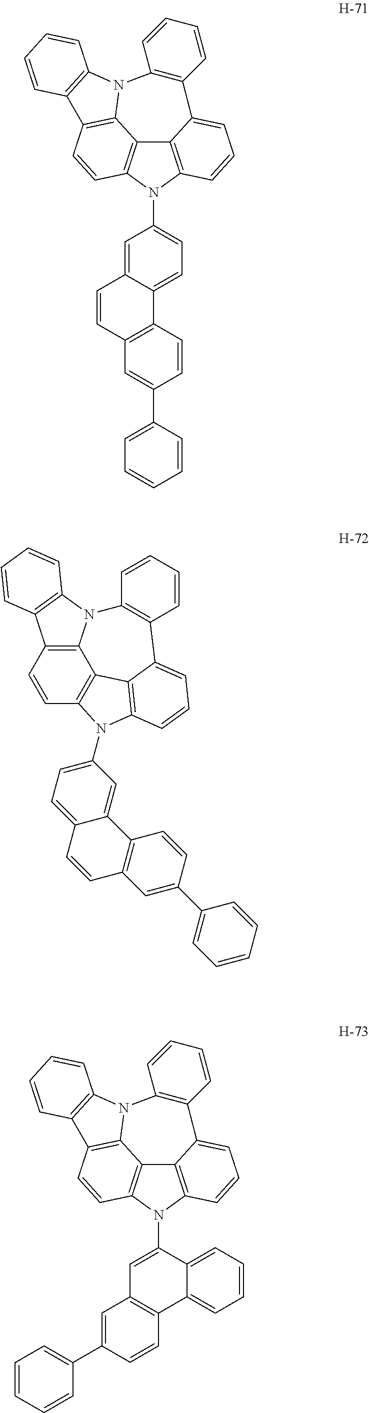 [SMILES: c1ccc(-c2ccc3c(ccc4cc(-n5c6cccc7c8ccccc8n8c9ccccc9c9ccc5c(c76)c98)ccc43)c2)cc1 ; c1ccc(-c2ccc3c(ccc4ccc(-n5c6cccc7c8ccccc8n8c9ccccc9c9ccc5c(c76)c98)cc43)c2)cc1 ; c1ccc(-c2ccc3c(c2)cc(-n2c4cccc5c6ccccc6n6c7ccccc7c7ccc2c(c54)c76)c2ccccc23)cc1] 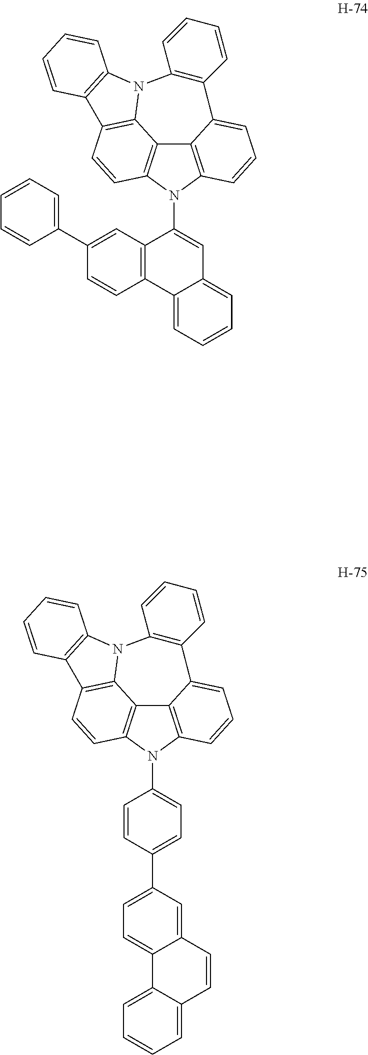 [SMILES: c1ccc(-c2ccc3c(c2)c(-n2c4cccc5c6ccccc6n6c7ccccc7c7ccc2c(c54)c76)cc2ccccc23)cc1 ; c1ccc2c(c1)ccc1cc(-c3ccc(-n4c5cccc6c7ccccc7n7c8ccccc8c8ccc4c(c65)c87)cc3)ccc12] 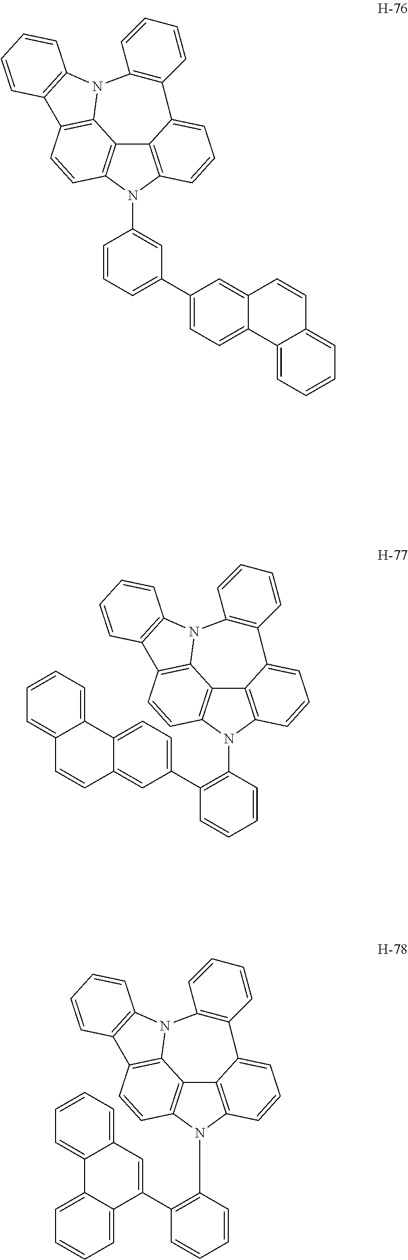 [SMILES: c1cc(-c2ccc3c(ccc4ccccc43)c2)cc(-n2c3cccc4c5ccccc5n5c6ccccc6c6ccc2c(c43)c65)c1 ; c1ccc(-n2c3cccc4c5ccccc5n5c6ccccc6c6ccc2c(c43)c65)c(-c2ccc3c(ccc4ccccc43)c2)c1 ; c1ccc(-n2c3cccc4c5ccccc5n5c6ccccc6c6ccc2c(c43)c65)c(-c2cc3ccccc3c3ccccc23)c1] 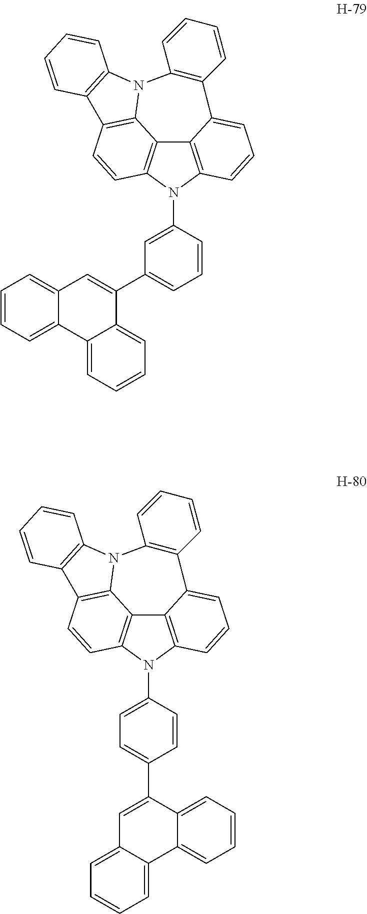 [SMILES: c1cc(-c2cc3ccccc3c3ccccc23)cc(-n2c3cccc4c5ccccc5n5c6ccccc6c6ccc2c(c43)c65)c1 ; c1ccc2c(c1)cc(-c1ccc(-n3c4cccc5c6ccccc6n6c7ccccc7c7ccc3c(c54)c76)cc1)c1ccccc12] 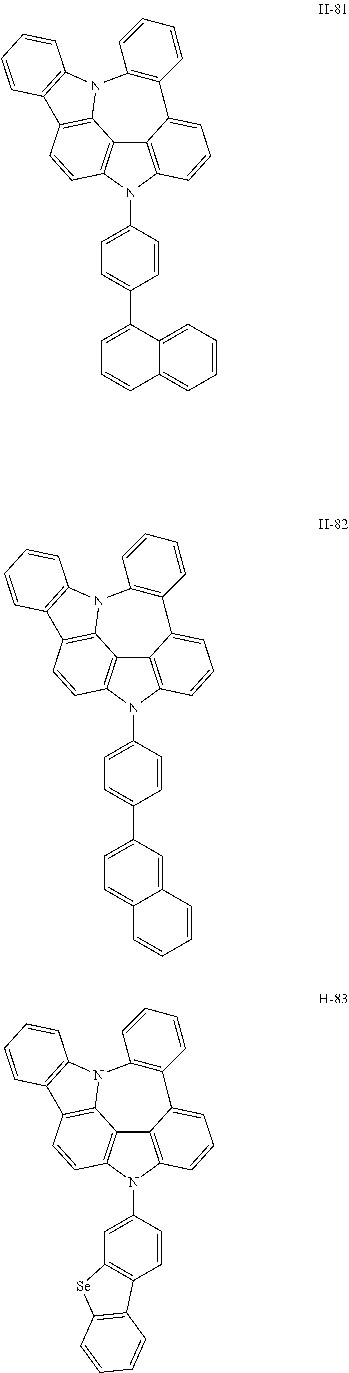 [SMILES: c1ccc2c(-c3ccc(-n4c5cccc6c7ccccc7n7c8ccccc8c8ccc4c(c65)c87)cc3)cccc2c1 ; c1ccc2cc(-c3ccc(-n4c5cccc6c7ccccc7n7c8ccccc8c8ccc4c(c65)c87)cc3)ccc2c1 ; c1ccc2c(c1)[se]c1cc(-n3c4cccc5c6ccccc6n6c7ccccc7c7ccc3c(c54)c76)ccc12] 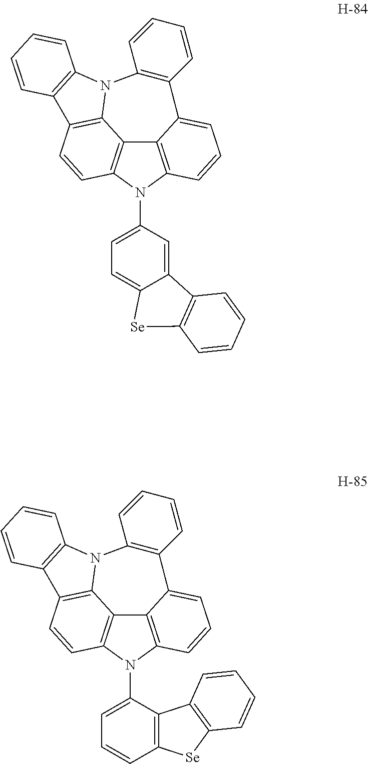 [SMILES: c1ccc2c(c1)[se]c1ccc(-n3c4cccc5c6ccccc6n6c7ccccc7c7ccc3c(c54)c76)cc12 ; c1ccc2c(c1)[se]c1cccc(-n3c4cccc5c6ccccc6n6c7ccccc7c7ccc3c(c54)c76)c12] 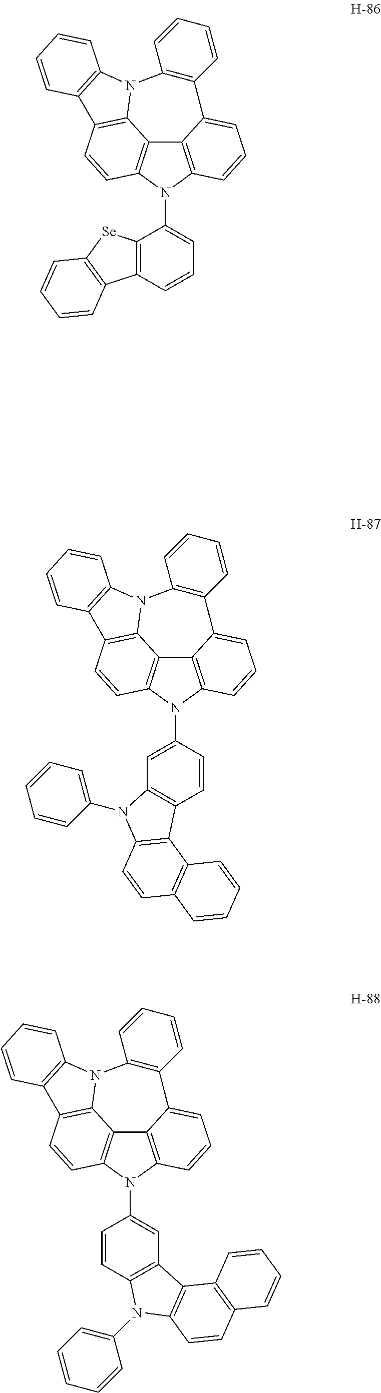 [SMILES: c1ccc2c(c1)[se]c1c(-n3c4cccc5c6ccccc6n6c7ccccc7c7ccc3c(c54)c76)cccc12 ; c1ccc(-n2c3cc(-n4c5cccc6c7ccccc7n7c8ccccc8c8ccc4c(c65)c87)ccc3c3c4ccccc4ccc32)cc1 ; c1ccc(-n2c3ccc(-n4c5cccc6c7ccccc7n7c8ccccc8c8ccc4c(c65)c87)cc3c3c4ccccc4ccc32)cc1] 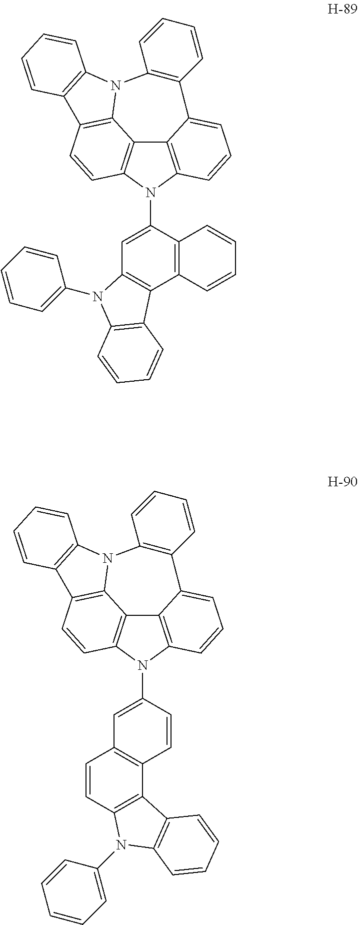 [SMILES: c1ccc(-n2c3ccccc3c3c4ccccc4c(-n4c5cccc6c7ccccc7n7c8ccccc8c8ccc4c(c65)c87)cc32)cc1 ; c1ccc(-n2c3ccccc3c3c4ccc(-n5c6cccc7c8ccccc8n8c9ccccc9c9ccc5c(c76)c98)cc4ccc32)cc1] 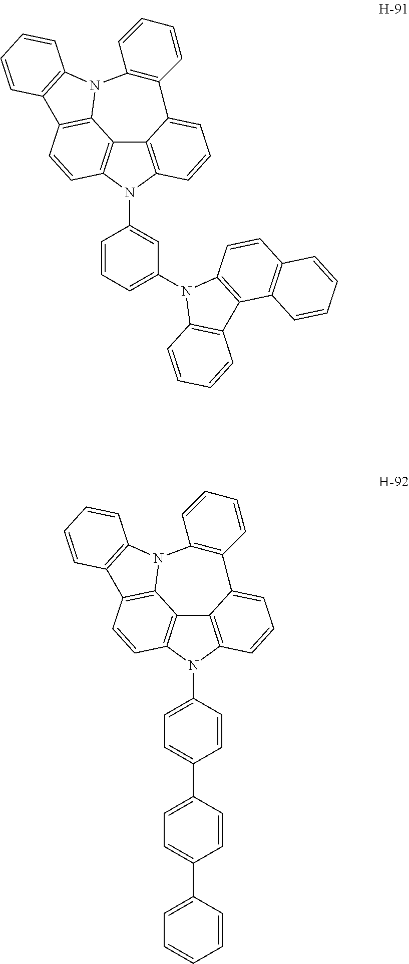 [SMILES: c1cc(-n2c3ccccc3c3c4ccccc4ccc32)cc(-n2c3cccc4c5ccccc5n5c6ccccc6c6ccc2c(c43)c65)c1 ; c1ccc(-c2ccc(-c3ccc(-n4c5cccc6c7ccccc7n7c8ccccc8c8ccc4c(c65)c87)cc3)cc2)cc1] 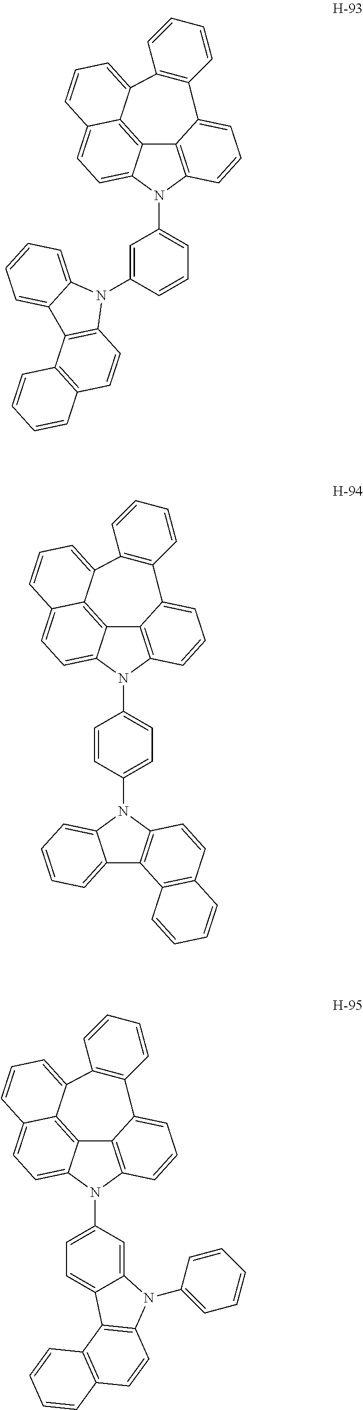 [SMILES: c1cc(-n2c3ccccc3c3c4ccccc4ccc32)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc(-n2c3ccccc3c3c4ccccc4ccc32)cc1 ; c1ccc(-n2c3cc(-n4c5cccc6c5c5c7c(cccc7ccc54)-c4ccccc4-6)ccc3c3c4ccccc4ccc32)cc1] 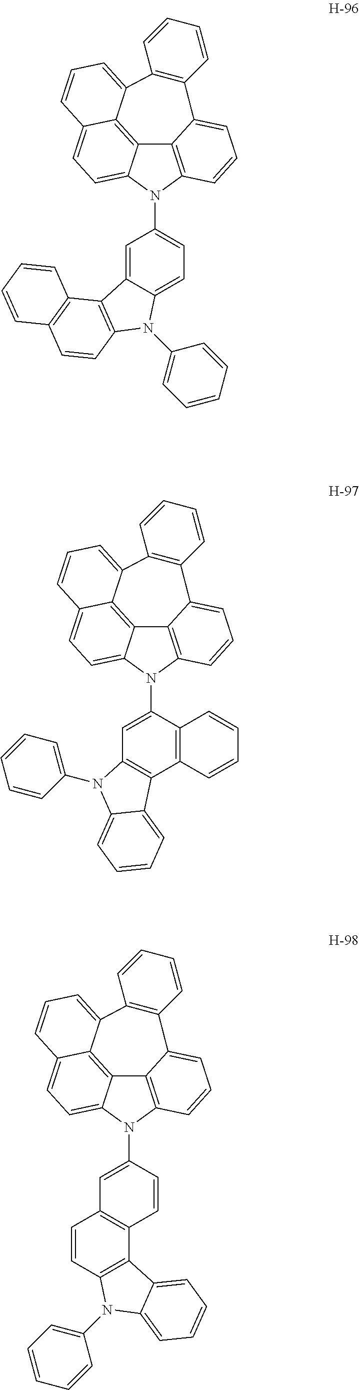 [SMILES: c1ccc(-n2c3ccc(-n4c5cccc6c5c5c7c(cccc7ccc54)-c4ccccc4-6)cc3c3c4ccccc4ccc32)cc1 ; c1ccc(-n2c3ccccc3c3c4ccccc4c(-n4c5cccc6c5c5c7c(cccc7ccc54)-c4ccccc4-6)cc32)cc1 ; c1ccc(-n2c3ccccc3c3c4ccc(-n5c6cccc7c6c6c8c(cccc8ccc65)-c5ccccc5-7)cc4ccc32)cc1] 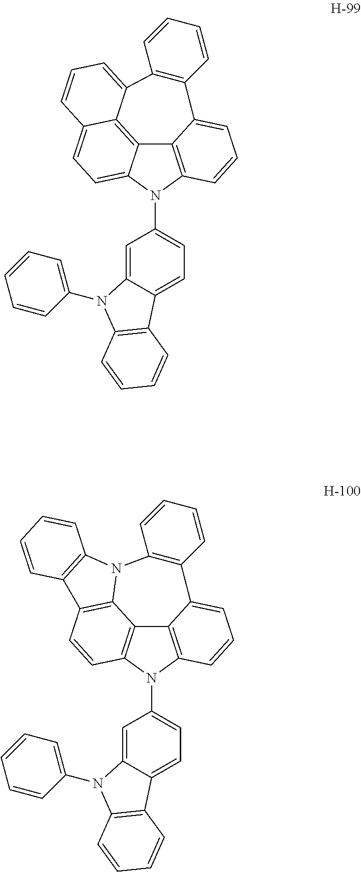 [SMILES: c1ccc(-n2c3ccccc3c3ccc(-n4c5cccc6c5c5c7c(cccc7ccc54)-c4ccccc4-6)cc32)cc1 ; c1ccc(-n2c3ccccc3c3ccc(-n4c5cccc6c7ccccc7n7c8ccccc8c8ccc4c(c65)c87)cc32)cc1] 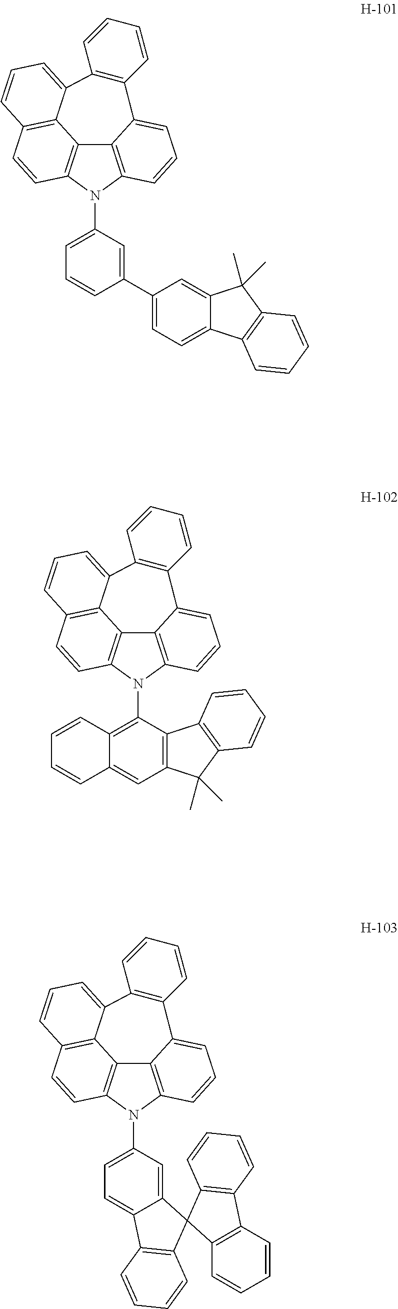 [SMILES: CC1(C)c2ccccc2-c2ccc(-c3cccc(-n4c5cccc6c5c5c7c(cccc7ccc54)-c4ccccc4-6)c3)cc21 ; CC1(C)c2ccccc2-c2c1cc1ccccc1c2-n1c2cccc3c2c2c4c(cccc4ccc21)-c1ccccc1-3 ; c1ccc2c(c1)-c1cccc3ccc4c(c13)c1c-2cccc1n4-c1ccc2c(c1)C1(c3ccccc3-c3ccccc31)c1ccccc1-2] 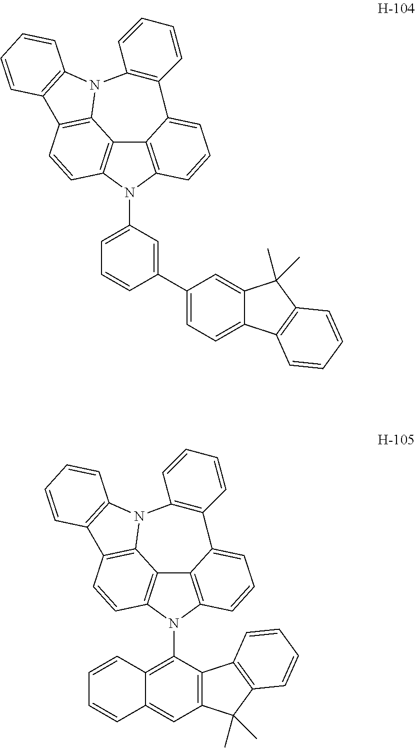 [SMILES: CC1(C)c2ccccc2-c2ccc(-c3cccc(-n4c5cccc6c7ccccc7n7c8ccccc8c8ccc4c(c65)c87)c3)cc21 ; CC1(C)c2ccccc2-c2c1cc1ccccc1c2-n1c2cccc3c4ccccc4n4c5ccccc5c5ccc1c(c32)c54] 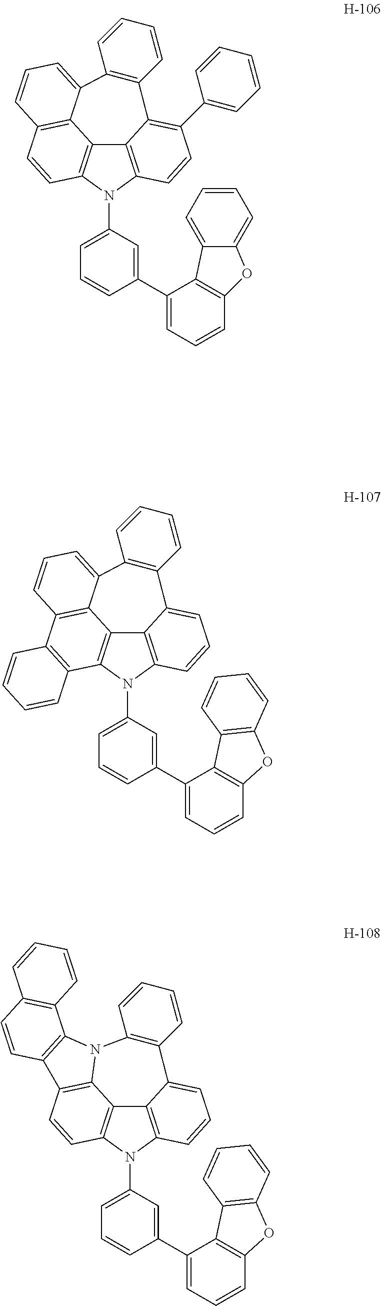 [SMILES: c1ccc(-c2ccc3c4c2-c2ccccc2-c2cccc5ccc(c4c25)n3-c2cccc(-c3cccc4oc5ccccc5c34)c2)cc1 ; c1cc(-c2cccc3oc4ccccc4c23)cc(-n2c3cccc4c3c3c5c(cccc5c5ccccc5c32)-c2ccccc2-4)c1 ; c1cc(-c2cccc3oc4ccccc4c23)cc(-n2c3cccc4c5ccccc5n5c6c7ccccc7ccc6c6ccc2c(c43)c65)c1] 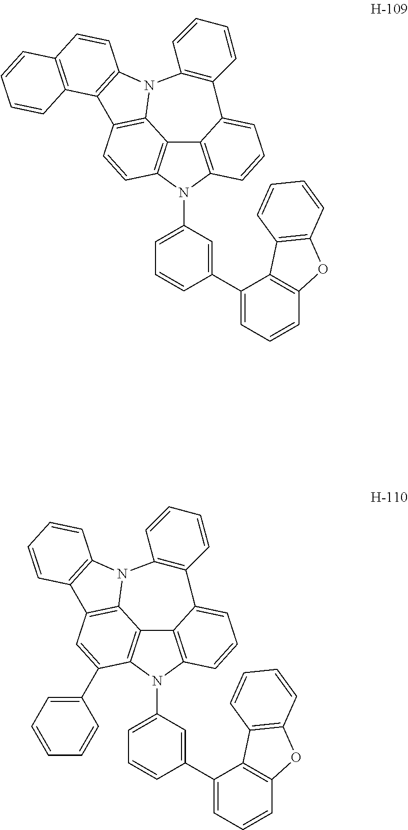 [SMILES: c1cc(-c2cccc3oc4ccccc4c23)cc(-n2c3cccc4c5ccccc5n5c6ccc7ccccc7c6c6ccc2c(c43)c65)c1 ; c1ccc(-c2cc3c4ccccc4n4c5ccccc5c5cccc6c5c(c2n6-c2cccc(-c5cccc6oc7ccccc7c56)c2)c34)cc1] 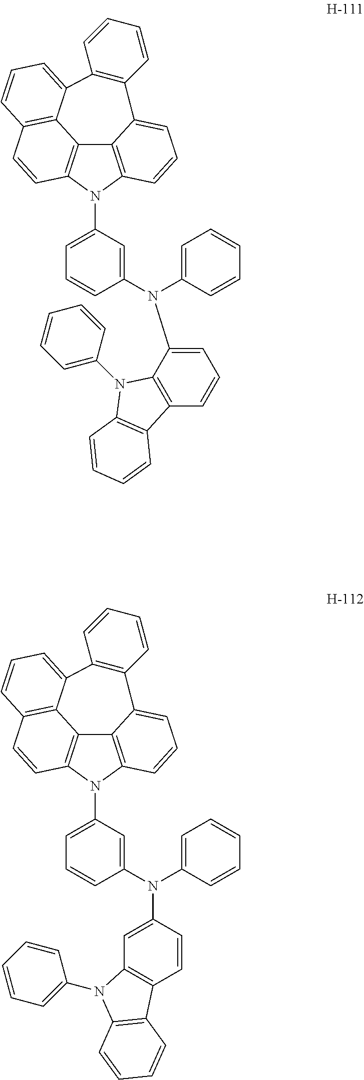 [SMILES: c1ccc(N(c2cccc(-n3c4cccc5c4c4c6c(cccc6ccc43)-c3ccccc3-5)c2)c2cccc3c4ccccc4n(-c4ccccc4)c23)cc1 ; c1ccc(N(c2cccc(-n3c4cccc5c4c4c6c(cccc6ccc43)-c3ccccc3-5)c2)c2ccc3c4ccccc4n(-c4ccccc4)c3c2)cc1] 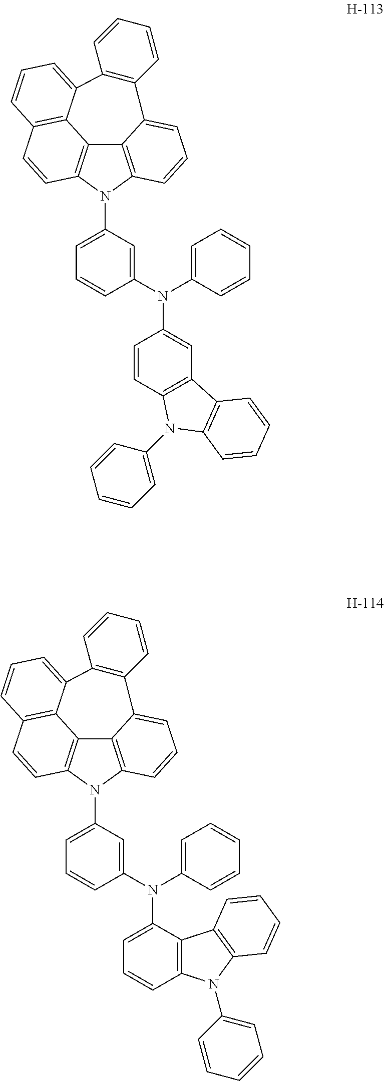 [SMILES: c1ccc(N(c2cccc(-n3c4cccc5c4c4c6c(cccc6ccc43)-c3ccccc3-5)c2)c2ccc3c(c2)c2ccccc2n3-c2ccccc2)cc1 ; c1ccc(N(c2cccc(-n3c4cccc5c4c4c6c(cccc6ccc43)-c3ccccc3-5)c2)c2cccc3c2c2ccccc2n3-c2ccccc2)cc1] 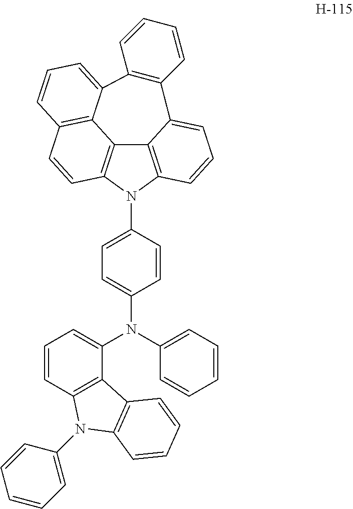 [SMILES: c1ccc(N(c2ccc(-n3c4cccc5c4c4c6c(cccc6ccc43)-c3ccccc3-5)cc2)c2cccc3c2c2ccccc2n3-c2ccccc2)cc1] 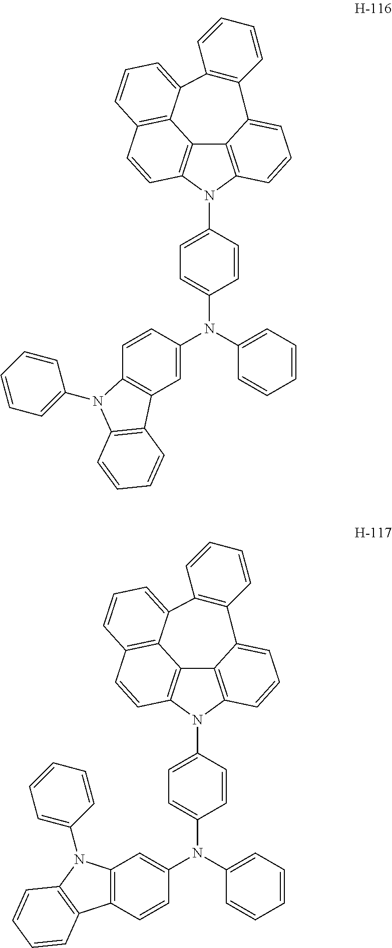 [SMILES: c1ccc(N(c2ccc(-n3c4cccc5c4c4c6c(cccc6ccc43)-c3ccccc3-5)cc2)c2ccc3c(c2)c2ccccc2n3-c2ccccc2)cc1 ; c1ccc(N(c2ccc(-n3c4cccc5c4c4c6c(cccc6ccc43)-c3ccccc3-5)cc2)c2ccc3c4ccccc4n(-c4ccccc4)c3c2)cc1] 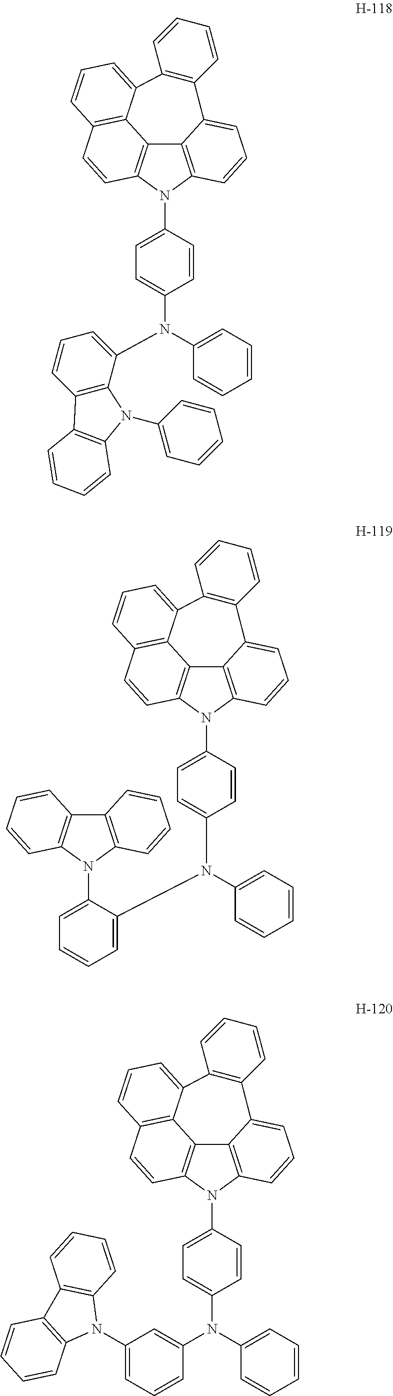 [SMILES: c1ccc(N(c2ccc(-n3c4cccc5c4c4c6c(cccc6ccc43)-c3ccccc3-5)cc2)c2cccc3c4ccccc4n(-c4ccccc4)c23)cc1 ; c1ccc(N(c2ccc(-n3c4cccc5c4c4c6c(cccc6ccc43)-c3ccccc3-5)cc2)c2ccccc2-n2c3ccccc3c3ccccc32)cc1 ; c1ccc(N(c2ccc(-n3c4cccc5c4c4c6c(cccc6ccc43)-c3ccccc3-5)cc2)c2cccc(-n3c4ccccc4c4ccccc43)c2)cc1] 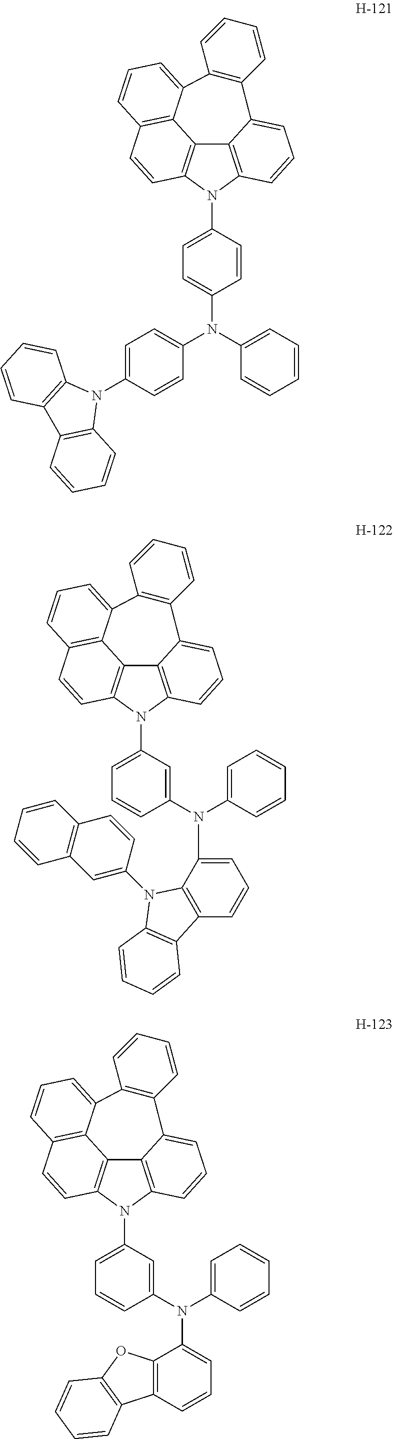 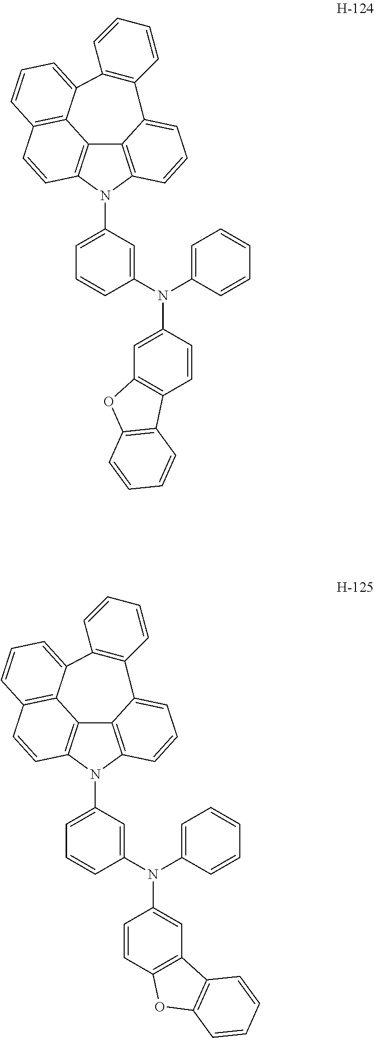 [SMILES: c1ccc(N(c2cccc(-n3c4cccc5c4c4c6c(cccc6ccc43)-c3ccccc3-5)c2)c2ccc3c(c2)oc2ccccc23)cc1 ; c1ccc(N(c2cccc(-n3c4cccc5c4c4c6c(cccc6ccc43)-c3ccccc3-5)c2)c2ccc3oc4ccccc4c3c2)cc1] 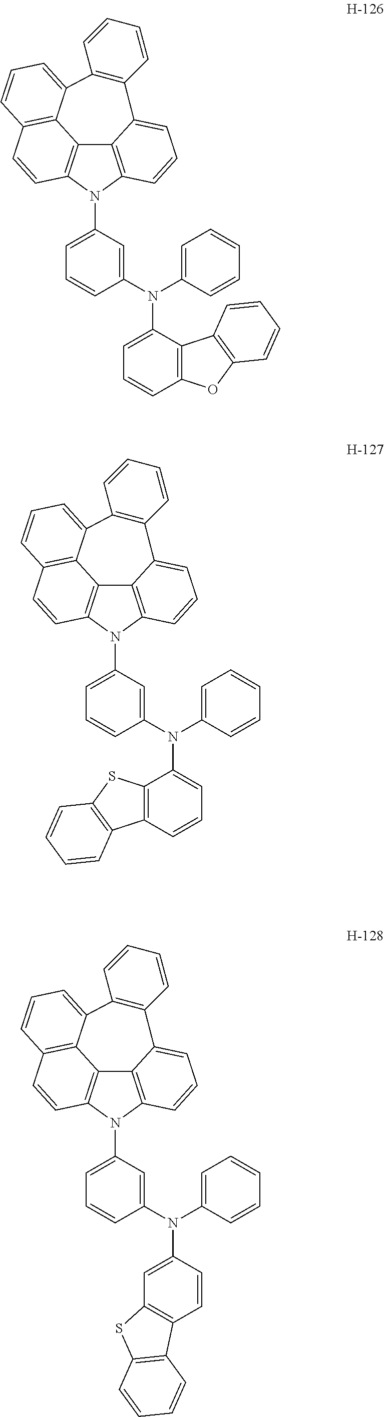 [SMILES: c1ccc(N(c2cccc(-n3c4cccc5c4c4c6c(cccc6ccc43)-c3ccccc3-5)c2)c2cccc3oc4ccccc4c23)cc1 ; c1ccc(N(c2cccc(-n3c4cccc5c4c4c6c(cccc6ccc43)-c3ccccc3-5)c2)c2cccc3c2sc2ccccc23)cc1 ; c1ccc(N(c2cccc(-n3c4cccc5c4c4c6c(cccc6ccc43)-c3ccccc3-5)c2)c2ccc3c(c2)sc2ccccc23)cc1] 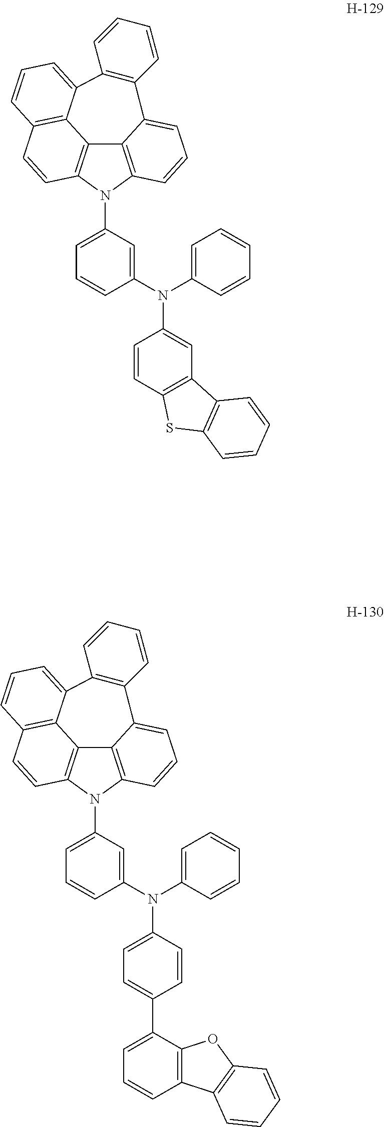 [SMILES: c1ccc(N(c2cccc(-n3c4cccc5c4c4c6c(cccc6ccc43)-c3ccccc3-5)c2)c2ccc3sc4ccccc4c3c2)cc1 ; c1ccc(N(c2ccc(-c3cccc4c3oc3ccccc34)cc2)c2cccc(-n3c4cccc5c4c4c6c(cccc6ccc43)-c3ccccc3-5)c2)cc1] 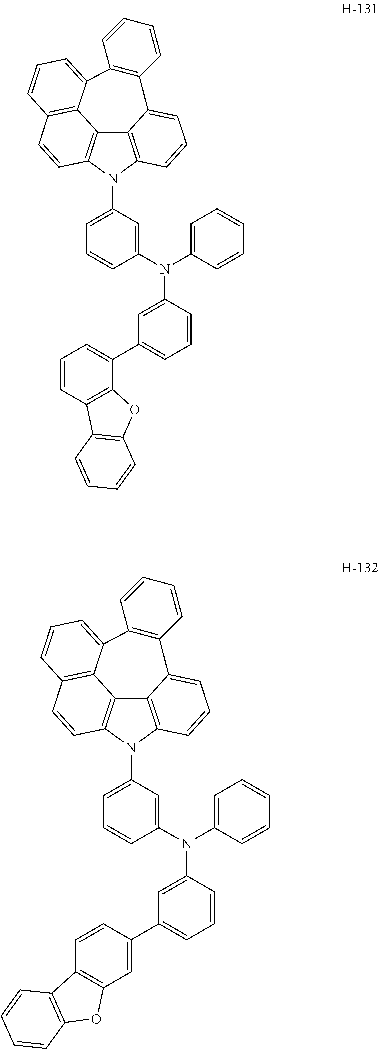 [SMILES: c1ccc(N(c2cccc(-c3cccc4c3oc3ccccc34)c2)c2cccc(-n3c4cccc5c4c4c6c(cccc6ccc43)-c3ccccc3-5)c2)cc1 ; c1ccc(N(c2cccc(-c3ccc4c(c3)oc3ccccc34)c2)c2cccc(-n3c4cccc5c4c4c6c(cccc6ccc43)-c3ccccc3-5)c2)cc1] 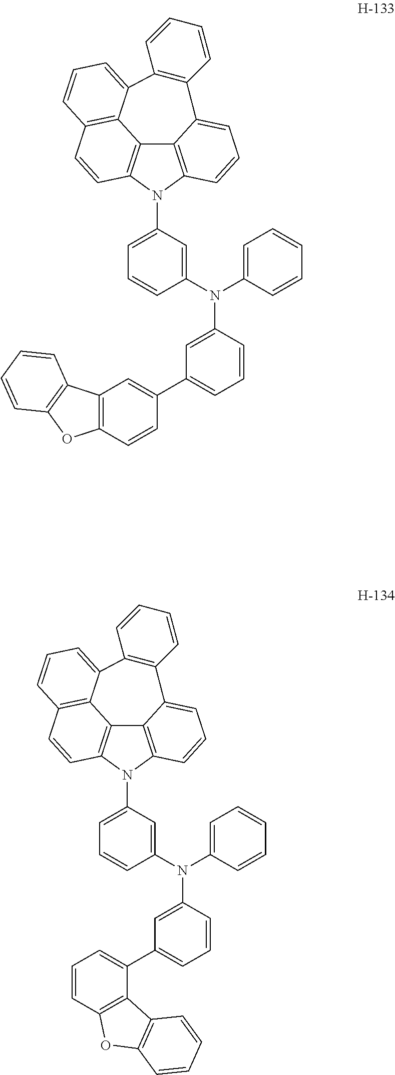 [SMILES: c1ccc(N(c2cccc(-c3ccc4oc5ccccc5c4c3)c2)c2cccc(-n3c4cccc5c4c4c6c(cccc6ccc43)-c3ccccc3-5)c2)cc1 ; c1ccc(N(c2cccc(-c3cccc4oc5ccccc5c34)c2)c2cccc(-n3c4cccc5c4c4c6c(cccc6ccc43)-c3ccccc3-5)c2)cc1] 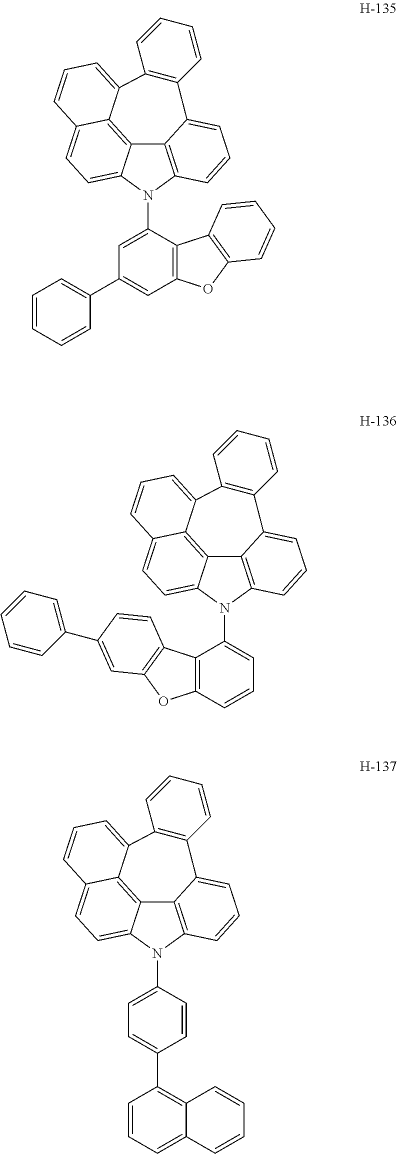 [SMILES: c1ccc(-c2cc(-n3c4cccc5c4c4c6c(cccc6ccc43)-c3ccccc3-5)c3c(c2)oc2ccccc23)cc1 ; c1ccc(-c2ccc3c(c2)oc2cccc(-n4c5cccc6c5c5c7c(cccc7ccc54)-c4ccccc4-6)c23)cc1 ; c1ccc2c(c1)-c1cccc3ccc4c(c13)c1c-2cccc1n4-c1ccc(-c2cccc3ccccc23)cc1] 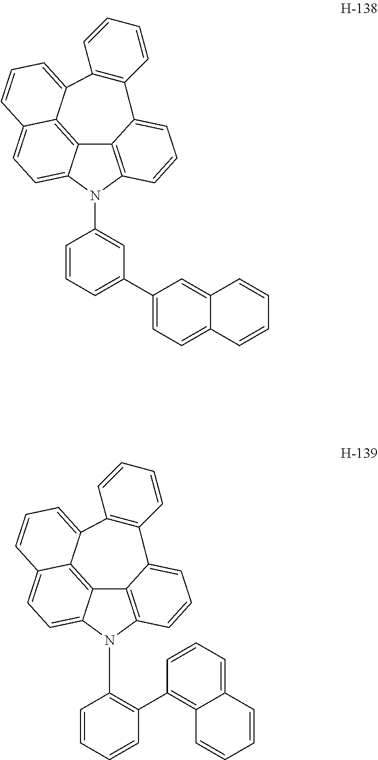 [SMILES: c1cc(-c2ccc3ccccc3c2)cc(-n2c3cccc4c3c3c5c(cccc5ccc32)-c2ccccc2-4)c1 ; c1ccc2c(c1)-c1cccc3ccc4c(c13)c1c-2cccc1n4-c1ccccc1-c1cccc2ccccc12] 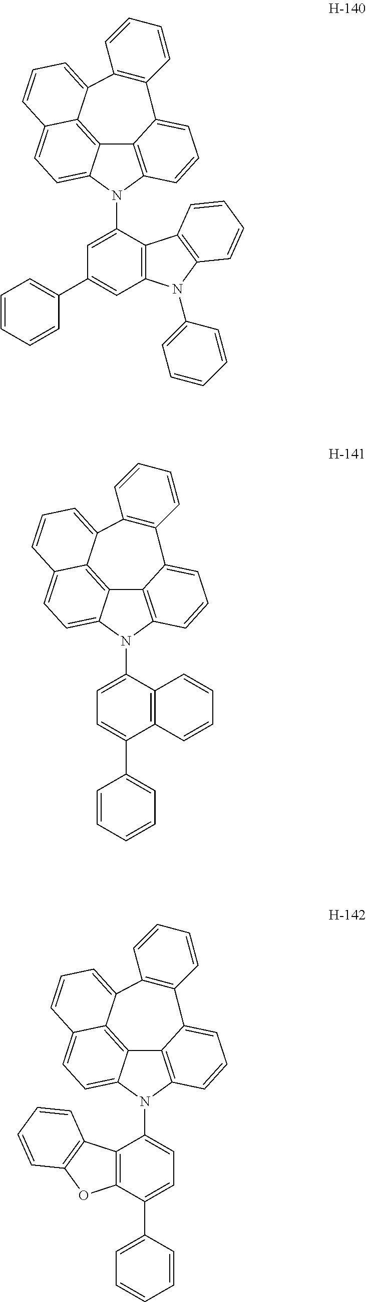 [SMILES: c1ccc(-c2cc(-n3c4cccc5c4c4c6c(cccc6ccc43)-c3ccccc3-5)c3c4ccccc4n(-c4ccccc4)c3c2)cc1 ; c1ccc(-c2ccc(-n3c4cccc5c4c4c6c(cccc6ccc43)-c3ccccc3-5)c3ccccc23)cc1 ; c1ccc(-c2ccc(-n3c4cccc5c4c4c6c(cccc6ccc43)-c3ccccc3-5)c3c2oc2ccccc23)cc1] 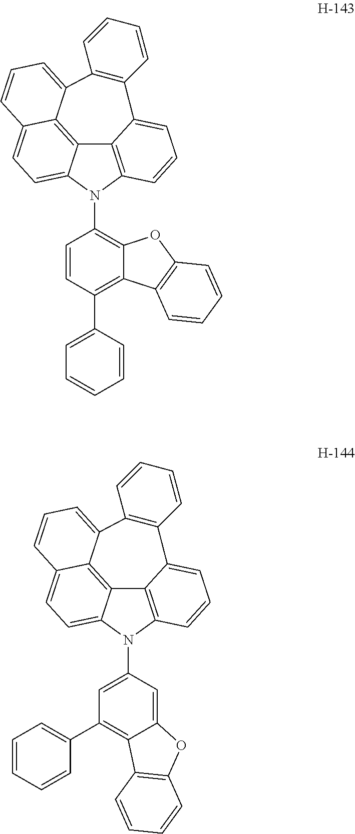 [SMILES: c1ccc(-c2ccc(-n3c4cccc5c4c4c6c(cccc6ccc43)-c3ccccc3-5)c3oc4ccccc4c23)cc1 ; c1ccc(-c2cc(-n3c4cccc5c4c4c6c(cccc6ccc43)-c3ccccc3-5)cc3oc4ccccc4c23)cc1] 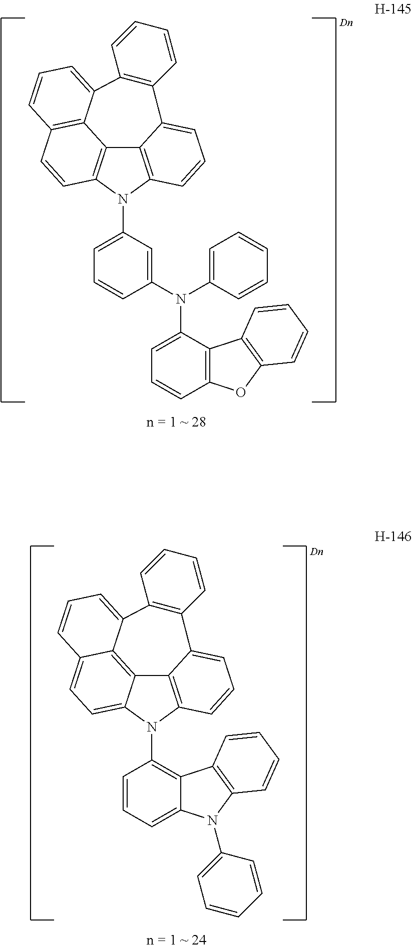 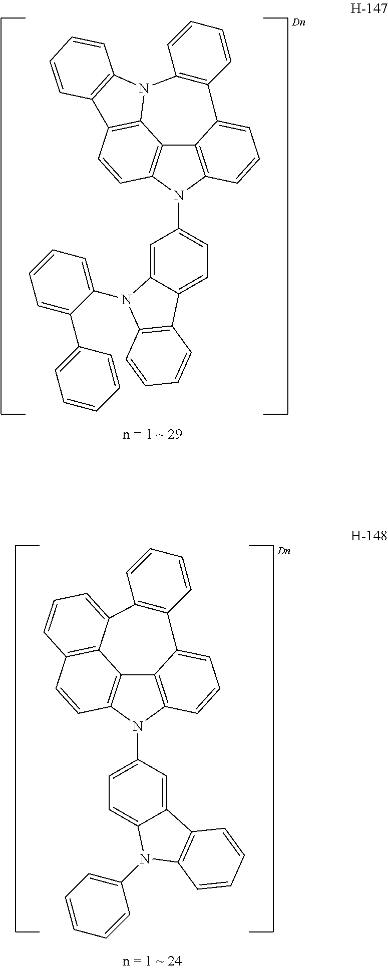 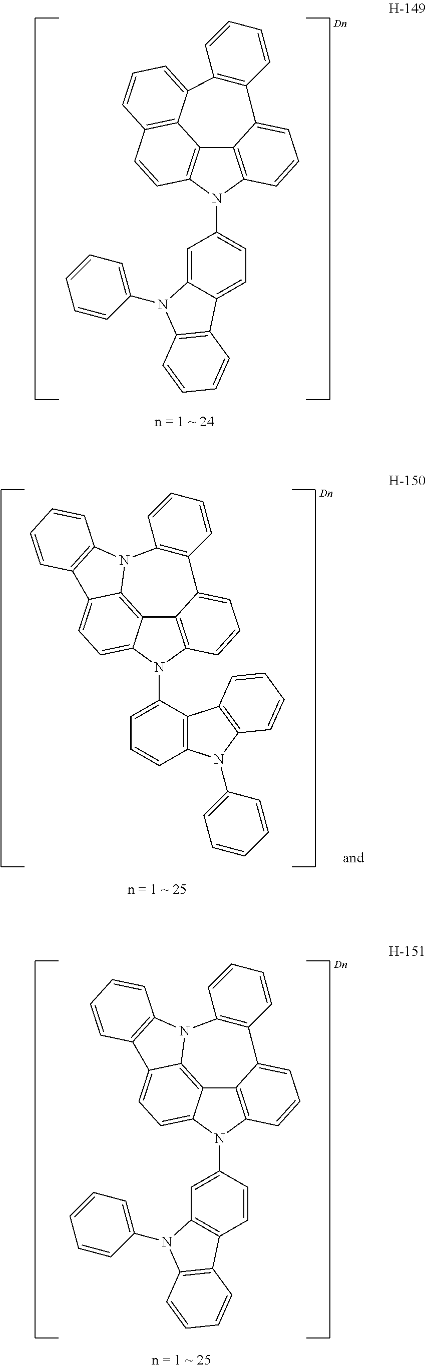
wherein in the compounds above, Dn represents that n number of hydrogens are replaced with deuterium.

7. The plurality of host materials according to claim 1, wherein the compound represented by Formula 2 is at least one selected from the following compounds.

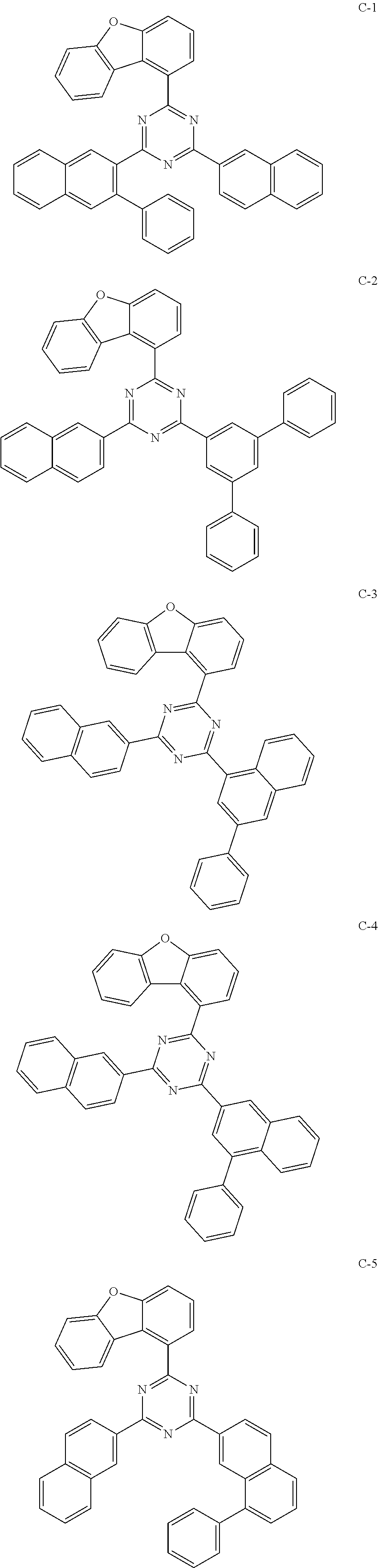

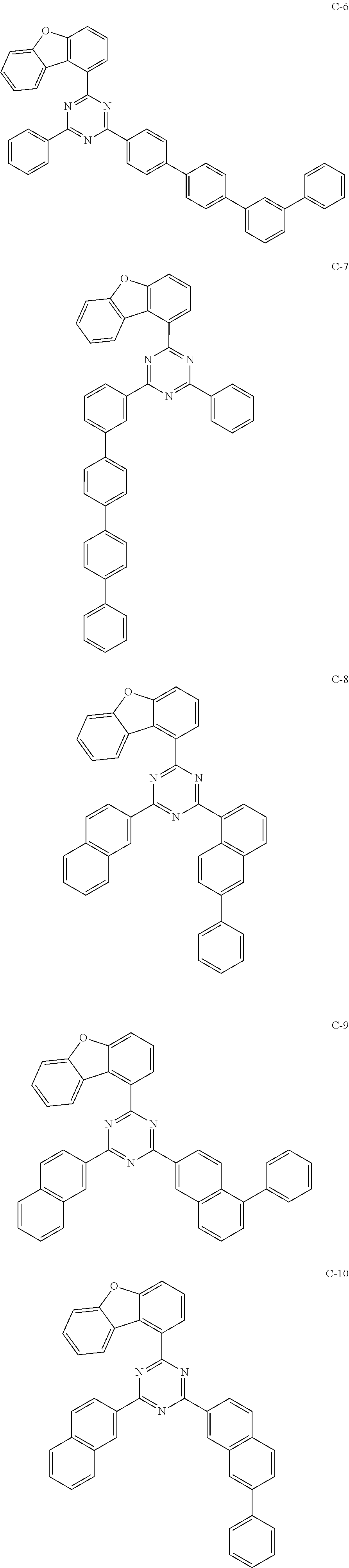 [SMILES: c1ccc(-c2cccc(-c3ccc(-c4ccc(-c5nc(-c6ccccc6)nc(-c6cccc7oc8ccccc8c67)n5)cc4)cc3)c2)cc1 ; c1ccc(-c2ccc(-c3ccc(-c4cccc(-c5nc(-c6ccccc6)nc(-c6cccc7oc8ccccc8c67)n5)c4)cc3)cc2)cc1 ; c1ccc(-c2ccc3c(-c4nc(-c5ccc6ccccc6c5)nc(-c5cccc6oc7ccccc7c56)n4)cccc3c2)cc1 ; c1ccc(-c2cccc3cc(-c4nc(-c5ccc6ccccc6c5)nc(-c5cccc6oc7ccccc7c56)n4)ccc23)cc1 ; c1ccc(-c2ccc3ccc(-c4nc(-c5ccc6ccccc6c5)nc(-c5cccc6oc7ccccc7c56)n4)cc3c2)cc1]

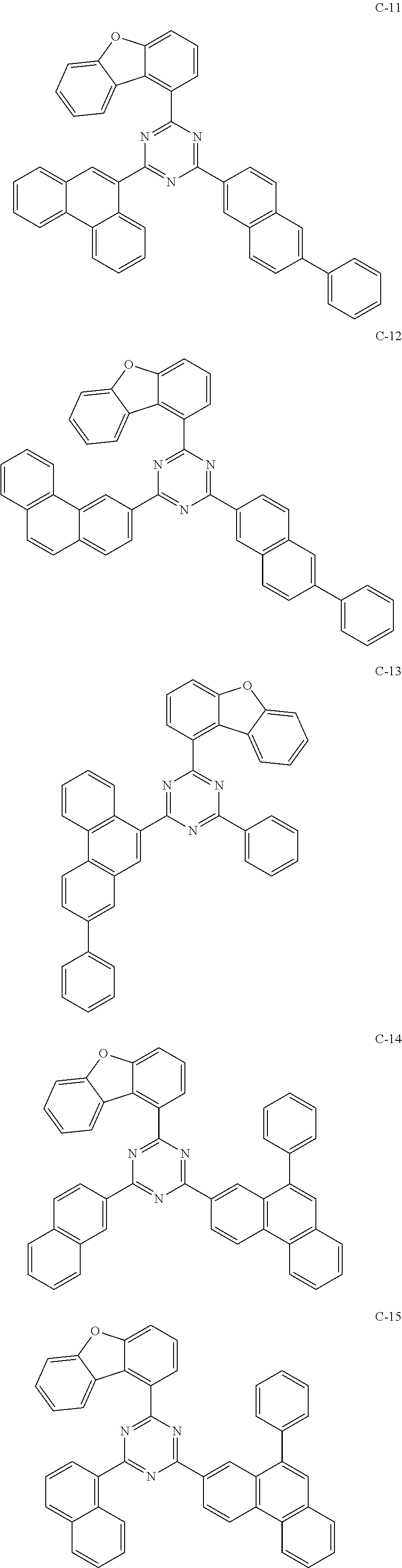 [SMILES: c1ccc(-c2ccc3cc(-c4nc(-c5cc6ccccc6c6ccccc56)nc(-c5cccc6oc7ccccc7c56)n4)ccc3c2)cc1 ; c1ccc(-c2ccc3cc(-c4nc(-c5ccc6ccc7ccccc7c6c5)nc(-c5cccc6oc7ccccc7c56)n4)ccc3c2)cc1 ; c1ccc(-c2ccc3c(c2)cc(-c2nc(-c4ccccc4)nc(-c4cccc5oc6ccccc6c45)n2)c2ccccc23)cc1 ; c1ccc(-c2cc3ccccc3c3ccc(-c4nc(-c5ccc6ccccc6c5)nc(-c5cccc6oc7ccccc7c56)n4)cc23)cc1 ; c1ccc(-c2cc3ccccc3c3ccc(-c4nc(-c5cccc6ccccc56)nc(-c5cccc6oc7ccccc7c56)n4)cc23)cc1]

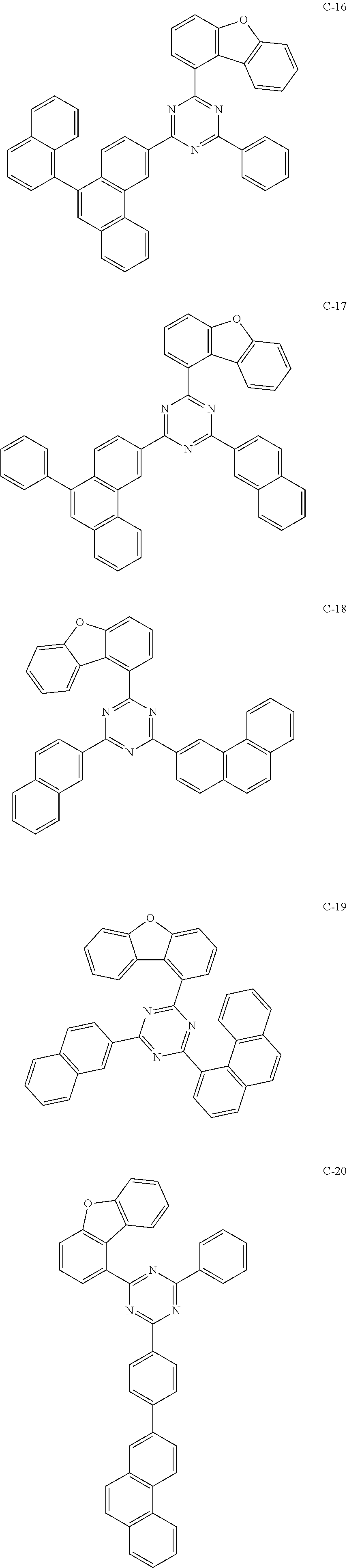 [SMILES: c1ccc(-c2nc(-c3ccc4c(-c5cccc6ccccc56)cc5ccccc5c4c3)nc(-c3cccc4oc5ccccc5c34)n2)cc1 ; c1ccc(-c2cc3ccccc3c3cc(-c4nc(-c5ccc6ccccc6c5)nc(-c5cccc6oc7ccccc7c56)n4)ccc23)cc1 ; c1ccc2cc(-c3nc(-c4ccc5ccc6ccccc6c5c4)nc(-c4cccc5oc6ccccc6c45)n3)ccc2c1 ; c1ccc2cc(-c3nc(-c4cccc5ccc6ccccc6c45)nc(-c4cccc5oc6ccccc6c45)n3)ccc2c1 ; c1ccc(-c2nc(-c3ccc(-c4ccc5c(ccc6ccccc65)c4)cc3)nc(-c3cccc4oc5ccccc5c34)n2)cc1]

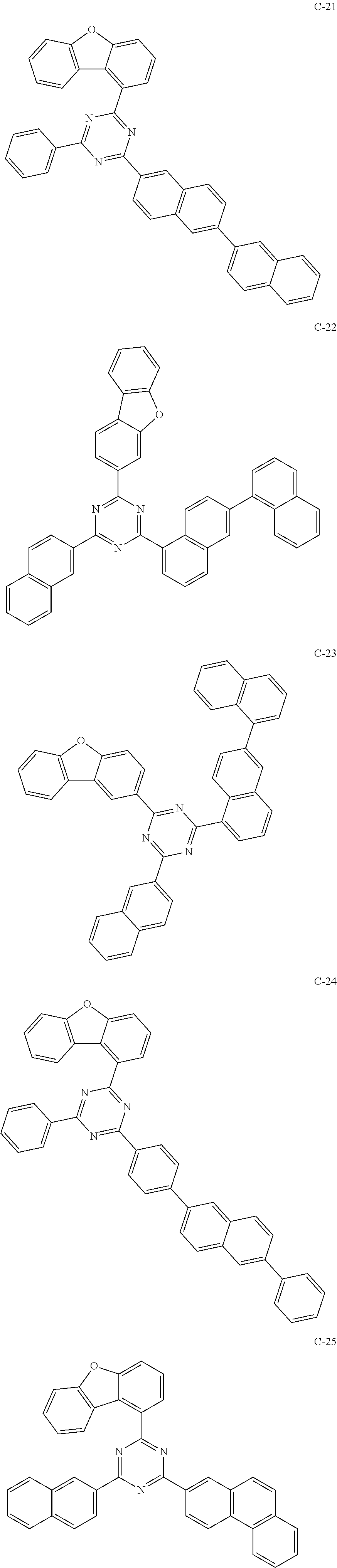 [SMILES: c1ccc(-c2nc(-c3ccc4cc(-c5ccc6ccccc6c5)ccc4c3)nc(-c3cccc4oc5ccccc5c34)n2)cc1 ; c1ccc2cc(-c3nc(-c4ccc5c(c4)oc4ccccc45)nc(-c4cccc5cc(-c6cccc7ccccc67)ccc45)n3)ccc2c1 ; c1ccc2cc(-c3nc(-c4ccc5oc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7oc8ccccc8c67)n5)cc4)ccc3c2)cc1 ; c1ccc2cc(-c3nc(-c4ccc5c(ccc6ccccc65)c4)nc(-c4cccc5oc6ccccc6c45)n3)ccc2c1]

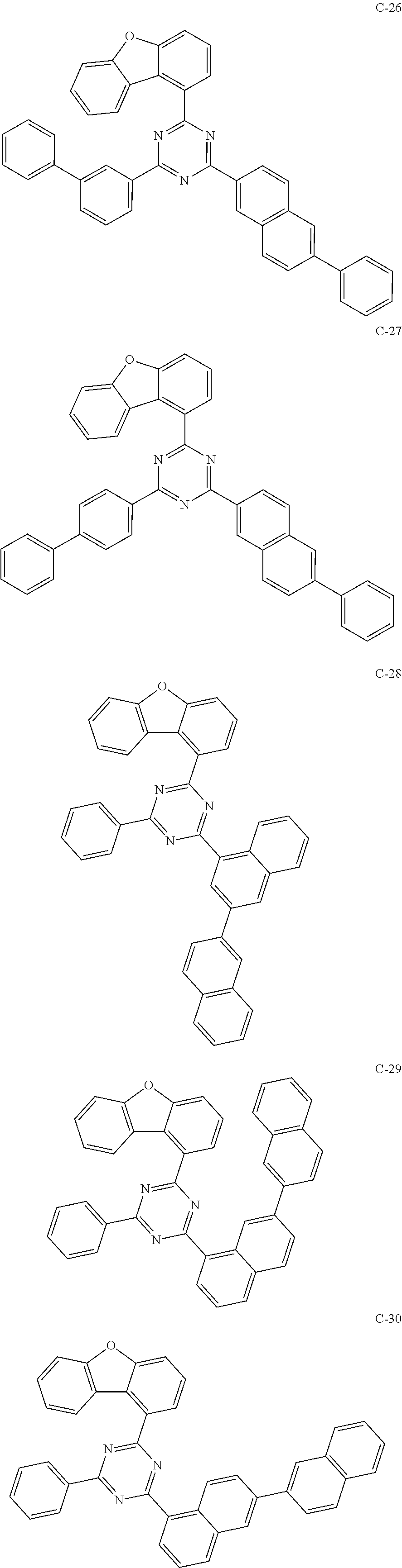 [SMILES: c1ccc(-c2cccc(-c3nc(-c4ccc5cc(-c6ccccc6)ccc5c4)nc(-c4cccc5oc6ccccc6c45)n3)c2)cc1 ; c1ccc(-c2ccc(-c3nc(-c4ccc5cc(-c6ccccc6)ccc5c4)nc(-c4cccc5oc6ccccc6c45)n3)cc2)cc1 ; c1ccc(-c2nc(-c3cc(-c4ccc5ccccc5c4)cc4ccccc34)nc(-c3cccc4oc5ccccc5c34)n2)cc1 ; c1ccc(-c2nc(-c3cccc4ccc(-c5ccc6ccccc6c5)cc34)nc(-c3cccc4oc5ccccc5c34)n2)cc1 ; c1ccc(-c2nc(-c3cccc4cc(-c5ccc6ccccc6c5)ccc34)nc(-c3cccc4oc5ccccc5c34)n2)cc1]

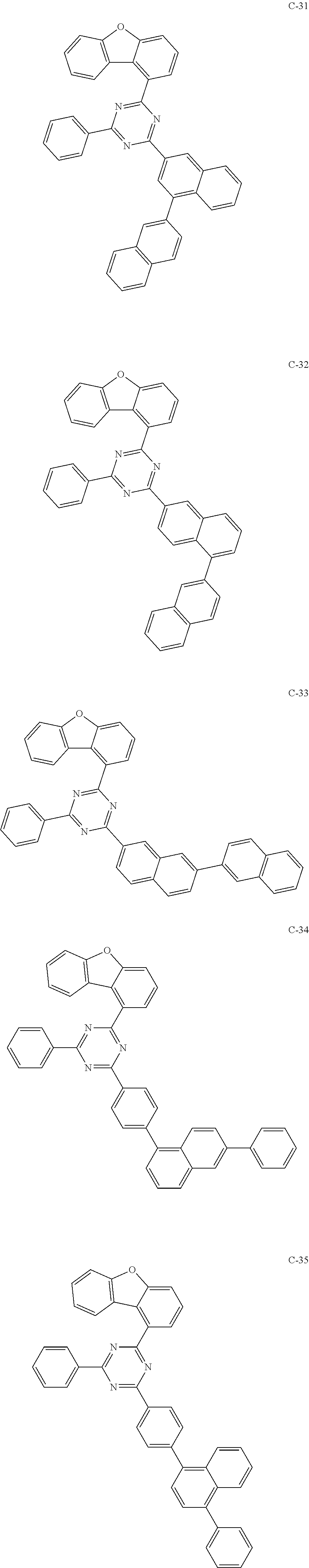 [SMILES: c1ccc(-c2nc(-c3cc(-c4ccc5ccccc5c4)c4ccccc4c3)nc(-c3cccc4oc5ccccc5c34)n2)cc1 ; c1ccc(-c2nc(-c3ccc4c(-c5ccc6ccccc6c5)cccc4c3)nc(-c3cccc4oc5ccccc5c34)n2)cc1 ; c1ccc(-c2nc(-c3ccc4ccc(-c5ccc6ccccc6c5)cc4c3)nc(-c3cccc4oc5ccccc5c34)n2)cc1 ; c1ccc(-c2ccc3c(-c4ccc(-c5nc(-c6ccccc6)nc(-c6cccc7oc8ccccc8c67)n5)cc4)cccc3c2)cc1 ; c1ccc(-c2nc(-c3ccc(-c4ccc(-c5ccccc5)c5ccccc45)cc3)nc(-c3cccc4oc5ccccc5c34)n2)cc1]

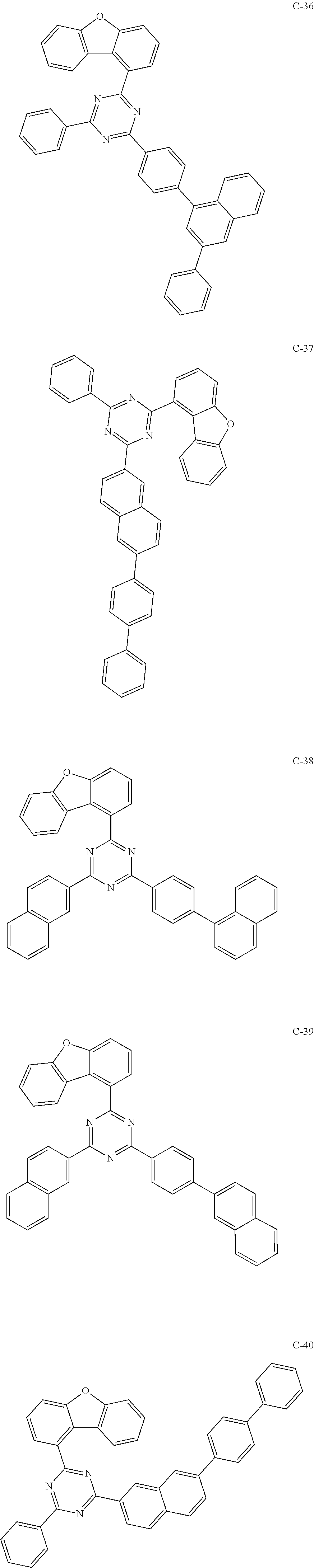 [SMILES: c1ccc(-c2cc(-c3ccc(-c4nc(-c5ccccc5)nc(-c5cccc6oc7ccccc7c56)n4)cc3)c3ccccc3c2)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc2cc(-c3nc(-c4ccc(-c5cccc6ccccc56)cc4)nc(-c4cccc5oc6ccccc6c45)n3)ccc2c1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3ccc4ccc(-c5nc(-c6ccccc6)nc(-c6cccc7oc8ccccc8c67)n5)cc4c3)cc2)cc1]

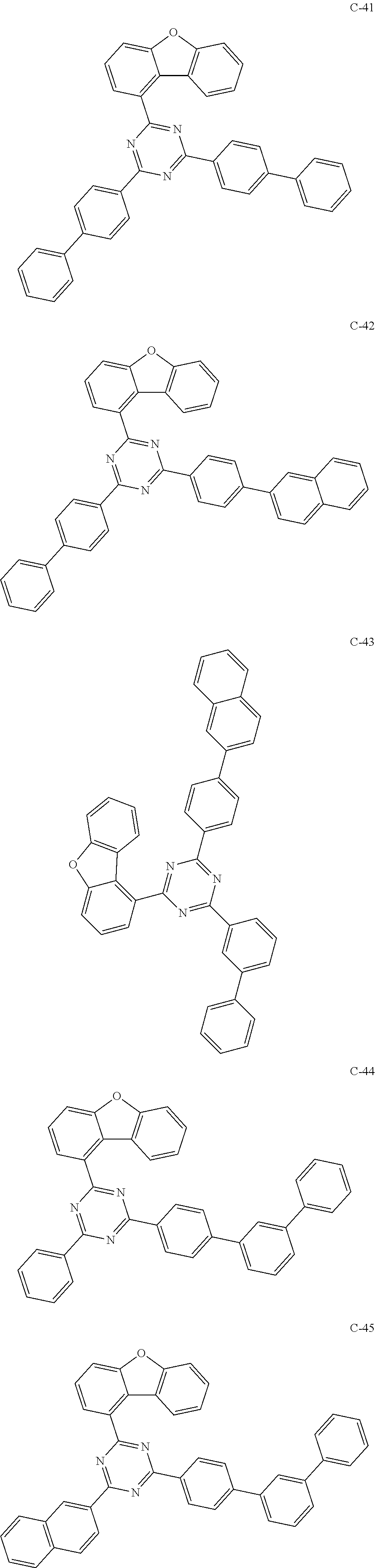 [SMILES: c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4ccc(-c5ccc6ccccc6c5)cc4)nc(-c4cccc5oc6ccccc6c45)n3)cc2)cc1 ; c1ccc(-c2cccc(-c3nc(-c4ccc(-c5ccc6ccccc6c5)cc4)nc(-c4cccc5oc6ccccc6c45)n3)c2)cc1 ; c1ccc(-c2cccc(-c3ccc(-c4nc(-c5ccccc5)nc(-c5cccc6oc7ccccc7c56)n4)cc3)c2)cc1 ; c1ccc(-c2cccc(-c3ccc(-c4nc(-c5ccc6ccccc6c5)nc(-c5cccc6oc7ccccc7c56)n4)cc3)c2)cc1]

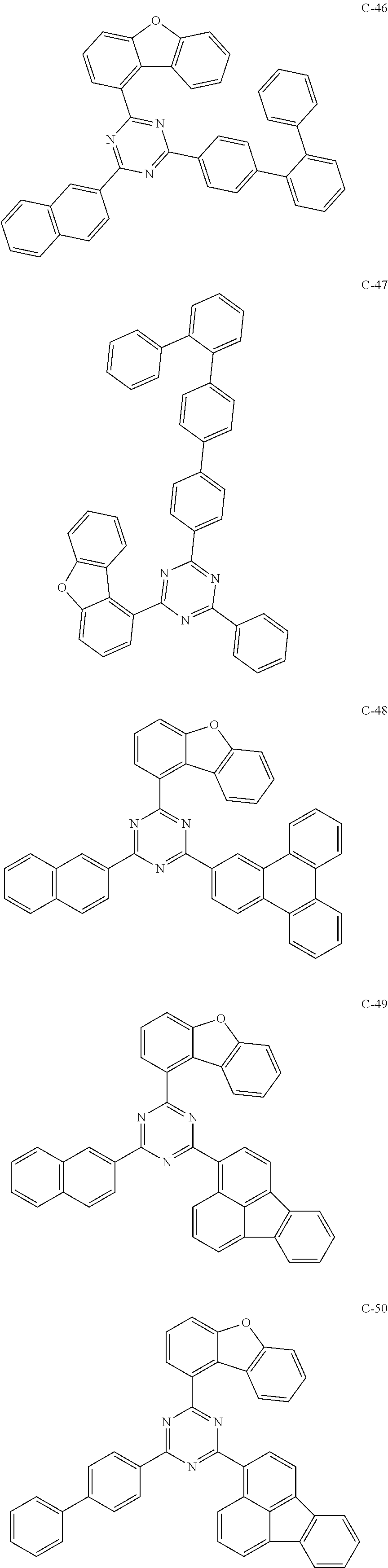 [SMILES: c1ccc(-c2ccccc2-c2ccc(-c3nc(-c4ccc5ccccc5c4)nc(-c4cccc5oc6ccccc6c45)n3)cc2)cc1 ; c1ccc(-c2nc(-c3ccc(-c4ccc(-c5ccccc5-c5ccccc5)cc4)cc3)nc(-c3cccc4oc5ccccc5c34)n2)cc1 ; c1ccc2cc(-c3nc(-c4ccc5c6ccccc6c6ccccc6c5c4)nc(-c4cccc5oc6ccccc6c45)n3)ccc2c1 ; c1ccc2c(c1)-c1cccc3c(-c4nc(-c5ccc6ccccc6c5)nc(-c5cccc6oc7ccccc7c56)n4)ccc-2c13 ; c1ccc(-c2ccc(-c3nc(-c4ccc5c6c(cccc46)-c4ccccc4-5)nc(-c4cccc5oc6ccccc6c45)n3)cc2)cc1]

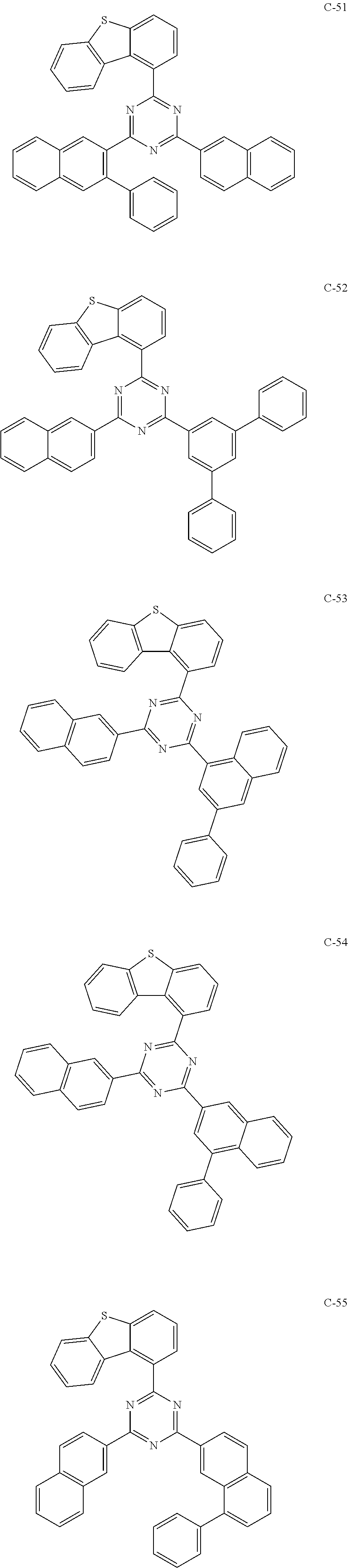 [SMILES: c1ccc(-c2cc3ccccc3cc2-c2nc(-c3ccc4ccccc4c3)nc(-c3cccc4sc5ccccc5c34)n2)cc1 ; c1ccc(-c2cc(-c3ccccc3)cc(-c3nc(-c4ccc5ccccc5c4)nc(-c4cccc5sc6ccccc6c45)n3)c2)cc1 ; c1ccc(-c2cc(-c3nc(-c4ccc5ccccc5c4)nc(-c4cccc5sc6ccccc6c45)n3)c3ccccc3c2)cc1 ; c1ccc(-c2cc(-c3nc(-c4ccc5ccccc5c4)nc(-c4cccc5sc6ccccc6c45)n3)cc3ccccc23)cc1 ; c1ccc(-c2cccc3ccc(-c4nc(-c5ccc6ccccc6c5)nc(-c5cccc6sc7ccccc7c56)n4)cc23)cc1]

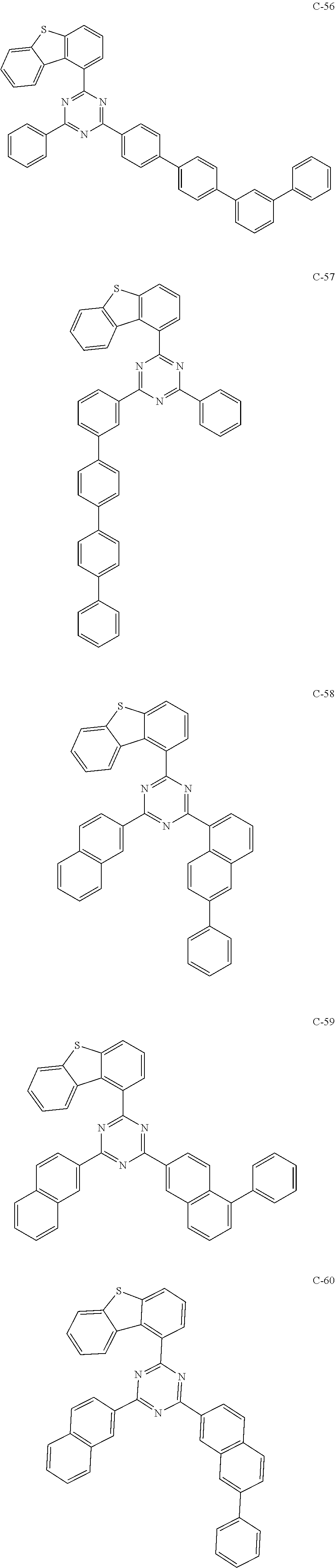 [SMILES: c1ccc(-c2cccc(-c3ccc(-c4ccc(-c5nc(-c6ccccc6)nc(-c6cccc7sc8ccccc8c67)n5)cc4)cc3)c2)cc1 ; c1ccc(-c2ccc(-c3ccc(-c4cccc(-c5nc(-c6ccccc6)nc(-c6cccc7sc8ccccc8c67)n5)c4)cc3)cc2)cc1 ; c1ccc(-c2ccc3c(-c4nc(-c5ccc6ccccc6c5)nc(-c5cccc6sc7ccccc7c56)n4)cccc3c2)cc1 ; c1ccc(-c2cccc3cc(-c4nc(-c5ccc6ccccc6c5)nc(-c5cccc6sc7ccccc7c56)n4)ccc23)cc1 ; c1ccc(-c2ccc3ccc(-c4nc(-c5ccc6ccccc6c5)nc(-c5cccc6sc7ccccc7c56)n4)cc3c2)cc1]

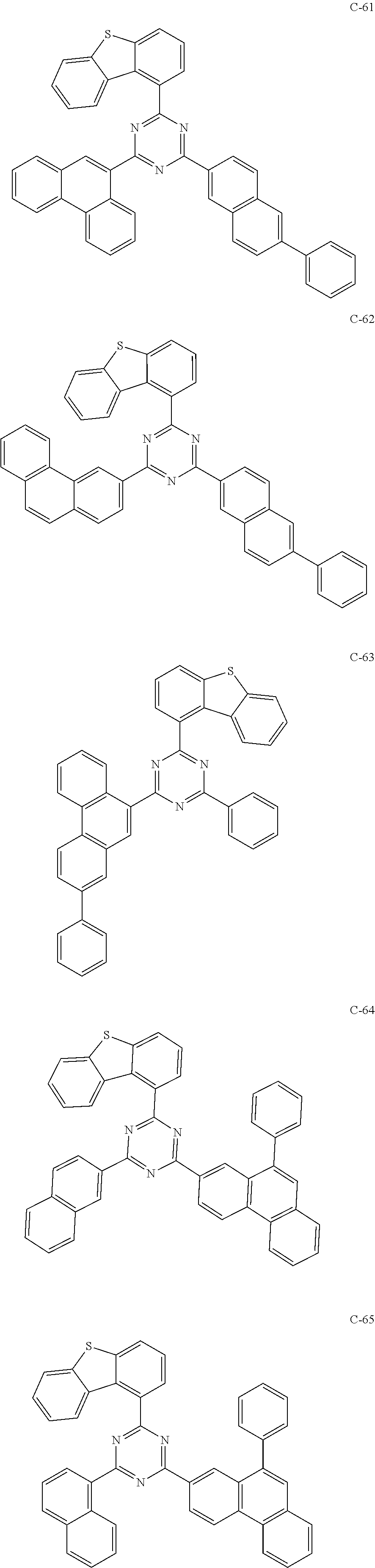 [SMILES: c1ccc(-c2ccc3cc(-c4nc(-c5cc6ccccc6c6ccccc56)nc(-c5cccc6sc7ccccc7c56)n4)ccc3c2)cc1 ; c1ccc(-c2ccc3cc(-c4nc(-c5ccc6ccc7ccccc7c6c5)nc(-c5cccc6sc7ccccc7c56)n4)ccc3c2)cc1 ; c1ccc(-c2ccc3c(c2)cc(-c2nc(-c4ccccc4)nc(-c4cccc5sc6ccccc6c45)n2)c2ccccc23)cc1 ; c1ccc(-c2cc3ccccc3c3ccc(-c4nc(-c5ccc6ccccc6c5)nc(-c5cccc6sc7ccccc7c56)n4)cc23)cc1 ; c1ccc(-c2cc3ccccc3c3ccc(-c4nc(-c5cccc6ccccc56)nc(-c5cccc6sc7ccccc7c56)n4)cc23)cc1]

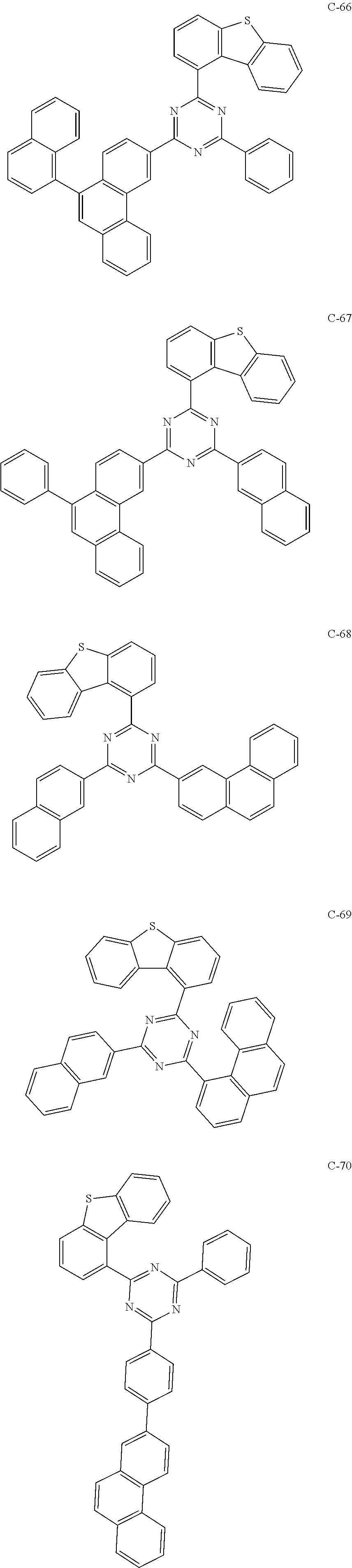 [SMILES: c1ccc(-c2nc(-c3ccc4c(-c5cccc6ccccc56)cc5ccccc5c4c3)nc(-c3cccc4sc5ccccc5c34)n2)cc1 ; c1ccc(-c2cc3ccccc3c3cc(-c4nc(-c5ccc6ccccc6c5)nc(-c5cccc6sc7ccccc7c56)n4)ccc23)cc1 ; c1ccc2cc(-c3nc(-c4ccc5ccc6ccccc6c5c4)nc(-c4cccc5sc6ccccc6c45)n3)ccc2c1 ; c1ccc2cc(-c3nc(-c4cccc5ccc6ccccc6c45)nc(-c4cccc5sc6ccccc6c45)n3)ccc2c1 ; c1ccc(-c2nc(-c3ccc(-c4ccc5c(ccc6ccccc65)c4)cc3)nc(-c3cccc4sc5ccccc5c34)n2)cc1]

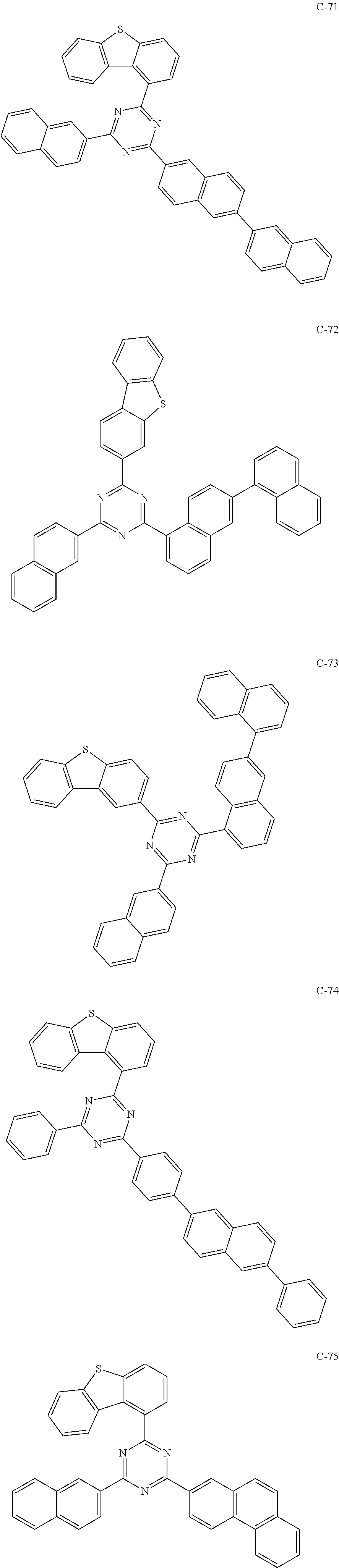 [SMILES: c1ccc2cc(-c3ccc4cc(-c5nc(-c6ccc7ccccc7c6)nc(-c6cccc7sc8ccccc8c67)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1 ; c1ccc2cc(-c3nc(-c4ccc5sc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6cccc7sc8ccccc8c67)n5)cc4)ccc3c2)cc1 ; c1ccc2cc(-c3nc(-c4ccc5c(ccc6ccccc65)c4)nc(-c4cccc5sc6ccccc6c45)n3)ccc2c1]

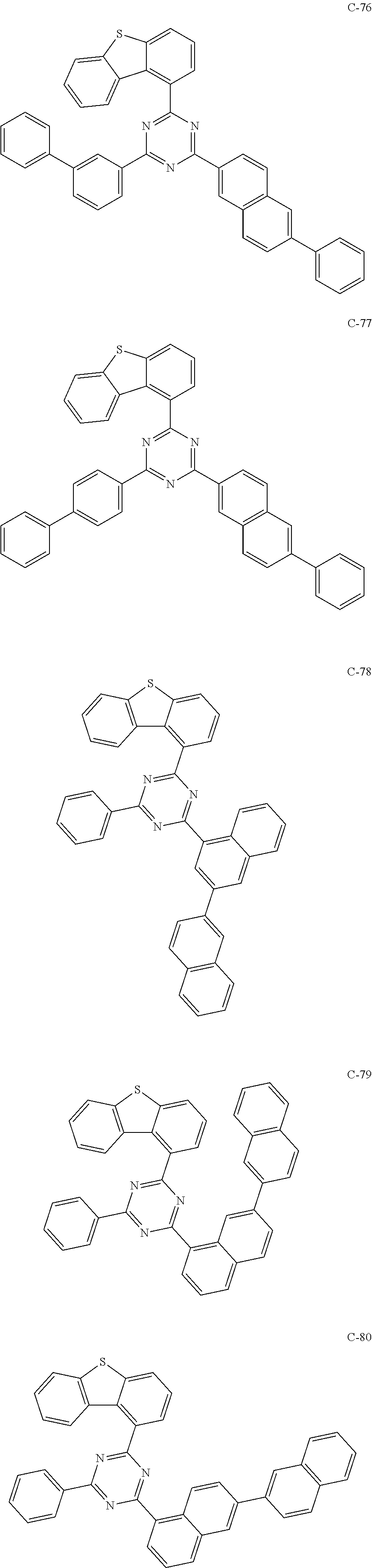 [SMILES: c1ccc(-c2cccc(-c3nc(-c4ccc5cc(-c6ccccc6)ccc5c4)nc(-c4cccc5sc6ccccc6c45)n3)c2)cc1 ; c1ccc(-c2ccc(-c3nc(-c4ccc5cc(-c6ccccc6)ccc5c4)nc(-c4cccc5sc6ccccc6c45)n3)cc2)cc1 ; c1ccc(-c2nc(-c3cc(-c4ccc5ccccc5c4)cc4ccccc34)nc(-c3cccc4sc5ccccc5c34)n2)cc1 ; c1ccc(-c2nc(-c3cccc4ccc(-c5ccc6ccccc6c5)cc34)nc(-c3cccc4sc5ccccc5c34)n2)cc1 ; c1ccc(-c2nc(-c3cccc4cc(-c5ccc6ccccc6c5)ccc34)nc(-c3cccc4sc5ccccc5c34)n2)cc1]

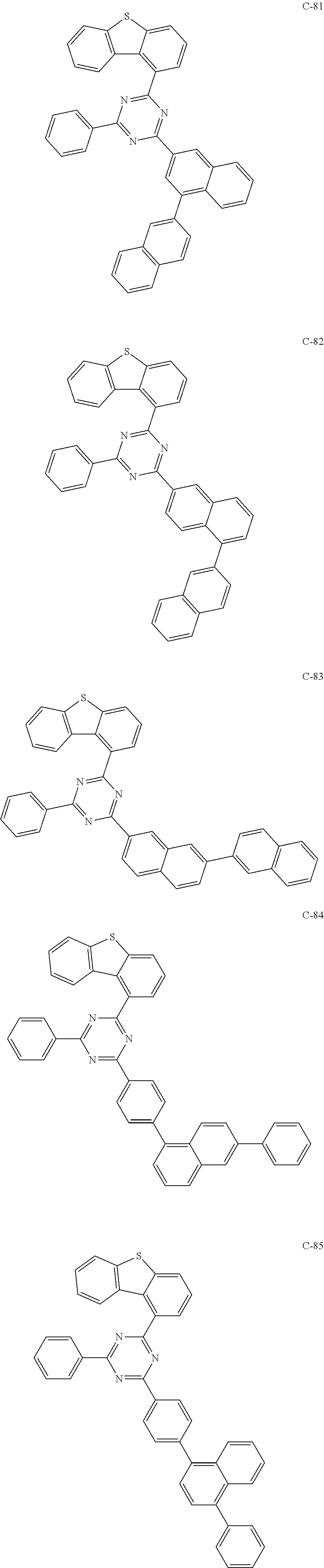 [SMILES: c1ccc(-c2nc(-c3cc(-c4ccc5ccccc5c4)c4ccccc4c3)nc(-c3cccc4sc5ccccc5c34)n2)cc1 ; c1ccc(-c2nc(-c3ccc4c(-c5ccc6ccccc6c5)cccc4c3)nc(-c3cccc4sc5ccccc5c34)n2)cc1 ; c1ccc(-c2nc(-c3ccc4ccc(-c5ccc6ccccc6c5)cc4c3)nc(-c3cccc4sc5ccccc5c34)n2)cc1 ; c1ccc(-c2ccc3c(-c4ccc(-c5nc(-c6ccccc6)nc(-c6cccc7sc8ccccc8c67)n5)cc4)cccc3c2)cc1 ; c1ccc(-c2nc(-c3ccc(-c4ccc(-c5ccccc5)c5ccccc45)cc3)nc(-c3cccc4sc5ccccc5c34)n2)cc1]

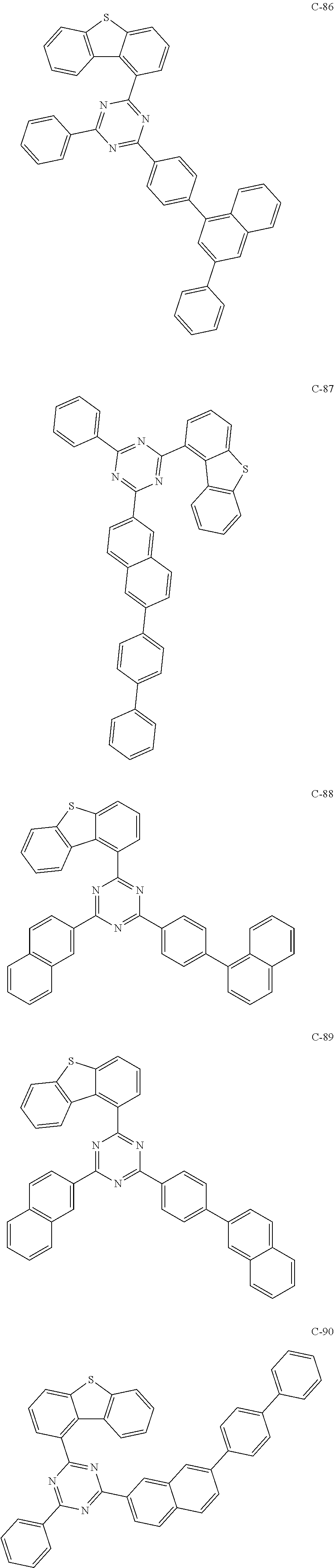 [SMILES: c1ccc(-c2cc(-c3ccc(-c4nc(-c5ccccc5)nc(-c5cccc6sc7ccccc7c56)n4)cc3)c3ccccc3c2)cc1 ; c1ccc(-c2ccc(-c3ccc4cc(-c5nc(-c6ccccc6)nc(-c6cccc7sc8ccccc8c67)n5)ccc4c3)cc2)cc1 ; c1ccc2cc(-c3nc(-c4ccc(-c5cccc6ccccc56)cc4)nc(-c4cccc5sc6ccccc6c45)n3)ccc2c1 ; c1ccc2cc(-c3ccc(-c4nc(-c5ccc6ccccc6c5)nc(-c5cccc6sc7ccccc7c56)n4)cc3)ccc2c1 ; c1ccc(-c2ccc(-c3ccc4ccc(-c5nc(-c6ccccc6)nc(-c6cccc7sc8ccccc8c67)n5)cc4c3)cc2)cc1]

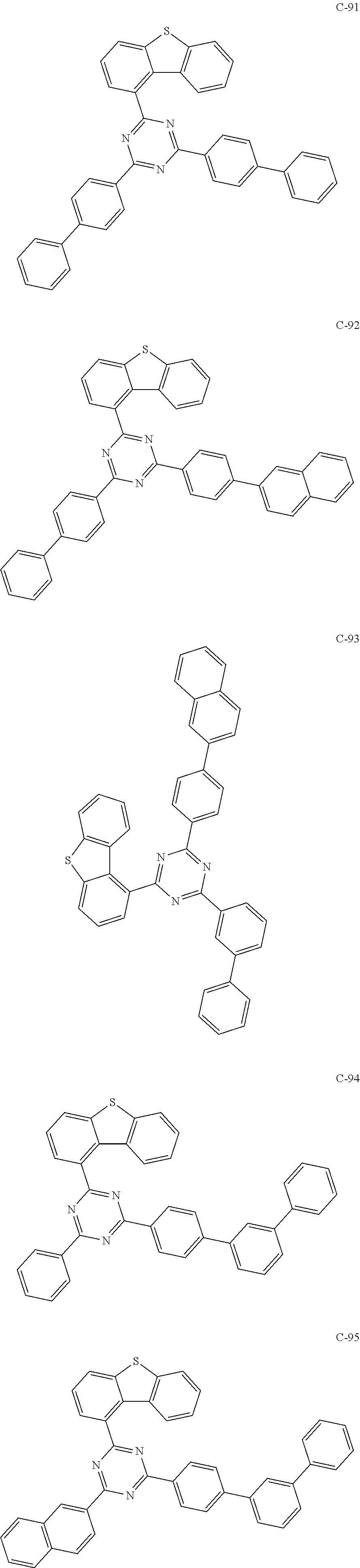 [SMILES: c1ccc(-c2ccc(-c3nc(-c4ccc(-c5ccccc5)cc4)nc(-c4cccc5sc6ccccc6c45)n3)cc2)cc1 ; c1ccc(-c2ccc(-c3nc(-c4ccc(-c5ccc6ccccc6c5)cc4)nc(-c4cccc5sc6ccccc6c45)n3)cc2)cc1 ; c1ccc(-c2cccc(-c3nc(-c4ccc(-c5ccc6ccccc6c5)cc4)nc(-c4cccc5sc6ccccc6c45)n3)c2)cc1 ; c1ccc(-c2cccc(-c3ccc(-c4nc(-c5ccccc5)nc(-c5cccc6sc7ccccc7c56)n4)cc3)c2)cc1 ; c1ccc(-c2cccc(-c3ccc(-c4nc(-c5ccc6ccccc6c5)nc(-c5cccc6sc7ccccc7c56)n4)cc3)c2)cc1]

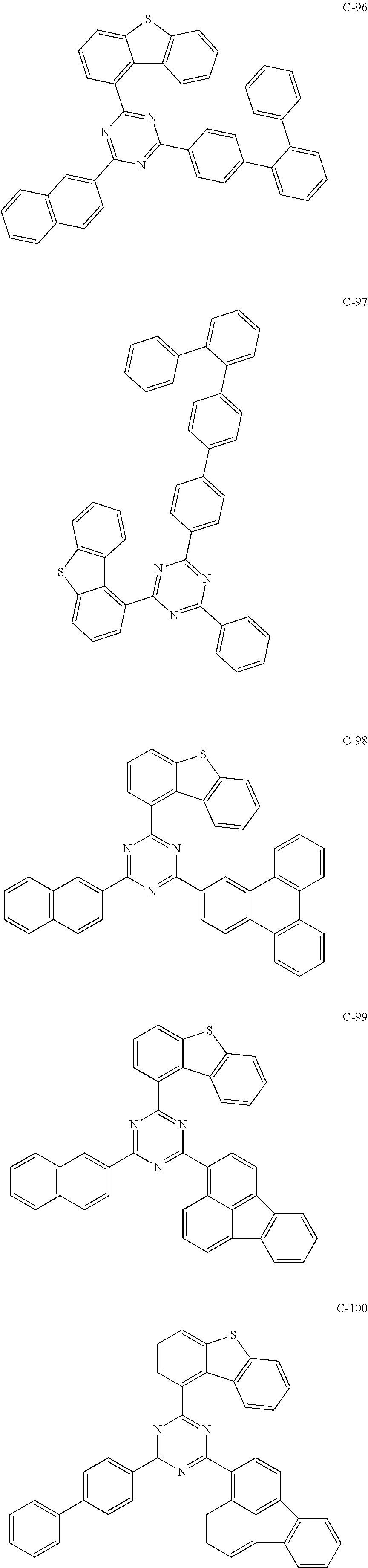 [SMILES: c1ccc(-c2ccccc2-c2ccc(-c3nc(-c4ccc5ccccc5c4)nc(-c4cccc5sc6ccccc6c45)n3)cc2)cc1 ; c1ccc(-c2nc(-c3ccc(-c4ccc(-c5ccccc5-c5ccccc5)cc4)cc3)nc(-c3cccc4sc5ccccc5c34)n2)cc1 ; c1ccc2cc(-c3nc(-c4ccc5c6ccccc6c6ccccc6c5c4)nc(-c4cccc5sc6ccccc6c45)n3)ccc2c1 ; c1ccc2c(c1)-c1cccc3c(-c4nc(-c5ccc6ccccc6c5)nc(-c5cccc6sc7ccccc7c56)n4)ccc-2c13 ; c1ccc(-c2ccc(-c3nc(-c4ccc5c6c(cccc46)-c4ccccc4-5)nc(-c4cccc5sc6ccccc6c45)n3)cc2)cc1]

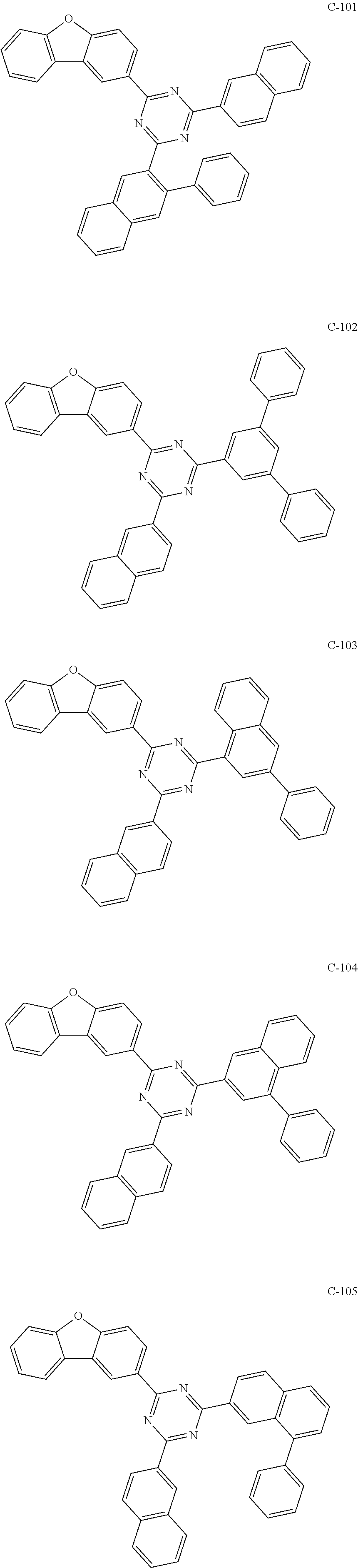 [SMILES: c1ccc(-c2cc3ccccc3cc2-c2nc(-c3ccc4ccccc4c3)nc(-c3ccc4oc5ccccc5c4c3)n2)cc1 ; c1ccc(-c2cc(-c3ccccc3)cc(-c3nc(-c4ccc5ccccc5c4)nc(-c4ccc5oc6ccccc6c5c4)n3)c2)cc1 ; c1ccc(-c2cc(-c3nc(-c4ccc5ccccc5c4)nc(-c4ccc5oc6ccccc6c5c4)n3)c3ccccc3c2)cc1 ; c1ccc(-c2cc(-c3nc(-c4ccc5ccccc5c4)nc(-c4ccc5oc6ccccc6c5c4)n3)cc3ccccc23)cc1 ; c1ccc(-c2cccc3ccc(-c4nc(-c5ccc6ccccc6c5)nc(-c5ccc6oc7ccccc7c6c5)n4)cc23)cc1]

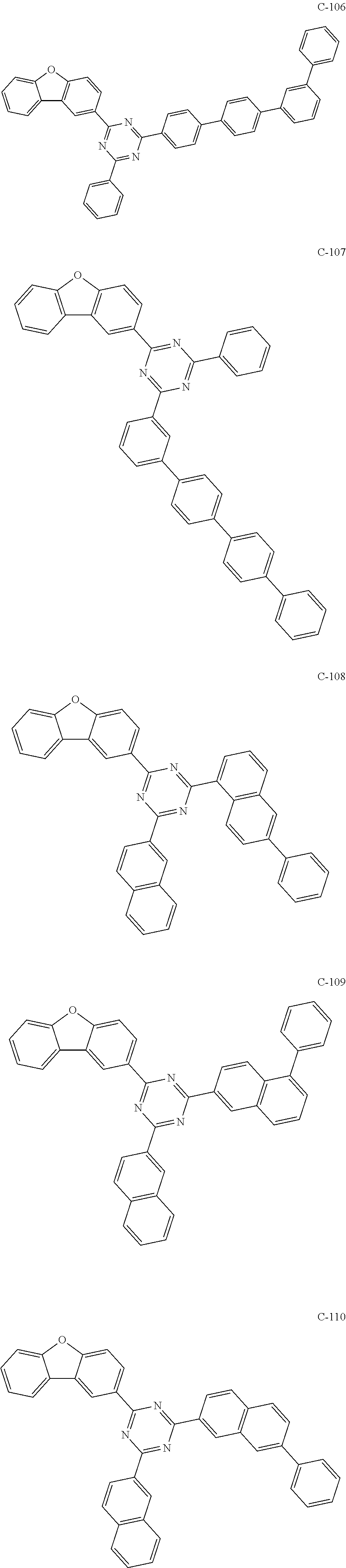 [SMILES: c1ccc(-c2cccc(-c3ccc(-c4ccc(-c5nc(-c6ccccc6)nc(-c6ccc7oc8ccccc8c7c6)n5)cc4)cc3)c2)cc1 ; c1ccc(-c2ccc(-c3ccc(-c4cccc(-c5nc(-c6ccccc6)nc(-c6ccc7oc8ccccc8c7c6)n5)c4)cc3)cc2)cc1 ; c1ccc(-c2ccc3c(-c4nc(-c5ccc6ccccc6c5)nc(-c5ccc6oc7ccccc7c6c5)n4)cccc3c2)cc1 ; c1ccc(-c2cccc3cc(-c4nc(-c5ccc6ccccc6c5)nc(-c5ccc6oc7ccccc7c6c5)n4)ccc23)cc1 ; c1ccc(-c2ccc3ccc(-c4nc(-c5ccc6ccccc6c5)nc(-c5ccc6oc7ccccc7c6c5)n4)cc3c2)cc1]

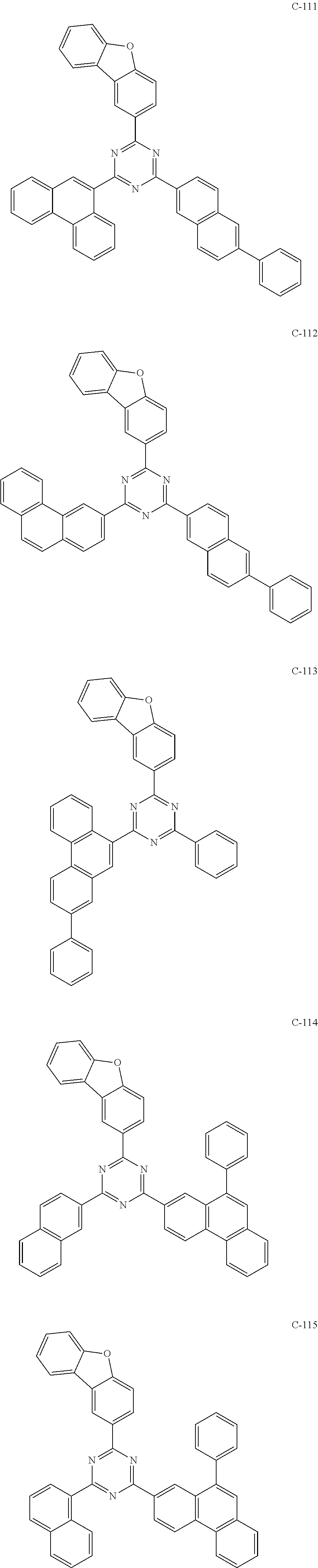 [SMILES: c1ccc(-c2ccc3cc(-c4nc(-c5ccc6oc7ccccc7c6c5)nc(-c5cc6ccccc6c6ccccc56)n4)ccc3c2)cc1 ; c1ccc(-c2ccc3cc(-c4nc(-c5ccc6ccc7ccccc7c6c5)nc(-c5ccc6oc7ccccc7c6c5)n4)ccc3c2)cc1 ; c1ccc(-c2ccc3c(c2)cc(-c2nc(-c4ccccc4)nc(-c4ccc5oc6ccccc6c5c4)n2)c2ccccc23)cc1 ; c1ccc(-c2cc3ccccc3c3ccc(-c4nc(-c5ccc6ccccc6c5)nc(-c5ccc6oc7ccccc7c6c5)n4)cc23)cc1 ; c1ccc(-c2cc3ccccc3c3ccc(-c4nc(-c5ccc6oc7ccccc7c6c5)nc(-c5cccc6ccccc56)n4)cc23)cc1]

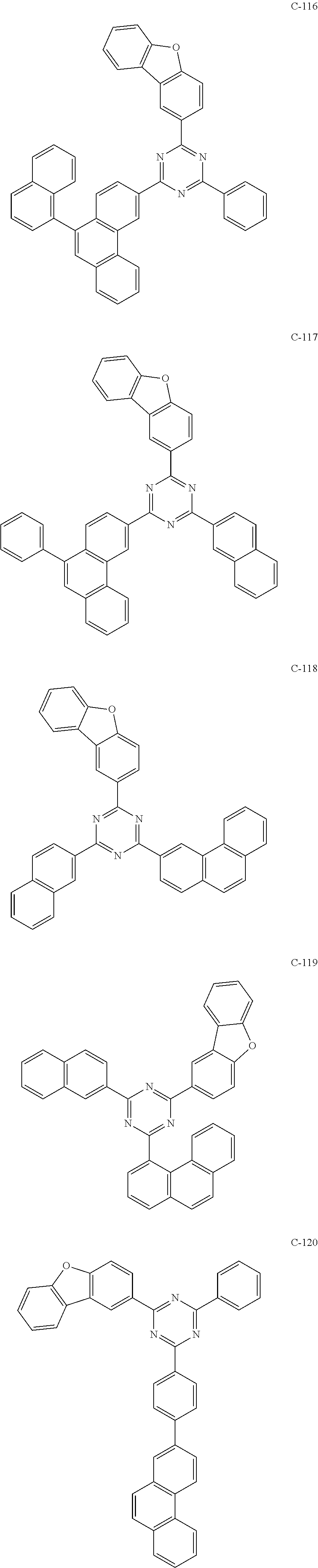 [SMILES: c1ccc(-c2nc(-c3ccc4oc5ccccc5c4c3)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6oc7ccccc7c6c5)n4)ccc23)cc1 ; c1ccc2cc(-c3nc(-c4ccc5ccc6ccccc6c5c4)nc(-c4ccc5oc6ccccc6c5c4)n3)ccc2c1 ; c1ccc2cc(-c3nc(-c4ccc5oc6ccccc6c5c4)nc(-c4cccc5ccc6ccccc6c45)n3)ccc2c1 ; c1ccc(-c2nc(-c3ccc(-c4ccc5c(ccc6ccccc65)c4)cc3)nc(-c3ccc4oc5ccccc5c4c3)n2)cc1]

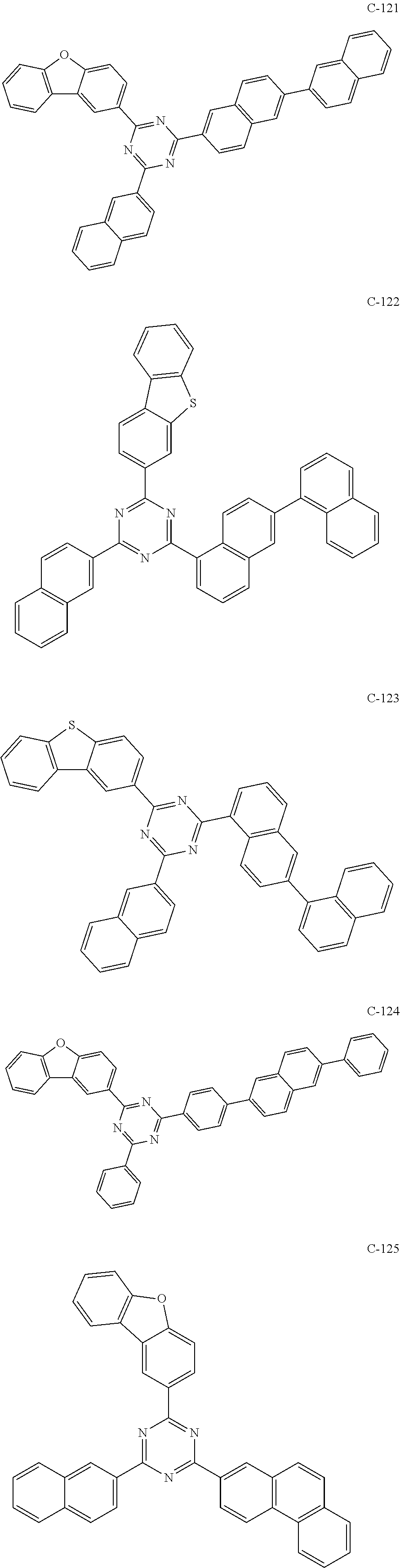 [SMILES: c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3nc(-c4ccc5c(c4)sc4ccccc45)nc(-c4cccc5cc(-c6cccc7ccccc67)ccc45)n3)ccc2c1 ; c1ccc2cc(-c3nc(-c4ccc5sc6ccccc6c5c4)nc(-c4cccc5cc(-c6cccc7ccccc67)ccc45)n3)ccc2c1 ; c1ccc(-c2ccc3cc(-c4ccc(-c5nc(-c6ccccc6)nc(-c6ccc7oc8ccccc8c7c6)n5)cc4)ccc3c2)cc1 ; c1ccc2cc(-c3nc(-c4ccc5c(ccc6ccccc65)c4)nc(-c4ccc5oc6ccccc6c5c4)n3)ccc2c1]

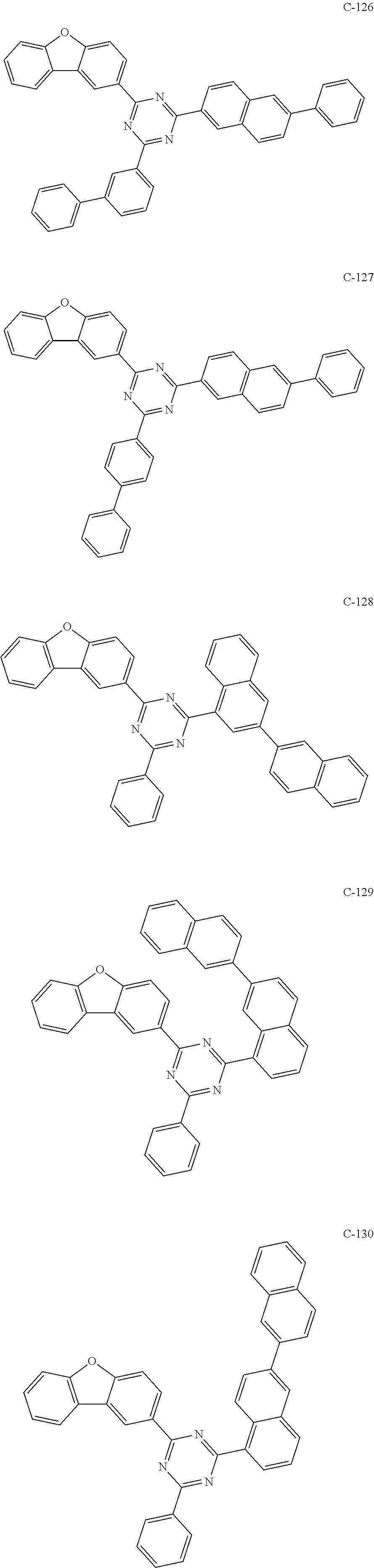 [SMILES: c1ccc(-c2cccc(-c3nc(-c4ccc5cc(-c6ccccc6)ccc5c4)nc(-c4ccc5oc6ccccc6c5c4)n3)c2)cc1 ; c1ccc(-c2ccc(-c3nc(-c4ccc5cc(-c6ccccc6)ccc5c4)nc(-c4ccc5oc6ccccc6c5c4)n3)cc2)cc1 ; c1ccc(-c2nc(-c3ccc4oc5ccccc5c4c3)nc(-c3cc(-c4ccc5ccccc5c4)cc4ccccc34)n2)cc1 ; c1ccc(-c2nc(-c3ccc4oc5ccccc5c4c3)nc(-c3cccc4ccc(-c5ccc6ccccc6c5)cc34)n2)cc1 ; c1ccc(-c2nc(-c3ccc4oc5ccccc5c4c3)nc(-c3cccc4cc(-c5ccc6ccccc6c5)ccc34)n2)cc1]

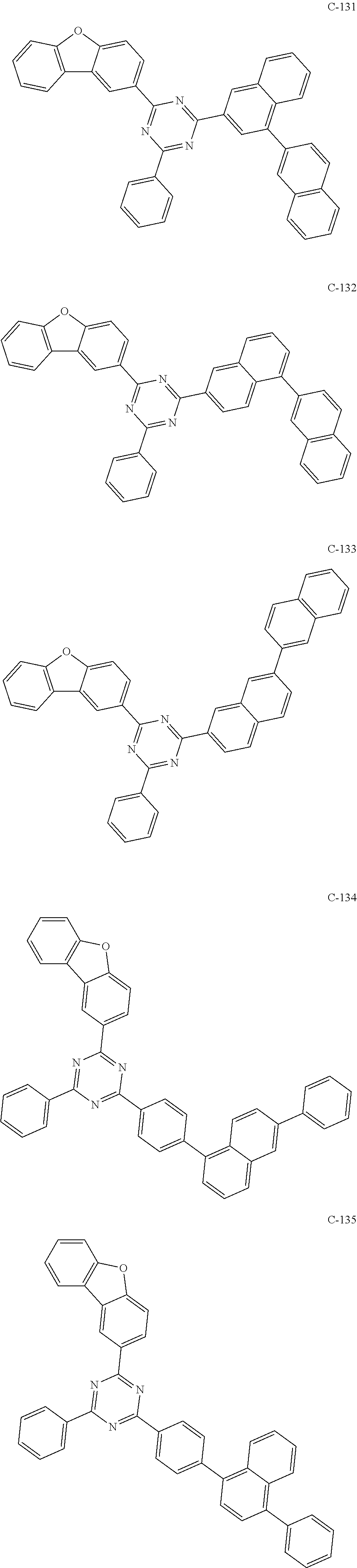 [SMILES: c1ccc(-c2nc(-c3cc(-c4ccc5ccccc5c4)c4ccccc4c3)nc(-c3ccc4oc5ccccc5c4c3)n2)cc1 ; c1ccc(-c2nc(-c3ccc4c(-c5ccc6ccccc6c5)cccc4c3)nc(-c3ccc4oc5ccccc5c4c3)n2)cc1 ; c1ccc(-c2nc(-c3ccc4ccc(-c5ccc6ccccc6c5)cc4c3)nc(-c3ccc4oc5ccccc5c4c3)n2)cc1 ; c1ccc(-c2ccc3c(-c4ccc(-c5nc(-c6ccccc6)nc(-c6ccc7oc8ccccc8c7c6)n5)cc4)cccc3c2)cc1 ; c1ccc(-c2nc(-c3ccc(-c4ccc(-c5ccccc5)c5ccccc45)cc3)nc(-c3ccc4oc5ccccc5c4c3)n2)cc1]

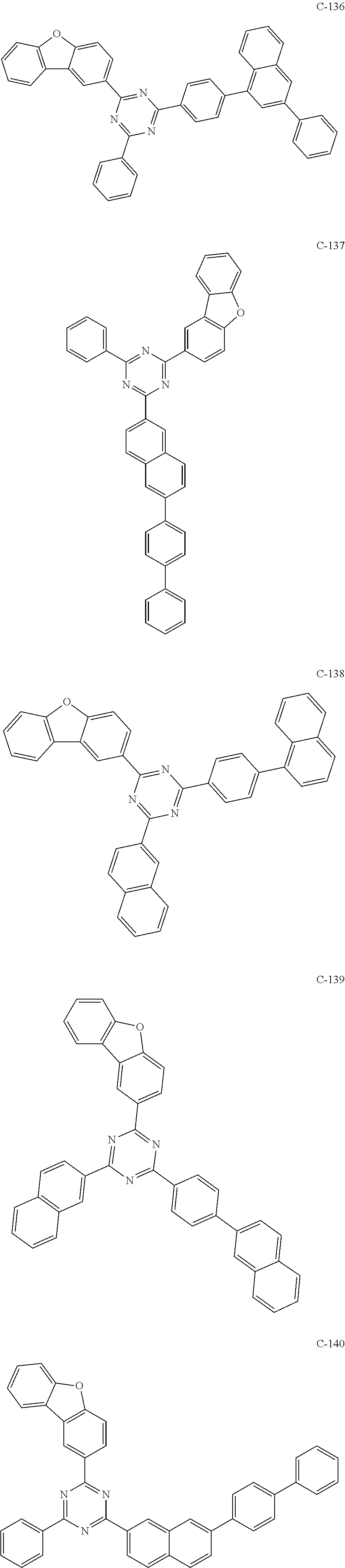 [SMILES: c1ccc(-c2cc(-c3ccc(-c4nc(-c5ccccc5)nc(-c5ccc6oc7ccccc7c6c5)n4)cc3)c3ccccc3c2)cc1 ; c1ccc(-c2ccc(-c3ccc4cc(-c5nc(-c6ccccc6)nc(-c6ccc7oc8ccccc8c7c6)n5)ccc4c3)cc2)cc1 ; c1ccc2cc(-c3nc(-c4ccc(-c5cccc6ccccc56)cc4)nc(-c4ccc5oc6ccccc6c5c4)n3)ccc2c1 ; c1ccc2cc(-c3ccc(-c4nc(-c5ccc6ccccc6c5)nc(-c5ccc6oc7ccccc7c6c5)n4)cc3)ccc2c1 ; c1ccc(-c2ccc(-c3ccc4ccc(-c5nc(-c6ccccc6)nc(-c6ccc7oc8ccccc8c7c6)n5)cc4c3)cc2)cc1]

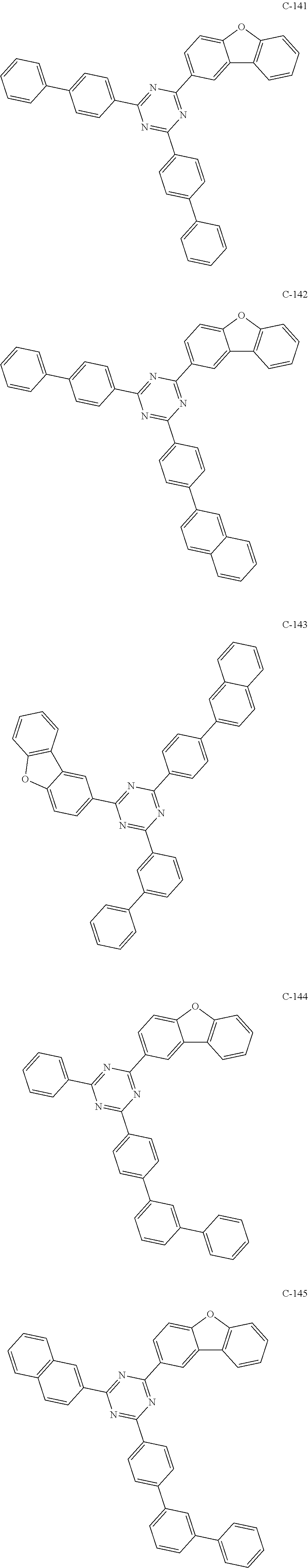 [SMILES: c1ccc(-c2ccc(-c3nc(-c4ccc(-c5ccccc5)cc4)nc(-c4ccc5oc6ccccc6c5c4)n3)cc2)cc1 ; c1ccc(-c2ccc(-c3nc(-c4ccc(-c5ccc6ccccc6c5)cc4)nc(-c4ccc5oc6ccccc6c5c4)n3)cc2)cc1 ; c1ccc(-c2cccc(-c3nc(-c4ccc(-c5ccc6ccccc6c5)cc4)nc(-c4ccc5oc6ccccc6c5c4)n3)c2)cc1 ; c1ccc(-c2cccc(-c3ccc(-c4nc(-c5ccccc5)nc(-c5ccc6oc7ccccc7c6c5)n4)cc3)c2)cc1 ; c1ccc(-c2cccc(-c3ccc(-c4nc(-c5ccc6ccccc6c5)nc(-c5ccc6oc7ccccc7c6c5)n4)cc3)c2)cc1]

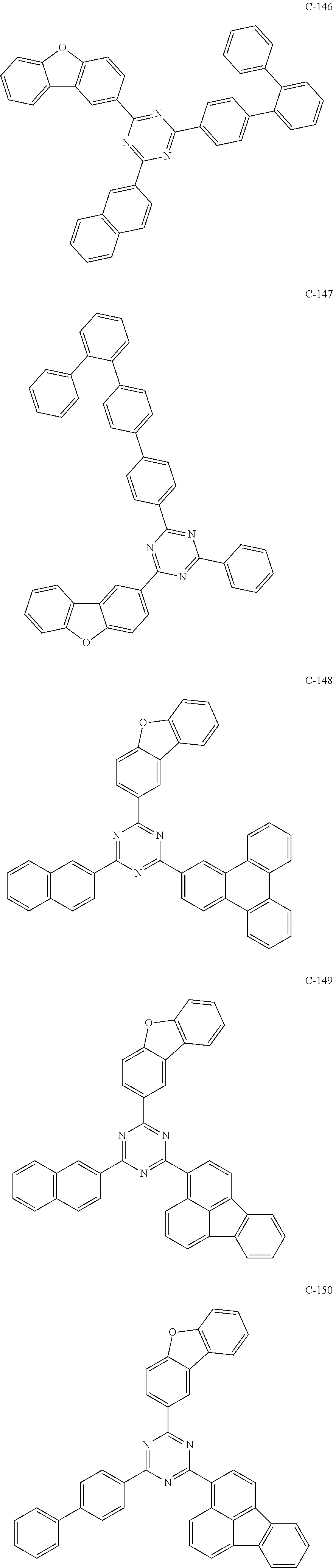 [SMILES: c1ccc(-c2ccccc2-c2ccc(-c3nc(-c4ccc5ccccc5c4)nc(-c4ccc5oc6ccccc6c5c4)n3)cc2)cc1 ; c1ccc(-c2nc(-c3ccc(-c4ccc(-c5ccccc5-c5ccccc5)cc4)cc3)nc(-c3ccc4oc5ccccc5c4c3)n2)cc1 ; c1ccc2cc(-c3nc(-c4ccc5oc6ccccc6c5c4)nc(-c4ccc5c6ccccc6c6ccccc6c5c4)n3)ccc2c1 ; c1ccc2c(c1)-c1cccc3c(-c4nc(-c5ccc6ccccc6c5)nc(-c5ccc6oc7ccccc7c6c5)n4)ccc-2c13 ; c1ccc(-c2ccc(-c3nc(-c4ccc5oc6ccccc6c5c4)nc(-c4ccc5c6c(cccc46)-c4ccccc4-5)n3)cc2)cc1]

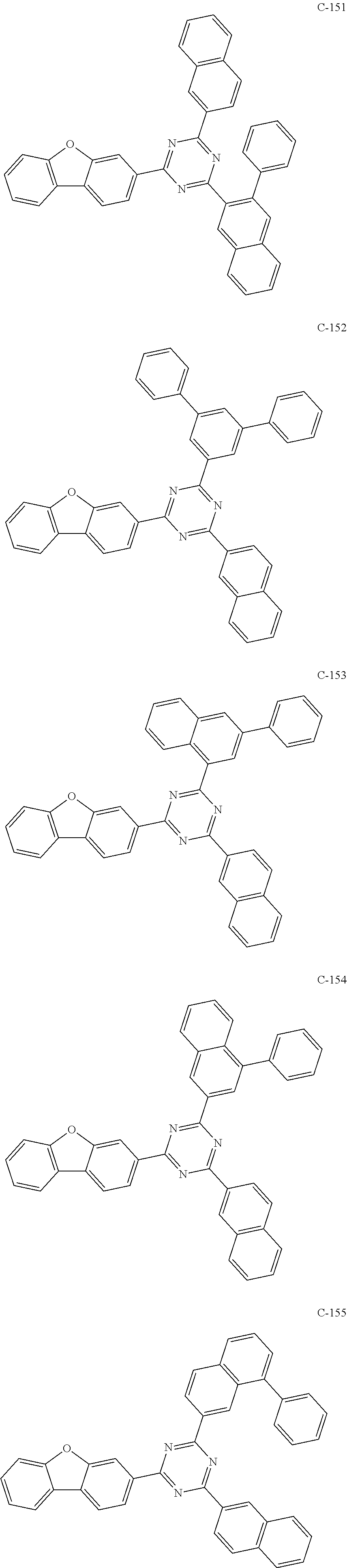 [SMILES: c1ccc(-c2cc3ccccc3cc2-c2nc(-c3ccc4ccccc4c3)nc(-c3ccc4c(c3)oc3ccccc34)n2)cc1 ; c1ccc(-c2cc(-c3ccccc3)cc(-c3nc(-c4ccc5ccccc5c4)nc(-c4ccc5c(c4)oc4ccccc45)n3)c2)cc1 ; c1ccc(-c2cc(-c3nc(-c4ccc5ccccc5c4)nc(-c4ccc5c(c4)oc4ccccc45)n3)c3ccccc3c2)cc1 ; c1ccc(-c2cc(-c3nc(-c4ccc5ccccc5c4)nc(-c4ccc5c(c4)oc4ccccc45)n3)cc3ccccc23)cc1 ; c1ccc(-c2cccc3ccc(-c4nc(-c5ccc6ccccc6c5)nc(-c5ccc6c(c5)oc5ccccc56)n4)cc23)cc1]

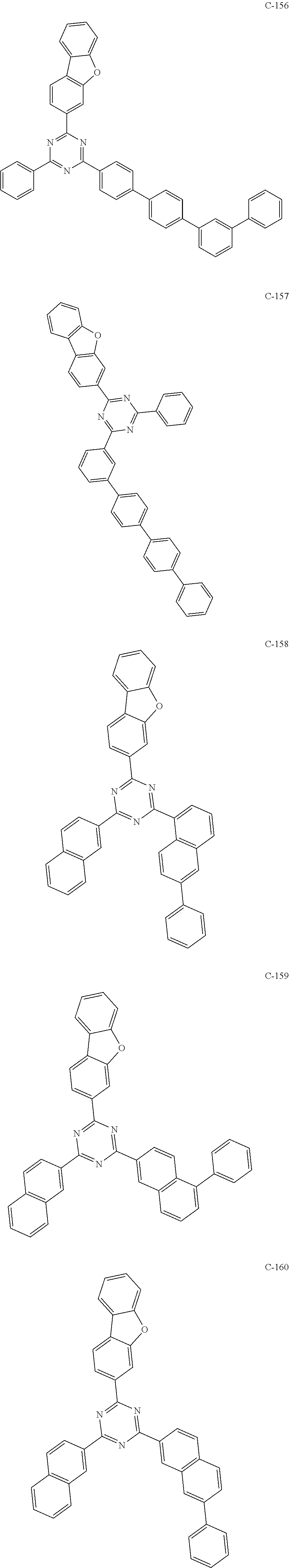 [SMILES: c1ccc(-c2cccc(-c3ccc(-c4ccc(-c5nc(-c6ccccc6)nc(-c6ccc7c(c6)oc6ccccc67)n5)cc4)cc3)c2)cc1 ; c1ccc(-c2ccc(-c3ccc(-c4cccc(-c5nc(-c6ccccc6)nc(-c6ccc7c(c6)oc6ccccc67)n5)c4)cc3)cc2)cc1 ; c1ccc(-c2ccc3c(-c4nc(-c5ccc6ccccc6c5)nc(-c5ccc6c(c5)oc5ccccc56)n4)cccc3c2)cc1 ; c1ccc(-c2cccc3cc(-c4nc(-c5ccc6ccccc6c5)nc(-c5ccc6c(c5)oc5ccccc56)n4)ccc23)cc1 ; c1ccc(-c2ccc3ccc(-c4nc(-c5ccc6ccccc6c5)nc(-c5ccc6c(c5)oc5ccccc56)n4)cc3c2)cc1]

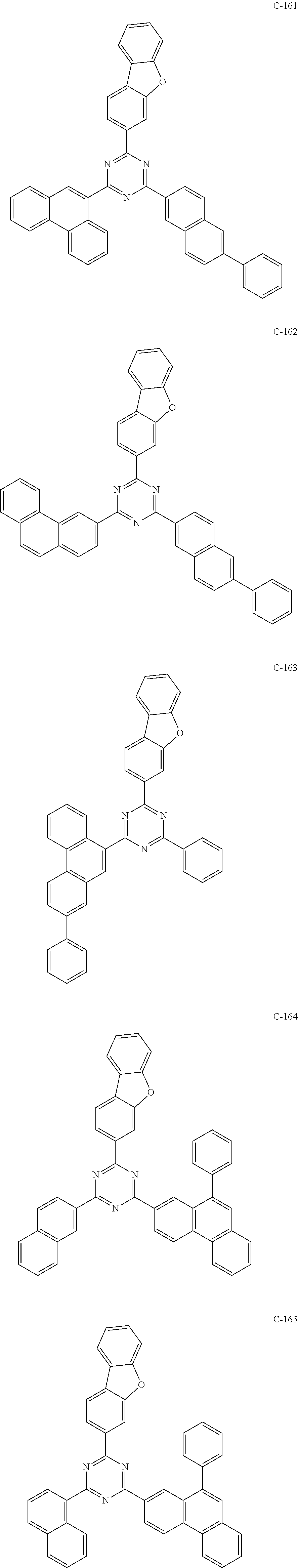 [SMILES: c1ccc(-c2ccc3cc(-c4nc(-c5ccc6c(c5)oc5ccccc56)nc(-c5cc6ccccc6c6ccccc56)n4)ccc3c2)cc1 ; c1ccc(-c2ccc3cc(-c4nc(-c5ccc6c(c5)oc5ccccc56)nc(-c5ccc6ccc7ccccc7c6c5)n4)ccc3c2)cc1 ; c1ccc(-c2ccc3c(c2)cc(-c2nc(-c4ccccc4)nc(-c4ccc5c(c4)oc4ccccc45)n2)c2ccccc23)cc1 ; c1ccc(-c2cc3ccccc3c3ccc(-c4nc(-c5ccc6ccccc6c5)nc(-c5ccc6c(c5)oc5ccccc56)n4)cc23)cc1 ; c1ccc(-c2cc3ccccc3c3ccc(-c4nc(-c5ccc6c(c5)oc5ccccc56)nc(-c5cccc6ccccc56)n4)cc23)cc1]

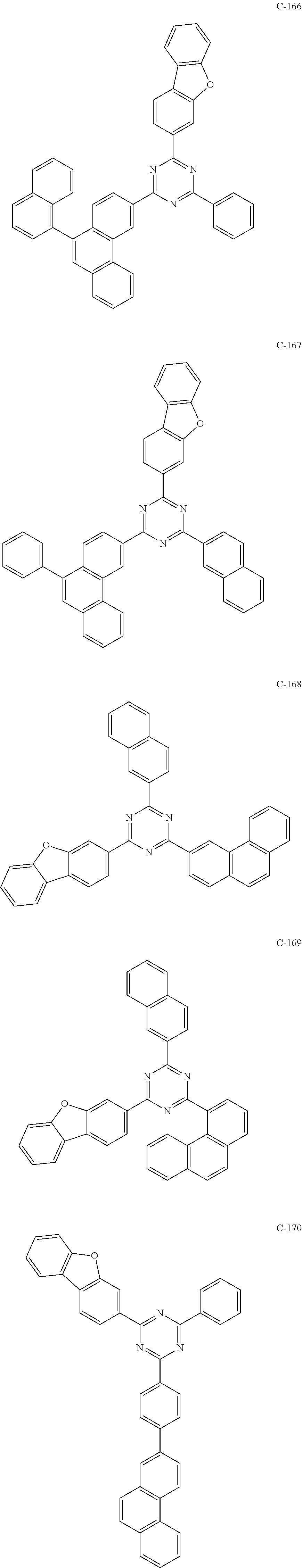 [SMILES: c1ccc(-c2nc(-c3ccc4c(c3)oc3ccccc34)nc(-c3ccc4c(-c5cccc6ccccc56)cc5ccccc5c4c3)n2)cc1 ; c1ccc(-c2cc3ccccc3c3cc(-c4nc(-c5ccc6ccccc6c5)nc(-c5ccc6c(c5)oc5ccccc56)n4)ccc23)cc1 ; c1ccc2cc(-c3nc(-c4ccc5c(c4)oc4ccccc45)nc(-c4ccc5ccc6ccccc6c5c4)n3)ccc2c1 ; c1ccc2cc(-c3nc(-c4ccc5c(c4)oc4ccccc45)nc(-c4cccc5ccc6ccccc6c45)n3)ccc2c1 ; c1ccc(-c2nc(-c3ccc(-c4ccc5c(ccc6ccccc65)c4)cc3)nc(-c3ccc4c(c3)oc3ccccc34)n2)cc1]

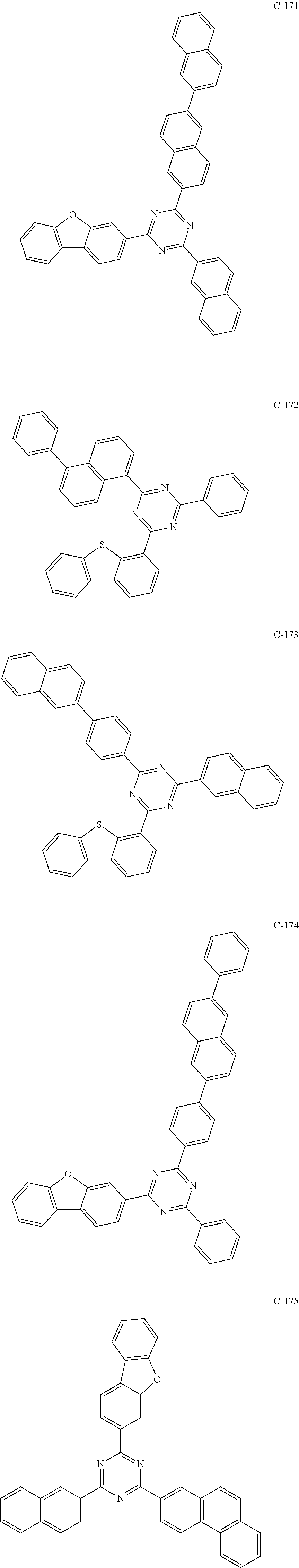 [SMILES: c1ccc2cc(-c3ccc4cc(-c5nc(-c6ccc7ccccc7c6)nc(-c6ccc7c(c6)oc6ccccc67)n5)ccc4c3)ccc2c1 ; c1ccc(-c2nc(-c3cccc4c(-c5ccccc5)cccc34)nc(-c3cccc4c3sc3ccccc34)n2)cc1 ; c1ccc2cc(-c3ccc(-c4nc(-c5ccc6ccccc6c5)nc(-c5cccc6c5sc5ccccc56)n4)cc3)ccc2c1 ; c1ccc(-c2ccc3cc(-c4ccc(-c5nc(-c6ccccc6)nc(-c6ccc7c(c6)oc6ccccc67)n5)cc4)ccc3c2)cc1 ; c1ccc2cc(-c3nc(-c4ccc5c(ccc6ccccc65)c4)nc(-c4ccc5c(c4)oc4ccccc45)n3)ccc2c1]

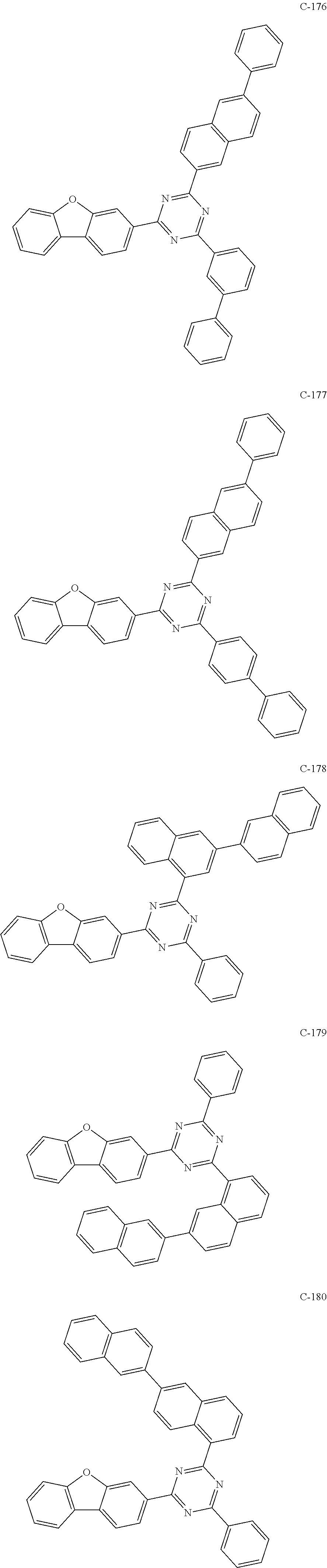 [SMILES: c1ccc(-c2cccc(-c3nc(-c4ccc5cc(-c6ccccc6)ccc5c4)nc(-c4ccc5c(c4)oc4ccccc45)n3)c2)cc1 ; c1ccc(-c2ccc(-c3nc(-c4ccc5cc(-c6ccccc6)ccc5c4)nc(-c4ccc5c(c4)oc4ccccc45)n3)cc2)cc1 ; c1ccc(-c2nc(-c3ccc4c(c3)oc3ccccc34)nc(-c3cc(-c4ccc5ccccc5c4)cc4ccccc34)n2)cc1 ; c1ccc(-c2nc(-c3ccc4c(c3)oc3ccccc34)nc(-c3cccc4ccc(-c5ccc6ccccc6c5)cc34)n2)cc1 ; c1ccc(-c2nc(-c3ccc4c(c3)oc3ccccc34)nc(-c3cccc4cc(-c5ccc6ccccc6c5)ccc34)n2)cc1]

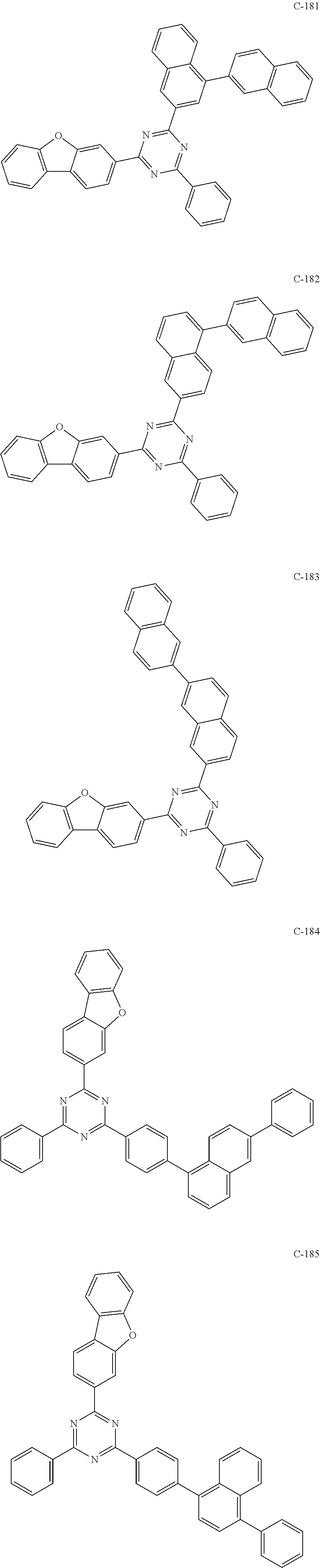 [SMILES: c1ccc(-c2nc(-c3cc(-c4ccc5ccccc5c4)c4ccccc4c3)nc(-c3ccc4c(c3)oc3ccccc34)n2)cc1 ; c1ccc(-c2nc(-c3ccc4c(-c5ccc6ccccc6c5)cccc4c3)nc(-c3ccc4c(c3)oc3ccccc34)n2)cc1 ; c1ccc(-c2nc(-c3ccc4ccc(-c5ccc6ccccc6c5)cc4c3)nc(-c3ccc4c(c3)oc3ccccc34)n2)cc1 ; c1ccc(-c2ccc3c(-c4ccc(-c5nc(-c6ccccc6)nc(-c6ccc7c(c6)oc6ccccc67)n5)cc4)cccc3c2)cc1 ; c1ccc(-c2nc(-c3ccc(-c4ccc(-c5ccccc5)c5ccccc45)cc3)nc(-c3ccc4c(c3)oc3ccccc34)n2)cc1]

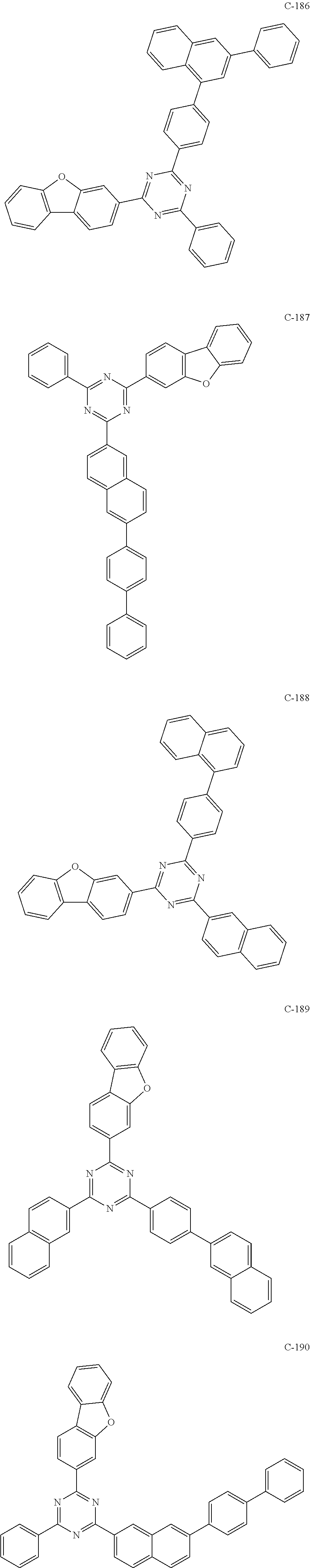 [SMILES: c1ccc(-c2cc(-c3ccc(-c4nc(-c5ccccc5)nc(-c5ccc6c(c5)oc5ccccc56)n4)cc3)c3ccccc3c2)cc1 ; c1ccc(-c2ccc(-c3ccc4cc(-c5nc(-c6ccccc6)nc(-c6ccc7c(c6)oc6ccccc67)n5)ccc4c3)cc2)cc1 ; c1ccc2cc(-c3nc(-c4ccc(-c5cccc6ccccc56)cc4)nc(-c4ccc5c(c4)oc4ccccc45)n3)ccc2c1 ; c1ccc2cc(-c3ccc(-c4nc(-c5ccc6ccccc6c5)nc(-c5ccc6c(c5)oc5ccccc56)n4)cc3)ccc2c1 ; c1ccc(-c2ccc(-c3ccc4ccc(-c5nc(-c6ccccc6)nc(-c6ccc7c(c6)oc6ccccc67)n5)cc4c3)cc2)cc1]

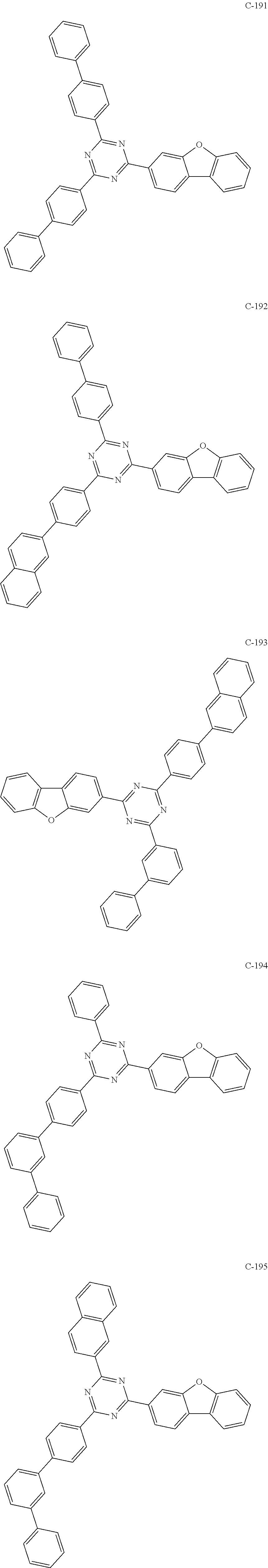 [SMILES: c1ccc(-c2ccc(-c3nc(-c4ccc(-c5ccccc5)cc4)nc(-c4ccc5c(c4)oc4ccccc45)n3)cc2)cc1 ; c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2cccc(-c3nc(-c4ccc(-c5ccc6ccccc6c5)cc4)nc(-c4ccc5c(c4)oc4ccccc45)n3)c2)cc1 ; c1ccc(-c2cccc(-c3ccc(-c4nc(-c5ccccc5)nc(-c5ccc6c(c5)oc5ccccc56)n4)cc3)c2)cc1 ; c1ccc(-c2cccc(-c3ccc(-c4nc(-c5ccc6ccccc6c5)nc(-c5ccc6c(c5)oc5ccccc56)n4)cc3)c2)cc1]

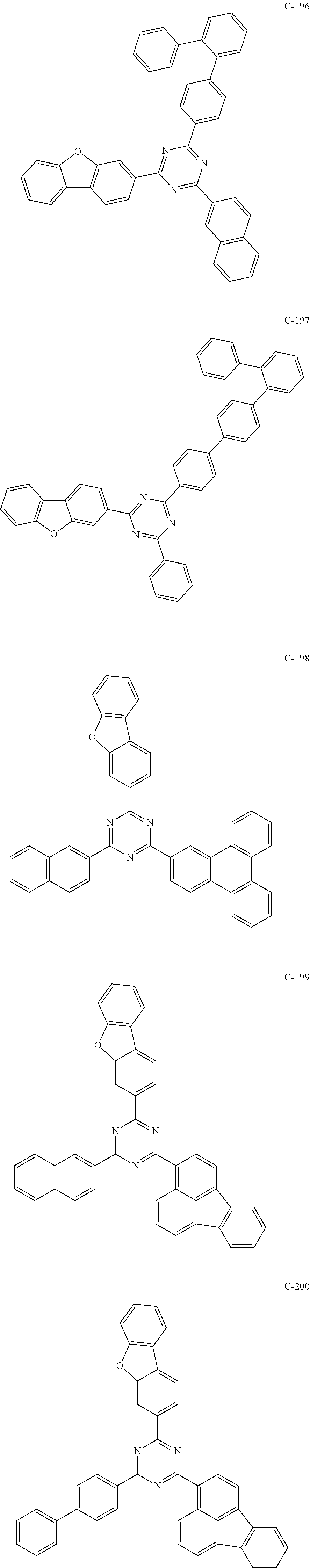 [SMILES: c1ccc(-c2ccccc2-c2ccc(-c3nc(-c4ccc5ccccc5c4)nc(-c4ccc5c(c4)oc4ccccc45)n3)cc2)cc1 ; c1ccc(-c2nc(-c3ccc(-c4ccc(-c5ccccc5-c5ccccc5)cc4)cc3)nc(-c3ccc4c(c3)oc3ccccc34)n2)cc1 ; c1ccc2cc(-c3nc(-c4ccc5c(c4)oc4ccccc45)nc(-c4ccc5c6ccccc6c6ccccc6c5c4)n3)ccc2c1 ; c1ccc2c(c1)-c1cccc3c(-c4nc(-c5ccc6ccccc6c5)nc(-c5ccc6c(c5)oc5ccccc56)n4)ccc-2c13 ; c1ccc(-c2ccc(-c3nc(-c4ccc5c(c4)oc4ccccc45)nc(-c4ccc5c6c(cccc46)-c4ccccc4-5)n3)cc2)cc1]

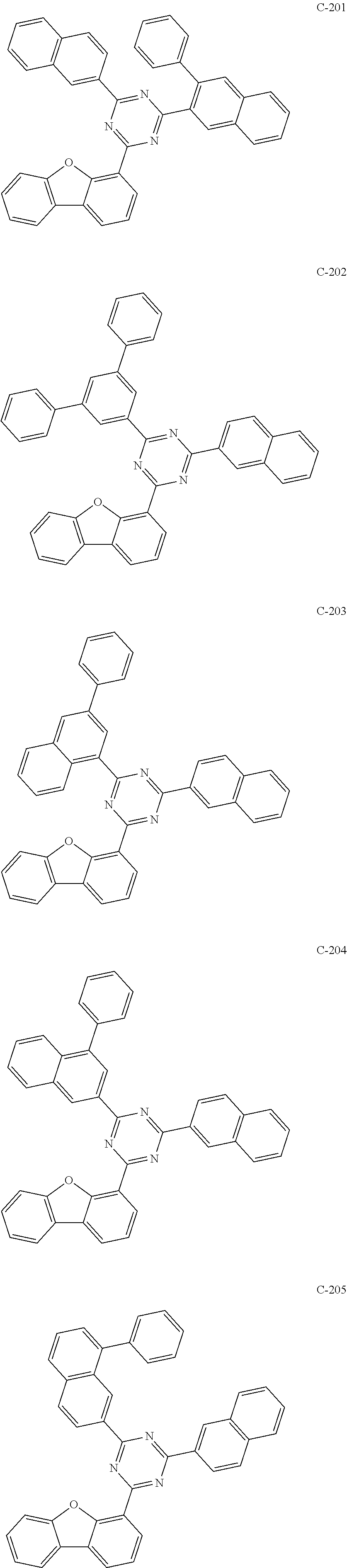 [SMILES: c1ccc(-c2cc3ccccc3cc2-c2nc(-c3ccc4ccccc4c3)nc(-c3cccc4c3oc3ccccc34)n2)cc1 ; c1ccc(-c2cc(-c3ccccc3)cc(-c3nc(-c4ccc5ccccc5c4)nc(-c4cccc5c4oc4ccccc45)n3)c2)cc1 ; c1ccc(-c2cc(-c3nc(-c4ccc5ccccc5c4)nc(-c4cccc5c4oc4ccccc45)n3)c3ccccc3c2)cc1 ; c1ccc(-c2cc(-c3nc(-c4ccc5ccccc5c4)nc(-c4cccc5c4oc4ccccc45)n3)cc3ccccc23)cc1 ; c1ccc(-c2cccc3ccc(-c4nc(-c5ccc6ccccc6c5)nc(-c5cccc6c5oc5ccccc56)n4)cc23)cc1]

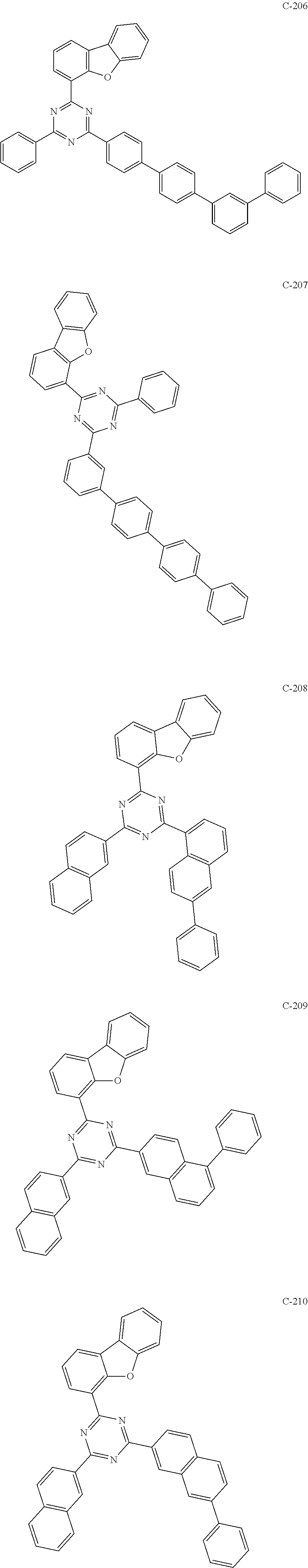 [SMILES: c1ccc(-c2cccc(-c3ccc(-c4ccc(-c5nc(-c6ccccc6)nc(-c6cccc7c6oc6ccccc67)n5)cc4)cc3)c2)cc1 ; c1ccc(-c2ccc(-c3ccc(-c4cccc(-c5nc(-c6ccccc6)nc(-c6cccc7c6oc6ccccc67)n5)c4)cc3)cc2)cc1 ; c1ccc(-c2ccc3c(-c4nc(-c5ccc6ccccc6c5)nc(-c5cccc6c5oc5ccccc56)n4)cccc3c2)cc1 ; c1ccc(-c2cccc3cc(-c4nc(-c5ccc6ccccc6c5)nc(-c5cccc6c5oc5ccccc56)n4)ccc23)cc1 ; c1ccc(-c2ccc3ccc(-c4nc(-c5ccc6ccccc6c5)nc(-c5cccc6c5oc5ccccc56)n4)cc3c2)cc1]

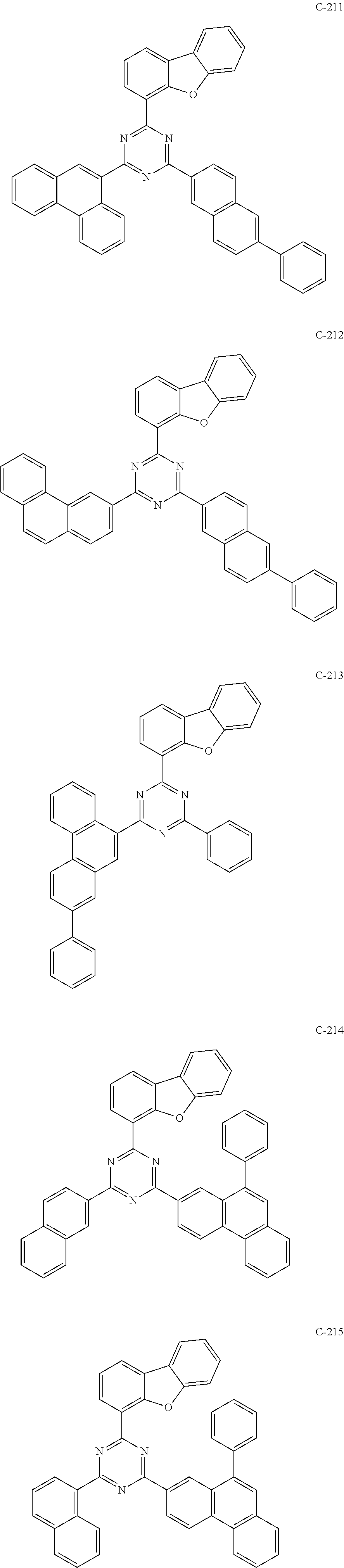 [SMILES: c1ccc(-c2ccc3cc(-c4nc(-c5cc6ccccc6c6ccccc56)nc(-c5cccc6c5oc5ccccc56)n4)ccc3c2)cc1 ; c1ccc(-c2ccc3cc(-c4nc(-c5ccc6ccc7ccccc7c6c5)nc(-c5cccc6c5oc5ccccc56)n4)ccc3c2)cc1 ; c1ccc(-c2ccc3c(c2)cc(-c2nc(-c4ccccc4)nc(-c4cccc5c4oc4ccccc45)n2)c2ccccc23)cc1 ; c1ccc(-c2cc3ccccc3c3ccc(-c4nc(-c5ccc6ccccc6c5)nc(-c5cccc6c5oc5ccccc56)n4)cc23)cc1 ; c1ccc(-c2cc3ccccc3c3ccc(-c4nc(-c5cccc6ccccc56)nc(-c5cccc6c5oc5ccccc56)n4)cc23)cc1]

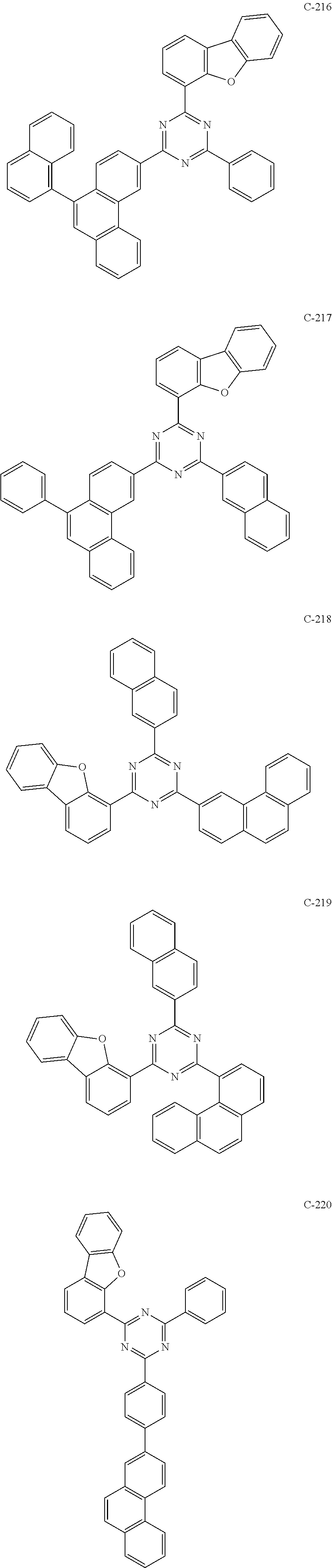 [SMILES: c1ccc(-c2nc(-c3ccc4c(-c5cccc6ccccc56)cc5ccccc5c4c3)nc(-c3cccc4c3oc3ccccc34)n2)cc1 ; c1ccc(-c2cc3ccccc3c3cc(-c4nc(-c5ccc6ccccc6c5)nc(-c5cccc6c5oc5ccccc56)n4)ccc23)cc1 ; c1ccc2cc(-c3nc(-c4ccc5ccc6ccccc6c5c4)nc(-c4cccc5c4oc4ccccc45)n3)ccc2c1 ; c1ccc2cc(-c3nc(-c4cccc5c4oc4ccccc45)nc(-c4cccc5ccc6ccccc6c45)n3)ccc2c1 ; c1ccc(-c2nc(-c3ccc(-c4ccc5c(ccc6ccccc65)c4)cc3)nc(-c3cccc4c3oc3ccccc34)n2)cc1]

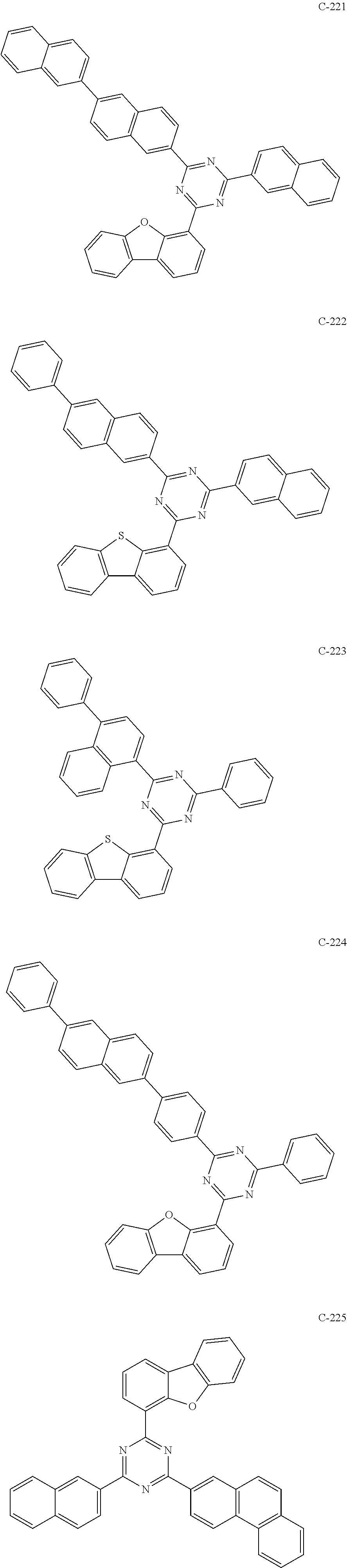 [SMILES: c1ccc2cc(-c3ccc4cc(-c5nc(-c6ccc7ccccc7c6)nc(-c6cccc7c6oc6ccccc67)n5)ccc4c3)ccc2c1 ; c1ccc(-c2ccc3cc(-c4nc(-c5ccc6ccccc6c5)nc(-c5cccc6c5sc5ccccc56)n4)ccc3c2)cc1 ; c1ccc(-c2nc(-c3ccc(-c4ccccc4)c4ccccc34)nc(-c3cccc4c3sc3ccccc34)n2)cc1 ; c1ccc(-c2ccc3cc(-c4ccc(-c5nc(-c6ccccc6)nc(-c6cccc7c6oc6ccccc67)n5)cc4)ccc3c2)cc1 ; c1ccc2cc(-c3nc(-c4ccc5c(ccc6ccccc65)c4)nc(-c4cccc5c4oc4ccccc45)n3)ccc2c1]

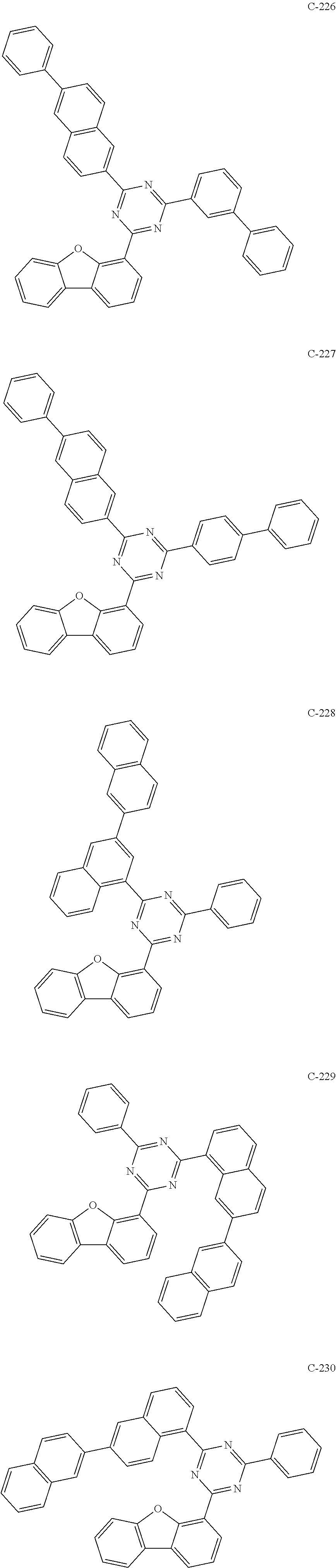 [SMILES: c1ccc(-c2cccc(-c3nc(-c4ccc5cc(-c6ccccc6)ccc5c4)nc(-c4cccc5c4oc4ccccc45)n3)c2)cc1 ; c1ccc(-c2ccc(-c3nc(-c4ccc5cc(-c6ccccc6)ccc5c4)nc(-c4cccc5c4oc4ccccc45)n3)cc2)cc1 ; c1ccc(-c2nc(-c3cc(-c4ccc5ccccc5c4)cc4ccccc34)nc(-c3cccc4c3oc3ccccc34)n2)cc1 ; c1ccc(-c2nc(-c3cccc4ccc(-c5ccc6ccccc6c5)cc34)nc(-c3cccc4c3oc3ccccc34)n2)cc1 ; c1ccc(-c2nc(-c3cccc4cc(-c5ccc6ccccc6c5)ccc34)nc(-c3cccc4c3oc3ccccc34)n2)cc1]

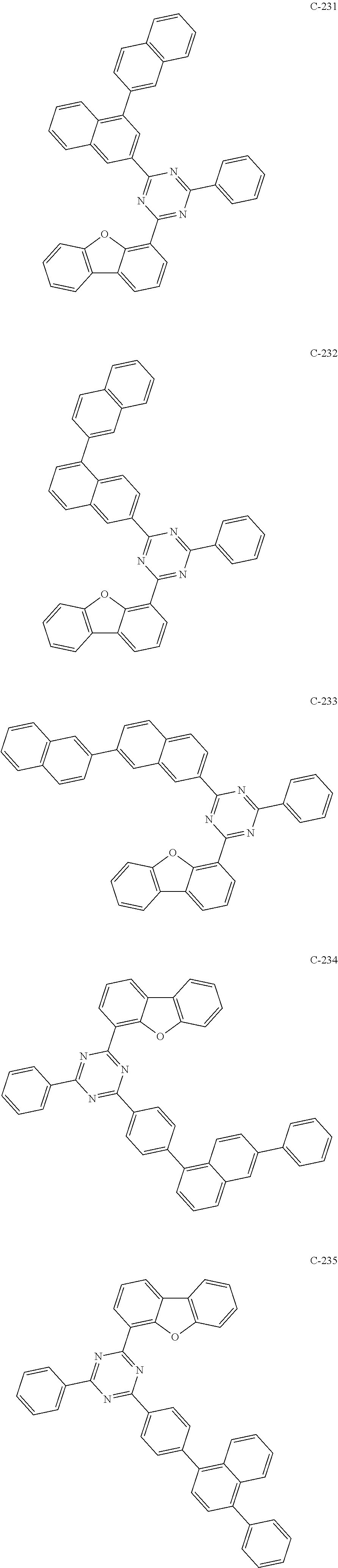 [SMILES: c1ccc(-c2nc(-c3cc(-c4ccc5ccccc5c4)c4ccccc4c3)nc(-c3cccc4c3oc3ccccc34)n2)cc1 ; c1ccc(-c2nc(-c3ccc4c(-c5ccc6ccccc6c5)cccc4c3)nc(-c3cccc4c3oc3ccccc34)n2)cc1 ; c1ccc(-c2nc(-c3ccc4ccc(-c5ccc6ccccc6c5)cc4c3)nc(-c3cccc4c3oc3ccccc34)n2)cc1 ; c1ccc(-c2ccc3c(-c4ccc(-c5nc(-c6ccccc6)nc(-c6cccc7c6oc6ccccc67)n5)cc4)cccc3c2)cc1 ; c1ccc(-c2nc(-c3ccc(-c4ccc(-c5ccccc5)c5ccccc45)cc3)nc(-c3cccc4c3oc3ccccc34)n2)cc1]

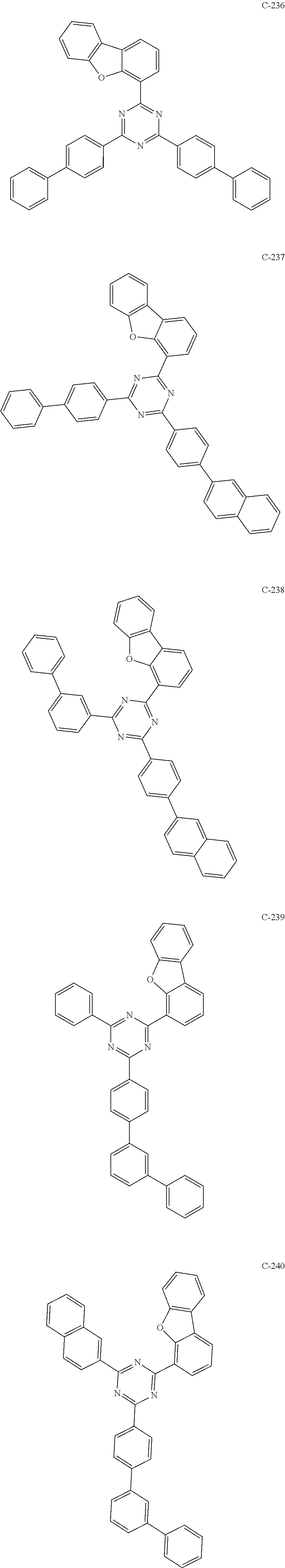 [SMILES: c1ccc(-c2ccc(-c3nc(-c4ccc(-c5ccccc5)cc4)nc(-c4cccc5c4oc4ccccc45)n3)cc2)cc1 ; c1ccc(-c2ccc(-c3nc(-c4ccc(-c5ccc6ccccc6c5)cc4)nc(-c4cccc5c4oc4ccccc45)n3)cc2)cc1 ; c1ccc(-c2cccc(-c3nc(-c4ccc(-c5ccc6ccccc6c5)cc4)nc(-c4cccc5c4oc4ccccc45)n3)c2)cc1 ; c1ccc(-c2cccc(-c3ccc(-c4nc(-c5ccccc5)nc(-c5cccc6c5oc5ccccc56)n4)cc3)c2)cc1 ; c1ccc(-c2cccc(-c3ccc(-c4nc(-c5ccc6ccccc6c5)nc(-c5cccc6c5oc5ccccc56)n4)cc3)c2)cc1]

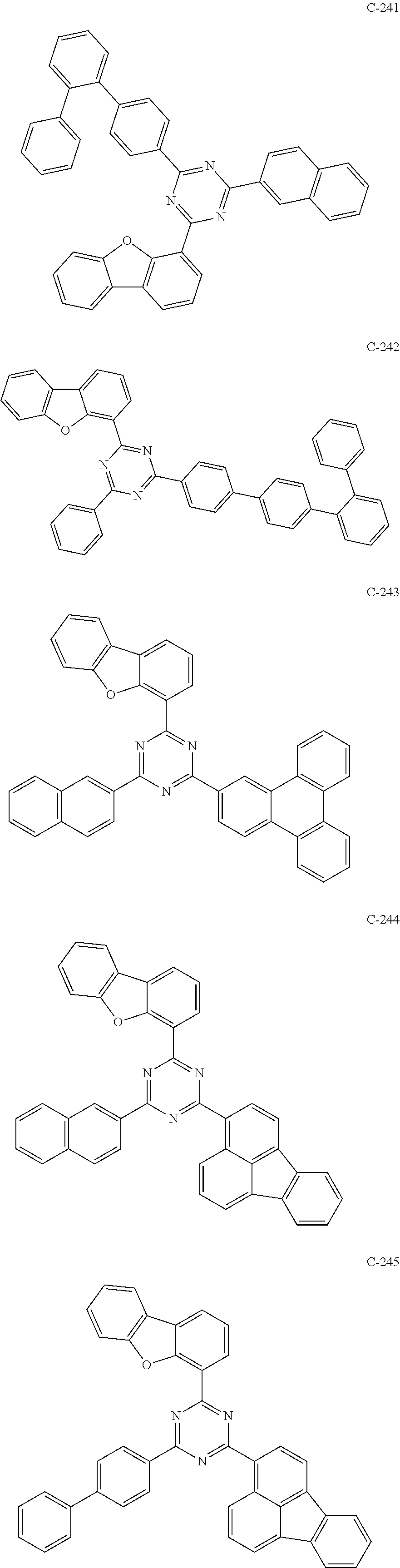 [SMILES: c1ccc(-c2ccccc2-c2ccc(-c3nc(-c4ccc5ccccc5c4)nc(-c4cccc5c4oc4ccccc45)n3)cc2)cc1 ; c1ccc(-c2nc(-c3ccc(-c4ccc(-c5ccccc5-c5ccccc5)cc4)cc3)nc(-c3cccc4c3oc3ccccc34)n2)cc1 ; c1ccc2cc(-c3nc(-c4ccc5c6ccccc6c6ccccc6c5c4)nc(-c4cccc5c4oc4ccccc45)n3)ccc2c1 ; c1ccc2c(c1)-c1cccc3c(-c4nc(-c5ccc6ccccc6c5)nc(-c5cccc6c5oc5ccccc56)n4)ccc-2c13 ; c1ccc(-c2ccc(-c3nc(-c4ccc5c6c(cccc46)-c4ccccc4-5)nc(-c4cccc5c4oc4ccccc45)n3)cc2)cc1]

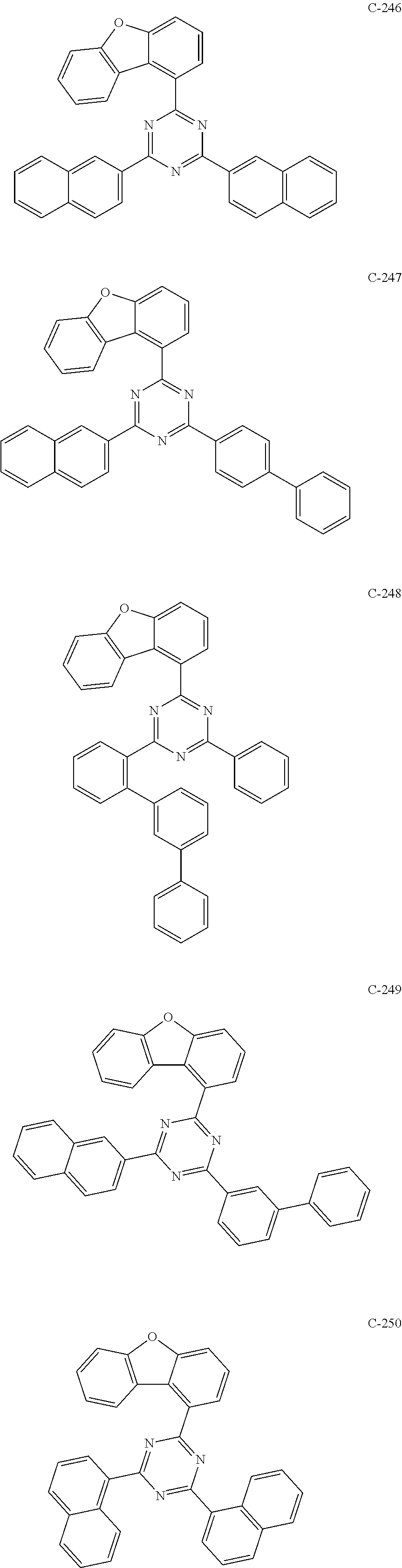 [SMILES: c1ccc2cc(-c3nc(-c4ccc5ccccc5c4)nc(-c4cccc5oc6ccccc6c45)n3)ccc2c1 ; c1ccc(-c2ccc(-c3nc(-c4ccc5ccccc5c4)nc(-c4cccc5oc6ccccc6c45)n3)cc2)cc1 ; c1ccc(-c2cccc(-c3ccccc3-c3nc(-c4ccccc4)nc(-c4cccc5oc6ccccc6c45)n3)c2)cc1 ; c1ccc(-c2cccc(-c3nc(-c4ccc5ccccc5c4)nc(-c4cccc5oc6ccccc6c45)n3)c2)cc1 ; c1ccc2c(-c3nc(-c4cccc5ccccc45)nc(-c4cccc5oc6ccccc6c45)n3)cccc2c1]

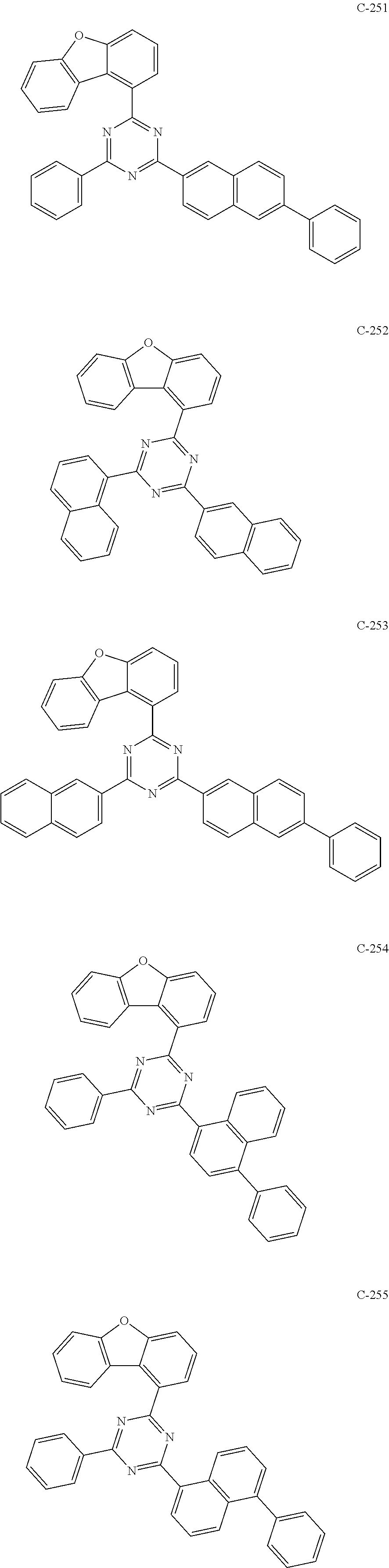 [SMILES: c1ccc(-c2ccc3cc(-c4nc(-c5ccccc5)nc(-c5cccc6oc7ccccc7c56)n4)ccc3c2)cc1 ; c1ccc2cc(-c3nc(-c4cccc5ccccc45)nc(-c4cccc5oc6ccccc6c45)n3)ccc2c1 ; c1ccc(-c2ccc3cc(-c4nc(-c5ccc6ccccc6c5)nc(-c5cccc6oc7ccccc7c56)n4)ccc3c2)cc1 ; c1ccc(-c2nc(-c3ccc(-c4ccccc4)c4ccccc34)nc(-c3cccc4oc5ccccc5c34)n2)cc1 ; c1ccc(-c2nc(-c3cccc4c(-c5ccccc5)cccc34)nc(-c3cccc4oc5ccccc5c34)n2)cc1]

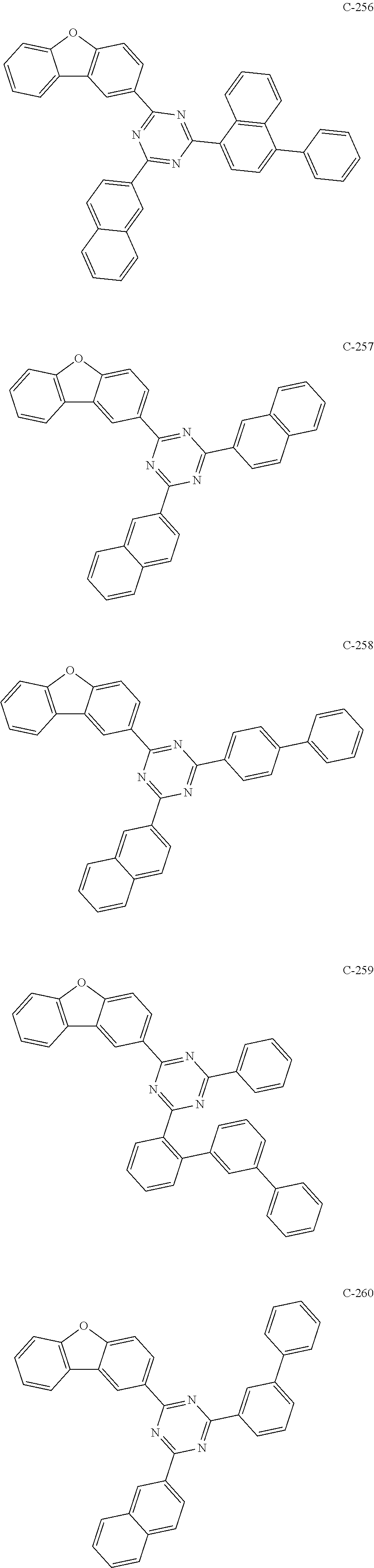 [SMILES: c1ccc(-c2ccc(-c3nc(-c4ccc5ccccc5c4)nc(-c4ccc5oc6ccccc6c5c4)n3)c3ccccc23)cc1 ; c1ccc2cc(-c3nc(-c4ccc5ccccc5c4)nc(-c4ccc5oc6ccccc6c5c4)n3)ccc2c1 ; c1ccc(-c2ccc(-c3nc(-c4ccc5ccccc5c4)nc(-c4ccc5oc6ccccc6c5c4)n3)cc2)cc1 ; c1ccc(-c2cccc(-c3ccccc3-c3nc(-c4ccccc4)nc(-c4ccc5oc6ccccc6c5c4)n3)c2)cc1 ; c1ccc(-c2cccc(-c3nc(-c4ccc5ccccc5c4)nc(-c4ccc5oc6ccccc6c5c4)n3)c2)cc1]

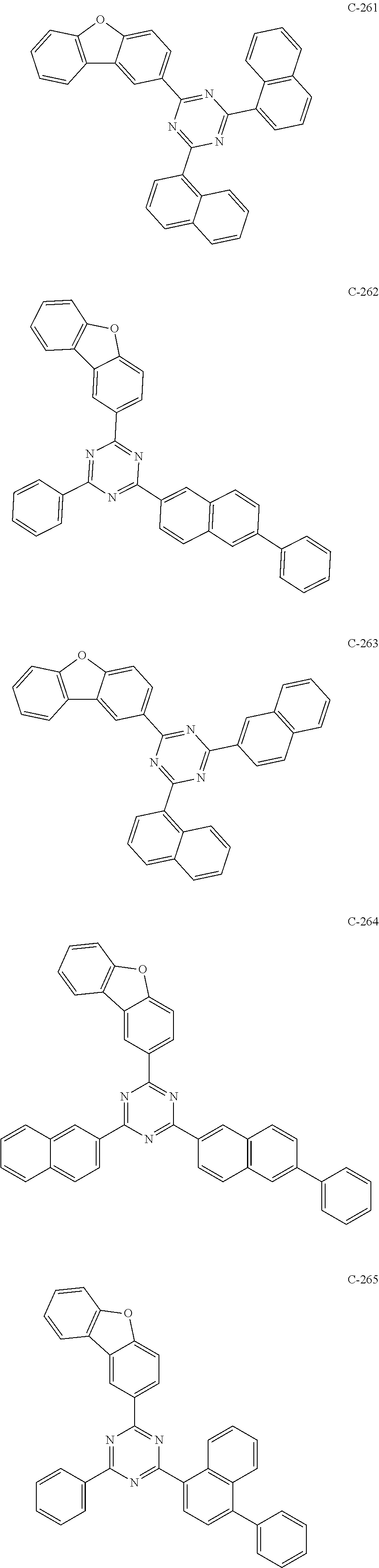 [SMILES: c1ccc2c(-c3nc(-c4ccc5oc6ccccc6c5c4)nc(-c4cccc5ccccc45)n3)cccc2c1 ; c1ccc(-c2ccc3cc(-c4nc(-c5ccccc5)nc(-c5ccc6oc7ccccc7c6c5)n4)ccc3c2)cc1 ; c1ccc2cc(-c3nc(-c4ccc5oc6ccccc6c5c4)nc(-c4cccc5ccccc45)n3)ccc2c1 ; c1ccc(-c2ccc3cc(-c4nc(-c5ccc6ccccc6c5)nc(-c5ccc6oc7ccccc7c6c5)n4)ccc3c2)cc1 ; c1ccc(-c2nc(-c3ccc4oc5ccccc5c4c3)nc(-c3ccc(-c4ccccc4)c4ccccc34)n2)cc1]

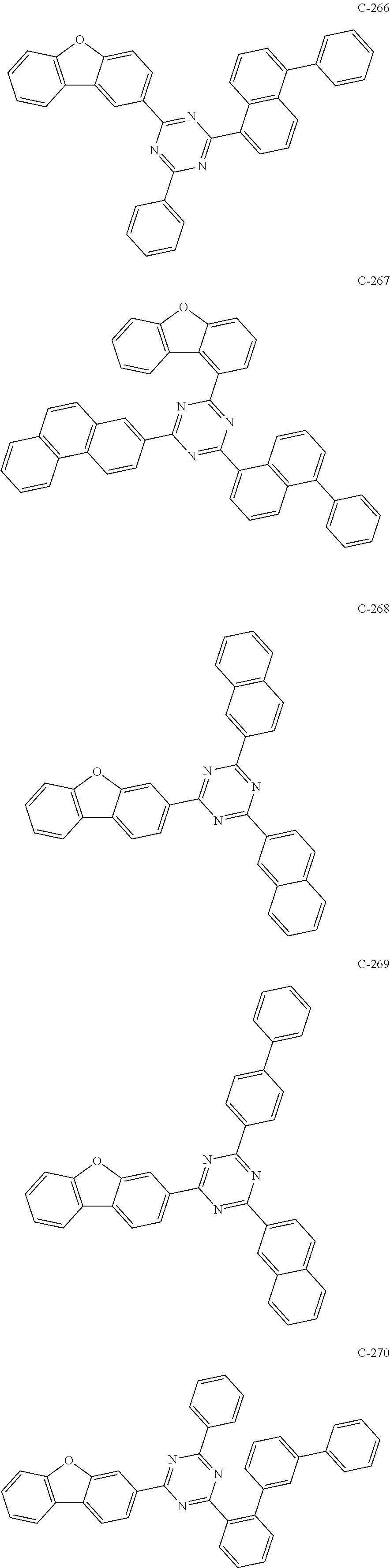 [SMILES: c1ccc(-c2nc(-c3ccc4oc5ccccc5c4c3)nc(-c3cccc4c(-c5ccccc5)cccc34)n2)cc1 ; c1ccc(-c2cccc3c(-c4nc(-c5ccc6c(ccc7ccccc76)c5)nc(-c5cccc6oc7ccccc7c56)n4)cccc23)cc1 ; c1ccc2cc(-c3nc(-c4ccc5ccccc5c4)nc(-c4ccc5c(c4)oc4ccccc45)n3)ccc2c1 ; c1ccc(-c2ccc(-c3nc(-c4ccc5ccccc5c4)nc(-c4ccc5c(c4)oc4ccccc45)n3)cc2)cc1 ; c1ccc(-c2cccc(-c3ccccc3-c3nc(-c4ccccc4)nc(-c4ccc5c(c4)oc4ccccc45)n3)c2)cc1]

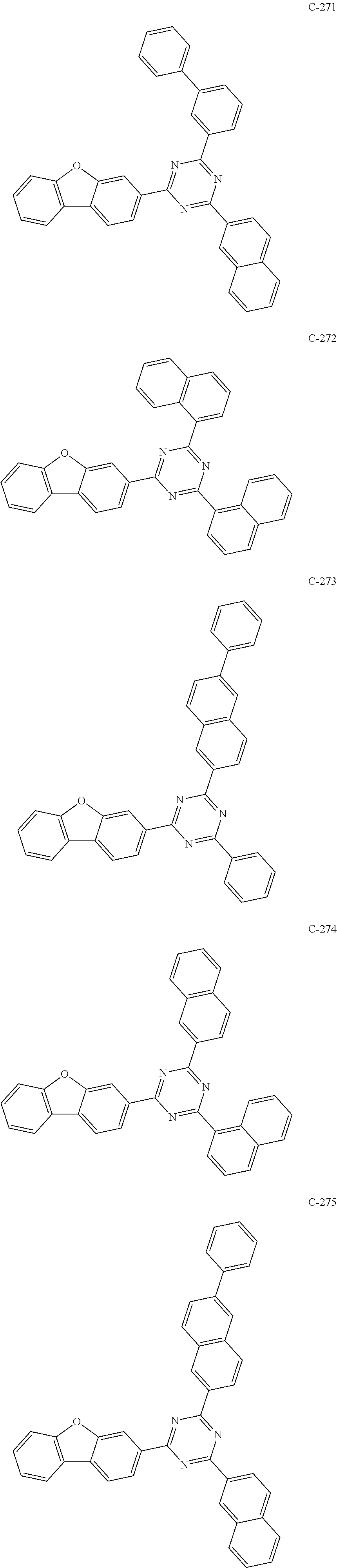 [SMILES: c1ccc(-c2cccc(-c3nc(-c4ccc5ccccc5c4)nc(-c4ccc5c(c4)oc4ccccc45)n3)c2)cc1 ; c1ccc2c(-c3nc(-c4ccc5c(c4)oc4ccccc45)nc(-c4cccc5ccccc45)n3)cccc2c1 ; c1ccc(-c2ccc3cc(-c4nc(-c5ccccc5)nc(-c5ccc6c(c5)oc5ccccc56)n4)ccc3c2)cc1 ; c1ccc2cc(-c3nc(-c4ccc5c(c4)oc4ccccc45)nc(-c4cccc5ccccc45)n3)ccc2c1 ; c1ccc(-c2ccc3cc(-c4nc(-c5ccc6ccccc6c5)nc(-c5ccc6c(c5)oc5ccccc56)n4)ccc3c2)cc1]

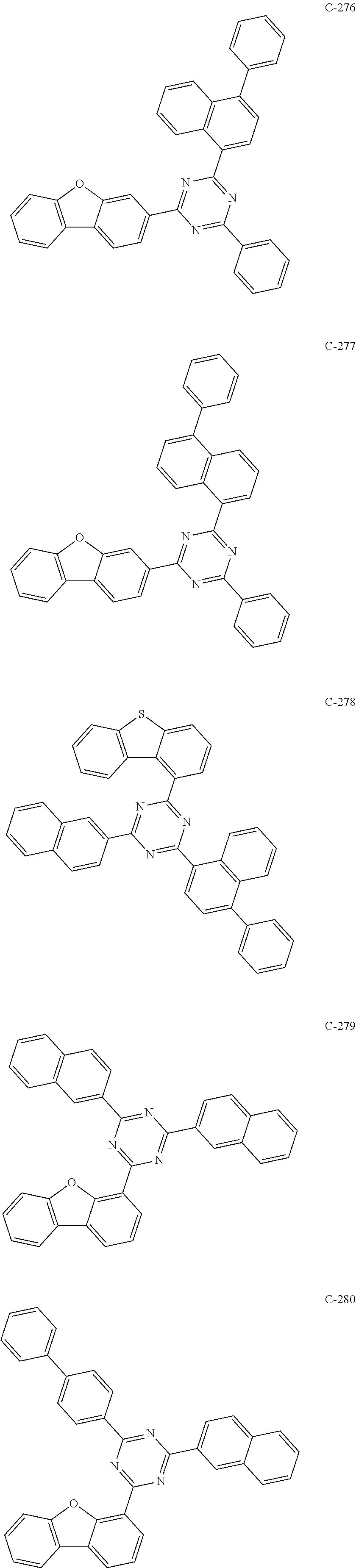 [SMILES: c1ccc(-c2nc(-c3ccc4c(c3)oc3ccccc34)nc(-c3ccc(-c4ccccc4)c4ccccc34)n2)cc1 ; c1ccc(-c2nc(-c3ccc4c(c3)oc3ccccc34)nc(-c3cccc4c(-c5ccccc5)cccc34)n2)cc1 ; c1ccc(-c2ccc(-c3nc(-c4ccc5ccccc5c4)nc(-c4cccc5sc6ccccc6c45)n3)c3ccccc23)cc1 ; c1ccc2cc(-c3nc(-c4ccc5ccccc5c4)nc(-c4cccc5c4oc4ccccc45)n3)ccc2c1 ; c1ccc(-c2ccc(-c3nc(-c4ccc5ccccc5c4)nc(-c4cccc5c4oc4ccccc45)n3)cc2)cc1]

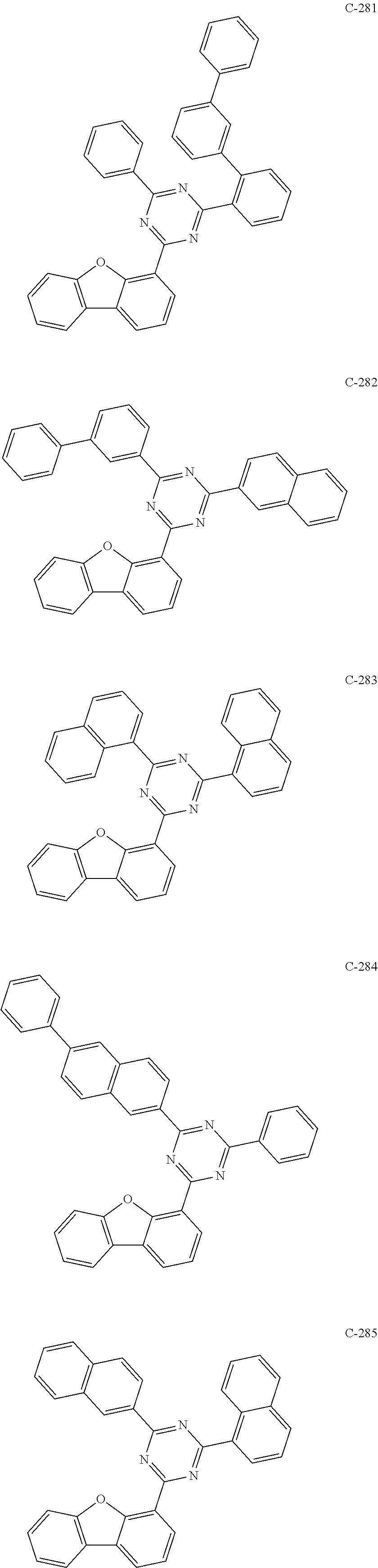 [SMILES: c1ccc(-c2cccc(-c3ccccc3-c3nc(-c4ccccc4)nc(-c4cccc5c4oc4ccccc45)n3)c2)cc1 ; c1ccc(-c2cccc(-c3nc(-c4ccc5ccccc5c4)nc(-c4cccc5c4oc4ccccc45)n3)c2)cc1 ; c1ccc2c(-c3nc(-c4cccc5ccccc45)nc(-c4cccc5c4oc4ccccc45)n3)cccc2c1 ; c1ccc(-c2ccc3cc(-c4nc(-c5ccccc5)nc(-c5cccc6c5oc5ccccc56)n4)ccc3c2)cc1 ; c1ccc2cc(-c3nc(-c4cccc5ccccc45)nc(-c4cccc5c4oc4ccccc45)n3)ccc2c1]

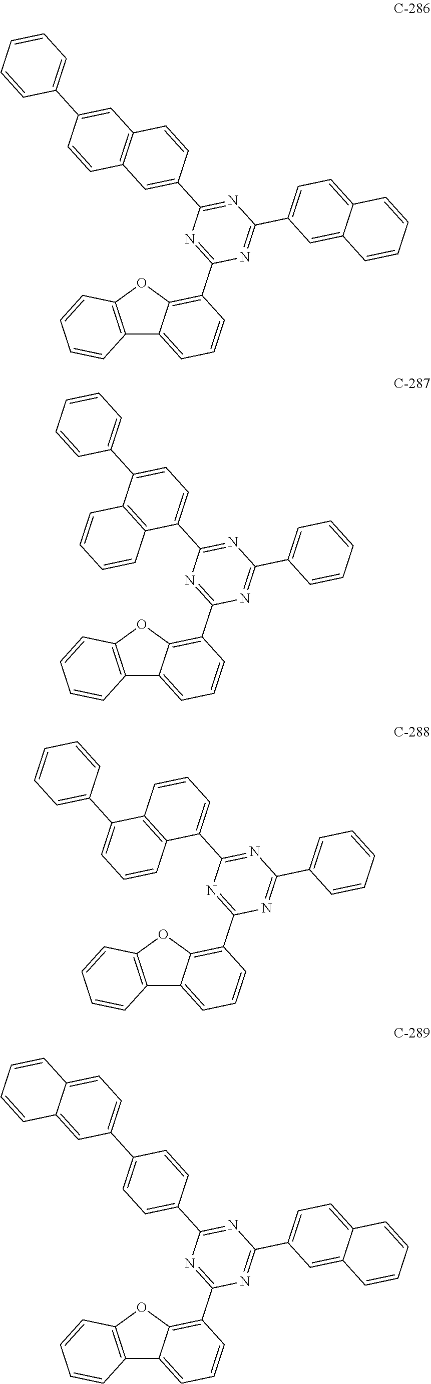

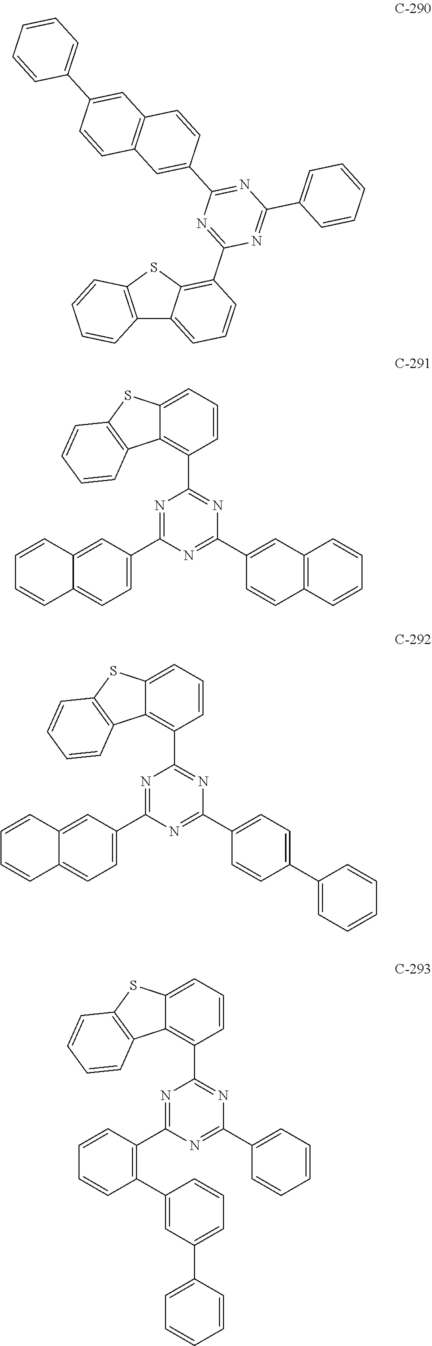

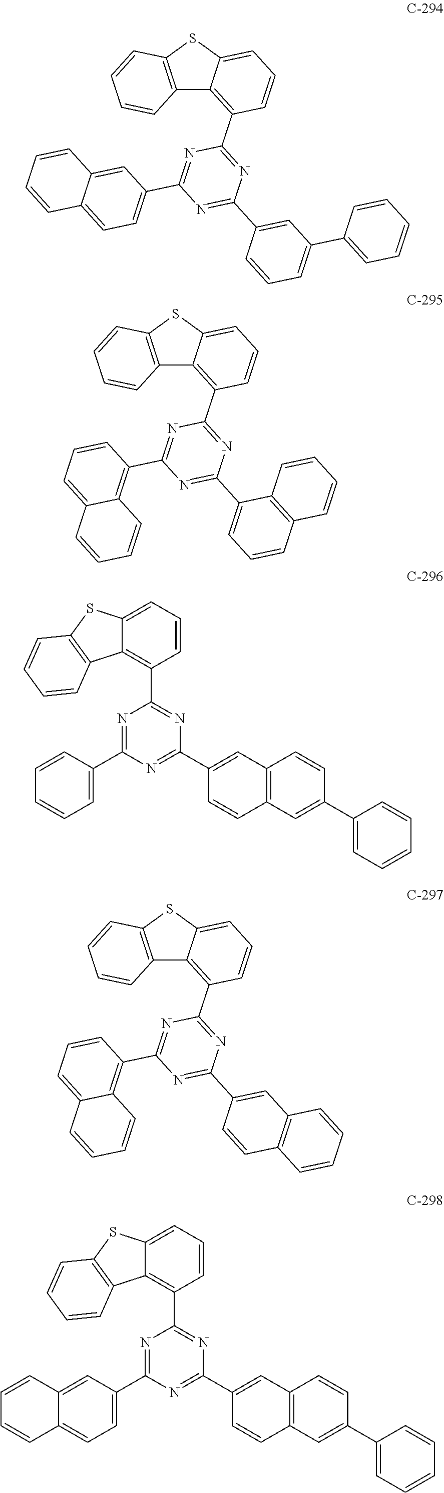

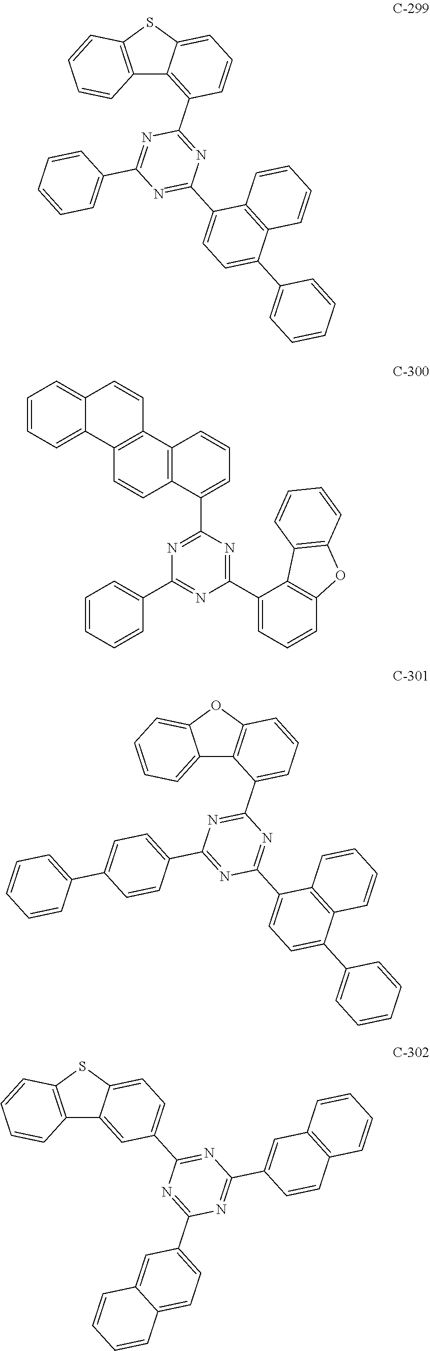 [SMILES: c1ccc(-c2nc(-c3ccc(-c4ccccc4)c4ccccc34)nc(-c3cccc4sc5ccccc5c34)n2)cc1 ; c1ccc(-c2nc(-c3cccc4c3ccc3c5ccccc5ccc43)nc(-c3cccc4oc5ccccc5c34)n2)cc1 ; c1ccc(-c2ccc(-c3nc(-c4ccc(-c5ccccc5)c5ccccc45)nc(-c4cccc5oc6ccccc6c45)n3)cc2)cc1 ; c1ccc2cc(-c3nc(-c4ccc5ccccc5c4)nc(-c4ccc5sc6ccccc6c5c4)n3)ccc2c1]

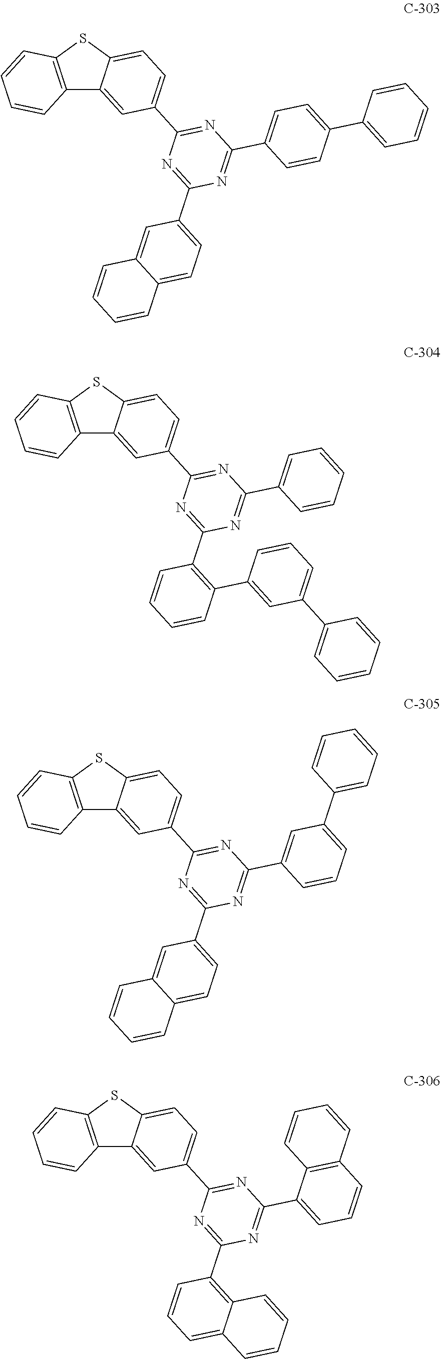 [SMILES: c1ccc(-c2ccc(-c3nc(-c4ccc5ccccc5c4)nc(-c4ccc5sc6ccccc6c5c4)n3)cc2)cc1 ; c1ccc(-c2cccc(-c3ccccc3-c3nc(-c4ccccc4)nc(-c4ccc5sc6ccccc6c5c4)n3)c2)cc1 ; c1ccc(-c2cccc(-c3nc(-c4ccc5ccccc5c4)nc(-c4ccc5sc6ccccc6c5c4)n3)c2)cc1 ; c1ccc2c(-c3nc(-c4ccc5sc6ccccc6c5c4)nc(-c4cccc5ccccc45)n3)cccc2c1]

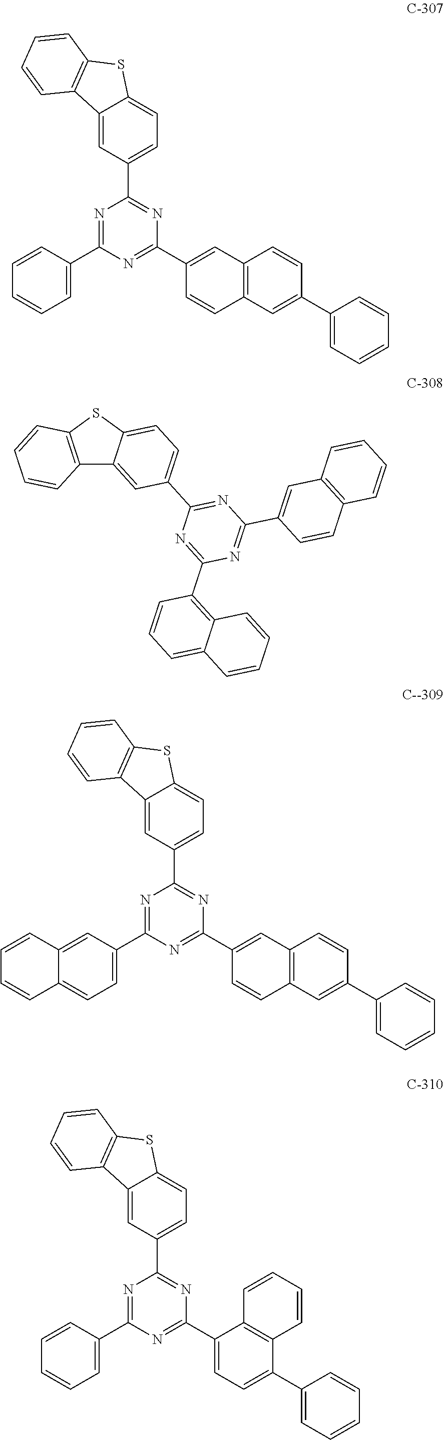 [SMILES: c1ccc(-c2ccc3cc(-c4nc(-c5ccccc5)nc(-c5ccc6sc7ccccc7c6c5)n4)ccc3c2)cc1 ; c1ccc2cc(-c3nc(-c4ccc5sc6ccccc6c5c4)nc(-c4cccc5ccccc45)n3)ccc2c1 ; c1ccc(-c2ccc3cc(-c4nc(-c5ccc6ccccc6c5)nc(-c5ccc6sc7ccccc7c6c5)n4)ccc3c2)cc1 ; c1ccc(-c2nc(-c3ccc4sc5ccccc5c4c3)nc(-c3ccc(-c4ccccc4)c4ccccc34)n2)cc1]

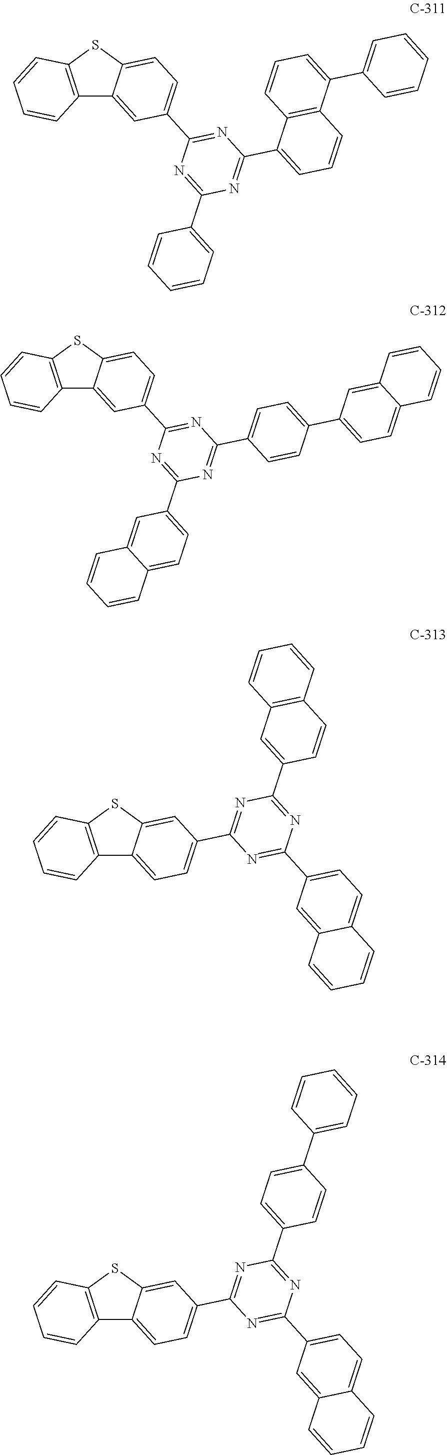 [SMILES: c1ccc(-c2nc(-c3ccc4sc5ccccc5c4c3)nc(-c3cccc4c(-c5ccccc5)cccc34)n2)cc1 ; c1ccc2cc(-c3ccc(-c4nc(-c5ccc6ccccc6c5)nc(-c5ccc6sc7ccccc7c6c5)n4)cc3)ccc2c1 ; c1ccc2cc(-c3nc(-c4ccc5ccccc5c4)nc(-c4ccc5c(c4)sc4ccccc45)n3)ccc2c1 ; c1ccc(-c2ccc(-c3nc(-c4ccc5ccccc5c4)nc(-c4ccc5c(c4)sc4ccccc45)n3)cc2)cc1]

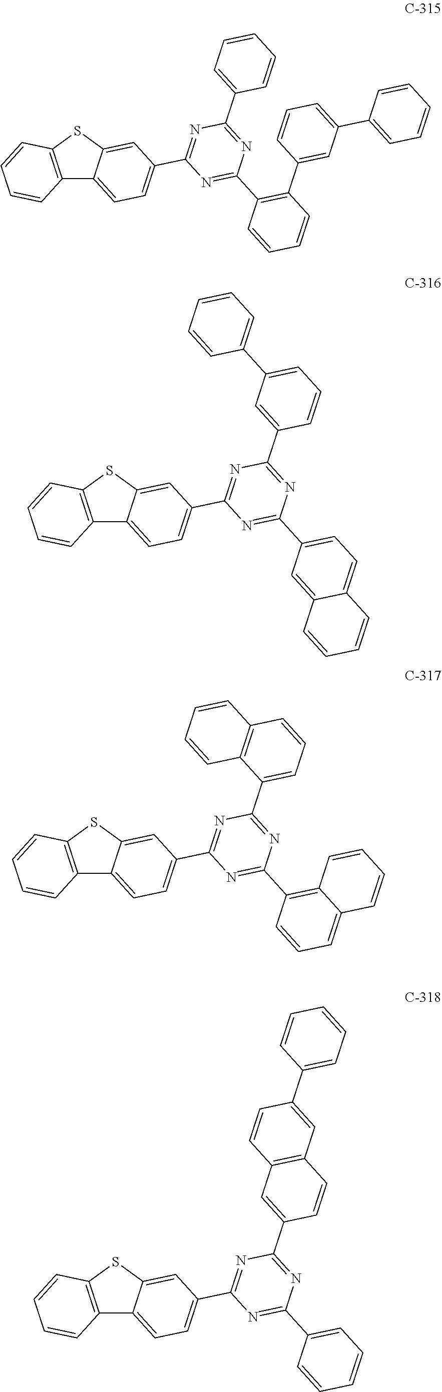 [SMILES: c1ccc(-c2cccc(-c3ccccc3-c3nc(-c4ccccc4)nc(-c4ccc5c(c4)sc4ccccc45)n3)c2)cc1 ; c1ccc(-c2cccc(-c3nc(-c4ccc5ccccc5c4)nc(-c4ccc5c(c4)sc4ccccc45)n3)c2)cc1 ; c1ccc2c(-c3nc(-c4ccc5c(c4)sc4ccccc45)nc(-c4cccc5ccccc45)n3)cccc2c1 ; c1ccc(-c2ccc3cc(-c4nc(-c5ccccc5)nc(-c5ccc6c(c5)sc5ccccc56)n4)ccc3c2)cc1]

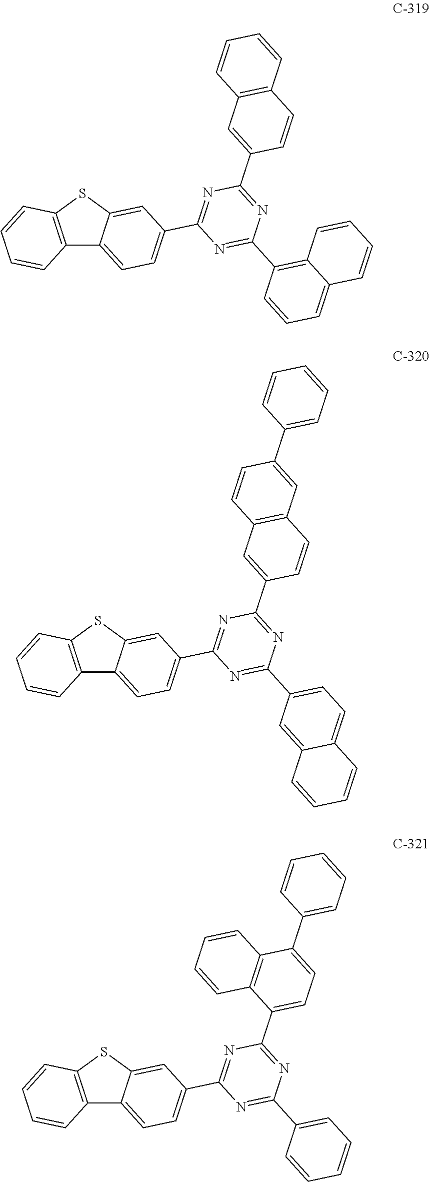

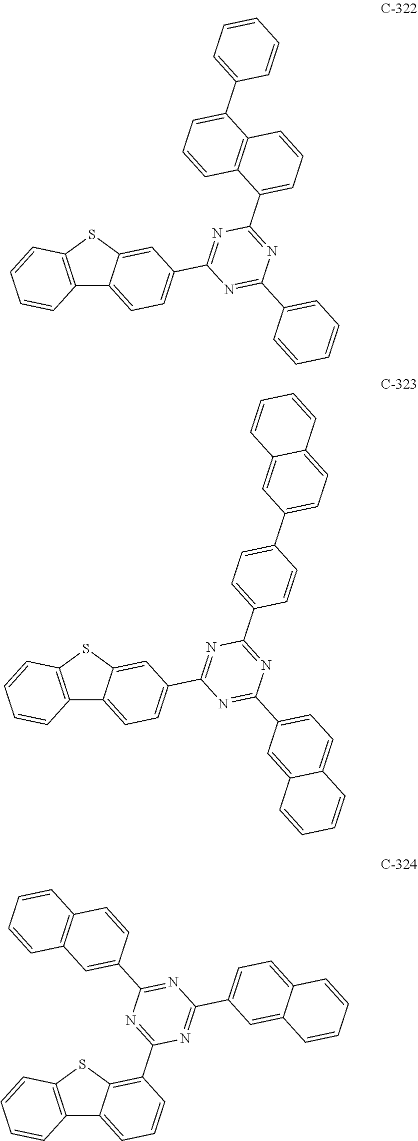

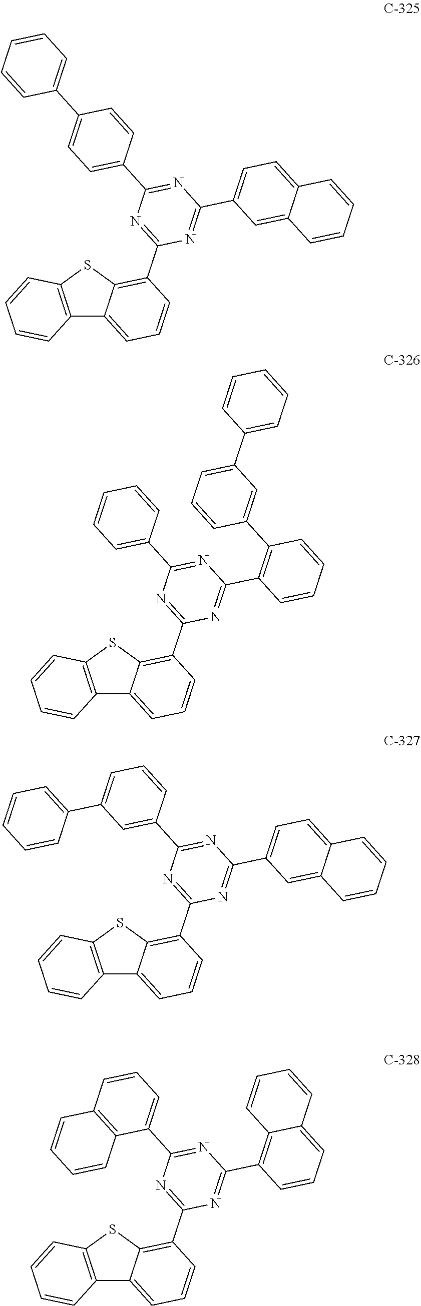 [SMILES: c1ccc(-c2ccc(-c3nc(-c4ccc5ccccc5c4)nc(-c4cccc5c4sc4ccccc45)n3)cc2)cc1 ; c1ccc(-c2cccc(-c3ccccc3-c3nc(-c4ccccc4)nc(-c4cccc5c4sc4ccccc45)n3)c2)cc1 ; c1ccc(-c2cccc(-c3nc(-c4ccc5ccccc5c4)nc(-c4cccc5c4sc4ccccc45)n3)c2)cc1 ; c1ccc2c(-c3nc(-c4cccc5ccccc45)nc(-c4cccc5c4sc4ccccc45)n3)cccc2c1]

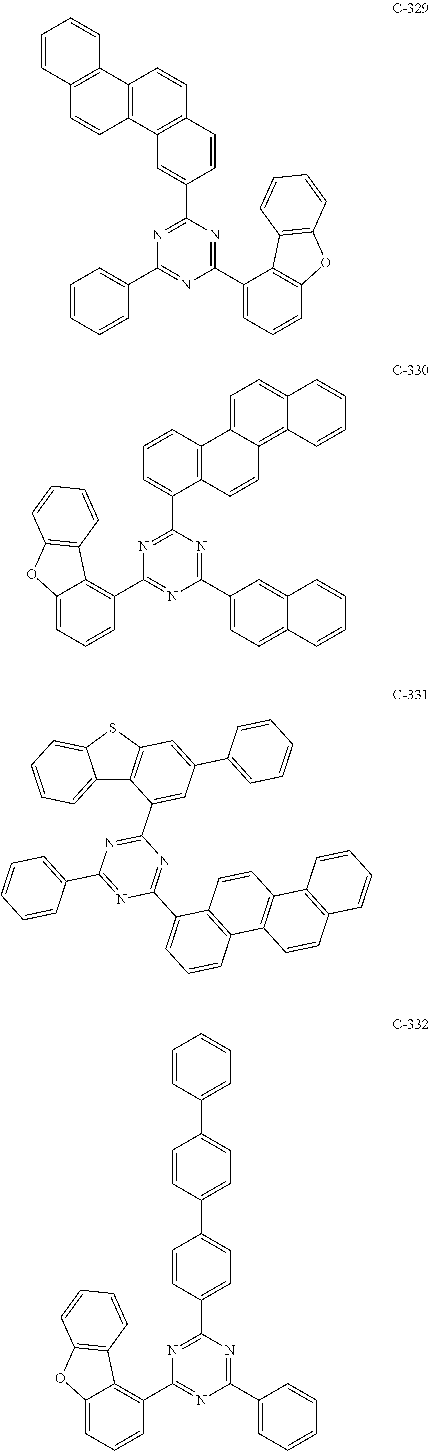 [SMILES: c1ccc(-c2nc(-c3ccc4ccc5c6ccccc6ccc5c4c3)nc(-c3cccc4oc5ccccc5c34)n2)cc1 ; c1ccc2cc(-c3nc(-c4cccc5c4ccc4c6ccccc6ccc54)nc(-c4cccc5oc6ccccc6c45)n3)ccc2c1 ; c1ccc(-c2cc(-c3nc(-c4ccccc4)nc(-c4cccc5c4ccc4c6ccccc6ccc54)n3)c3c(c2)sc2ccccc23)cc1 ; c1ccc(-c2ccc(-c3ccc(-c4nc(-c5ccccc5)nc(-c5cccc6oc7ccccc7c56)n4)cc3)cc2)cc1]

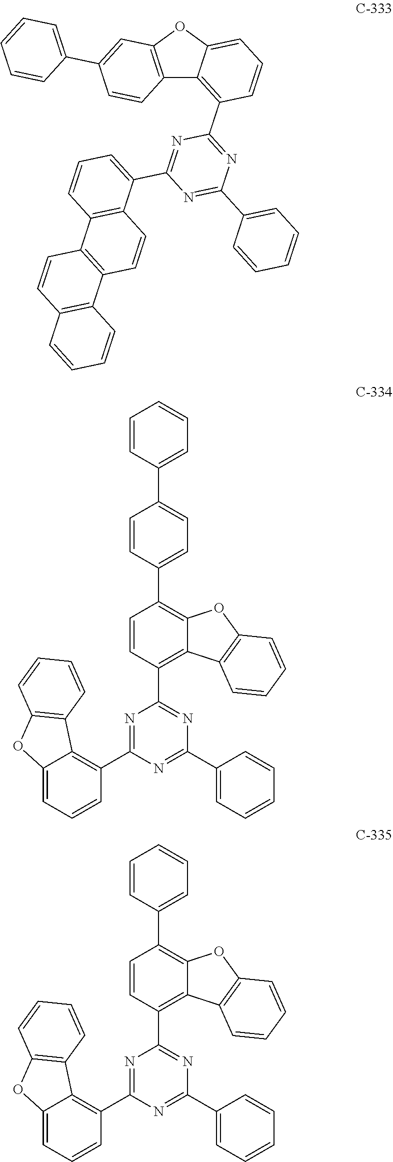 [SMILES: c1ccc(-c2ccc3c(c2)oc2cccc(-c4nc(-c5ccccc5)nc(-c5cccc6c5ccc5c7ccccc7ccc65)n4)c23)cc1 ; c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1]

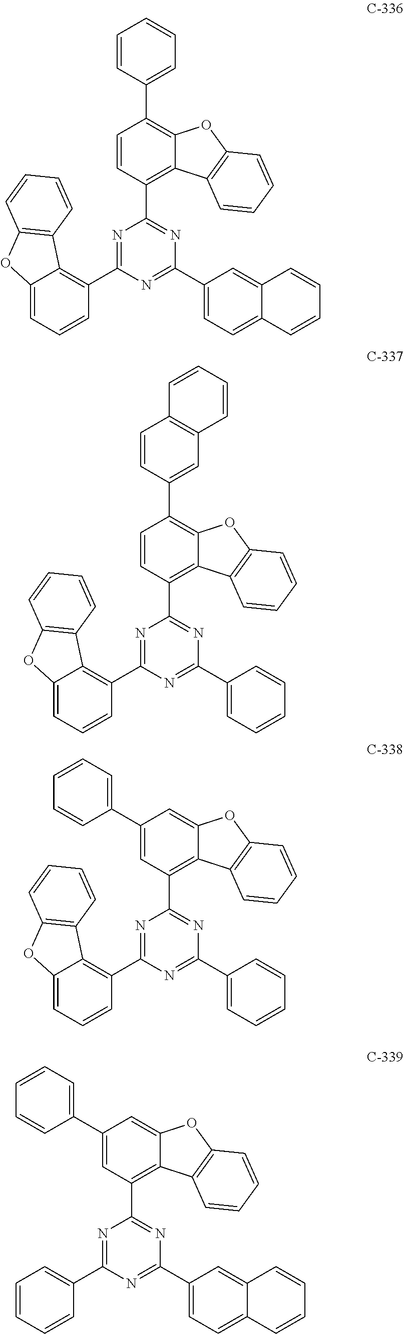

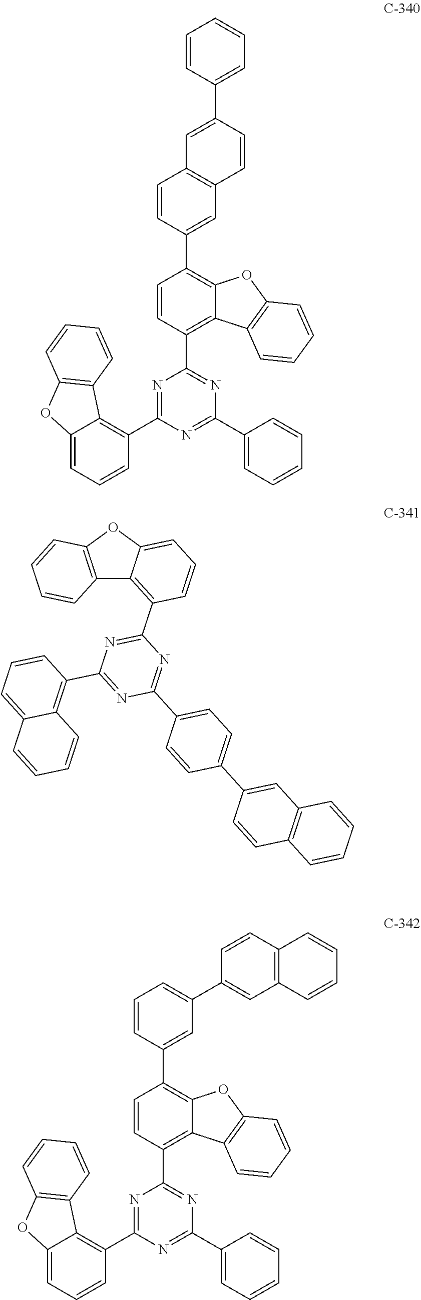

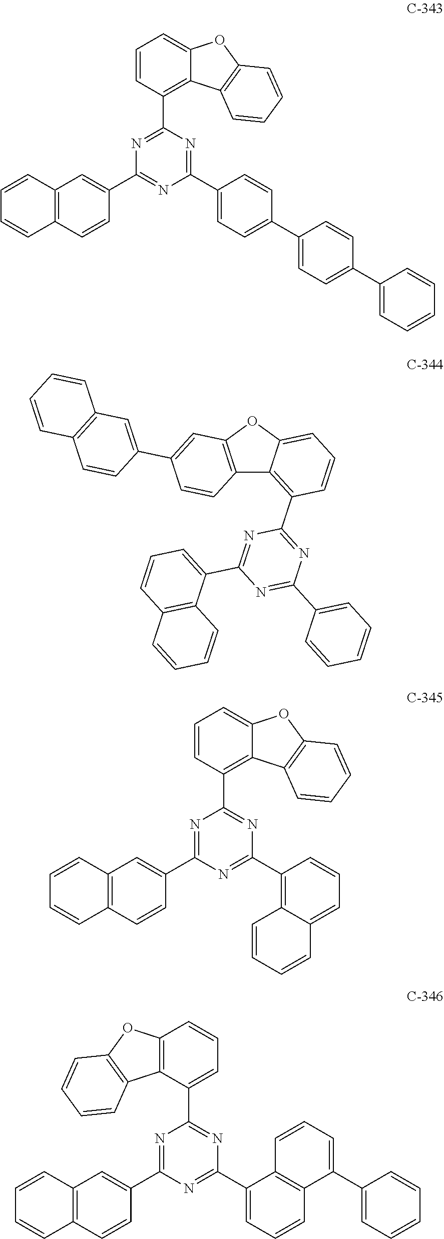 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2nc(-c3cccc4ccccc34)nc(-c3cccc4oc5cc(-c6ccc7ccccc7c6)ccc5c34)n2)cc1 ; c1ccc2cc(-c3nc(-c4cccc5ccccc45)nc(-c4cccc5oc6ccccc6c45)n3)ccc2c1 ; c1ccc(-c2cccc3c(-c4nc(-c5ccc6ccccc6c5)nc(-c5cccc6oc7ccccc7c56)n4)cccc23)cc1]

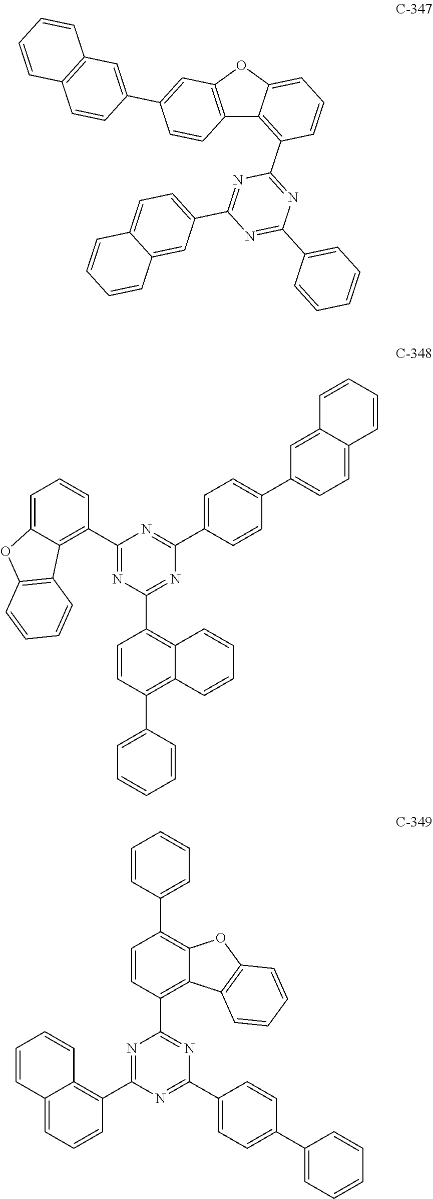

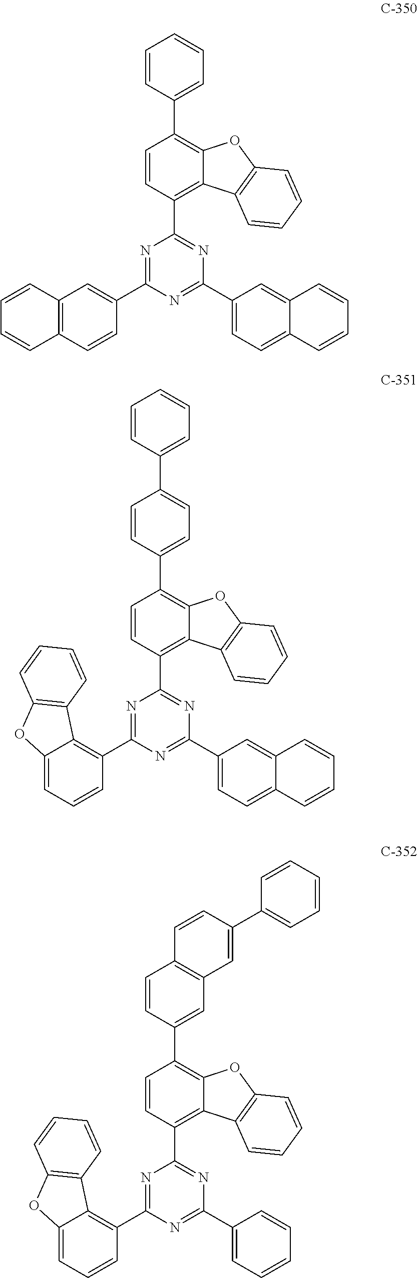 [SMILES: c1ccc(-c2ccc(-c3nc(-c4ccc5ccccc5c4)nc(-c4ccc5ccccc5c4)n3)c3c2oc2ccccc23)cc1 ; c1ccc(-c2ccc(-c3ccc(-c4nc(-c5ccc6ccccc6c5)nc(-c5cccc6oc7ccccc7c56)n4)c4c3oc3ccccc34)cc2)cc1 ; c1ccc(-c2ccc3ccc(-c4ccc(-c5nc(-c6ccccc6)nc(-c6cccc7oc8ccccc8c67)n5)c5c4oc4ccccc45)cc3c2)cc1]

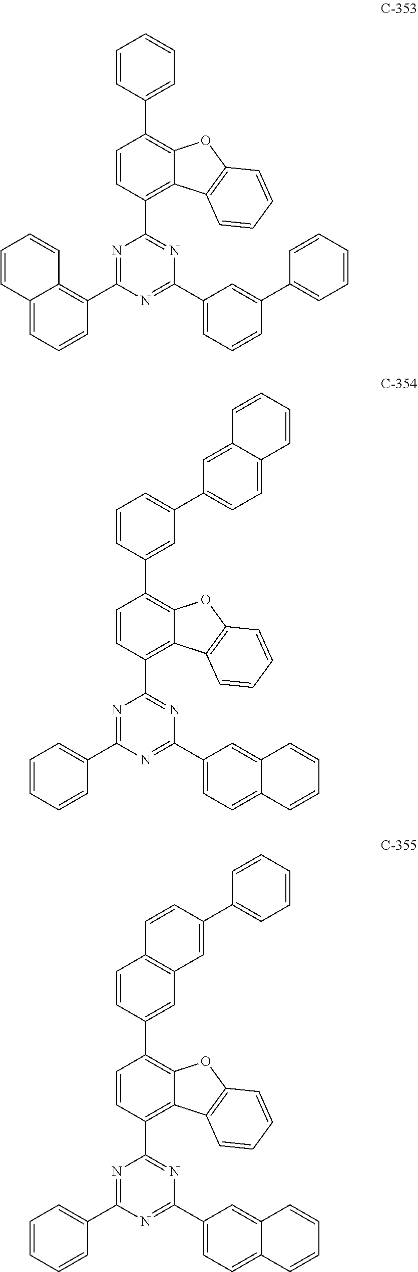 [SMILES: c1ccc(-c2cccc(-c3nc(-c4cccc5ccccc45)nc(-c4ccc(-c5ccccc5)c5oc6ccccc6c45)n3)c2)cc1 ; c1ccc(-c2nc(-c3ccc4ccccc4c3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccc7ccccc7c6)n5)c5c4oc4ccccc45)cc3c2)cc1]

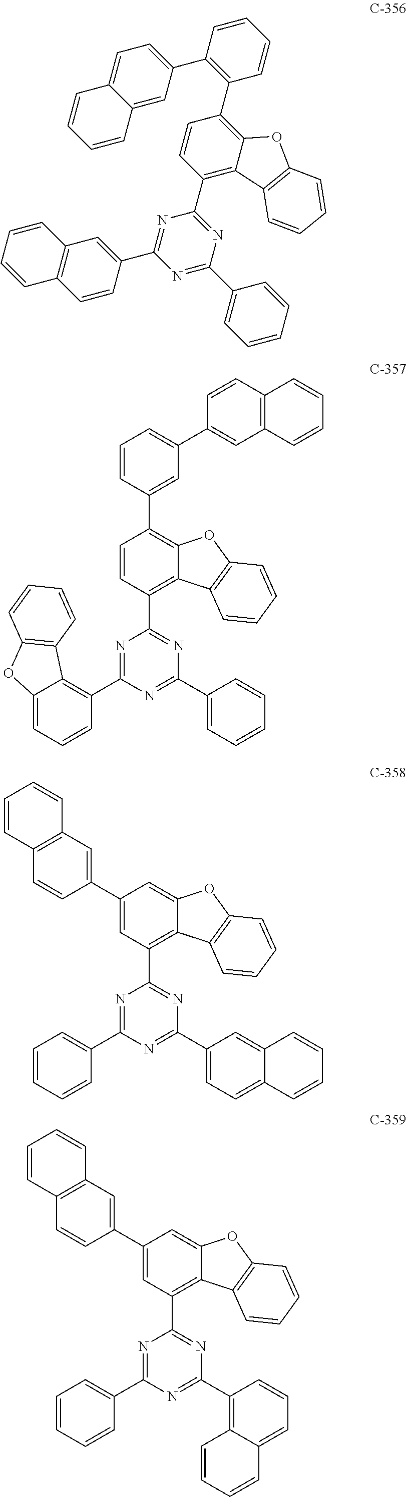

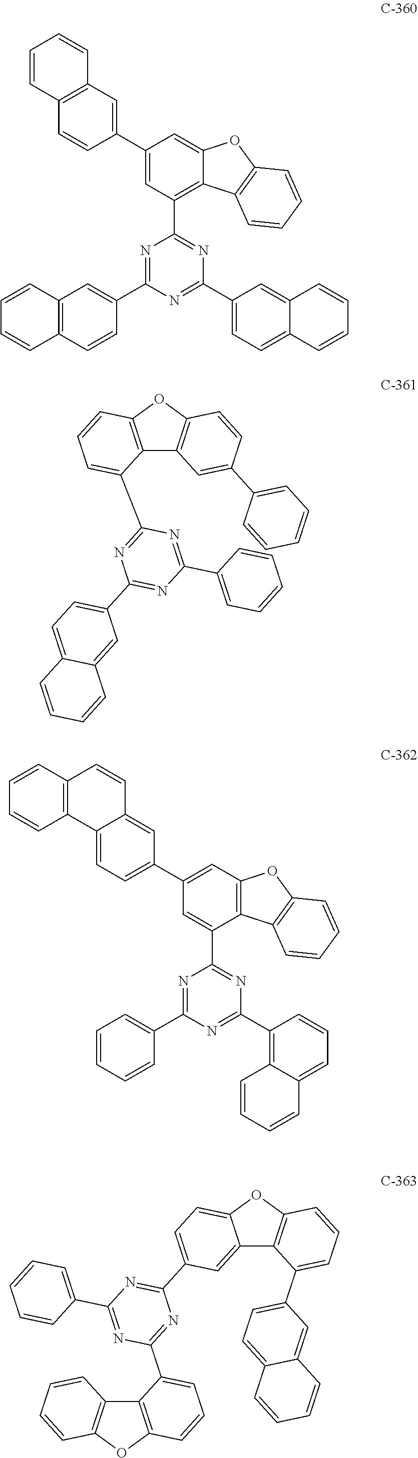

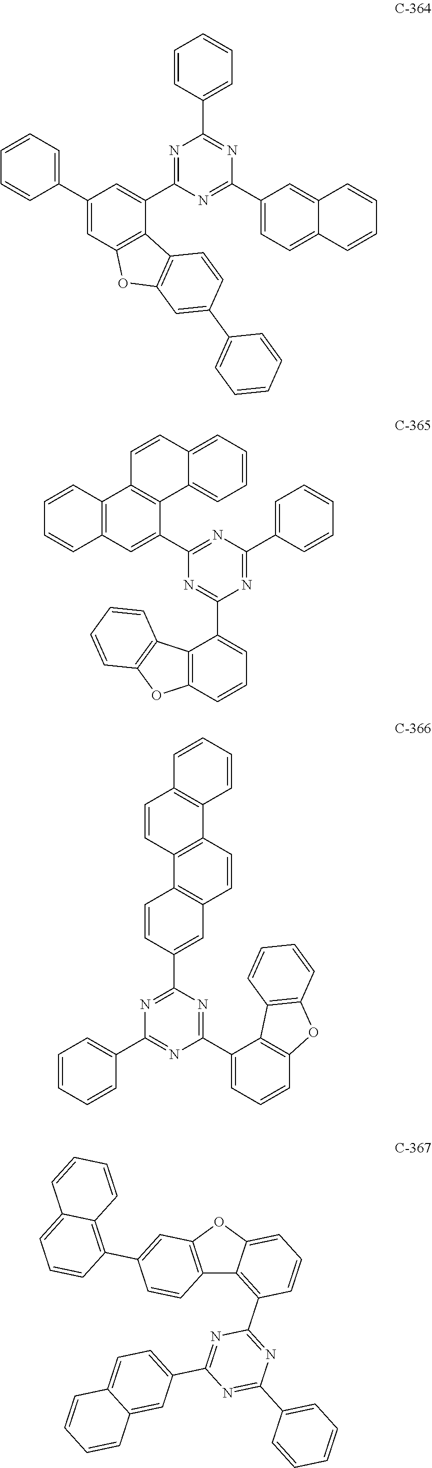

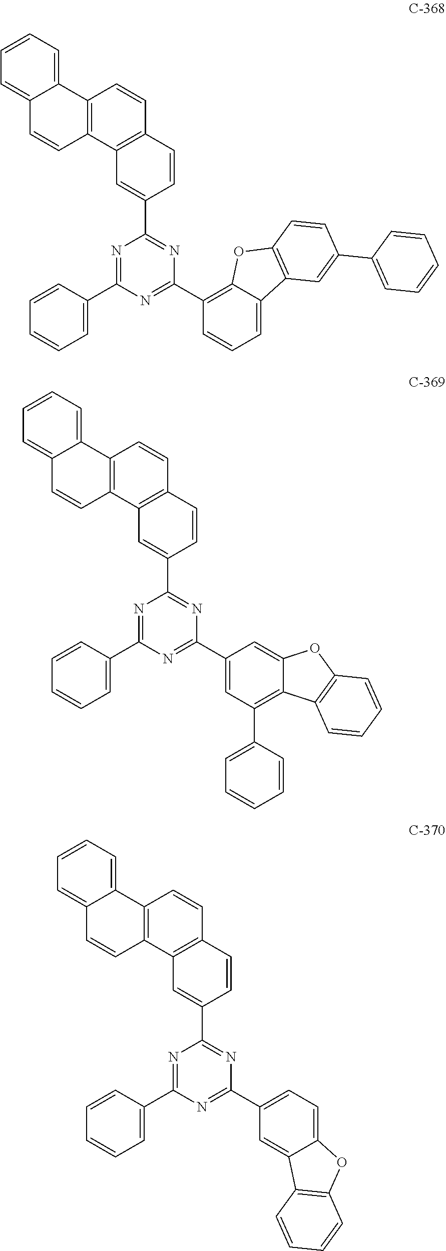

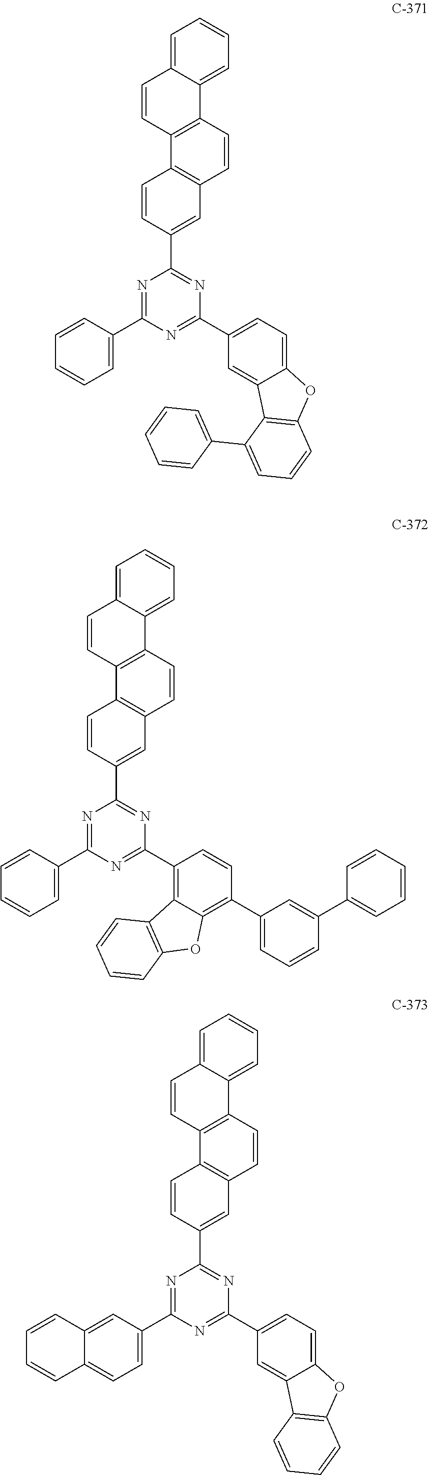 [SMILES: c1ccc(-c2nc(-c3ccc4c(ccc5c6ccccc6ccc45)c3)nc(-c3ccc4oc5cccc(-c6ccccc6)c5c4c3)n2)cc1 ; c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc2cc(-c3nc(-c4ccc5c(ccc6c7ccccc7ccc56)c4)nc(-c4ccc5oc6ccccc6c5c4)n3)ccc2c1]

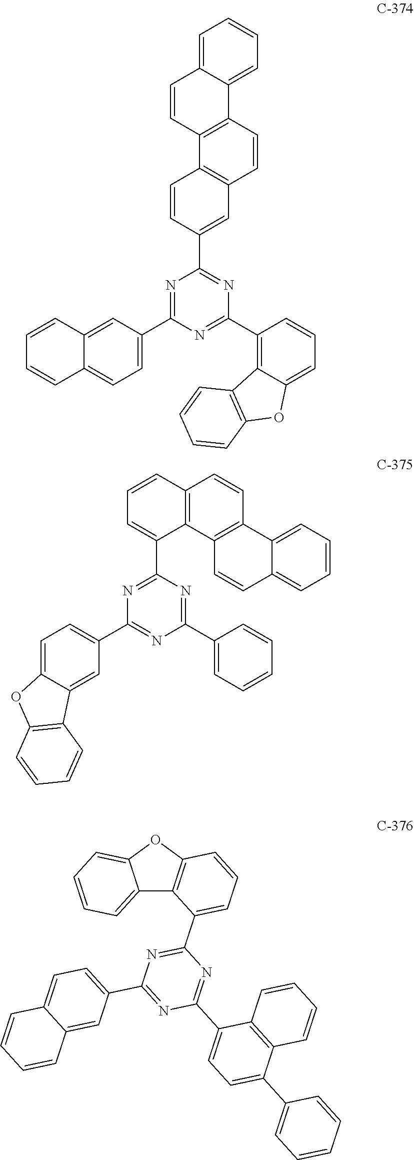

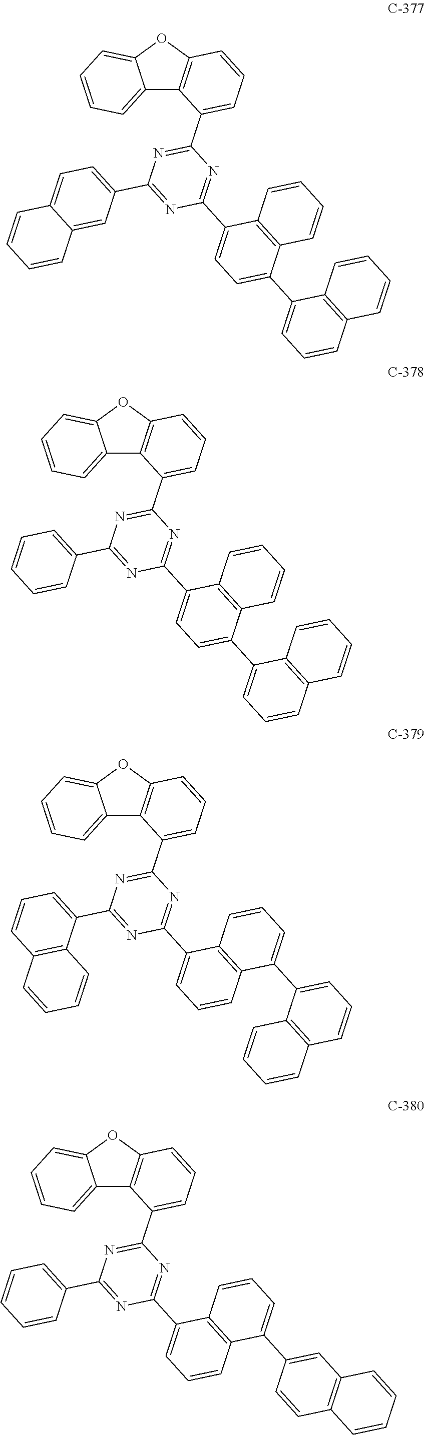 [SMILES: c1ccc2cc(-c3nc(-c4ccc(-c5cccc6ccccc56)c5ccccc45)nc(-c4cccc5oc6ccccc6c45)n3)ccc2c1 ; c1ccc(-c2nc(-c3ccc(-c4cccc5ccccc45)c4ccccc34)nc(-c3cccc4oc5ccccc5c34)n2)cc1 ; c1ccc2c(-c3nc(-c4cccc5c(-c6cccc7ccccc67)cccc45)nc(-c4cccc5oc6ccccc6c45)n3)cccc2c1 ; c1ccc(-c2nc(-c3cccc4c(-c5ccc6ccccc6c5)cccc34)nc(-c3cccc4oc5ccccc5c34)n2)cc1]

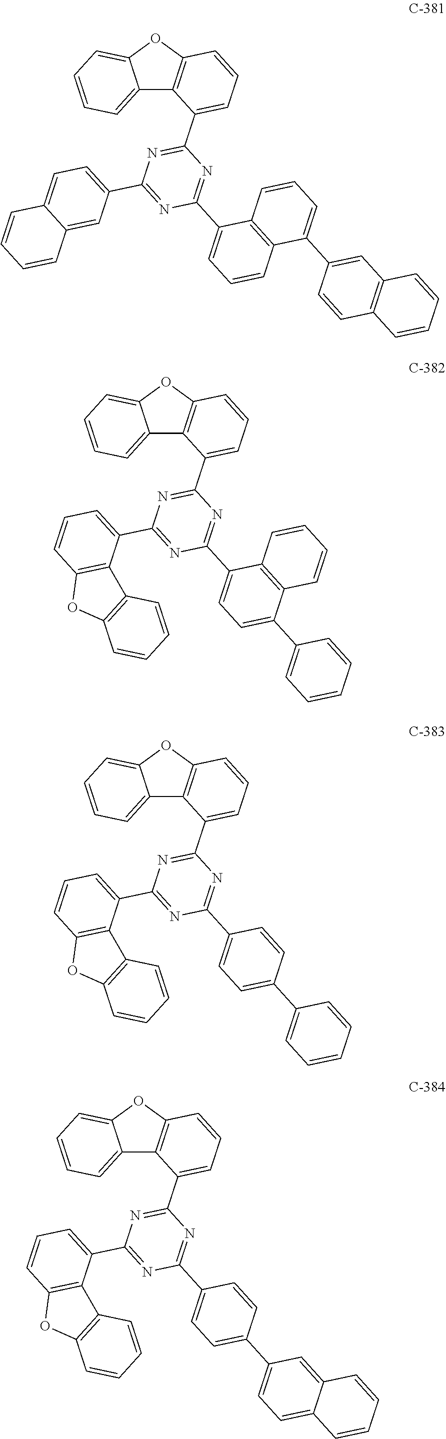 [SMILES: c1ccc2cc(-c3nc(-c4cccc5c(-c6ccc7ccccc7c6)cccc45)nc(-c4cccc5oc6ccccc6c45)n3)ccc2c1 ; c1ccc(-c2ccc(-c3nc(-c4cccc5oc6ccccc6c45)nc(-c4cccc5oc6ccccc6c45)n3)c3ccccc23)cc1 ; c1ccc(-c2ccc(-c3nc(-c4cccc5oc6ccccc6c45)nc(-c4cccc5oc6ccccc6c45)n3)cc2)cc1 ; c1ccc2cc(-c3ccc(-c4nc(-c5cccc6oc7ccccc7c56)nc(-c5cccc6oc7ccccc7c56)n4)cc3)ccc2c1]

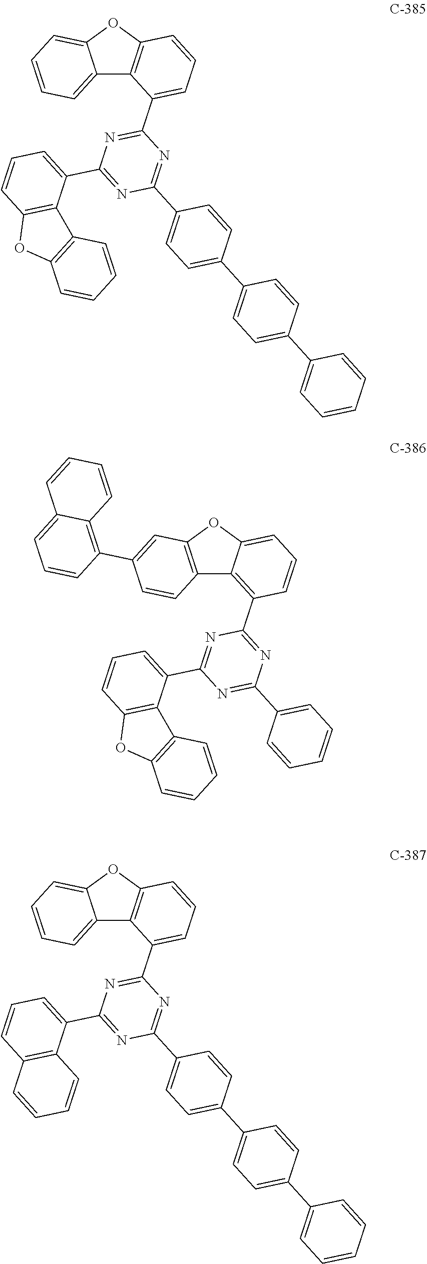 [SMILES: c1ccc(-c2ccc(-c3ccc(-c4nc(-c5cccc6oc7ccccc7c56)nc(-c5cccc6oc7ccccc7c56)n4)cc3)cc2)cc1 ; c1ccc(-c2nc(-c3cccc4oc5ccccc5c34)nc(-c3cccc4oc5cc(-c6cccc7ccccc67)ccc5c34)n2)cc1 ; c1ccc(-c2ccc(-c3ccc(-c4nc(-c5cccc6ccccc56)nc(-c5cccc6oc7ccccc7c56)n4)cc3)cc2)cc1]

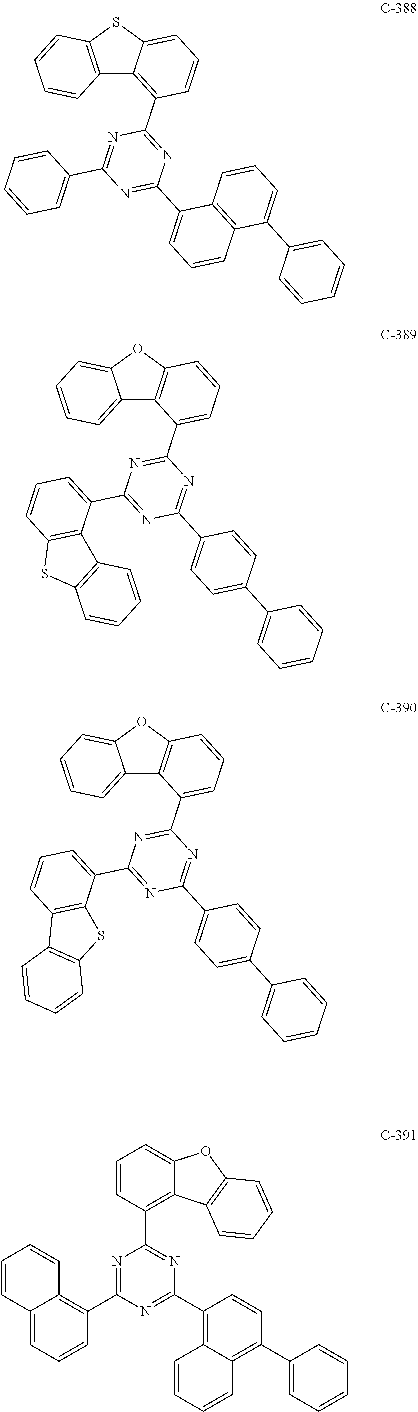 [SMILES: c1ccc(-c2nc(-c3cccc4c(-c5ccccc5)cccc34)nc(-c3cccc4sc5ccccc5c34)n2)cc1 ; c1ccc(-c2ccc(-c3nc(-c4cccc5oc6ccccc6c45)nc(-c4cccc5sc6ccccc6c45)n3)cc2)cc1 ; c1ccc(-c2ccc(-c3nc(-c4cccc5c4sc4ccccc45)nc(-c4cccc5oc6ccccc6c45)n3)cc2)cc1 ; c1ccc(-c2ccc(-c3nc(-c4cccc5ccccc45)nc(-c4cccc5oc6ccccc6c45)n3)c3ccccc23)cc1]

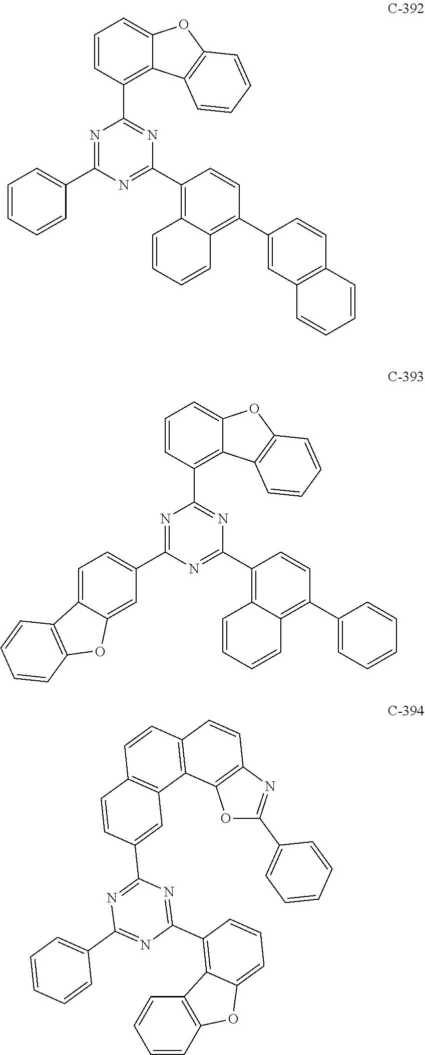 [SMILES: c1ccc(-c2nc(-c3ccc(-c4ccc5ccccc5c4)c4ccccc34)nc(-c3cccc4oc5ccccc5c34)n2)cc1 ; c1ccc(-c2ccc(-c3nc(-c4ccc5c(c4)oc4ccccc45)nc(-c4cccc5oc6ccccc6c45)n3)c3ccccc23)cc1 ; c1ccc(-c2nc(-c3ccc4ccc5ccc6nc(-c7ccccc7)oc6c5c4c3)nc(-c3cccc4oc5ccccc5c34)n2)cc1]

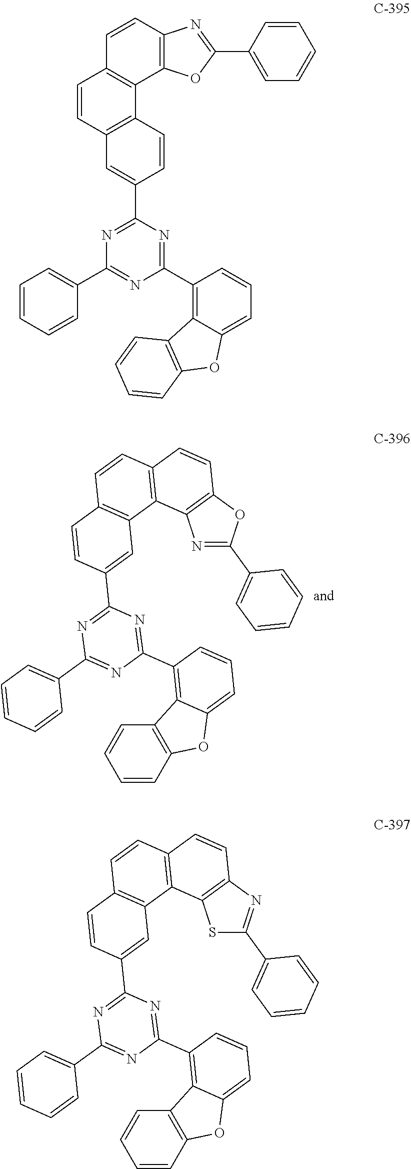 [SMILES: c1ccc(-c2nc(-c3ccc4c(ccc5ccc6nc(-c7ccccc7)oc6c54)c3)nc(-c3cccc4oc5ccccc5c34)n2)cc1 ; c1ccc(-c2nc(-c3ccc4ccc5ccc6oc(-c7ccccc7)nc6c5c4c3)nc(-c3cccc4oc5ccccc5c34)n2)cc1 ; c1ccc(-c2nc(-c3ccc4ccc5ccc6nc(-c7ccccc7)sc6c5c4c3)nc(-c3cccc4oc5ccccc5c34)n2)cc1]

8. An organic electroluminescent device comprising an anode; a cathode; and at least one light-emitting layer between the anode and the cathode, wherein the at least one light-emitting layer comprises the plurality of host materials according to claim 1.
